SEQUENCE LISTING (2 sequences)

SEQ ID NO: 1 - protein, 6 aa, Artificial
  Synthetic peptide
LXXLAP

SEQ ID NO: 2 - protein, 19 aa, Artificial
  Synthetic peptide
DLDLEALAPYIPADDDFQL

(19) United States
(12) Patent Application Publication
(10) Pub. No.: US 2009/0011051 A1
(43) Pub. Date: Jan. 8, 2009

(54) Methods, Compositions and Articles of Manufacture for HIF Modulating Compounds

(76) Inventors: Mark B. Roth, Seattle, WA (US); Mark Budde, Seattle, WA (US)

Correspondence Address: FULBRIGHT & JAWORSKI L.L.P., 600 CONGRESS AVE., SUITE 2400, AUSTIN, TX, 78701, US

(21) Appl. No.: 11/864,355

(22) Filed: Sep. 28, 2007

Related U.S. Application Data
(60) Provisional application No. 60/827,337, filed on Sep. 28, 2006.

(51) Int. Cl. A61K 33/04; A61K 31/47; A61K 31/17; A61K 31/145; A61K 31/473; A61K 31/502; C12N 5/02; A61K 33/00

(52) U.S. Cl. 424/699; 514/311; 514/597; 514/608; 514/292; 514/248; 435/374; 424/713

(57) ABSTRACT

The present invention generally relates to the modulation of hypoxia-inducible factor (HIF) using the compounds and methods disclosed herein. These compounds and methods can be applied to the prevention, pretreatment, and/or treatment of conditions or states associated with HIF, such as hypoxia- and ischemia-related conditions and the induction of stasis.

This application claims priority to U.S. provisional patent application 60/827,337 filed on Sep. 28, 2006, which is hereby incorporated by reference in its entirety.

This invention was made with government support under grant number GM048435 from the National Institute of General Medical Sciences (NIGMS). The government has certain rights in the invention.

### BACKGROUND OF THE INVENTION

I. Field of the Invention

The present invention relates generally to the field of cell biology and physiology. More particularly, it concerns methods, compositions and apparatuses for enhancing survivability of and/or reducing damage to cells, tissues, organs, and organisms, particularly under adverse conditions, including but not limited to hypoxic or ischemic states, using one or more substances, including those that compete with oxygen. In certain embodiments, the present invention includes methods, compositions and apparatuses for treating, preventing, and diagnosing diseases and conditions by exposing a subject to a compound, such as an oxygen antagonist, protective metabolic agent, or other chemical compound discussed herein, or a precursor thereof, that can achieve its stated goal (collectively referred to as “Effective Compounds”). More broadly, the methods, compositions and apparatuses of the present invention relate to the modulation of the α-subunit of hypoxia inducible factor (HIF), an oxygen-responsive transcriptional activator.

II. Description of Related Art

As discussed herein, hypoxia and ischemia are two physiological states characterized by reductions in oxygen and blood flow, respectively. Typically, hypoxic and ischemic states are unfavorable, and treatments exist to combat these states and their related conditions, such as anemia, myocardial infarction, stroke and occlusive arterial disease. However, situations may exist wherein hypoxic and/or ischemic states may be preferable, such as during organ transplantation or prior to the inducement of trauma.

Currently, treatment of ischemic and hypoxic disorders is focused on relief of symptoms and treatment of causative disorders. For example, treatments for myocardial infarction include nitroglycerin and analgesics to control pain and relieve the workload of the heart. Other medications, including digoxin, diuretics, amrinone, β-blockers, lipid-lowering agents and angiotensin-converting enzyme inhibitors, are used to stabilize the condition, but none of these therapies directly address the tissue damage produced by the ischemia and hypoxia.

Due to deficiencies in current treatments, there remains a need for compounds that are effective in treating hypoxia- and ischemia-related conditions. For example, there is a need for compounds that are effective in treating erythropoietin-associated conditions, which can be categorized as hypoxia-related conditions, such as anemia, including anemia associated with diabetes, ulcers, kidney failure, cancer, infection, dialysis, surgery, and chemotherapy. Other conditions involving ischemia and hypoxia include, for example, occlusive arterial disease, angina pectoris, intestinal infarctions, pulmonary infarctions, cerebral ischemia, and myocardial infarction. There is also a need for compounds that are effective in the prevention of tissue damage caused by these conditions.

One oxygen-regulated protein involved in hypoxia and ischemia is hypoxia inducible factor (HIF), a protein comprised of two subunits, the α-subunit of which is oxygen-regulated. As described in more detail herein, under normoxic conditions, HIFα is hydroxylated and degraded but under hypoxic conditions, HIFα is not degraded and forms a complex with HIFβ. HIFα/β then regulates the expression of certain genes, such as erythropoietin (EPO). Since the accumulation of HIFα is associated with hypoxic and ischemic conditions, HIFα presents itself as a viable target for treatment of deleterious hypoxic and ischemic conditions as well as for the intentional inducement of such conditions that may be beneficial for a tissue, organ or organism. Indeed, compounds have been found that can modulate these conditions via HIF regulation. See, e.g., U.S. Pat. Appln. 2003/0176317; U.S. Pat. Appln. 2004/0254215, each of which is incorporated herein by reference in its entirety.

Stasis is another physiological state that may be associated with reductions in oxygen and blood flow. As described herein, in “stasis” or “suspended animation,” a cell, tissue or organ, or organism (collectively referred to as “biological material”) is living, but cellular functions necessary for cell division, developmental progression, and/or metabolic state are slowed or even stopped. This state is desirable in a number of contexts. Stasis can be used as a method of preservation by itself, or it may be induced as part of a cryopreservation regimen. For example, biological materials may be preserved for research use, for transportation, for transplantation, for therapeutic treatment (such as ex vivo therapy), and to prevent the onset of trauma. In another example, tissue culture cells are often stored for periods of time in tanks that hold liquid nitrogen; however, these tanks frequently require that the liquid nitrogen in the unit be periodically replaced, otherwise it becomes depleted and the temperature is not maintained. Furthermore, damage to cells and tissue occurs as a result of the freeze/thaw process. Thus, improved techniques are needed.

Stasis with respect to entire organisms has similar uses. For instance, transportation of organisms could be facilitated if they had entered stasis. This might reduce physical and physiological damage to the organism by reducing or eliminating stress or physical injury. Stasis may be beneficial by decreasing the need of the biological material for oxygen and, therefore, bloodflow. Moreover, the lack of ability to control cellular and physiologic metabolism in whole organisms subjected to traumas such as amputation and hypothermia is a key shortcoming in the medical field. On the other hand, the anecdotal evidence discussed above and herein strongly suggests that if properly understood and regulated, it is possible to induce stasis in cells, tissues and whole organisms. Thus, there is a great need for improved methods for controlling metabolic processes particularly under traumatic conditions. As with the HIFα-related conditions described above, compounds have been found that can modulate stasis. See, e.g., U.S. Appln. 11/408,734, incorporated herein by reference in its entirety.

As discussed, the conditions of hypoxia, ischemia and stasis all involve reductions in physiological aspects such as oxygen, blood flow and metabolic rates. Thus far, no intercorrelation between each of these conditions and HIF has been suggested or detected. If such a connection could be found, the physiology of these conditions as related to HIF behavior would become clearer, and application and treatment options could be expanded. For example, compounds used in the treatment of one condition, such as a hypoxia-related condition, may prove useful for the inducement of stasis as well.

### SUMMARY OF THE INVENTION

The inventors have discovered that certain compounds shown to affect metabolic activity also act to stabilize HIF, thereby implicating such compounds as preventative and treatment regimes for hypoxia, ischemia, stasis, and/or any other condition or state associated with HIF stabilization, such as hemorrhagic shock. See, e.g., FIG. 1 and FIG. 2. In general aspects, then, the present invention relates to the interrelationship among hypoxic, ischemic and stasis conditions, based on the common feature of cellular HIF modulation by compounds and methods of the present invention. Thus, the invention is generally drawn to compounds and methods that modulate HIF activity, such as by stabilizing HIF via the inhibition of HIF hydroxylation and thereby, in some embodiments, activating HIF-regulated gene expression. The methods can be applied to the prevention, pretreatment, and/or treatment of conditions or states associated with HIF, such as stasis and anemic, ischemic and hypoxic conditions. These methods can be applied in vivo or in vitro.

This application is related by inventor and/or subject matter to U.S. Provisional Patent Applications 60/673,037 and 60/673,295 both filed on Apr. 20, 2005, as well as U.S. Provisional Patent Application 60/713,073, filed Aug. 31, 2005, U.S. Provisional Patent Application 60/731,549, filed Oct. 28, 2005, and U.S. Provisional Patent Application 60/762,462, filed on Jan. 26, 2006, all of which are hereby incorporated by reference in their entirety.

### I. STABILIZATION OF HIF

The transcription factor HIF (hypoxia inducible factor) system is a key regulator of responses to hypoxia, occupying a central position in oxygen homeostasis in a wide range of organisms. A large number of transcriptional targets have been identified, with critical roles in angiogenesis, erythropoiesis, energy metabolism, inflammation, vasomotor function, and apoptotic/proliferative responses. The system is essential for normal development, and plays a key role in pathophysiological responses to ischaemia/hypoxia. HIF is also important in cancer, in which it is commonly upregulated, has major effects on tumor growth and angiogenesis. The HIF DNA binding complex consists of a heterodimer of α and β subunits. Regulation by oxygen occurs through hydroxylation of the α-subunits, typically of one or more proline residues, which are then rapidly destroyed by the proteasome in oxygenated cells. This involves binding of HIFα-subunits by the von Hippel-Lindau tumor suppressor protein (pVHL or VHL), with pVHL acting as the, or part of the, recognition component for a ubiquitin ligase that promotes ubiquitin dependent proteolysis through interaction with a specific sequence or sequences in HIF-α-subunits. In hypoxia, this process is suppressed, thereby stabilizing HIFα and permitting transcriptional activation via the HIFα/β heterodimer.

Accordingly, the present invention relates generally to methods of stabilizing the α-subunit of HIF. In certain embodiments of the present invention, the HIFα is selected from the group consisting of HIF-1α, HIF-2α, HIF-3α and any fragment thereof. The present invention provides, in one aspect, methods for stabilizing endogenous HIFα. Thus, in a particular embodiment, the HIFα is endogenous to the subject. In certain embodiments, stabilization occurs via inhibition of HIF prolyl hydroxlation. Embodiments of the present invention include methods for stabilizing HIFα in which a compound that stabilizes HIFα is administered to a subject in vivo. The subject can be, for example, an animal, such as a mammal, wherein the mammal may be, for example, a human. Methods of ex vivo administration are also contemplated. In such methods, the subject can be, e.g., a cell, tissue, or organ, etc. In certain embodiments, the subject is a cell, tissue, or organ derived from a system such as the renal, cardiac, hepatic, pulmonary, hematopoietic, gastrointestinal, neuronal, or musculoskeletal system, etc.

Methods for treating, preventing, or pretreating a HIF-associated condition are also provided. In particular, the present invention provides a method for treating, preventing, or pretreating a HIF-associated condition, the method comprising stabilizing HIFα. In specific aspects, the invention provides a method for treatment, prevention, or pretreatment/preconditioning of a HIF-associated condition in a subject, the method comprising stabilization of HIFα. In various aspects, the HIF-associated condition is associated with ischemia or hypoxia. In other aspects, the HIF-associated condition is associated with stasis. In some aspects, the method comprises administering to the subject an Effective Compound, as described herein, that stabilizes HIFα. Methods of administration of one or more Effective Compounds are described herein.

The following sections briefly describe certain embodiments of the present invention. Further details and additional embodiments are provided later in this disclosure.

A. Inhibition of HIF Hydroxylation

Inhibition of HIFα hydroxylation has been shown to result in therapeutic benefits. See, e.g., Freeman et al., 2003; Welsh et al., 2003; Siddiq et al., 2005. As described herein, the purpose of the modulation (e.g., interaction, disruption, interference) of the hydroxylation of HIFα may be to, for example, induce stasis and/or modulate cellular functions such as angiogenesis, erythropoiesis, energy metabolism, inflammation, matrix metabolism, vasomotor function, and apoptotic/proliferative responses and pathophysiological responses to ischemia/hypoxia, all of which are mediated by HIFα as discussed herein.

Accordingly, in one embodiment of the present invention, the method of stabilizing the α-subunit of HIF (HIFα) comprises administering to a subject a compound that inhibits hydroxylation of HIFα. In a further embodiment, the method comprises administering to a subject a compound that inhibits 2-oxoglutarate dioxygenase enzyme activity. In various embodiments, the 2-oxoglutarate dioxygenase enzyme is selected from the group consisting of EGLN1, EGLN2, EGLN3, EGLN9 (also called Egl-9), procollagen prolyl 4-hydroxylase, procollagen prolyl 3-hydroxylase, procollagen lysyl hydroxylase, PHD4, FIH-1, and any subunit or fragment thereof, respectively.

In particular methods for stabilizing HIFα according to the present invention, the methods comprise inhibiting HIF prolyl hydroxylase enzyme activity. In further embodiments, the HIF prolyl hydroxylase enzyme is selected from the group consisting of EGLN1, EGLN2, EGLN3, EGLN9, and any subunit or fragment thereof, respectively.

In one method of stabilizing HIFα according to the present invention, the compound stabilizes HIFα by specifically inhibiting hydroxylation of at least one amino acid residue in HIFα. In a further aspect, the amino acid residue is selected from the group consisting of proline and asparagine.

In one specific embodiment, the agent inhibits hydroxylation of the HIF-1α P₅₆₄ residue or a homologous proline in another HIFα isoform. In another specific embodiment, the agent inhibits hydroxylation of the HIF-1α P₄₀₂ residue or a homologous proline in another HIFα isoform. In yet another embodiment, the compound may additionally inhibit hydroxylation of one or more HIFα asparagine residues. In one specific embodiment, the agent inhibits hydroxylation of the HIF-1α N₈₀₃ residue or a homologous asparagine residue in another HIFα isoform.

B. Methods of Treating, Preventing or Pretreating Certain HIF-Associated Conditions

Methods for treating, preventing, or pretreating a HIF-associated condition in a subject, the methods comprising inhibiting 2-oxoglutarate dioxygenase enzyme activity, are also provided, and include methods in which the HIF-associated condition is one associated with ischemia, hypoxia, or stasis, or any other condition described herein. In one aspect, the present invention provides a method for treating, preventing, or pretreating a HIF-associated condition, the method comprising administering to the subject a compound that inhibits 2-oxoglutarate dioxygenase enzyme activity.

In some embodiments, the present invention provides a method of treating, preventing, or pretreating a HIF-associated condition in a subject, the method comprising inhibiting HIF prolyl hydroxylase enzyme activity. Again, HIF-associated conditions include those associated with hypoxia, ischemia, or with stasis, etc., as described herein. In a particular embodiment, the method comprises administering to the subject a compound that inhibits HIF prolyl hydroxylase activity.

In a further embodiment, the method further comprises administering a second compound. In particular embodiments, the second compound inhibits 2-oxoglutarate dioxygenase enzyme activity, or the compound and the second compound inhibit the activities of different 2-oxoglutarate dioxygenase enzymes, or the second compound is selected from the group consisting of an ACE inhibitor (ACEI), angiotensin-II receptor blocker (ARB), diuretic, digoxin, statin, or carnitine, etc.

In specific embodiments, HIF-associated conditions include disorders such as pulmonary disorders, e.g., pulmonary embolism, etc., cardiac disorders, e.g., myocardial infarction, congestive heart failure, etc., neurological disorders and shock, e.g., hemorrhagic shock, and the like. The present invention thus clearly contemplates methods that can be applied to the treatment, prevention, or pretreatment/preconditioning of a HIF-associated condition associated with any ischemic event, whether acute or transient, or chronic. Acute ischemic events can include those associated with surgery, organ transplantation, infarction (e.g., cerebral, intestinal, myocardial, pulmonary, etc.), trauma, insult, or injury, etc. Chronic events associated with ischemia can include hypertension, diabetes, occlusive arterial disease, chronic venous insufficiency, Raynaud's disease, cirrhosis, congestive heart failure, systemic sclerosis, etc.

In specific embodiments, the HIF-associated condition induces stasis. In some embodiments, treatment of an HIF-associated condition with an Effective Compound, as described herein, induces stasis. Accordingly, in certain embodiments, the present invention provides methods, compositions, articles of manufacture, and apparatuses to induce stasis in cells, tissues and organs located within or derived from an organism, as well as in the organism itself. Such methods, compositions, articles of manufacture, and apparatuses can be employed to protect biological matter, as well as to prevent, treat, or diagnose diseases and conditions in the organism. In addition, such methods may directly induce stasis themselves, or they may act indirectly by not inducing stasis themselves, but by enhancing the ability of biological matter to enter stasis in response to an injury or disease condition, e.g., by reducing the time or level of injury or disease required to achieve stasis. Such a condition may be referred to as pre-stasis. Details of such applications and other uses are described below.

Methods of preconditioning or pretreating are specifically contemplated. In one embodiment, the invention provides methods of pretreating or preconditioning wherein HIFα is stabilized prior to the occurrence of an event associated with a HIF-associated condition, e.g., ischemia, etc., or the development of a HIF-associated condition. Ischemias can be induced by acute events. Such events can include, for example, surgery, e.g., angioplasty, organ transplantation, etc., and related procedures such as administration of anesthesia, etc. Furthermore, in certain specific embodiments, the methods of pretreating or preconditioning are applied in situations where a subject has a disorder predictive of the development of a HIF-associated condition, e.g., transient ischemic attack or angina pectoris, indicative of stroke and myocardial infarction, respectively, in order to prevent the development of or reduce the degree of development of the HIF-associated condition. In a particular embodiment, a compound that stabilizes HIFα is administered to a subject in order to increase preconditioning factors for ischemia, for example, EPO, etc.

In particular embodiments, methods and/or compounds of the present invention are used to induce stasis or pre-stasis in biological matter, e.g., cells, tissues, organs, and/or organisms, after an injury (e.g., a traumatic injury) or after the onset or progression of a disease, in order to protect the biological matter from damage associated with the injury or disease prior to, during, or following treatment of the injury or disease. In other embodiments, methods of the present invention are used to induce or promote stasis or pre-stasis in biological matter prior to subjection to an injurious event (e.g., an elective surgery) or prior to the onset or progression of a disease, in order to protect the biological matter from damage associated with adverse conditions such as injury or disease. In such cases, such methods are generally referred to as “pre-treatment” with an Effective Compound. Pre-treatment may include methods wherein biological matter is provided with an Effective Compound both before and during, and before, during and after biological matter is subjected to adverse conditions (e.g., an injury or onset or the progression of a disease), and methods wherein biological matter is provided with an Effective Compound only before biological matter is subjected to adverse conditions.

In another aspect, the methods of the invention are used to prevent tissue damage caused by HIF-associated disorders including, but not limited to, ischemic and hypoxic disorders. In one embodiment, treatment is predicated on predisposing conditions, e.g., hypertension, diabetes, occlusive arterial disease, chronic venous insufficiency, Raynaud's disease, cirrhosis, congestive heart failure, and/or systemic sclerosis.

In yet another aspect, the methods of the invention can be used as a pretreatment to decrease or prevent the tissue damage caused by HIF-associated disorders including, but not limited to, ischemic and hypoxic disorders. In one embodiment, the need for pretreatment is based on a patient's history of recurring episodes of an ischemic condition, e.g., myocardial infarction or transient ischemic attacks, or has symptoms of impending ischemia, e.g., angina pectoris, etc. In another embodiment, the need for pretreatment is based on physical parameters implicating possible or likely ischemia or hypoxia, such as is the case with, e.g., individuals placed under general anesthesia or temporarily working at high altitudes. In yet another embodiment, the methods may be used in the context of organ transplants to pretreat organ donors and to maintain organs removed from the body prior to implantation in a recipient.

C. Methods for Increasing HIF-Related Factors

Methods for increasing expression of various HIF-related factors are specifically contemplated herein. In one aspect, the present invention provides a method for increasing expression of angiogenic factors in a subject, the method comprising stabilizing HIFα. In another aspect, the present invention provides a method of increasing expression of glycolytic factors in a subject, the method comprising stabilizing HIFα. In a further aspect, the invention provides a method of increasing expression of factors associated with oxidative stress in a subject, the method comprising stabilizing HIFα. A method of treating a subject having a disorder associated with ischemic reperfusion injury, the method comprising stabilizing HIFα, is also contemplated.

D. Methods for Identifying Compounds that Stabilize HIFα

Methods for identifying compounds that stabilize HIFα are also provided herein. For example, the present invention provides a method of identifying a compound that stabilizes HIFα, the method comprising: (a) administering a compound of interest to a subject or to a sample from a subject; (b) measuring the HIFα level in the subject or in the sample; and (c) comparing the HIFα level in the subject or in the sample to a standard level, wherein an increase in the HIFα level in the subject or the sample is indicative of a compound that stabilizes HIFα.

E. Administration of HIFα-Stabilizing Compounds

In another aspect, the invention provides compounds that stabilize HIFα and methods of using the compounds to prevent, pretreat, or treat HIF-associated conditions such as those described herein. In one embodiment, a therapeutically effective amount of the compound or a pharmaceutically acceptable salt thereof, alone or in combination with a pharmaceutically acceptable excipient, is administered to a subject having a HIF-associated condition. In one specific embodiment, the compound is administered immediately following the diagnosis of an acute ischemic disorder. In another specific embodiment, the compound is administered to a subject during the course of a chronic ischemic condition. In yet another specific embodiment, the ischemia is due to a transient or acute trauma, insult, or injury such as, e.g., a spinal cord injury. In a specific embodiment, the compound is administered to a patient in need following diagnosis of a pulmonary disorder such as COPD and the like.

In one aspect, the compound can be administered based on predisposing conditions, e.g., chronic conditions, or as a pretreatment to decrease or prevent tissue damage caused by HIF-associated disorders. In a specific aspect, the compound is administered to a subject who has a history of recurring episodes of an ischemic condition, e.g., myocardial infarction or transient ischemic attacks, or has symptoms of impending ischemia, e.g., angina pectoris. In another specific embodiment, the compound is administered based on physical parameters implicating possible ischemia or hypoxia, such as is the case with, e.g., individuals placed under general anesthesia or temporarily working at high altitudes. In yet another embodiment, the compounds may be used in the context of organ transplants to pretreat organ donors and to maintain organs removed from the body prior to implantation in a recipient.

### II. STASIS

In certain embodiments, a compound of the present invention induces stasis, as described herein. Stasis has been found to be modulated by HIF and also affects HIF prolyl hydroxylase activity (e.g., Egl-9). Therefore, the present invention provides methods, compositions, articles of manufacture, and apparatuses to induce stasis via modulation of HIF in cells, tissues and organs located within or derived from an organism, as well as in the organism itself. Such methods, compositions, articles of manufacture, and apparatuses can be employed to protect biological matter, as well as to prevent, treat, or diagnose diseases and conditions in the organism. In addition, such methods may directly induce stasis themselves, or they may act indirectly by not inducing stasis themselves, but by enhancing the ability of biological matter to enter stasis in response to an injury or disease condition, e.g., by reducing the time or level of injury or disease required to achieve stasis. Such a condition may be referred to as pre-stasis. Details of such applications and other uses are described below.

The invention is based, in part, on studies with compounds that were determined to have a protective function, and thus, serve as protective agents via modulation of HIF. Moreover, the overall results of studies involving different compounds indicate that compounds with an available electron donor center are particularly effective in inducing stasis or pre-stasis. In addition, these compounds induce reversible stasis, meaning they are not so toxic to the particular biologic matter that the matter dies or decomposes. It is further contemplated that the present invention can be used to enhance survivability of and/or to prevent or reduce damage to biological matter, which may be subject to or under adverse conditions.

In particular embodiments, methods of the present invention are used to induce stasis or pre-stasis in biological matter, e.g., cells, tissues, organs, and/or organisms, after an injury (e.g., a traumatic injury) or after the onset or progression of a disease, in order to protect the biological matter from damage associated with the injury or disease prior to, during, or following treatment of the injury or disease. These and other methods of the present invention involve modulation of HIF. In other embodiments, methods of the present invention are used to induce or promote stasis or pre-stasis in biological matter prior to subjection to an injurious event (e.g., an elective surgery) or prior to the onset or progression of a disease, in order to protect the biological matter from damage associated with adverse conditions such as injury or disease. Such methods are generally referred to as “pre-treatment” with an Effective Compound. Pre-treatment includes methods wherein biological matter is provided with an Effective Compound both before and during, and before, during and after biological matter is subjected to adverse conditions (e.g., an injury or onset or the progression of a disease), and methods wherein biological matter is provided with an Effective Compound only before biological matter is subjected to adverse conditions.

According to various embodiments of the methods of the present invention, stasis may be induced by treating biological matter with an Effective Compound that induces stasis directly itself or, alternatively, by treating biological matter with an Effective Compound that does not itself induce stasis, but instead, promotes or enhances the ability of or decreases the time required for the biological matter to achieve stasis in response to another stimuli, such as, but not limited to, an injury, a disease, hypoxia, excessive bleeding, or treatment with another Effective Compound.

In particular embodiments, treatment with an Effective Compound induces “pre-stasis,” which refers to a hypometabolic state through which biological matter must transition to reach stasis. Pre-stasis is characterized by a reduction in metabolism within the biological material of a magnitude that is less than that defined as stasis. Further, pre-stasis can be considered an HIF-associated condition. In order to achieve stasis using an Effective Compound, the biological matter necessarily must transition through a graded hypometabolic state in which oxygen consumption and CO₂ production are reduced less than two-fold in the biological matter. Such a continuum, in which metabolism or cellular respiration is reduced by an Effective Compound to a degree less than two-fold, can be described as a state of “pre-stasis”.

To the extent that stasis comprises a two-fold reduction (i.e., a reduction to 50% or less) in either CO₂ production or O₂ consumption, direct measurement of these parameters in the biological matter using methods known to those in the art in which a reduction of less than two-fold is detected is indicative of pre-stasis. Accordingly, certain measurements of carbon dioxide and oxygen levels in the blood as well as other markers of metabolic rate familiar to those skilled in the art including, but not limited to, blood pO₂, VO₂, pCO₂, pH, and lactate levels, may be used in the instant invention to monitor the onset or progression of pre-stasis. While indicators of metabolic activity, e.g., CO₂ production via cellular respiration and O₂ consumption, are reduced less than two-fold as compared to normal conditions, pre-stasis may be associated with an at least 10%, 15%, 20%, 25%, 30%, 35%, 40%, 45%, or 50% reduction in CO₂ evolution, which refers to the amount of CO₂ released from the lungs. In addition, in various embodiments, pre-stasis is characterized by a reduction in one or more indicators of metabolic activity that is less than or equal to 1%, 2%, 5%, 10%, 15%, 20%, 25%, 30%, 35%, 40%, 45%, or 49% as compared to normal physiological conditions. In other embodiments, pre-stasis is characterized by its ability to enhance or promote entry into stasis in response to another stimuli (wherein the another stimuli may include prolonged treatment with the same active agent), or its ability to enhance survival of or protect biological matter from damage resulting from an injury, the onset or progression of the disease, or bleeding, particularly bleeding that can lead to irreversible tissue damage, hemorrhagic shock, or lethality.

While methods of the present invention explicitly exemplified herein may refer to inducing “stasis,” it is understood that these methods may be readily adapted to induce “pre-stasis,” and that such methods of inducing pre-stasis are contemplated by the present invention. In addition, the same Effective Compounds used to induce stasis may also be used to induce pre-stasis, by providing them to biological matter at, for example, a lower dosage and/or for a shorter time than used to induce stasis.

In certain embodiments, the present invention involves exposing biological matter to an amount of an agent, so as to achieve stasis of the biological matter via modulation of HIF. In some embodiments, the present invention concerns methods for inducing stasis in in vivo biological matter comprising: a) identifying an organism in which stasis is desired; and, b) exposing the organism to an effective amount of an Effective Compound, such as an oxygen antagonist, to induce stasis in the in vivo biological matter. “Inducing stasis” in biological matter means that the matter is alive but is characterized by one or more of the following: at least a two-fold reduction in the rate or amount of carbon dioxide production by the biological matter; at least a two-fold (i.e., 50%) reduction in the rate or amount of oxygen consumption by the biological matter; and at least a 10% decrease in movement or motility (applies only to cells or tissue that move, such as sperm cells or a heart or a limb, or when stasis is induced in the entire organism) (collectively referred to as “cellular respiration indicators”). In certain embodiments of the invention, it is contemplated that there is about, at least about, or at most about a 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, 1000-, 1100-, 1200-, 1300-, 1400-, 1500-, 1600-, 1700-, 1800-, 1900-, 2000-, 2100-, 2200-, 2300-, 2400-, 2500-, 2600-, 2700-, 2800-, 2900-, 3000-, 3100-, 3200-, 3300, 3400-, 3500-, 3600-, 3700-, 3800-, 3900-, 4000-, 4100-, 4200-, 4300-, 4400-, 4500-, 5000-, 6000-, 7000-, 8000-, 9000-, or 10000-fold or more reduction in the rate of oxygen consumption by the biological matter, or any range derivable therein. Alternatively, it is contemplated that embodiments of the invention may be discussed in terms of a reduction in the rate of oxygen consumption by the biological matter as about, at least about, or at most about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or more, or any range derivable therein. It is contemplated that any assay to measure oxygen consumption may be employed, and a typical assay will involve utilizing a closed environment and measuring the difference between the oxygen put into the environment and oxygen that is left in the environment after a period of time. It is further contemplated that carbon dioxide production can be measured to determine the amount of oxygen consumption by biological matter. Thus, there may be decreases in carbon dioxide production, which would correspond to the decreases in oxygen consumption discussed above.

In methods of the invention, stasis or pre-stasis is temporary and/or reversible, meaning that the biological matter no longer exhibits the characteristics of stasis at some later point in time. In some embodiments of the invention, instead of an oxygen antagonist, a compound that is not does not qualify as an oxygen antagonist is administered. It is contemplated that methods discussed with respect to oxygen antagonists may be applied with respect to any compound that is an oxygen antagonist, protective metabolic agent, compound with the structure of Formula I, Ia-Id, II, III, IIIa, IV, V, VI, VII, VIII, IX, X, XI, any other compound discussed herein, or a salt or precursor thereof. In particular embodiments, induction of stasis is desired in which case the compound may be referred to as an “effective stasis compound.” It is contemplated that in some embodiments of the invention, a method is achieved by inducing stasis. For example, therapeutic methods may involve inducing stasis, in which case the Effective Compound is an effective stasis compound. It is specifically contemplated that in embodiments in which Effective Compounds are discussed, the invention includes, and may be limited to, oxygen antagonists.

In certain embodiments of the present invention, biological matter is treated with an Effective Compound that does not induce stasis by itself (at least not at the level and/or duration of time provided), but rather induces biological matter to enter a pre-stasis state that has therapeutic benefits and that enhances the ability of the biological matter to achieve stasis in response to another stimuli, such as, e.g., an injury, disease state, or treatment with another Effective Compound or the same Effective Compound If used for a longer duration or greater dosage.

The term “biological matter,” as described in more detail herein, refers to any living biological material (such as mammalian biological material, in some embodiments) including cells, tissues, organs, and/or organisms, and any combination thereof. It is contemplated that stasis may be induced in a part of an organism (such as in cells, in tissue, and/or in one or more organs), whether that part remains within the organism or is removed from the organism, or the whole organism will be placed in a state of stasis. Moreover, it is contemplated in the context of cells and tissues that homogenous and heterogeneous cell populations may be the subject of embodiments of the invention. The term “in vivo biological matter” refers to biological matter that is in vivo, i.e., still within or attached to an organism. Moreover, the term “biological matter” will be understood as synonymous with the term “biological material.” In certain embodiments, it is contemplated that one or more cells, tissues, or organs is separate from an organism. The term “isolated” can be used to describe such biological matter. It is contemplated that stasis may be induced in isolated biological matter.

An organism or other biological matter in need of stasis is an organism or biological matter in which stasis of all or part of the organism may yield direct or indirect physiological benefits. For example, a patient at risk for hemorrhagic shock may be considered in need of stasis, or a patient who will undergo coronary artery bypass surgery may benefit from protecting the heart from ischemia/reperfusion injury. Other applications are discussed throughout the application. In some cases, an organism or other biological matter is identified or determined to be in need of stasis based on one or more tests, screens, or evaluations that indicate a condition or disease, or the risk of a condition or disease that can be prevented or treated by undergoing stasis. Alternatively, the taking of a patient medical or family medical history (patient interview) may yield information that an organism or other biological matter is in need of stasis. As would be evident to one skilled in the art, one application of the present invention would be to reduce the overall energy demands of a biological material by inducing stasis.

Alternatively, an organism or other biological matter may be in need of an Effective Compound to enhance survivability. For instance, a patient may need treatment for an injury or disease or any other application discussed herein. They may be determined to be in need of enhanced survivability or treatment based on methods discussed in the previous paragraph, such as by taking a patient medical or family medical history.

It will be understood that when inducing stasis in a tissue or organ, an effective amount is one that induces stasis in the tissue or organ as determined by the collective amount of cellular respiration of the tissue or organ. Accordingly, for example, if the level of oxygen consumption by a heart (collectively with respect to cells of the heart) is decreased at least about 2-fold (i.e., 50%) after exposure to a particular amount of a certain Effective Compound, such as an oxygen antagonist or an effective stasis compound, it will be understood that that was an effective amount to induce stasis in the heart. Similarly, an effective amount of an agent that induces stasis in an organism is one that is evaluated with respect to the collective or aggregate level of a particular parameter of stasis. It will be also understood that when inducing stasis in an organism, an effective amount is one that induces stasis generally of the whole organism, unless a particular part of the organism was targeted. In addition, it is understood that an effective amount may be an amount sufficient to induce stasis by itself, or it may be an amount sufficient to induce stasis in combination with another agent or stimuli, e.g., another Effective Compound, an injury, or a disease state.

The concept of an effective amount of a particular compound is related, in some embodiments, to how much utilizable oxygen there is available to the biological matter. Generally, stasis can be induced when there is about 100,000 ppm or less of oxygen in the absence of any oxygen antagonist (room air has about 210,000 ppm oxygen). The oxygen antagonist serves to alter how much oxygen is effectively available. At concentration of 10 ppm of oxygen, suspended animation is induced. Thus, while the actual concentration of oxygen that biological matter is exposed to may be higher, even much higher, than 10 ppm, stasis can be induced because of the competitive effect of an oxygen antagonist with oxygen for binding to essential oxygen metabolizing proteins in the biological matter. In other words, an effective amount of an oxygen antagonist reduces the effective oxygen concentration to a point where the oxygen that is present cannot be used. This will happen when the amount of an oxygen antagonist reduces the effective oxygen concentration below the Kₘ of oxygen binding to essential oxygen metabolizing proteins (i.e., comparable to 10 ppm of oxygen). Accordingly, in some embodiments, an oxygen antagonist reduces the effective concentration of oxygen by about or at least about 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, 1000-, 1100-, 1200-, 1300-, 1400-, 1500-, 1600-, 1700-, 1800-, 1900-, 2000-, 2100-, 2200-, 2300-, 2400-, 2500-, 2600-, 2700-, 2800-, 2900-, 3000-, 3100-, 3200-, 3300, 3400-, 3500-, 3600-, 3700-, 3800-, 3900-, 4000-, 4100-, 4200-, 4300-, 4400-, 4500-, 5000-, 6000-, 7000-, 8000-, 9000-, or 10000-fold or more, or any range derivable therein. Alternatively, it is contemplated that embodiments of the invention may be discussed in terms of a reduction in effective oxygen concentration as about, at least about, or at most about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or more, or any range derivable therein. It is understood that this is another way of indicating a decrease in cellular respiration.

Furthermore, in some embodiments, stasis can be measured indirectly by a drop in core body temperature of an organism. It is contemplated that a reduction in core body temperature of about, at least about, or at most about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50° F. or more, or any range derivable therein may be observed in methods of the invention. In some embodiments of the invention, hypothermia can be induced, such as moderate hypothermia (at least 10° F. reduction) or severe hypothermia (at least 20° F. reduction).

Moreover, the effective amount can be expressed as a concentration with or without a qualification on length of time of exposure. In some embodiments, it is generally contemplated that to induce stasis or achieve other stated goals of the invention, the biological matter is exposed to an oxygen antagonist or other Effective Compound for about, at least about, or at most about 5, 10, 15, 20, 25, 30, 35, 40, 45, 50, 55, 60 seconds, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, 1, 2, 3, 4, 5, 6, 7, 8, 9, 10, 11, 12 months, 1, 2, 3, 4, 5, 6, 7, 8, 9, 10, 11, 12, 13, 14, 15, 16, 17, 18, 19, 20 or more years, and any combination or range derivable therein. It is further contemplated that the amount of time may be indefinite, depending on the reason or purpose for administering the oxygen antagonist or other Effective Compound. Thereafter, biological matter may continue to be exposed to an oxygen antagonist or other Effective Compound, or, in other embodiments of the invention, the biological matter may no longer be exposed to the oxygen antagonist or other Effective Compound. This latter step can be achieved either by removing or effectively removing the oxygen antagonist or other Effective Compound from the presence of the biological matter in which stasis was desired, or the biological matter may be removed from an environment containing the oxygen antagonist or other Effective Compound. Additionally, matter may be exposed to or provided with any Effective Compound continuously (a period of time without a break in exposure), intermittently (exposure on multiple occasions), or on a periodic basis (exposure on multiple occasions on a regular basis). The dosages of the Effective Compound on these different bases may the same or they may vary. In certain embodiments, an Effective Compound is provided periodically by providing or exposing biological matter to an Effective Compound 1, 2, 3, 4, 5, 6, 7, 8, 9, or 10 times every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, 1, 2, 3, 4, 5, 6, 7, 8, 9, 10, 11, 12 months, 1, 2, 3, 4, 5, 6, 7, 8, 9, 10, 11, 12, 13, 14, 15, 16, 17, 18, 19, 20 or more years, or any range derivable therein.

Furthermore, in some embodiments of the invention, biological matter is exposed to or provided with an Effective Compound for a sustained period of time, where “sustained” means a period of time of at least about 2 hours. In other embodiments, biological matter may be exposed to or provided with an Effective Compound on a sustained basis for more than a single day. In such circumstances, the biological matter is provided with an Effective Compound on a continuously sustained basis. In certain embodiments, biological matter may be exposed to or provided with an Effective Compound for about 2, 3, 4, 5, 6, 7, 8, 9, 10, 11, 12 or more hours (or any range derivable therein) for 2, 3, 4, 5, 6, 7 days, and/or 1, 2, 3, 4, 5 weeks, and/or 1, 2, 3, 4, 5, 6, 7, 8, 9, 10, 11, 12 months, and/or 1, 2, 3, 4, 5, 6, 7, 8, 9, 10, 11, 12, 13, 14, 15, 16, 17, 18, 19, 20 or more years (or any range derivable therein) continuously, intermittently (exposure on multiple occasions), or on a periodic basis (exposure on a recurring regular basis).

In some embodiments, biological matter may be exposed to or provided with an Effective Compound at least before and during; before, during, and after; during and after; or solely after a particular injury, trauma, or treatment (for instance, surgery), adverse condition or other relevant event or situation. This exposure may or may not be sustained.

The dosages of the Effective Compound on these different bases may the same or they may vary.

Moreover, in certain embodiments, an Effective Compound may be provided on a continously sustained basis at level that is considered “low,” meaning a level that is less than the amount that causes metabolic flexibility such as seen with drop in CBT, heart rate, or CO₂ or O₂ consumption or production.

In certain embodiments, biological matter is exposed or provided an Effective Compound, such as a metabolic agent, in an amount that exceeds what was previously understood to be the maximum tolerated dose before adverse physiological ramifications such as apnea (“period of time during which breathing is markedly reduced such that the subject takes 10% or fewer number of breaths”), lack of observable skeletal muscle movement, dystonia, and/or hyperactivity. Such an amount may be particularly relevant to increasing survivability in some embodiments of the invention, for instance, to increase the chances of surviving adverse conditions, such as those that would induce death from hemorrhagic shock.

A physiological state can be induced by Effective Compounds of the present invention which enhances survivability in an organism in need of survivability enhancement and comprises a set of observable physiological changes in response to an effective dose of an Effective Compound, said changes may comprise one, more or all of hyperpnea, apnea and the concomitant or subsequent loss of neuromuscular tone or voluntary control of movement with continued heartbeat. A transient and measurable change in arterial blood color may also be observed. Hyperpnea refers to rapid, shallow breathing. Apnea refers to a cessation of breathing or the reduction as described above.

In certain embodiments, the subject becomes apnic, which is marked by a cessation in breathing and then an apnic breath after a short period of time. In rats, this occurs after approximately 20 seconds. Thus, it is contemplated that a subject induced into apnea may exhibit 0, 1, 2, 3, 4, 5, 6, 7, 8, 9, 10% the number of breaths subsequent to exposure to an Effective Compound. The subject may have an occasional breath, which may be considered an apnic breath, thereafter. In certain embodiments of the invention, apnea continues until the subject is no longer exposed to the Effective Compound.

In some embodiments of the invention, an effective amount may be expressed as LD₅₀, which refers to the “median lethal dose,” which means the dose that is administered that kills half the population of animals (causes 50% mortality). Moreover, in further embodiments, an effective amount may be independent of the weight of the biological matter (“weight independent”). In rodents and humans, for example, the LD₅₀ of H₂S gas is approximately 700 ppm before adverse physiological effects occur. Moreover, in some embodiments of the invention, increasing survivability refers generally to living longer, which is an embodiment of the invention.

The present invention also concerns methods for inducing apnea in an organism comprising administering to the organism an effective amount of an Effective Compound. In certain embodiments, the organism also does not exhibit any skeletal muscle movement as a result of the Effective Compound. It is specifically contemplated that the organism may be mammal, including a human. In other embodiments, an effective amount exceeds what is considered a lethal concentration. In further embodiments, the concentration may be a lethal amount though the exposure time may be about, at least about, or at most about 10, 15, 20, 25, 30, 35, 40, 45, 50, 55, 60 seconds, 1, 2, 3, 4, 5 minutes or more (or any range derivable therein or any other time period specified in this disclosure). In particular embodiments, a mammal is exposed to at least about 600 ppm of an Effective Compound in a gaseous form, such as H₂S.

Additionally, in certain embodiments, there is a step of identifying an animal in need of treatment. In other embodiments, there is a step of observing apnea in the organism. In even further embodiments, methods involving obtaining a blood sample from the organism and/or evaluating the color of the organism's blood. It has been observed that exposure to H₂S changes the color of blood from a mammal; it goes from bright red to a darker, red wine color and then to brick red. Evaluating the color may be done visually without any instruments or machines, while in other embodiments, an instrument may be used, such as a spectrophotometer. Furthermore, a blood sample may be obtained from an organism and other types of analysis may be done on it. Alternatively, a blood sample may not be needed and instead, blood may be evaluated without the sample. For instance, a modified pulse-oximeter that shines IR or visible light through the finger may be employed to monitor color changes in the blood.

In certain embodiments, biological matter is exposed to an effective amount of an Effective Compound that does not lead to stasis or pre-stasis. In some embodiments, there may be no evidence of a reduction in oxygen consumption or carbon dioxide production while the Effective Compound is present.

In additional embodiments, an organism may be exposed to the Effective Compound while sleeping. Moreover, as discussed above, the exposure may be regular, such as daily (meaning exposure at least once a day).

It is specifically contemplated that in some embodiments an Effective Compound is provided to a subject by nebulizer. This may be applied with any embodiment of the invention. In certain cases, the nebulizer is used for the treatment of hemorrhagic shock. In further embodiments, the Effective Compound is provided as a single dose to the subject. In specific cases, a single dose or multiple doses is one that would induce apnea in a subject. In some embodiments, a subject is given at least about 1,000, 2,000, 3,000, 4,000, 5,000, 6,000, 7,000, 8,000, 9,000, 10,000, 11,000, 12,000 or more ppm H₂S gas. The exposure time may be any of the times discussed herein, including about or about at most 10, 9, 8, 7, 6, 5, 4, 3, 2, 1, 0.5, 0.1 minutes or less (or any range derivable therein).

In further embodiments, after exposure to an Effective Compound the metabolic rate of biologic matter may change. In certain embodiments, the RQ ratio (CO₂ production/O₂ consumption) of the biological matter changes after exposure to an Effective Compound. This may occur after an initial exposure or repeated exposure or after an acute exposure. In some embodiments, the RQ ratio decreases after exposure. The decrease may be a decrease of about, at least about or at most about 5, 10, 15, 20, 25, 30, 35, 40, 45, 50, 55, 60, 65, 70, 75, 80% or more, or any range derivable therein. The decrease may be a result of O₂ consumption increasing of CO₂ production decreasing in relation to O₂ consumption.

In some embodiments, no other physiological change is observed in biological matter exposed to the Effective Compound except that its RQ ratio changes after the exposure. Therefore, in some embodiments of the invention, methods involve measuring an RQ ratio in a subject. This may occur before and/or after exposure to the Effective Compound.

Therefore, in some embodiments of the invention, stasis is induced, and a further step in methods of the invention is to maintain the relevant biological matter in a state of stasis. This can be accomplished by continuing to expose the biological matter to an oxygen antagonist or other Effective Compound and/or exposing the biological matter to a nonphysiological temperature or another oxygen antagonist or other Effective Compound. Alternatively, the biological matter may be placed in a preservation agent or solution, or be exposed to normoxic or hypoxic conditions. It is contemplated that biological matter may be maintained in stasis for about, at least about, or at most about 30 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, 1, 2, 3, 4, 5, 6, 7, 8, 9, 10, 11, 12 months, 1, 2, 3, 4, 5, 6, 7, 8, 9, 10, 11, 12, 13, 14, 15, 16, 17, 18, 19, 20 or more years, and any combination or range derivable therein. Moreover, it is contemplated that in addition to or instead of changing the temperature, other changes in the environment may be implemented such as a change in pressure or to effect a cryoprotectant or cryopreservation environment (e.g., one containing glycerol).

It will be appreciated that “stasis” with respect to a whole animal and “stasis” with respect to cells or tissues may require different lengths of time in stasis. Thus, with respect to human subjects, e.g., subjects undergoing a surgical treatment, treatment for malignant hyperthermia, or trauma victim, a time of stasis of up to 12, 18, or 24 hours is generally contemplated. With respect to non-human animal subjects, e.g. non-human animals shipped or stored for commercial purposes, stasis for a period of 2 or 4 days, 2 or 4 weeks, or longer is contemplated.

The term “expose” is used according to its ordinary meaning to indicate that biological matter is subjected to an oxygen antagonist or other Effective Compound. This can be achieved in some embodiments by contacting biological matter with an oxygen antagonist or Effective Compound. In other embodiments, this is achieved by contacting the biological matter with an Effective Compound, which may or may not be an oxygen antagonist. In the case of in vivo cells, tissues, or organs, “expose” may further mean “to lay open” these materials so that it can be contacted with an oxygen antagonist or other Effective Compound. This can be done, for example, surgically. Exposing biological matter to an oxygen antagonist or other Effective Compound can be by incubation in or with (includes immersion) the antagonist, perfusion or infusion with the antagonist, injection of biological matter with an oxygen antagonist or other Effective Compound, or applying an oxygen antagonist or other Effective Compound to the biological matter. In addition, if stasis of the entire organism is desirable, inhalation or ingestion of the oxygen antagonist or other Effective Compound, or any other route of pharmaceutical administration is contemplated for use with oxygen antagonists or other Effective Compound. Furthermore, the term “provide” is used according to its ordinary and plain meaning to mean “to supply.” It is contemplated that a compound may be provided to biological matter in one form and be converted by chemical reaction to its form as an Effective Compound. The term “provide” encompasses the term “expose” in the context of the term “effective amount,” according to the present invention.

In some embodiments, an effective amount is characterized as a sublethal dose of the oxygen antagonist or other Effective Compound. In the context of inducing stasis of cells, tissues, or organs (not the whole organism), a “sublethal dose” means a single administration of the oxygen antagonist or Effective Compound that is less than half of the amount of the oxygen antagonist or Effective Compound that would cause at least a majority of cells in a biological matter to die within 24 hours of the administration. If stasis of the entire organism is desired, then a “sublethal dose” means a single administration of the oxygen antagonist or Effective Compound that is less than half of the amount of the oxygen antagonist or Effective Compound that would cause the organism to die within 24 hours of the administration. In other embodiments, an effective amount is characterized as a near-lethal dose of the oxygen antagonist or Effective Compound. Similarly, in the context of inducing stasis of cells, tissues, or organs (not the whole organism), a “near lethal dose” means a single administration of the oxygen antagonist or Effective Compound that is within 25% of the amount of the inhibitor that would cause at least a majority of cell(s) to die within 24 hours of the administration. If stasis of the entire organism is desired, then a “near lethal dose” means a single administration of the oxygen antagonist or Effective Compound that is within 25% of the amount of the inhibitor that would cause the organism to die within 24 hours of the administration. In some embodiments a sublethal dose is administered by administering a predetermined amount of the oxygen antagonist or Effective Compound to the biological material. It is specifically contemplated that this may be implemented with respect to any Effective Compound.

Furthermore, it is contemplated that in some embodiments an effective amount is characterized as a supralethal dose of the oxygen antagonist or other Effective Compound. In the context of inducing stasis of cells, tissues, or organs (not the whole organism), a “supra-lethal dose” means a single administration of an Effective Compound that is at least 1.5 times (1.5×) the amount of the Effective Compound that would cause at least a majority of cells in a biological matter to die within 24 hours of the administration. If stasis of the entire organism is desired, then a “supra-lethal dose” means a single administration of the Effective Compound that is at least 1.5 times the amount of the Effective Compound that would cause the organism to die within 24 hours of the administration. It is specifically contemplated that the supra-lethal dose can be about, at least about, or at most about 1.5×, 2×, 3×, 4×, 5×, 10×, 20×, 30×, 40×, 50×, 60×, 70×, 80×, 90×, 100×, 150×, 200×, 250×, 300×, 400×, 500×, 600×, 700×, 800×, 900×, 1000×, 1100×, 1200×, 1300×, 1400×, 1500×, 1600×, 1700×, 1800×, 1900×, 2000×, 3000×, 4000×, 5000×, 6000×, 7000×, 8000×, 9000×, 10,000× or more, or any range derivable therein, the amount of the Effective Compound that would cause at least a majority of cells in a biological matter (or the entire organism) to die within 24 hours of the administration.

The amount of the Effective Compound that is provided to biological matter can be about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mg, mg/kg, or mg/m2, or any range derivable therein. Alternatively, the amount may be expressed as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mM or M, or any range derivable therein.

In some embodiments an effective amount is administered by monitoring, alone or in combination, the amount of oxygen antagonist or other Effective Compound administered, monitoring the duration of administration of the oxygen antagonist or other Effective Compound, monitoring a physiological response (e.g., pulse, respiration, pain response, movement or motility, metabolic parameters such as cellular energy production or redox state, etc.) of the biological material to the administration of the oxygen antagonist or other Effective Compound and reducing, interrupting or ceasing administration of the compound(s) when a predetermined floor or ceiling for a change in that response is measured, etc. Moreover, these steps can be employed additionally in any method of the invention.

Tissue in a state of stasis or that has undergone stasis can be used in a number of applications. They can be used, for example, for transfusion or transplantation (therapeutic applications, including organ transplants); for research purposes; for screening assays to identify, characterize, or manufacture other compounds that induce stasis; for testing a sample from which the tissue was obtained (diagnostic applications); for preserving or preventing damage to the tissue that will be placed back into the organism from which they were derived (preventative applications); and for preserving or preventing damage to them during transport or storage. Details of such applications and other uses are described below. The term “isolated tissue” means that the tissue is not located within an organism. In some embodiments, the tissue is all or part of an organ. The terms “tissue” and “organ” are used according to their ordinary and plain meanings. Though tissue is composed of cells, it will be understood that the term “tissue” refers to an aggregate of similar cells forming a definite kind of structural material. Moreover, an organ is a particular type of tissue.

The present invention concerns methods for inducing stasis in isolated tissue comprising: a) identifying the tissue in which stasis is desired; and, b) exposing the tissue to an effective amount of an oxygen antagonist or other Effective Compound to induce stasis.

Compositions, methods, and articles of manufacture of the invention can be used on biological matter that will be transferred back into the donor organism from which it was derived (autologous) or a different recipient (heterologous) subject. In some embodiments, biological matter is obtained directly from a donor organism. In others, the biological matter is placed in culture prior to exposure to an oxygen antagonist or other Effective Compound. In some situations, the biological matter is obtained from a donor organism administered extracorporeal membrane oxygenation prior to retrieval of the biological matter, which is a technique implemented to aid in the preservation of biological matter. Moreover, methods include administering or implanting the biological matter in which stasis was induced to a live recipient organism.

In some embodiments, an organ or tissue to be retrieved and then transplanted is exposed to the oxygen antagonist or other Effective Compound while still in the donor subject. It is contemplated that in some cases, the vasculature of the donor is used to expose the organ or tissue to the oxygen antagonist or other Effective Compound. This can be done if the heart is still pumping or a pump, catheter, or syringe can be used to administer the oxygen antagonist or other Effective Compound into the vasculature for delivery to the organ or tissue

Methods of the invention also concern inducing stasis in isolated tissue comprising incubating the tissue with an oxygen antagonist or other effective stasis compound that creates hypoxic conditions for an effective amount of time for the tissue to enter stasis.

Cells in a state of stasis or that have undergone stasis can be used in a number of applications. They can be used, for example, for transfusion or transplantation (therapeutic applications); for research purposes; for screening assays to identify, characterize, or manufacture other compounds that induce stasis; for testing a sample from which the cells were obtained (diagnostic applications); for preserving or preventing damage to the cells that will be placed back into the organism from which they were derived (preventative applications); and for preserving or preventing damage to cells during transport or storage. Details of such applications and other uses are described below.

The present invention concerns methods for inducing stasis in one or more cells separate from an organism comprising: a) identifying the cell(s) in which stasis is desired; and, b) exposing the cell(s) to an effective amount of an oxygen antagonist or other effective stasis compound to induce stasis.

It is contemplated that the cell may be any oxygen-utilizing cell. The cell may be eukaryotic or prokaryotic. In certain embodiments, the cell is eukaryotic. More particularly, in some embodiments, the cell is a mammalian cell. Mammalian cells contemplated for use with the invention include, but are not limited to those that are from a: human, monkey, mouse, rat, rabbit, hamster, goat, pig, dog, cat, ferret, cow, sheep, and horse.

Moreover, cells of the invention may be diploid but in some cases, the cells are haploid (sex cells). Additionally, cells may be polyploid, aneuploid, or anucleate. The cell can be from a particular tissue or organ, such as one from the group consisting of: heart, lung, kidney, liver, bone marrow, pancreas, skin, bone, vein, artery, cornea, blood, small intestine, large intestine, brain, spinal cord, smooth muscle, skeletal muscle, ovary, testis, uterus, and umbilical cord. Moreover, the cell can also be characterized as one of the following cell types: platelet, myelocyte, erythrocyte, lymphocyte, adipocyte, fibroblast, epithelial cell, endothelial cell, smooth muscle cell, skeletal muscle cell, endocrine cell, glial cell, neuron, secretory cell, barrier function cell, contractile cell, absorptive cell, mucosal cell, limbus cell (from cornea), stem cell (totipotent, pluripotent or multipotent), unfertilized or fertilized oocyte, or sperm.

The present invention also provides methods, compositions, and apparati for enhancing survivability of and/or reducing damage to biological matter under adverse conditions by reducing metabolic demand, oxygen requirements, temperature, or any combination thereof in the biological matter of interest. In some embodiments of the invention, survivability of biological matter is enhanced by providing it with an effective amount of a protective metabolic agent. The agent enhances survivability by preventing or reducing damage to the biological matter, preventing all or part of the matter from dying or senescing, and/or extending the lifespan of all or part of the biological matter, relative to biological matter not exposed to the agent. Alternatively, in some embodiments the agent prolongs survival of tissue and/or an organism that would otherwise not survive without the agent.

It is contemplated that a “protective metabolic agent” is a substance or compound capable of reversibly altering the metabolism of biological matter that is exposed to or contacted with it and that promotes or enhances the survivability of the biological matter.

In certain embodiments, the protective metabolic agent induces stasis in the treated biological matter; while, in other embodiments, the protective metabolic agent does not directly itself induce stasis in the treated biological matter. Protective metabolic agents, and other Effective Compounds, may enhance survivability and/or reduce damage to biological matter without inducing stasis in the biological matter per se, but rather by reducing cellular respiration and corresponding metabolic activity to a degree that is less than about a fifty percent decrease in oxygen consumption or carbon dioxide production. Additionally, such compounds may cause the biological matter to more quickly, easily, or effectively enter into or achieve stasis in response to an injury or disease state, e.g., by inducing the biological matter to achieve a state of pre-stasis.

Survivability includes survivability when the matter is under adverse conditions—that is, conditions under which there can be adverse and nonreversible damage or injury to biological matter. Adverse conditions can include, but are not limited to, when oxygen concentrations are reduced in the environment (hypoxia or anoxia, such as at high altitudes or under water); when the biological matter is incapable of receiving that oxygen (such as during ischemia), which can be caused by i) reduced blood flow to organs (e.g., heart, brain, and/or kidneys) as a result of blood vessel occlusion (e.g., myocardial infarction, and/or stroke), ii) extracorporeal blood shunting as occurs during heart/lung bypass surgery (e.g., “pumphead syndrome” in which heart or brain tissue is damaged as a result of cardiopulmonary bypass), or iii) as a result of blood loss due to trauma (e.g., hemorrhagic shock or surgery); hypothermia, where the biological material is subjected to sub-physiological temperatures, due to exposure to cold environment or a state of low temperature of the biological material, such that it is unable to maintain adequate oxygenation of the biological materials; hyperthermia, whereby temperatures where the biological material is subjected to supra-physiological temperatures, due to exposure to hot environment or a state of high temperature of the biological material such as by a malignant fever; conditions of excess heavy metals, such as iron disorders (genetic as well as environmental) such as hemochromatosis, acquired iron overload, sickle-cell anemia, juvenile hemochromatosis African siderosis, thalassemia, porphyria cutanea tarda, sideroblastic anemia, iron-deficiency anemia and anemia of chronic disease. It is contemplated that a protective metabolic agent is an oxygen antagonist in certain embodiments of the invention. It is also contemplated that in certain other embodiments, an oxygen antagonist is not a protective metabolic agent. In other embodiments of the invention, one or more compounds may be used to increase or enhance survivability of biological matter; reversibly inhibit the metabolism and/or activity of biological matter; reduce the oxygen requirement of biological matter; reduce or prevent damage to biological matter under adverse conditions; prevent or reduce damage or injury to biological matter; prevent aging or senescence of biological matter; and, provide a therapeutic benefit as described throughout the application with respect to oxygen antagonists. It is contemplated that embodiments relating to inducing stasis are applicable to these other embodiments as well. Therefore, any embodiment discussed with respect to stasis may be implemented with respect to these other embodiments.

An Effective Compound used for inducing stasis or any of these other embodiments may lead or provide their desired effect(s), in some embodiments, only when they are in the context of the biological matter (i.e., have no lasting effect) and/or they may provide for these effect(s) for more than 24 hours after the biological matter is no longer exposed to it. Moreover, this can also be the case when a combination of Effective Compounds is used.

In certain embodiments, biological matter is exposed to an amount of an oxygen antagonist or other Effective Compound that reduces the rate or amount of carbon dioxide production by the biological matter at least 2-fold, but also by about, at least about, or at most about 3-, 4-, 5-, 6-, 7-, 8-, 9-, 10-, 15-, 20-, 25-, 30-, 35-, 40-, 45-, 50-, 100-, 200-, 300-, 400-, 500-fold of more, or any range derivable therein. Alternatively, it is contemplated that embodiments of the invention may be discussed in terms of a reduction in the rate or amount of carbon dioxide production by the biological matter as about, at least about, or at most about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or more, or any range derivable therein. In still further embodiments, biological matter is exposed to an amount of an oxygen antagonist or other Effective Compound that reduces the rate or amount of oxygen consumption by the biological matter at least 2-fold, but also by about, at least about, or at most about 3-, 4-, 5-, 6-, 7-, 8-, 9-, 10-, 15-, 20-, 25-, 30-, 35-, 40-, 45-, 50-, 100-, 200-, 300-, 400-, 500-fold of more, or any range derivable therein. Alternatively, it is contemplated that embodiments of the invention may be discussed in terms of a reduction in the rate or amount of oxygen consumption by the biological matter as about, at least about, or at most about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or more, or any range derivable therein. In still further embodiments, biological matter is exposed to an amount of an oxygen antagonist or other Effective Compound that decreases movement or motility by at least 10%, but also by about, at least about, or at most about 15, 20, 25, 30, 35, 40, 45, 50, 55, 60, 65, 70, 75, 80, 85, 90, 95, 99, or 100%, or any range derivable therein. As with other embodiments, these characteristics and parameters are in the context of whichever biological matter is induced into a state of stasis. Thus, if stasis is induced in an organism's heart, these parameters would be evaluated for the heart, and not the whole organism. In the context of organisms, a reduction in oxygen consumption on the order of roughly 8-fold is a kind of stasis referred to as “hibernation.” Moreover, it will be understood in this application that a reduction in oxygen consumption on the order of around 1000-fold can be considered “suspended animation.” It will be understood that embodiments of the invention concerning stasis can be achieved at the level of hibernation or suspended animation, if appropriate. It is understood that a “-fold reduction” is relative to the reduced amount; for example, if a non-hibernating animal consumes 800 units of oxygen, the hibernating animal consumes 100 units of oxygen.

Additionally, in some embodiments of the invention, methods are provided for reducing cellular respiration, which may or may not be as high as that needed to reach stasis. A reduction in oxygen consumption by about, at least about, or at most about 1, 2, 5, 10, 15, 20, 25, 30, 35, 40, 45, 50, 55, 60, 65, 70, 75, 80, 85, 90, 95, or 100% is provided in methods of the invention. This can also be expressed and assessed in terms of any cellular respiration indicator.

It is contemplated that biological matter may be exposed to one or more oxygen antagonists or other Effective Compounds more than one time. It is contemplated that biological matter may be exposed to one or more Effective Compounds 1, 2, 3, 4, 5, 6, 7, 8, 9, 10 or more times, meaning when a biological matter is exposed multiple times that there are periods of respite (with respect to exposure to the Effective Compound) in between.

It is also contemplated that an Effective Compound may be administered before, during, after, or any combination thereof, in relation to the onset or progression of an injurious insult or disease condition. In certain embodiments, pre-treatment of biological matter to an Effective Compound is sufficient to enhance survivability and/or reduce damage from an injurious or disease insult. Pre-treatment is defined as exposure of the biological matter to the Effective Compound before the onset or detection of the injurious or disease insult. Pre-treatment can be followed by termination of exposure at or near the onset of the insult or continued exposure after the onset of insult.

In certain embodiments, methods including pre-exposure to an Effective Compound (i.e., pre-treatment) are used to treat conditions in which an injurious or disease insult is 1) scheduled or elected in advance, or 2) predicted in advance to likely occur. Examples meeting condition 1 include, but are not limited to, major surgery where blood loss may occur spontaneously or as a result of a procedure, cardiopulmonary bypass in which oxygenation of the blood may be compromised or in which vascular delivery of blood may be reduced (as in the setting of coronary artery bypass graft (CABG) surgery), or in the treatment of organ donors prior to removal of donor organs for transport and transplantation into a recipient in need of an organ transplant. Examples meeting condition 2 include, but are not limited to, medical conditions in which a risk of injury or disease progression is inherent (e.g., in the context of unstable angina, following angioplasty, bleeding aneurysms, hemorrhagic strokes, following major trauma or blood loss), or in which the risk can be diagnosed using a medical diagnostic test.

Exposure to the Effective Compound may enhance survivability or reduce damage when exposure occurs after the onset or detection of the injurious or disease insult to achieve a therapeutic effect. Exposure to the Effective Compound may be brief or extended. The exposure duration may be only for as long as needed to reach an indicator of stasis activity or pre-stasis (e.g., blood pCO₂, pO₂, pH, lactate, or sulfhemoglobin levels, or body temperature), or it may be longer. In certain embodiments, exposure occurs following traumatic injury (including iatrogenic and/or non-iatrogenic injuries) to an organism and is used to induce stasis or pre-stasis in the entire organism or injured tissue therein, so as to prevent or minimize damage, e.g., ischemic and reperfusion injury prior to, during, and/or following treatment.

In one embodiment, the present invention includes a method of protecting a mammal from suffering cellular damage from a surgery, comprising providing to the mammal an amount of hydrogen sulfide or other Effective Compound sufficient to induce the mammal to enter pre-stasis prior to the surgery. The surgery may be elective, planned, or emergency surgery, such as, e.g., cardiopulmonary surgery. The hydrogen sulfide may be administered by any means available in the art, including, e.g., intravenously or by inhalation.

In another embodiment, the present invention includes a method of protecting a mammal from suffering cellular damage from a disease or adverse medical condition, comprising providing to the mammal an amount of hydrogen sulfide or other Effective Compound sufficient to induce the mammal to enter pre-stasis or stasis prior to the onset or progression of the disease or adverse medical condition. This embodiment may be used in the context of a variety of different diseases and adverse medical conditions, including, e.g., unstable angina, post-angioplasty, aneurism, hemorrhagic stroke or shock, trauma, and blood loss.

In specific embodiments, the invention concerns methods of preventing an organism, such as a mammal, from bleeding to death or suffering irreversible tissue damage as a result of bleeding by providing to the mammal an amount of hydrogen sulfide or other Effective Compound sufficient to prevent the animal from bleeding to death. In certain additional embodiments, the organism may go into hemorrhagic shock but not die from excessive bleeding. The terms “bleeding” and “hemorrhaging” are used interchangeably to refer to any discharge of blood from a blood vessel. It includes, but is not limited to, internal and external bleeding, bleeding from an injury (which may be from an internal source, or from an external physical source such as from a gunshot, stabbing, physical trauma, etc.).

Moreover, additional embodiments of the invention concern prevention of death or irreversible tissue damage from blood loss or other lack of oxygenation to cells or tissue, such as from lack of an adequate blood supply. This may be the result of, for example, actual blood loss, or it may be from conditions or diseases that prevent cells or tissue from being perfused (e.g., reperfusion injury), that cause blockage of blood to cells or tissue, that reduce blood pressure locally or overall in an organism, that reduce the amount of oxygen is carried in the blood, or that reduces the number of oxygen carrying cells in the blood. Conditions and diseases that may be involved include, but are not limited to, blood clots and embolisms, cysts, growths, tumors, anemia (including sickle cell anemia), hemophilia, other blood clotting diseases (e.g., von Willebrand, ITP), and atherosclerosis. Such conditions and diseases also include those that create essentially hypoxic or anoxic conditions for cells or tissue in an organism because of an injury, disease, or condition.

In some cases, a sublethal collective dose or a nonlethal collective dose is administered to the biological matter. As discussed above, with respect to inducing stasis in biological matter that is not an entire organism, a “sublethal collective dose” means an amount of multiple administrations of the Effective Compound that collectively is less than half of the amount of the Effective Compound that would cause at least a majority of cell(s) to die within 24 hours of one of the administrations. In other embodiments, an effective amount is characterized as a near-lethal dose of the oxygen antagonist or other Effective Compound. Likewise, a “near lethal collective dose” means an amount of multiple administrations of the oxygen antagonist or other Effective Compound that is within 25% of the amount of the Effective Compound that would cause at least a majority of cell(s) to die within 24 hours of the one of the administrations. Also, a “supra-lethal collective dose” means an amount of multiple administrations of the Effective Compound that is at least 1.5 times the amount of the Effective Compound that would cause at least a majority of cell(s) (or the entire organism) to die within 24 hours of the one of the administrations. It is contemplated that multiple doses can be administered so as to induce stasis in the whole organism. The definition for “sub-lethal collective dose,” “near-lethal collective dose” and “supra-lethal collective dose” can be extrapolated based on the individual doses discussed earlier for stasis in whole organisms.

With respect to any method of the present invention, biological matter may be exposed to or contacted with more than one oxygen antagonist or other Effective Compound. Biological matter may be exposed to at least one Effective Compound, including 2, 3, 4, 5, 6, 7, 8, 9, 10 or more oxygen antagonists or other Effective Compound, or any range derivable therein. With multiple Effective Compounds, the term “effective amount” refers to the collective amount of Effective Compounds. For example, the biological matter may be exposed to a first Effective Compound and then exposed to a second Effective Compound. Alternatively, biological matter may be exposed to more than one Effective Compound at the same time or in an overlapping manner. Furthermore, it is contemplated that more than one Effective Compounds may be comprised or mixed together, such as in a single composition to which biological matter is exposed. Therefore, it is contemplated that, in some embodiments, a combination of Effective Compounds is employed in compositions, methods, and articles of manufacture of the invention.

Biological matter may be provided with or exposed to an Effective Compound through inhalation, injection, catheterization, immersion, lavage, perfusion, topical application, absorption, adsorption, or oral administration. Moreover, biological matter may be provided with or exposed to an Effective Compound by administration to the biological matter intravenously, intradermally, intraarterially, intraperitoneally, intralesionally, intracranially, intraarticularly, intraprostaticaly, intrapleurally, intratracheally, intranasally, intrathecally, intravitreally, intravaginally, intrarectally, topically, intratumorally, intramuscularly, intraperitoneally, intraocularly, subcutaneously, subconjunctival, intravesicularlly, mucosally, intrapericardially, intraumbilically, intraocularally, orally, topically, locally, by inhalation, by injection, by infusion, by continuous infusion, by localized perfusion, via a catheter, or via a lavage.

Methods and apparatuses of the invention involve a protective agent that in some embodiments is an oxygen antagonist. In still further embodiments, the oxygen antagonist is a reducing agent. Additionally, the oxygen antagonist can be characterized as a chalcogenide compound. It will be understood that Effective Compounds may also be protective agents. Moreover, any chalcogenide compound can be considered an Effective Compound so long as it achieves a goal of the invention, regardless of whether it is an oxygen antagonist.

In certain embodiments, the chalcogenide compound comprises sulfur, while in others, it comprises selenium, tellurium, or polonium. In certain embodiments, a chalcogenide compound contains one or more exposed sulfide groups. It is contemplated that this chalcogenide compounds contains 1, 2, 3, 4, 5, 6 or more exposed sulfide groups, or any range derivable therein. In particular embodiments, such a sulfide-containing compound is CS₂ (carbon disulfide).

Moreover, it is contemplated that in some embodiments of the invention, biological matter is provided with a precursor compound that becomes the active version of any compound of the present invention by exposure to biological matter, such as by chemical or enzymatic means. In addition, the compound may be provided to the biological matter as a salt of the compound in the form of a free radical, or a negatively charged, positively charged or multiply charged species. Some compounds qualify as both a Formula I and a Formula IV compound and in such cases, the use of the phrase “Formula I or Formula IV” is not intended to connote the exclusion of such compounds.

A compound identified by the structure of Formula I or Formula IV (or any other compound of the present invention) may also, in certain embodiments, be characterized as an oxygen antagonist, protective metabolic agent, or a precursor, prodrug, or salt thereof. It is further contemplated that the compound need not be characterized as such or qualify as such to be a compound used in the invention, so long as it achieves a particular method of the invention. In some other embodiments, the compound may be considered a chalcogenide compound. It is specifically contemplated that any compound identified by the structure of Formula I or Formula IV or any other compound set forth in this disclosure may be used instead of or in addition to an oxygen antagonist in methods, compositions, and apparatuses of the invention; similarly, any embodiments discussed with respect to any of structure having Formula I or Formula IV or otherwise set forth in this disclosure may be may be used instead of or in addition to an oxygen antagonist. Moreover, any compound set forth in this disclosure may be combined with any oxygen antagonist or any other Effective Compound described herein. It is also contemplated that any combination of such compounds may be provided or formulated together, sequentially (overlapping or nonoverlapping), and/or in an overlapping sequential manner (the administration of one compound is initiated and before that is complete, administration of another compound is initiated) in methods, compositions, and other articles of manufacture of the invention to achieve the desired effects set forth herein.

In certain embodiments, more than one compound with the structure of Formula I, Ia-Id, II, III, IIIa, IV, V, VI, VII, VIII, IX, X, XI, or any other compound of the present invention, is provided. In certain embodiments, multiple different compounds with a structure from the same formula (e.g., Formula I or Formula IV) are employed, while in other embodiments, when multiple different compounds are employed, they are from different formulas.

In specific embodiments, it is contemplated that multiple Effective Compounds are used, wherein one of the compounds is carbon dioxide (CO₂). It is contemplated that least one other compound is also a Formula I and/or Formula IV compound in some embodiments. In certain cases, carbon dioxide is provided to biological matter in combination with H₂S or an H₂S precursor (together, sequentially, or in an overlapping sequential manner).

The amount of carbon dioxide to which the biological matter may be exposed is about, at least about, or at most about, 2, 3, 4, 5, 6, 7, 8, 9, 10, 11, 12, 13, 14, 15, 16, 17, 18, 19, 20, 21, 22, 23, 24, 25, 26, 27, 28, 29, 30% or more, or any range derivable therein. In certain embodiments, the amount is expressed in terms of ppm, such as about, at least about, or at most about 350, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 60000, 70000, 80000, 90000, 100000, 110000, 120000, 130000, 140000, 150000, 160000, 170000, 180000, 190000, 200000, 210000, 220000, 230000, 240000, 250000, 260000, 270000, 280000, 290000, 300000 or more ppm, or any range derivable therein, as well as an molar equivalents. It is contemplated that these concentrations could apply to any other Effective Compound in gaseous form.

In other embodiments, it is specifically contemplated that the Effective Compound is sodium sulfide, sodium thiomethoxide, cysteamine, sodium thiocyanate, cysteamine-5-phosphate sodium salt, or tetrahydrothiopyran-4-ol. In additional embodiments, the Effective Compound is dimethylsulfoxide, thioacetic acid, selenourea, 2-(3-aminopropyl)-aminoethanethiol-dihydrogen-phosphate-ester, 2-mercapto-ethanol, thioglycolicether, sodium selenide, sodium methane sulfinate, thiourea, or dimethylsulfide. It is specifically contemplated that these compounds, or any others discussed herein including any compound with Formula I, Ia-Id, II, III, IIIa, IV, V, VI, VII, VIII, IX, X, XI, may be provided or administered to biological matter in an amount that is about, at least about, or at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000 mM or mmol/kg (of biological matter), or any range derivable therein.

It is specifically contemplated that any subset of Effective Compounds identified by name or structure may be used in methods, compositions and articles of manufacture. It is also specifically contemplated that any subset of these compounds may be disclaimed as not constituting embodiments of the invention. The present invention also concerns pharmaceutical compositions comprising a therapeutically effective amount of one or more Effective Compounds. It is understood that such pharmaceutical compositions are formulated in pharmaceutically acceptable compositions. For example, the composition may include a pharmaceutically acceptable diluent.

In certain embodiments, the pharmaceutical composition contains an effective dose of an active to provide when administered to a patient a Cₘₐₓ or a steady state plasma concentration of the Effective Compound to produce a therapeutically effective benefit. In certain embodiments, the Cₘₐₓ or steady state plasma concentration to be achieved is about, at least about, or at most about 0.01, 0.1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 3000, 4000, 5000, 6000, 7000, 8000, 9000, 10000 μM or more, or any range derivable therein. In certain embodiments, such as with H₂S, the desired Cₘₐₓ or steady state plasma concentration is about between 10 μM to about 10 mM, or between about 100 μM to about 1 mM, or between about 200 μM to about 800 μM. Appropriate measures may be taken to consider and evaluate levels of the compound already in the blood, such as sulfur.

In certain embodiments, the pharmaceutical composition provides an effective dose of H₂S to provide when administered to a patient a Cₘₐₓ or a steady state plasma concentration of between 10 μM to 10 mM, between about 100 μM to about 1 mM, or between about 200 μM to about 800 μM. In relating dosing of hydrogen sulfide to dosing with sulfide salts, in typical embodiments, the dosing of the salt is based on administering approximately the same sulfur equivalents as the dosing of the H₂S. Appropriate measures will be taken to consider and evaluate levels of sulfur already in the blood.

In certain embodiments, the composition comprises a gaseous form of one or more of the Effective Compounds specified herein. In another embodiment, the composition comprises a salt of one or more of these compounds. In one particular embodiment, a pharmaceutical composition comprises a gaseous form of Formula I, Ia-Id, II, III, IIIa, IV, V, VI, VII, VIII, IX, X or XI, or a salt of Formula I, Ia-Id, II, III, IIIa, IV, V, VI, VII, VIII, IX, X or XI. A gaseous form or salt of H₂S is specifically contemplated in some aspects of the invention. It is contemplated that the amount of gas to which biological matter is provided is about, at least about, or at most about 5, 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 60000, 70000, 80000, 90000, 100000, 110000, 120000, 130000, 140000, 150000, 160000, 170000, 180000, 190000, 200000, 210000, 220000, 230000, 240000, 250000, 260000, 270000, 280000, 290000, 300000, 310000, 320000, 330000, 340000, 350000, 360000, 370000, 380000, 390000, 400000 or more ppm, or any range derivable therein. Alternatively, the effective amount of gas(es) may be expressed as about, at least about, or at most about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any range derivable therein, with respect to the concentration in the air to which the biological matter is exposed. Moreover, it is contemplated that with some embodiments, the amount of gas to which biological matter is provided is about, at least about, or at most about 5, 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 parts per billion (ppb) or any range derivable therein. In particular embodiments, the amount of hydrogen selenide provided to biological matter is on this order of magnitude.

In some embodiments of the invention, the pharmaceutical composition is a liquid. As discussed elsewhere, the composition may be a liquid with the relevant compound(s) dissolved or bubbled into the composition. In some cases, the pharmaceutical composition is a medical gas. According to the United States Food and Drug Administration, “medical gases” are those gases that are drugs within the meaning of §201(g)(l) of the Federal Food, Drug and Cosmetic Act (“the Act”) (21 U.S.C. §321(g) and pursuant to §503(b)(l)(A) of the Act (21 U.S.C. §353(b)(l)(A) are required to be dispensed by prescription. As such, such medical gases require an appropriate FDA label. A medical gas includes at least one Effective Compound.

The present invention further comprises apparatuses and articles of manufacture comprising packaging material and, contained within the packaging material, an effective stasis compound, wherein the packaging material comprises a label that indicates that it can be used for inducing stasis in in vivo biological matter.

In some embodiments, the apparatus or article of manufacture further includes a pharmaceutically acceptable diluent. In particular other embodiments, the apparatus or article of manufacture has a buffering agent. The Effective Compound is provided in a first sealed container and the pharmaceutically acceptable diluent is provided in a second sealed container. In other embodiments, the device or article further has instructions for mixing the Effective Compound and the diluent. Additionally, the Effective Compound can be reconstituted for achieving any method of the invention, such as for inducing stasis in in vivo biological matter. It is contemplated that any label would specify the result to be achieved and the use of the compound for patients in need of that result.

The present invention also concerns an article of manufacture comprising packed together: an Effective Compound, instructions for use of the effective stasis compound, comprising: (a) identifying in vivo tissue in need of stasis treatment; and (b) administering an effective amount of the Effective Compound to the in vivo biological matter.

In further embodiments of the invention, there is an article of manufacture comprising a medical gas including an Effective Compound and a label comprising details or use and administration for inducing stasis in a biological matter or any other method of the invention.

The present invention also concerns kits and methods of using these kits. In some embodiments, there are kits for the delivery of an Effective Compound to a tissue site in need of stasis treatment, or any other treatment of the claimed invention, comprising: a drape adapted for forming a sealed envelope against a tissue site; a container comprising an Effective Compound; and an inlet in the drape, wherein the container comprising the Effective Compound is in communication with the inlet. In certain embodiments, the kit includes an outlet in the drape wherein the outlet is in communication with a negative pressure source. In some cases, the drape comprises elastomeric material and/or has a pressure sensitive adhesive covering the periphery of the drape. The outlet may be placed in fluid communication with the negative pressure source, which may or may not be a vacuum pump. There may also be a flexible conduit communicating between the outlet and the negative pressure source. In some embodiments, the kit includes a canister, which may or may not be removable, in fluid communication between the outlet and the negative pressure source. It is contemplated that the container includes an Effective Compound that is in gaseous communication with the inlet. In certain embodiments, the container includes an Effective Compound that is a gas or a liquid gas. The kit may also include a vaporizer in communication between the container comprising an oxygen antagonist and the inlet. In addition, it may have a return outlet in communication with the container comprising the Effective Compound.

In particular embodiments, the Effective Compound in the kits is carbon monoxide, carbon dioxide, H₂Se, and/or H₂S. In certain embodiments, the tissue site for which the kit or method is applied is wounded.

Moreover, it will be generally understood that any compound discussed herein as an oxygen antagonist can be provided in prodrug form to the biological matter, meaning that the biological matter or other substance(s) in the environment of the biological matter alters the prodrug into its active form, that is, into an oxygen antagonist. It is contemplated that the term “precursor” covers compounds that are considered “prodrugs.”

The oxygen antagonist or other Effective Compound may be or may be provided as a gas, semi-solid liquid (such as a gel or paste), liquid, or solid. It is contemplated that biological matter may be exposed to more than one such Effective Compound and/or to that Effective Compound in more than one state. Moreover, the Effective Compound may be formulated for a particular mode of administration, as is discussed herein. In certain embodiments, the Effective Compound is in pharmaceutical acceptable formulation for intravenous delivery.

In certain embodiments, the Effective Compound is a gas. In particular embodiments, the gaseous Effective Compound includes carbon monoxide, carbon dioxide, nitrogen, sulfur, selenium, tellurium, or polonium, or a mixture thereof. Moreover, it is specifically contemplated that the Effective Compound may, in certain embodiments, be a chalcogenide compound as a gas. In some embodiments, the Effective Compound is in a gas mixture comprising more than one gas. The other gas(es) is a non-toxic and/or a non-reactive gas in some embodiments. In some embodiments, the other gas is a noble gas (helium, neon, argon, krypton, xenon, radon, or ununoctium), nitrogen, nitrous oxide, hydrogen, or a mixture thereof. For instance, the non-reactive gas may simply be a mixture that constitutes “room air,” which is a mixture of nitrogen, oxygen, argon and carbon dioxide, as well as trace amounts of other atoms/molecules such as neon, helium, methane, krypton, and hydrogen. The precise amounts of each varies, though a typical sample might contain about 78% nitrogen, 21% oxygen, 0.9% argon, and 0.04% carbon dioxide. It is contemplated that in the context of the present invention, “room air” is a mixture containing about 75 to about 81% nitrogen, about 18 to about 24% oxygen, about 0.7 to about 1.1% argon, and about 0.02% to about 0.06% carbon dioxide. A gaseous Effective Compound may be first diluted with a non-toxic and/or non-reactive gas prior to administration or exposure to biological matter. Additionally or alternatively, any gaseous Effective Compound may be mixed with room air prior to administration or exposure to biological matter or the compound may be administered or exposed to the biological matter in room air.

In some instances, the gas mixture also contains oxygen. An Effective Compound gas is mixed with oxygen to form an oxygen gas (O₂) mixture in other embodiments of the invention. Specifically contemplated is an oxygen gas mixture in which the amount of oxygen in the oxygen gas mixture is less than the total amount of all other gas or gases in the mixture.

In some embodiments, the Effective Compound gas is carbon monoxide and the amount of carbon monoxide is about the same or exceeds any amount of oxygen in the oxygen gas mixture. In particular embodiments, carbon monoxide is employed with blood-free biological matter. The term “blood-free biological matter” refers to cells and organs whose oxygenation is not dependent, or no longer dependent, on the vasculature, such as an organ for transplant. The atmosphere may be 100% CO, but as will be evident to one skilled in the art, the amount of CO may be balanced with gases other than oxygen providing that the amount of usable oxygen is reduced to a level that prevents cellular respiration. In this context, the ratio of carbon monoxide-to-oxygen may be 85:15 or greater, 199:1 or greater or 399:1 or greater. In certain embodiments, the ratio is about, at least about, or at most about 1:1, 2:1, 2.5:1, 3:1, 4:1, 5:1, 6:1, 7:1, 8:1, 9:1, 10:1, 15:1, 20:1, 25:1, 30:1. 35:1, 40:1, 45:1, 50:1, 55:1, 60:1, 65:1, 70:1, 75:1, 80:1, 85:1, 90:1, 95:1, 100:1, 110:1, 120:1, 130:1, 140:1, 150:1, 160:1, 170:1, 180:1, 190:1, 200:1, 210:1, 220:1, 230:1, 240:1, 250:1, 260:1, 270:1, 280:1, 290:1, 300:1, 310:1, 320:1, 330:1, 340:1, 350:1, 360:1, 370:1, 380:1, 390:1, 400:1, 410:1, 420:1, 430:1, 440:1, 450:1, 460:1, 470:1, 480:1, 490:1, 500:1 or more, or any range derivable therein.

In still further embodiments, the above numbers pertain to the ratio of carbon monoxide to a mixture of oxygen and one or more other gases. In some cases, it is contemplated that the other gas is a nonreactive gas such as nitrogen (N₂). Thus, in other embodiments of the invention, the above numbers apply to ratios of carbon monoxide to a combination of oxygen and nitrogen (O₂/N₂) that can be used in methods and apparatuses of the invention. Accordingly, it will be understood that other gases may or may not be present. In some embodiments, the CO:oxygen ratio is balanced with one or more other gases (non-carbon monoxide and non-oxygen gases). In particular embodiments, the CO:oxygen ratio is balanced with nitrogen. In still further embodiments, the amount of CO is a ratio of CO compared to room air, as is described by the numbers above.

In some cases, the amount of carbon monoxide is relative to the amount of oxygen, while in others, it is an absolute amount. For example, in some embodiments of the invention, the amount of oxygen is in terms of “parts per million (ppm)” which is a measure of the parts in volume of oxygen in a million parts of air at standard temperature and pressure of 20° C. and one atmosphere pressure and the balance of the gas volume is made up with carbon monoxide. In this context, the amount of carbon monoxide to oxygen is related in terms of parts per million of oxygen balanced with carbon monoxide. It is contemplated that the atmosphere to which the biological material is exposed or incubated may be at least 0, 50, 100, 200, 300, 400, 500, 1000, or 2000 parts per million (ppm) of oxygen balanced with carbon monoxide and in some cases carbon monoxide mixed with a non-toxic and/or non-reactive gas The term “environment” refers to the immediate environment of the biological matter, that is, the environment with which it is in direct contact. Thus, the biological material must be directly exposed to carbon monoxide, and it is insufficient that a sealed tank of carbon monoxide be in the same room as the biological matter and be considered to be incubated an “environment” according to the invention. Alternatively, the atmosphere may be expressed in terms of kPa. It is generally understood that 1 million parts=101 kPa at 1 atmosphere. In embodiments of the invention, the environment in which a biological material is incubated or exposed to is about, at least about, or at most about 0.001, 0.005, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20. 0.21, 0.22, 0.23, 0.24, 0.25, 0.26, 0.27, 0.28, 0.29, 0.30, 0.35, 0.40, 0.45, 0.50, 0.55, 0.60, 0.65, 0.70, 0.75, 0.80, 0.5, 0.90, 0.95, 1.0 kPa or more O₂, or any range derivable therein. As described above, such levels can be balanced with carbon monoxide and/or other non-toxic and/or non-reactive gas(es) Also, the atmosphere may be defined in terms of CO levels in kPa units. In certain embodiments, the atmosphere is about, at least about, or at most about 1, 5, 10, 15, 20, 25, 30, 35, 40, 45, 50, 55, 60, 65, 70, 75, 80, 85, 90, 95, 101, 101.3 kPa CO, or any range derivable therein. In particular embodiments, the partial pressure is about or at least about 85, 90, 95, 101, 101.3 kPa CO, or any range derivable therein.

The amount of time the sample is incubated or exposed to carbon monoxide can also vary in embodiments of the invention. In some embodiments, the sample is incubated or exposed to carbon monoxide for about, for at least about, or for at most about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more minutes and/or, 1, 2, 3, 4, 5, 6, 7, 8, 9, 10, 11, 12, 13, 14, 15, 16, 17, 18, 19, 20, 21, 22, 23, 24 hours, and/or 1, 2, 3, 4, 5, 6, 7, 8, 9, 10 or more days.

In some embodiments, the invention concerns compositions and articles of manufacture that contain one or more Effective Compounds. In certain embodiments, a composition has one or more of these Effective Compounds as a gas that is bubbled in it so that the composition provides the compound to the biological matter when it is exposed to the composition. Such compounds may be gels, liquids, or other semi-solid material. In certain embodiments, a solution has an oxygen antagonist as a gas bubbled through it. It is contemplated that the amount bubbled in the gas will provide the appropriate amount of the compound to biological material exposed to the solution. In certain embodiments, the amount of gas bubbled into the solution is about, at least about, or at most about 0.5, 1.0, 1.5, 2.0. 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, 20, 25, 30, 35, 40, 45, 50, 55, 60, 65, 70, 75, 80, 85, 90, 95, 100 times or more, or any range derivable therein, than the amount to which the biological matter is effectively provided.

Biological matter is exposed to the gas in a closed container in some embodiments of the invention. In some cases, the closed container can maintain a particular environment or modulate the environment as is desired. The environment refers to the amount of oxygen antagonist that the biological matter is exposed and/or the temperature, gas composition, or pressure of the environment. In some cases, the biological matter is placed under a vacuum before, during, or after exposure to an oxygen antagonist or other Effective Compound. Moreover, in other cases, the biological matter is exposed to a normoxic environment after being exposed to an oxygen antagonist or other Effective Compound. In certain embodiments, the present invention includes methods for inducing stasis or protecting biological matter from injury or disease that include providing an Effective Compound to the biological matter in combination with providing another stasis-inducing Effective Compound or environmental condition to the biological matter. Such combination treatment may occur in any order, e.g., simultaneously or sequentially. In certain embodiments, an Effective Compound is provided to biological matter, and the biological matter is subsequently placed under hypoxic conditions, such as 5% O₂, or sequentially exposed to increasingly hypoxic conditions, such as 5% O₂ followed by 4% O₂, 3% O₂, 2% O₂, 1% O₂, or O₂-free conditions, or any sequential combination of such conditions.

Moreover, in other embodiments, the environment containing the biological matter cycles at least once to a different amount or concentration of the oxygen antagonist or other Effective Compound, wherein the difference in amount or concentration is by at least one percentage difference. The environment may cycle back and forth between one or more amounts or concentrations of the oxygen antagonist or other Effective Compound, or it may gradually increase or decrease the amount or concentrations of an that compound. In some cases, the different amount or concentration is between about 0 and 99.9% of the amount or concentration of the oxygen antagonist or other Effective Compound to which the biological matter was initially exposed. It is contemplated that the difference in amount and/or concentration is about, at least about, or at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or more, or any range derivable therein.

Methods of the invention can also include a step of subjecting biological matter to a controlled temperature environment. In certain embodiments, the biological matter is exposed to a temperature that is a “nonphysiological temperature environment,” which refers to a temperature in which the biological matter cannot live in for more than 96 hours. The controlled temperature environment can have a temperature of about, at least about, or at most about −210, −200, −190, −180, −170, −160, −150, −140, −130, −120, −110, −100, −90, −80, −70, −60, −50, −40, −30, −20, −10, −5, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200° C. or more, or any range derivable therein. Biological matter may also be exposed to an oxygen antagonist or other Effective Compound at room temperature, which means a temperature between about 20° C. and about 25° C. Furthermore, it is contemplated the biological matter achieves a core temperature of any amount or range of amounts discussed.

It is contemplated that the biological matter can be subjected to a nonphysiological temperature environment or a controlled temperature environment before, during or after exposure to the oxygen antagonist(s) or other Effective Compound(s). Furthermore, in some embodiments, the biological matter is subjected to a nonphysiological temperature environment or a controlled temperature environment for a period of time between about one minute and about one year. The amount of time may be about, at least about, or at most about 30 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, 1, 2, 3, 4, 5, 6, 7, 8, 9, 10, 11, 12 months, 1, 2, 3, 4, 5, 6, 7, 8, 9, 10, 11, 12, 13, 14, 15, 16, 17, 18, 19, 20 or more years, and any combination or range derivable therein. Moreover, there may also be a step of increasing the ambient temperature relative to the reduced temperature.

Moreover, it is contemplated that the temperature may be altered or cycled during the process in which temperature is controlled. In some embodiments, the temperature of the biological matter may first be reduced before it is placed in the environment that has the oxygen antagonist or other Effective Compound, while in others, the biological matter may be cooled by placing it in the environment with the Effective Compound, that is below the temperature of the biological matter. The biological matter and/or environment may be cooled or heated gradually, such that the temperature of the biological matter or environment starts at one temperature but then reaches another temperature.

Methods of the invention can also include a step of subjecting biological matter to a controlled pressure environment. In certain embodiments, the biological matter is exposed to pressure that is lower than the pressure under which the organism is typically under. In certain embodiments, the biological matter is subjected to a “nonphysiological pressure environment,” which refers to a pressure under which the biological matter cannot live under for more than 96 hours. The controlled pressure environment can have a pressure of about, at least about, or at most 10⁻¹⁴, 10⁻¹³, 10⁻¹², 10⁻¹¹, 10¹⁰, 10⁻⁹, 10⁻⁸, 10⁻⁷, 10⁻⁶, 10⁻⁵, 10⁻⁴, 10⁻³, 10⁻², 10⁻¹, 0.2, 0.3, 0.4 or 0.5 atm or more, or any range derivable therein.

It is contemplated that the biological matter can be subjected to a nonphysiological pressure environment or a controlled pressure environment before, during or after exposure to the Effective Compound(s). Furthermore, in some embodiments, the biological matter is subjected to a nonphysiological pressure environment or a controlled pressure environment for a period of time between about one minute and about one year. The amount of time may be about, at least about, or at most about 30 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, 1, 2, 3, 4, 5, 6, 7, 8, 9, 10, 11, 12 months, 1, 2, 3, 4, 5, 6, 7, 8, 9, 10, 11, 12, 13, 14, 15, 16, 17, 18, 19, 20 or more years, and any combination or range derivable therein.

Moreover, it is contemplated that the pressure may be altered or cycled during the process in which pressure is controlled. In some embodiments, the pressure to which the biological matter is exposed may first be reduced before it is placed in the environment that has the Effective Compound, while in others, the biological matter placed under pressure after exposure to an Effective Compound. The pressure may be reduced gradually, such that the pressure of the environment starts at one pressure but then reaches another pressure within 10, 20, 30, 40, 50, 60 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, and/or 1, 2, 3, 4, 5, 6, 7 days or more, and any combination or range derivable therein. In certain embodiments, methods include modulating environmental oxygen levels or removing the biological material from an environment having oxygen. Operationally, exposing biological material to an environment in which oxygen is diminished or absent may mimic exposure of the biological material to an oxygen antagonist. It is contemplated that in some embodiments of the invention, biological matter is exposed to or provided with an Effective Compound under conditions in which the environment of the biological matter is hypoxic or anoxic, as described in further detail below. This may be intentional or nonintentional. Thus, in some embodiments of the invention, biological matter is intentionally placed in an environment that is anoxic or hypoxic or in an environment that is made anoxic or hypoxic. In other embodiments, the biological matter is under such conditions as a result of an unintended situation, for example, if the biological matter is under ischemic or potentially ischemic conditions. Therefore, it is contemplated in some cases that the hypoxic or anoxic conditions would damage the matter in the absence of the Effective Compound.

In certain methods of the invention, there also is a step of assessing the level of the oxygen antagonist and/or oxidative phosphorylation in the biological matter in which stasis was induced. Moreover, in some embodiments of the invention, there is a step of assessing the level of cellular metabolism that is generally occurring in the biological matter. In some cases, the amount of the Effective Compound in the biological matter is measured and/or a reduction in the temperature of the biological matter is assessed. Moreover, in some methods of the invention, the extent of one or more therapeutic effects is evaluated.

In certain other embodiments, any toxicity effect on the biological matter from an Effective Compound and/or environmental change (temperature, pressure) is monitored or controlled for. It is contemplated that toxicity can be controlled for by altering the level, amount, duration, or frequency of an Effective Compound and/or environmental change to which the biological matter is exposed. In certain embodiments the alteration is a reduction, while in certain other embodiments, the alteration is an increase. It is contemplated that the skilled artisan is aware of a number of ways of evaluating toxicity effects in biological matter.

Other optional steps for methods of the invention include identifying an appropriate Effective Compound; diagnosing the patient; taking a patient history and/or having one or more tests done on the patient prior to administering or prescribing an Effective Compound to the patient.

Compositions, methods, and articles of manufacture of the invention can be used on biological matter that will be transferred back into the donor organism from which it was derived (autologous) or a different recipient (heterologous) subject. In some embodiments, biological matter is obtained directly from a donor organism. In others, the biological matter is placed in culture prior to exposure to an oxygen antagonist or other Effective Compound. In some situations, the biological matter is obtained from a donor organism administered extracorporeal membrane oxygenation prior to retrieval of the biological matter, which is a technique implemented to aid in the preservation of biological matter. Moreover, methods include administering or implanting the biological matter in which stasis was induced to a live recipient organism.

Methods of the invention also concern inducing stasis in in vivo biological matter comprising incubating the biological matter with an oxygen antagonist or other Effective Compound that creates hypoxic conditions for an effective amount of time for the biological matter to enter stasis.

Furthermore, other embodiments of the invention include methods of reducing oxygen demand in in vivo biological matter comprising contacting the biological matter with an effective amount of an oxygen antagonist or other Effective Compound to reduce their oxygen demand. It is contemplated that oxygen demand is reduced about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or any range derivable therein, with respect to the amount of oxygen demand in cells of the biological matter or a representative sample of cells from the biological matter not exposed or no longer exposed to the oxygen antagonist or other Effective Compound.

Other aspects of the invention concern methods for preserving in vivo biological matter comprising exposing the in vivo biological matter to an effective amount of an oxygen antagonist or other Effective Compound to preserve the biological matter in vivo.

The present invention also concerns a method of delaying or reducing the effects of trauma on or in an organism comprising exposing biological matter at risk for trauma to an effective amount of an oxygen antagonist or other Effective Compound.

In other aspects of the invention, there are methods for treating or preventing hemorrhagic shock in a patient comprising exposing the patient to an effective amount of an oxygen antagonist or other Effective Compound. Alternatively, in some embodiments methods prevents lethality in the patient as a result of the bleeding and/or hemorrhagic shock. In such methods of preventing a patient from bleeding to death or prevent lethality in a bleeding patient, steps include exposing the patient to an effective amount of an oxygen antagonist or other Effective Compound. In certain embodiments, the oxygen antagonist is specifically contemplated to be a chalcogenide compound such as H₂S.

Methods for reducing heart rate in an organism are also included as part of the invention. Such methods involve contacting the biological sample or organism with an effective amount of an oxygen antagonist or other Effective Compound.

One embodiment of the invention relates to a method of inducing hibernation in a mammal comprising contacting the mammal with an effective amount of an oxygen antagonist or other Effective Compound.

In another embodiment, there is a method of anesthetizing an organism comprising exposing biological matter in which anesthesia is desired to an effective amount of an oxygen antagonist or other Effective Compound. It is contemplated that the anesthesia may be similar to local or general anesthesia.

The present invention further includes methods of protecting a mammal from radiation therapy or chemotherapy comprising contacting the mammal with an effective amount of an oxygen antagonist or other Effective Compound prior to or during radiation therapy or chemotherapy. With local administration of the cancer therapy, it is specifically contemplated that the oxygen antagonist or other Effective Compound may also be administered locally to the affected organ, tissue, and/or cells. In certain embodiments, methods can be used for preventing or reducing hair loss in a chemotherapy patient. It is contemplated that such a patient may have already received chemotherapy or be a candidate for chemotherapy. In particular cases, it is contemplated that an Effective Compound is provided to the patient as a topical gel to be applied where the hair loss is anticipated or present.

In additional embodiments, there are methods of treating a hyperproliferative disease (e.g., cancer) in a mammal comprising contacting the mammal with an effective amount of an oxygen antagonist or other Effective Compound and subjecting the mammal to hyperthermia therapy.

While methods of the invention may be applied to preserving organs for transplant, other aspects of the invention concern the recipient organism. In some embodiments, there are methods of inhibiting rejection of an organ transplant in a mammal comprising providing the mammal with an effective amount of an oxygen antagonist or other Effective Compound.

Temperature regulation can be achieved in organisms by employing oxygen antagonists or other Effective Compounds. In some embodiments, there is a method of treating a subject with hypothermia comprising (a) contacting the subject with an effective amount of an oxygen antagonist, and then (b) subjecting the subject to an environmental temperature above that of the subject. In other embodiments, the present invention includes a method of treating a subject with hyperthermia comprising (a) contacting the subject with an effective amount of an oxygen antagonist or other Effective Compound. In some cases, treatment of hyperthermia also includes (b) subjecting the subject to an environmental temperature that is at least about 20° C. below that of the subject. As discussed above, exposing the subject to nonphysiological or a controlled temperature environment can be used in additional embodiments. It is contemplated that this method may be achieved with Effective Compounds generally.

In some cases, the invention concerns a method for inducing cardioplegia in a patient undergoing bypass surgery comprising administering to the patient an effective amount of an oxygen antagonist or other Effective Compound. It is contemplated that administration may be local to the heart so as to protect it.

Other aspects of the invention relate to a method for preventing hematologic shock in a patient comprising administering to the patient an effective amount of an oxygen antagonist or other Effective Compound.

Moreover, there are methods for promoting wound healing in an organism comprising administering to the organism or wound an effective amount of an oxygen antagonist or other Effective Compound.

In addition, the present invention covers a method for preventing or treating neurodegeneration in a mammal comprising administering to the mammal an effective amount of an oxygen antagonist or other Effective Compound.

The present invention also covers reducing the oxygen requirement of biological matter, meaning that the amount of oxygen required by the biological matter to survive is reduced. This can be achieved by providing an effective amount of one or more Effective Compounds. It is generally known how much oxygen particular biological matter require to survive, which can also be dependent on time, pressure, and temperature. In certain embodiments of the invention, the oxygen requirement of the biological matter is reduced by about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any range derivable therein, as compared to the requirement of the biological matter in the absence of the effective amount of the Effective Compound(s).

Additional embodiments of the invention concern methods for preventing hair loss, such as from chemotherapy, by administering to a patient who has or will undergo chemotherapy an effective amount of at least one Effective Compound.

In cases in which biological matter is being protected from damage or further damage, it is contemplated that the biological matter may be exposed to an oxygen antagonist within about, within at least about, or within at most about 30 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, 1, 2, 3, 4, 5, 6, 7, 8, 9, 10, 11, 12 months, 1, 2, 3, 4, 5, 6, 7, 8, 9, 10, 11, 12, 13, 14, 15, 16, 17, 18, 19, 20 or more years, and any combination or range derivable therein, after initial damage (trauma or wound or degeneration) occurs. Thus in additional embodiments of the invention, methods include an initial assessment of any damage, trauma, a wound, or degeneration.

In certain embodiments of the invention there are methods for treating a patient affected with a hematological disorder, which means a disease, disorder or condition that affects any hematopoietic cells or tissue. Examples include sickle cell disease and thalassemia. Thus, in some embodiments, there are methods of treating a patient with sickle cell disease or thalassemia with an effective amount of an Effective Compound. In other embodiments, there are methods for enhancing survivability in a patient with cystic fibrosis (CF) by administering or providing an effective amount of an Effective Compound. In other methods of the invention, there are methods for treating cynanide poisoning in a subject comprising administering an effective amount of an Effective Compound. In certain embodiments, the compound is H₂S.

Other aspects of the invention concern methods for preserving one or more cells that are separate from an organism comprising contacting the cell(s) with an effective amount of an oxygen antagonist or other Effective Compound to preserve the one or more cells. In addition to the cells and cell types discussed above and elsewhere in this application, it is contemplated that shrimp embryos are specifically contemplated for use with the present invention.

Moreover, in some embodiments of the invention, there are methods for preserving platelets. Shortcomings of the prior art are reduced or eliminated using techniques of this disclosure. Embodiments concerning platelets and oxygen reduction find wide application including but not limited to any application that would benefit from longer-lasting storage of platelets.

In one embodiment, oxygen reduction techniques can be embodied in a kit. For example, the kit currently sold under product number 261215, available from Becton Dickinson, makes use of select techniques described here. That kit includes an anaerobic generator (e.g., a hydrogen gas generator), Palladium Catalysts, an anaerobic indicator, and a gas impermeable, sealable, “BioBag” into which the above components (together with platelets in a gas-permeable bag) are placed and sealed.

In other embodiments of the invention, there are methods for reversibly inhibiting metabolism of a cell and/or organism by providing an effective amount of an Effective Compound. It is specifically contemplated that rotenone is not the compound employed in this method, or possibly other methods of the invention. Moreover, it is also contemplated that in some embodiments, rotenone is excluded as an Effective Compound. Similarly, it is contemplated that nitric oxide may be excluded as an Effective Compound.

In other embodiments of the invention, methods are provided for enhancing the ability of biological matter to enter stasis in response to an injury or disease by providing an effective amount of an Effective Compound, thereby protecting the biological matter from damage or injury, thereby enhancing survival of biological matter. Related embodiments include methods of preparing or priming biological matter for entry into stasis in response to an injury or disease by providing an effective amount of an Effective Compound. Other related embodiments include method of inducing biological matter into pre-stasis, thereby protecting the biological matter from damage or injury. For example, treatment with an Effective Compound at a dosage or for a time less than required to induce stasis enables the biological matter to more readily or more completely achieve a beneficial state of stasis in response to an injury or disease, while in the absence of treatment with the Effective Compound, the biological matter would die or suffer damage or injury before it reached a protective level of stasis, e.g., a level sufficient to render the biological matter resistant to lethal hypoxia.

Certain injuries and disease states cause biological matter to reduce its metabolism and/or temperature to degrees that may not achieve stasis. For example, hypoxia, ischemia, and blood loss all reduce the amount of oxygen available and supplied to oxygen utilizing biological matter, thereby reducing oxygen utilization in cells of the biological matter, reducing energy production derived from oxidative phosphorylation, and thereby decreasing thermogenesis, leading to hypothermia. Depending on the severity or time elapsed following the onset or progression of the injurious or disease insult, “stasis” may or may not have been achieved. Treatment with an Effective Compound lowers the threshold (i.e., the severity or duration of the insult that is needed to achieve stasis) for induction of stasis, or it may add to or synergize with the injurious or disease stimuli to induce stasis in biological matter under injurious conditions that would not have resulted in stasis were it not for the Effective Compound treatment. Such activity of Effective Compounds is determined by comparing the stasis-inducing effects (magnitude, kinetics) of injurious or disease stimuli alone with those in which the biological matter was pre-exposed, exposed concomitantly, exposed after, or any combination thereof, to the Effective Compound.

In other aspects of the invention, there are methods for inducing sleep in an organism comprising exposing the organism to an effective amount of an Effective Compound, wherein the effective amount is less than an amount that can induce stasis in the organism. The term “sleep” is used according to its ordinary and plain meaning in a medical context. Sleep is distinguishable from other states of unconsciousness, which are also contemplated as states that can be achieved using methods of the invention.

The present invention also concerns methods for anesthetizing biological matter comprising exposing the matter to an effective amount of an Effective Compound, wherein the effective amount is less than an amount that can induce stasis in the organism.

In the methods discussed above, an effective amount that is less than an amount that can induce stasis in an organism may be reduced with respect to duration and/or amount. That reduction may be a reduction in amount by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent, or any range derivable therein, of the amount to induce stasis. A reduction may be a reduction in duration (length of exposure time) by 1, 2, 3, 4, 5, 6, 7, 8, 9, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7, days, 1, 2, 3, 4, 5 weeks, and/or 1, 2, 3, 4, 5, 6, 7, 8, 9, 10, 11, 12 months, or any range derivable therein. Alternatively, the reduction may be in terms of the overall effective amount provided to the biological matter, which may be a reduction of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent, or any range derivable therein, relative to the overall effective amount to induce stasis in an organism of that species and/or size.

It is specifically contemplated that the present invention can be used for preserving organisms that are used for consumption or laboratory research, such as flies, frogs, fish, mice, rats, dogs, shrimp, and embryos thereof.

Methods of the invention can involve employing an apparatus or system that maintains the environment in which biological matter is placed or exposed to. The invention includes an apparatus in which an oxygen antagonist or other Effective Compound, particularly as a gas, is supplied. In some embodiments, the apparatus includes a container with a sample chamber for holding the biological matter, wherein the container is connected to a supply of gas comprising the oxygen antagonist(s). It is specifically contemplated that the container may be a solid container or it may flexible, such as a bag.

In some embodiments, the invention is an apparatus for preserving cell(s), the apparatus comprising: a container having a sample chamber with a volume of no greater than 775 liters; and a first gas supply in fluid communication with the sample chamber, the first gas supply including carbon monoxide. In further embodiments, the apparatus also includes a cooling unit that regulates the temperature inside the sample chamber and/or a gas regulator that regulates the amount of oxygen antagonist or other Effective Compound in the chamber or the amount of oxygen antagonist or other Effective Compound in a solution that is in the chamber.

It is contemplated that there may be a gas supply for a second or additional gas or a second or additional gas supply for the oxygen antagonist or other Effective Compound. The second gas supply may be connected with the sample chamber or it may be connected with the first gas supply. The additional gas, as discussed above, may be a non-toxic and/or non-reactive gas.

A gas regulator is part of the apparatus in some embodiments of the invention. One, two, three, or more gas regulators may be employed. In some cases, the gas regulator regulates the gas supplied to the sample chamber from the first gas supply. Alternatively, it regulates the gas supplied to the sample chamber or first gas supply from the second gas supply, or there may be a regulator for both the first and second gas supplies. It is further contemplated that any gas regulator can be programmed to control the amount of gas supplied to the sample chamber and/or to another gas supply. The regulation may or may not be for a specified period of time. There may be a gas regulator, which may or may not be programmable, for any gas supply directly or indirectly connected to the sample chamber. In some cases, the gas regulator is electronically programmable.

In some cases, the pressure and/or the temperature inside the chamber can be regulated with either a pressure regulator or temperature regulator, respectively. As with the gas regulator, these regulators may be electronically programmable. The apparatus of the invention may also have a cooling and/or heating unit to achieve the temperatures discussed above. The unit may or may not be electronically programmable.

In additional embodiments, the apparatus includes a wheeled cart on which the container rests or it may have one or more handles.

It is specifically contemplated that the invention includes an apparatus for cell(s), tissues, organs, and even whole organisms, in which the apparatus has: a container having a sample chamber; a first gas supply in fluid communication with the sample chamber, the first gas supply including the oxygen antagonist(s) or other Effective Compound(s); and an electronically-programmable gas regulator that regulates gas supplied to the sample chamber from the first gas supply.

In some embodiments, the apparatus also has a structure configured to provide a vacuum within the sample chamber.

Moreover, any oxygen antagonist or other Effective Compound described in this application is contemplated for use with apparatuses of the invention. In specific embodiments, carbon monoxide can be administered using this apparatus. In other cases, a chalcogenide compound can be administered or a compound having the reducing agent structure. In still further embodiments, an Effective Compound is administered using the apparatus. In specific embodiments, the invention covers a device or its use. In certain embodiments, the device is single dose delivery device. In other embodiments, the device is an inhaler or nebulizer. In even further embodiments, other devices include, but are not limited to, an injection device such as a pen, a pump such as an infusion pump, or a patch. Moreover, it is contemplated that these devices may or may not be single dose delivery devices.

Additionally, the present invention concerns screening assays. In some embodiments, a candidate substance is screened for the ability to act as an oxygen antagonist or Effective Compound, specifically including a protective metabolic agent. This can be done using any assay described herein, such as by measuring carbon dioxide output. Any substance identified as having exhibiting characteristics of an oxygen antagonist or other Effective Compound can be further characterized or tested. Moreover, it is contemplated that such a substance can be administered to biological matter to induce stasis or manufactured thereafter.

In certain embodiments, there are screening methods for Effective Compounds, including effective stasis compounds. Furthermore, the methods of screening may be for oxygen antagonists or for any other compounds that can effect the methods discussed herein. In some embodiments, there are screening methods involving a) exposing a zebrafish embryo to a substance; b) measuring the heart rate of the embryo; c) comparing the heart rate of the embryo in the presence of the substance to the heart rate in the absence of the substance, wherein a reduction of heart rate, such as by 50% or more, identifies the substance as a candidate Effective Compound. Instead of zebrafish embryos, it is contemplated that other non-human organisms may be used as well, such as fish, frogs, flies, shrimp, or their embryos. In further embodiments, the heart rate of the embryo is measured by counting the number of heartbeats. This can be done, in some cases, by viewing the embryo under a dissecting microscope.

Other screening embodiments involve: a) exposing a nematode to a substance; b) assaying one or more of the following cellular respiration factors: i) core body temperature; ii) oxygen consumption; iii) motility; or, iv) carbon dioxide production; c) comparing the cellular respiration factor of the nematode in the presence of the substance to the cellular respiration factor in the absence of the substance, wherein a reduction of the characteristic identifies the substance as a candidate Effective Compound. It is specifically contemplated that motility of the nematodes is assayed in some methods of the invention.

In some embodiments, the methods first involve identifying an appropriate substance to screen. In certain embodiments, the substance will be a chalcogenide, reducing agent, or have the structure of Formula I or Formula IV, or any other compound discussed herein.

It is further contemplated that subsequent screens can be done in organisms considered higher or more complex than those used in preliminary or initial screens. Thus, it is contemplated that one or more cellular respiration factors will be assayed in these other organisms to further evaluate a candidate compound. In certain embodiments, subsequent screens involve the use of mice, rats, dogs, etc.

It is contemplated that a number of different organisms or biological matter (other cells or tissues) could be used and a number of different cellular respiration factors could be assayed in screening methods of the invention. In addition, it is contemplated that multiple such screens are performed at the same time in some embodiments of the invention.

It will of course be understood that in order for the substance to be considered a candidate Effective Compound (or oxygen antagonist, or stasis inducer or protective metabolic agent, or any other subset of Effective Compounds) the substance must not kill the organism or cells in the assay and the effect must be reversible (that is, the characteristic that is altered needs to resume to its level before the exposure to the substance).

It is of course understood that any method of treatment can be used in the context of a preparation of a medicament for the treatment of or protection against the specified disease or condition. This includes, but is not limited to, the preparation of a medicament for the treatment of hemorrhagic or hematologic shock, wounds and tissue damage, hyperthermia, hypothermia, neurodegeneration, sepsis, cancer, and trauma. Moreover, the invention includes, but is not limited to, the preparation of a medicament for a treatment to prevent death, shock, trauma, organ or tissue rejection, damage from cancer therapy, neurodegeneration, and wound or tissue damage.

As discussed above, organismal stasis is not any of the following states: sleep, comatose, death, anesthetized, or grand mal seizure. However, it is contemplated in some embodiments of the invention, that such states are the desired goal of employing methods, compositions and articles of manufacture of the invention. Any embodiment discussed with respect to one aspect of the invention applies to other aspects of the invention as well. Moreover, embodiments may be combined.

Any embodiment involving “exposing” biological matter to an Effective Compound may also be implemented so that biological matter is provided with the Effective Compound or administered the Effective Compound. The term “provide” is used according to its ordinary and plain meaning: “to supply or furnish for use” (Oxford English Dictionary), which, in the case of patients, may refer to the action performed by a doctor or other medical personnel who prescribes a particular Effective Compound or administers it directly to the patient.

### III. EFFECTIVE COMPOUNDS

In general, compounds of the present invention modulate HIF and can thereby affect hypoxia, ischemia, and/or stasis as well as any other condition associated with HIFα stabilization as described herein, such as hemorrhagic shock. Collectively, these compounds are called the Effective Compounds. Any compound as described herein may or may not be considered an Effective Compound.

In certain embodiments, the Effective Compounds may be represented by any one or more of the compounds of Formulas I, Ia-Id, II, III, IIIa, IV, V, VI, VII, VIII, IX, X, XI, carbon monoxide, chalcogenide compounds, H₂S and other sulfur containing compounds, protective metabolic agents and/or oxygen antagonists as described herein. It is specifically contemplated that any one or more of the compounds as described herein may not be considered an Effective Compound. Subsets of the Effective Compounds are also specifically contemplated.

In some embodiments, one or more Effective Compounds modulates a hypoxia-related condition. In some embodiments, one or more Effective Compounds modulates an ischemia-related condition. In some embodiments, one or more Effective Compounds modulates EPO. In other embodiments, one or more Effective Compounds induce stasis. In yet other embodiments, one or more Effective Compounds modulates hemorrhagic shock.

As used herein the term “Effective Compound” refers to any molecule that may modulate HIF in biological matter by, for example, altering core body temperature. An Effective Compound may be a protein or fragment thereof, a small molecule, or even a nucleic acid molecule. One may also acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Effective Compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the Effective Compound identified by the present invention may be peptide, polypeptide, polynucleotide, small molecule inhibitors or any other compounds that may be designed through rational drug design starting from known inhibitors or stimulators.

Other suitable Effective Compounds include antisense molecules, siRNAs, ribozymes, and antibodies (including single chain antibodies), each of which would be specific for the target molecule. Such compounds are described in greater detail elsewhere in this document.

In addition to the Effective Compounds initially identified, the inventor also contemplates that other structurally similar compounds may be formulated to mimic the key portions of the structure of the Effective Compounds. Such compounds, which may include peptidomimetics of peptide modulators, may be used in the same manner as the initial Effective Compounds.

Further details and embodiments of the Effective Compounds are described more fully later in this disclosure.

The Effective Compounds can be administered singly or in combination with various other therapeutic approaches. In one embodiment, the compound is administered with another 2-oxoglutarate dioxygenase inhibitor, wherein the two compounds have differential specificity for individual 2-oxoglutarate dioxygenase family members. The two compounds may be administered at the same time as a ratio of one relative to the other or may be administered consecutively during a treatment time course, e.g., following myocardial infarction. In one specific embodiment, one compound specifically inhibits HIF prolyl hydroxylase activity, and a second compound specifically inhibits procollagen prolyl 4-hydroxylase activity. In another embodiment, the compound is administered with another therapeutic agent having a different mode of action, e.g., an ACE inhibitor (ACEI), angiotensin-II receptor blocker (ARB), diuretic, and/or digoxin. In yet another embodiment, the compound is administered with carnitine.

In one aspect, a compound of the invention inhibits one or more 2-oxoglutarate dioxygenase enzymes. In one embodiment, the compound inhibits at least two 2-oxoglutarate dioxygenase family members, e.g., HIF prolyl hydroxylase and procollagen prolyl 4-hydroxylase, with either the same specificity or with differential specificity. In another embodiment, the compound is specific for one 2-oxoglutarate dioxygenase, e.g., HIF prolyl hydroxylase, and shows little to no specificity for other family members.

Some embodiments of the invention comprise methods using oral and transdermal delivery mechanisms. Thus, the present invention also provides an oral formulation comprising a compound of the invention. In another embodiment, the present methods involve transdermal administration of a compound of the invention. Thus, the present invention also provides a transdermal patch or pad comprising a compound of the invention.

These and other embodiments of the subject invention will readily occur to those of skill in the art in light of the disclosure herein, and all such embodiments are specifically contemplated.

### IV. VARIOUS EMBODIMENTS OF THE PRESENT INVENTION

The following embodiments further take advantage of the discovery that certain compounds shown to affect metabolic activity also act to stabilize HIF.

One general embodiment of the present invention contemplates a method for enhancing survivability of biological matter comprising:

(i) identifying biological matter in need of enhanced survivability; and

(ii) providing to the biological matter at least one compound with a chemical formula selected from the following groups:
(a) compound of Formula (Ia);
(b) compound of Formula (Ib);
(c) compound of Formula (Ic);
(d) compound of Formula (Id);
(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
   or a salt, ester, or precursor thereof, wherein induction of stasis or pre-stasis is achieved.

Another general embodiment of the present invention contemplates a method for preventing or reducing damage to biological matter comprising:

(i) identifying biological matter in need of prevented or reduced damage; and

(ii) providing to the biological matter at least one compound with a chemical formula selected from the following groups:
(a) compound of Formula (Ia);
(b) compound of Formula (Ib);
(c) compound of Formula (Ic);
(d) compound of Formula (Id);
(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
   or a salt, ester, or precursor thereof; wherein induction of stasis or pre-stasis is achieved.

Another general embodiment of the present invention contemplates a method for creating a preserved stock of organisms comprising:

(i) identifying an organism in need of preservation; and

(ii) providing to the organism at least one compound with a chemical formula selected from the following groups:
(a) compound of Formula (Ia);
(b) compound of Formula (Ib);
(c) compound of Formula (Ic);
(d) compound of Formula (Id);
(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
   or a salt, ester, or precursor thereof; wherein induction of stasis or pre-stasis is achieved.

Another general embodiment of the present invention contemplates a method for reversibly inhibiting metabolism in an organism comprising:

(i) identifying an organism in need of reversible metabolism inhibition; and;

(ii) providing to the organism at least one compound with a chemical formula selected from the following groups:
(a) compound of Formula (Ia);
(b) compound of Formula (Ib);
(c) compound of Formula (Ic);
(d) compound of Formula (Id);
(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
   or a salt, ester, or precursor thereof; wherein stabilization of the alpha-subunit of hypoxia inducible factor (HIFα) or induction of stasis or pre-stasis is achieved. is achieved.

Another general embodiment of the present invention contemplates a method for inducing sleep in an organism comprising:

(i) identifying an organism in need of sleep; and;

(ii) providing to the organism at least one compound with a chemical formula selected from the following groups:
(a) compound of Formula (Ia);
(b) compound of Formula (Ib);
(c) compound of Formula (Ic);
(d) compound of Formula (Id);
(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
   or a salt, ester, or precursor thereof; wherein stabilization of the alpha-subunit of hypoxia inducible factor (HIFα) or induction of stasis or pre-stasis is achieved.

Another general embodiment of the present invention contemplates a method for anesthetizing biological matter comprising:

(i) identifying biological matter in need of anesthesia and;

(ii) providing to the biological matter at least one compound with a chemical formula selected from the following groups:
(a) compound of Formula (Ia);
(b) compound of Formula (Ib);
(c) compound of Formula (Ic);
(d) compound of Formula (Id);
(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
   or a salt, ester, or precursor thereof; wherein stabilization of the alpha-subunit of hypoxia inducible factor (HIFα) is achieved.

Another general embodiment of the present invention contemplates a method of protecting biological matter from an injury, the onset or progression of a disease, or death comprising:

(i) identifying biological matter in need of protection from an injury, the onset or progression of a disease, or death; and

(ii) providing to the biological matter at least one compound with a chemical formula selected from the following groups:
(a) compound of Formula (Ia);
(b) compound of Formula (Ib);
(c) compound of Formula (Ic);
(d) compound of Formula (Id);
(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
   or a salt, ester, or precursor thereof; wherein induction of stasis or pre-stasis is achieved.

Another general embodiment of the present invention contemplates a method of preventing an organism from bleeding to death comprising:

(i) identifying an organism in need of prevention from bleeding to death; and

(ii) providing to the organism at least one compound with a chemical formula selected from the following groups:
(a) compound of Formula (Ia);
(b) compound of Formula (Ib);
(c) compound of Formula (Ic);
(d) compound of Formula (Id);
(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
   or a salt, ester, or precursor thereof; wherein stabilization of the alpha-subunit of hypoxia inducible factor (HIFα) or induction of stasis or pre-stasis is achieved.

Another general embodiment of the present invention contemplates a method for inducing stasis in biological matter comprising:

(i) identifying biological matter in which stasis is desired; and

(ii) providing to the biological matter at least one compound with a chemical formula selected from the following groups:
(a) compound of Formula (Ia);
(b) compound of Formula (Ib);
(c) compound of Formula (Ic);
(d) compound of Formula (Id);
(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
   or a salt, ester, or precursor thereof; wherein induction of stasis or pre-stasis is achieved.

Another general embodiment of the present invention contemplates a method of protecting biological matter from suffering cellular damage from a surgery comprising:

(i) identifying biological matter in need of protection from suffering cellular damage from a surgery; and

(ii) providing to the biological matter at least one compound with a chemical formula selected from the following groups:
(a) compound of Formula (Ia);
(b) compound of Formula (Ib);
(c) compound of Formula (Ic);
(d) compound of Formula (Id);
(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
   or a salt, ester, or precursor thereof; wherein induction of the mammal into pre-stasis or stasis is achieved prior to the surgery.

Another general embodiment of the present invention contemplates a method of protecting biological matter from suffering cellular damage from a disease or adverse medical condition comprising:

(i) identifying biological matter in need of protection from suffering cellular damage from a disease or adverse medical condition; and

(ii) providing to the biological matter at least one compound with a chemical formula selected from the following groups:
(a) compound of Formula (Ia);
(b) compound of Formula (Ib);
(c) compound of Formula (Ic);
(d) compound of Formula (Id);
(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
   or a salt, ester, or precursor thereof; wherein induction of the mammal into pre-stasis or stasis is achieved prior to the onset or progression of the disease or adverse medical condition.

Another general embodiment of the present invention contemplates a method for inducing apnea in an organism comprising:

(i) identifying an organism in need of apnea induction and;

(ii) providing to the organism at least one compound with a chemical formula selected from the following groups:
(a) compound of Formula (Ia);
(b) compound of Formula (Ib);
(c) compound of Formula (Ic);
(d) compound of Formula (Id);
(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
   or a salt, ester, or precursor thereof; wherein stabilization of the alpha-subunit of hypoxia inducible factor (HIFα) or induction of stasis or pre-stasis is achieved.

Another general embodiment of the present invention contemplates a method of reducing oxygen demand in biological matter comprising:

(i) identifying biological matter in need of reduced oxygen; and

(ii) contacting the biological matter with at least one compound with a chemical formula selected from the following groups:
(a) compound of Formula (Ia);
(b) compound of Formula (Ib);
(c) compound of Formula (Ic);
(d) compound of Formula (Id);
(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
   or a salt, ester, or precursor thereof; wherein induction of stasis or pre-stasis is achieved.

Another general embodiment of the present invention contemplates a method of delaying the effects of a trauma on biological matter comprising:

(i) identifying biological matter in need of said delayed effects; and

(ii) contacting the biological matter with at least one compound with a chemical formula selected from the following groups:
(a) compound of Formula (Ia);
(b) compound of Formula (Ib);
(c) compound of Formula (Ic);
(d) compound of Formula (Id);
(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
   or a salt, ester, or precursor thereof; wherein induction of stasis or pre-stasis is achieved.

Another general embodiment of the present invention contemplates a method for treating or preventing hemorrhagic shock in an organism comprising:

(i) identifying organism in need of said treatment or prevention; and

(ii) contacting the organism with at least one compound with a chemical formula selected from the following groups:
(a) compound of Formula (Ia);
(b) compound of Formula (Ib);
(c) compound of Formula (Ic);
(d) compound of Formula (Idina/(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
   or a salt, ester, or precursor thereof; wherein induction of stasis or pre-stasis is achieved.

Another general embodiment of the present invention contemplates a method of inducing hibernation in an organism comprising:

(i) identifying an organism in need of induced hibernation; and

(ii) contacting the organism with at least one compound with a chemical formula selected from the following groups:
(a) compound of Formula (Ia);
(b) compound of Formula (Ib);
(c) compound of Formula (Ic);
(d) compound of Formula (Id);
(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
   or a salt, ester, or precursor thereof; wherein stabilization of the alpha-subunit of hypoxia inducible factor (HIFα) or induction of stasis or pre-stasis is achieved.

Another general embodiment of the present invention contemplates a method of protecting an organism from radiation therapy or chemotherapy comprising:

(i) identifying an organism in need of said protection; and

(ii) contacting the organism with at least one compound with a chemical formula selected from the following groups:
(a) compound of Formula (Ia);
(b) compound of Formula (Ib);
(c) compound of Formula (Ic);
(d) compound of Formula (Id);
(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
   or a salt, ester, or precursor thereof; wherein induction of stasis or pre-stasis is achieved.

Another general embodiment of the present invention contemplates a method of treating a hyperproliferative disease in an organism comprising:

(i) identifying an organism in need of treatment; and

(ii) contacting the organism with at least one compound with a chemical formula selected from the following groups:
(a) compound of Formula (Ia);
(b) compound of Formula (Ib);
(c) compound of Formula (Ic);
(d) compound of Formula (Id);
(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
   or a salt, ester, or precursor thereof; wherein stabilization of the alpha-subunit of hypoxia inducible factor (HIFα) or induction of stasis or pre-stasis is achieved.

Another general embodiment of the present invention contemplates a method of inhibiting rejection of an organ transplant in an organism comprising:

(i) identifying an organism in need of an organ transplant; and

(ii) providing the organism with at least one compound with a chemical formula selected from the following groups:
(a) compound of Formula (Ia);
(b) compound of Formula (Ib);
(c) compound of Formula (Ic);
(d) compound of Formula (Id);
(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
   or a salt, ester, or precursor thereof; wherein induction of stasis or pre-stasis is achieved.

Another general embodiment of the present invention contemplates a method of treating an organism with hypothermia comprising:

(i) identifying an organism in need of treatment;

(ii) contacting the organism with at least one compound with a chemical formula selected from the following groups:
(a) compound of Formula (Ia);
(b) compound of Formula (Ib);
(c) compound of Formula (Ic);
(d) compound of Formula (Id);
(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
   or a salt, ester, or precursor thereof, and

(ii) subjecting the organism to an environmental temperature above that of the organism;

wherein stabilization of the alpha-subunit of hypoxia inducible factor (HIFα) or induction of stasis or pre-stasis is achieved.

Another general embodiment of the present invention contemplates a method for inducing cardioplegia in an organism undergoing bypass surgery comprising:

(i) identifying an organism in need of cardioplegia; and

(ii) providing the organism at least one compound with a chemical formula selected from the following groups:
(a) compound of Formula (Ia);
(b) compound of Formula (Ib);
(c) compound of Formula (Ic);
(d) compound of Formula (Id);

(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
   or a salt, ester, or precursor thereof; wherein stabilization of the alpha-subunit of hypoxia inducible factor (HIFα) or induction of stasis or pre-stasis is achieved.

Another general embodiment of the present invention contemplates a method of treating an organism with hyperthermia comprising:

(i) identifying an organism in need of treatment; and

(ii) contacting the organism with at least one compound with a chemical formula selected from the following groups:
(a) compound of Formula (Ia);
(b) compound of Formula (Ib);
(c) compound of Formula (Ic);
(d) compound of Formula (Id);
(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
   or a salt, ester, or precursor thereof; wherein stabilization of the alpha-subunit of hypoxia inducible factor (HIFα) or induction of stasis or pre-stasis is achieved.

Another general embodiment of the present invention contemplates a method for preventing hematologic shock in an organism comprising:

(i) identifying an organism in need of said prevention; and

(ii) providing the organism with at least one compound with a chemical formula selected from the following groups:
(a) compound of Formula (Ia);
(b) compound of Formula (Ib);
(c) compound of Formula (Ic);
(d) compound of Formula (Id);
(e) compound of Formula (II);
(f) compound of Formula (III);
(g) compound of Formula (V);
(h) compound of Formula (VI);
(i) compound of Formula (VII);
(i) compound of Formula (VIII);
(k) compound of Formula (IX);
(l) compound of Formula (X);
(m) compound of Formula (XI);
   or a salt, ester, or precursor thereof; wherein induction of stasis or pre-stasis is achieved.

Another general embodiment of the present invention contemplates a method for promoting wound healing in an organism comprising:

(i) identifying an organism with a wound in need of healing; and

(ii) providing the organism or wound at least one compound with a chemical formula selected from the following groups:
(a) compound of Formula (Ia);
(b) compound of Formula (Ib);
(c) compound of Formula (Ic);
(d) compound of Formula (Id);
(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
   or a salt, ester, or precursor thereof; wherein induction of stasis or pre-stasis is achieved.

Another general embodiment of the present invention contemplates a method for preventing or treating neurodegeneration in an organism comprising:

(i) identifying an organism in need of treatment; and

(ii) providing the organism at least one compound with a chemical formula selected from the following groups:
(a) compound of Formula (Ia);
(b) compound of Formula (Ib);
(c) compound of Formula (Ic);
(d) compound of Formula (Id);
(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
   or a salt, ester, or precursor thereof; wherein induction of stasis or pre-stasis is achieved.

Another general embodiment of the present invention contemplates a method for preserving biological matter comprising:

(i) identifying biological matter in need of preservation; and

(ii) providing the biological matter at least one compound with a chemical formula selected from the following groups:
(a) compound of Formula (Ia);
(b) compound of Formula (Ib);
(c) compound of Formula (Ic);
(d) compound of Formula (Id);
(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
   or a salt, ester, or precursor thereof; wherein induction of stasis or pre-stasis is achieved.

Another general embodiment of the present invention contemplates a method for cryopreserving biological matter comprising:

(i) first identifying biological matter in need of cryopreservation;

(ii) second contacting the biological matter with at least one compound with a chemical formula selected from the following groups:
(a) compound of Formula (Ia);
(b) compound of Formula (Ib);
(c) compound of Formula (Ic);
(d) compound of Formula (Id);
(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
   or a salt, ester, or precursor thereof; for an effective amount of time for the biological matter to enter stasis; and

(iii) then cryopreserving the biological matter;

wherein stabilization of the alpha-subunit of hypoxia inducible factor (HIFα) or induction of stasis or pre-stasis is achieved. In certain embodiments, the cryopreservation of the biological material comprises perfusing the biological matter with a cryoprotectant and lowering the temperature of the biological matter.

In certain embodiments of the present invention, the biological matter or organism is provided or contacted with at least one Effective Compound in an amount and for a time sufficient to cause the biological matter or organism to enter stasis. The biological matter or organism may be provided or contacted with the compound in an amount and for a time sufficient to stabilize the alpha-subunit of hypoxia inducible factor (HIFα) in cells of the biological matter or organism.

Certain embodiments of the present invention comprise incubating the biological matter or organism under hypoxic conditions and/or anoxic conditions. In some embodiments, the hypoxic and/or anoxic conditions would damage the biological matter or organism in the absence of an Effective Compound.

In certain embodiments of the present invention, the biological matter or organism is exposed to at least one Effective Compound. The biological matter or organism may be exposed to an amount of compound that reduces the rate or amount of CO₂ production by the biological matter or organism by at least about two-fold. The biological matter or organism may be exposed to an amount of compound that reduces the rate or amount of oxygen consumption by the biological matter or organism by at least about two-fold. In certain embodiments, the biological matter or organism is exposed to an amount of compound that decreases movement or motility by at least about 10%.

Some embodiments of the present invention contemplate providing a combination of Effective Compounds to the biological matter or organism. Such compounds may be selected from, for example, at least one compound with a chemical formula selected from the following groups:

(n) Formula (I);

(j) Formula (IV);

(p) carbon monoxide;

(q) chalcogenide compound;

(r) H₂S or other sulfur containing compound;

(s) protective metabolic agent;

(t) oxygen antagonist;

or a salt, ester, or precursor thereof. The biological matter or organism may be provided with the compound before, during, or after an injury, the onset or progression of a disease, or hemorrhaging in the biological matter or organism. The injury may be from an external source. The compound may be provided before the injury or before the onset or progression of the disease. In certain embodiments, the compound is not provided during or after the injury or the onset or progression of the disease. The injury or disease may be associated with a reduction in metabolism or temperature of the biological matter or organism. The injury may be a surgery. The compound may be provided during the progression of the disease, such as thalassemia, sickle cell disease, or cystic fibrosis. The biological matter or organism may be provided with the compound in an amount and for a time sufficient to reduce CO₂ evolution by at least 25%. The biological matter or organism may be provided with the compound in an amount and for a time that protects the matter from damage or death resulting from the injury or the onset or progression of the disease. The biological matter may be provided with the compound in an amount and for a time sufficient to increase the rate of entry of the matter into stasis following the injury or the onset or progression of the disease.

In certain embodiments, the biological matter or organism is provided the compound by administration to the biological matter or organism intravenously, intradermally, intraarterially, intraperitoneally, intralesionally, intracranially, intraarticularly, intraprostaticaly, intrapleurally, intratracheally, intranasally, intrathecally, intravitreally, intravaginally, intrarectally, intratumorally, intramuscularly, intraperitoneally, intraocularly, subcutaneously, subconjunctival, intravesicularlly, mucosally, intrapericardially, intraumbilically, intraocularally, orally, topically, locally, by inhalation, by injection, by infusion, by continuous infusion, by localized perfusion, via a catheter, via a lavage, or through catheterization, immersion, absorption, or adsorption.

Certain embodiments of the present invention further comprise exposing the biological matter to a controlled temperature and/or pressure environment. The biological matter may achieve a non-physiological core temperature. The biological matter may be is exposed to a controlled temperature environment that is less than about 20° C. The biological matter may be exposed to the controlled temperature environment prior to and/or concurrent with being provided with one or more compounds.

In certain embodiments, one or more Effective Compounds comprises a cationic structure capable of targeting the one or more compounds to mitochondria.

In certain embodiments, the biological matter or organism is to be preserved, such as for future consumption. The biological matter or organism may be used for research purposes, for example. The biological matter to be preserved may include platelets, or the biological matter may be used for transplantation purposes.

In certain embodiments, cardioplegia is induced in the biological matter or organism.

In certain embodiments, the biological matter or organism is at risk for hemorrhagic shock.

Certain methods of the present invention further comprise monitoring the biological matter or organism for toxicity from the compound.

In certain embodiments, the biological matter or organism is hemorrhaging.

In certain embodiments, the biological matter or organism goes into hemorrhagic shock.

In certain embodiments wherein the method contemplates a disease or adverse medical condition, the disease or adverse medical condition may be selected from the group consisting of: hemorrhagic shock, myocardial infarction, acute coronary syndrome, cardiac arrest, neonatal hypoxia/ischemia, ischemic reperfusion injury, unstable angina, post-angioplasty, aneurysm, trauma, blood loss, an infectious disease, a hyperproliferative disease, a neurodegenerative disease, or an inflammatory disease.

In certain embodiments wherein the method contemplates trauma, the trauma may be surgery, stroke, heart attack, bone fracture, soft tissue damage, internal bleeding, organ damage, amputation, concussion, burns, and/or is caused by gunshot, a shrapnel wound, or a knife wound.

Another general embodiment of the present invention contemplates a method for stabilizing the alpha-subunit of hypoxia inducible factor (HIFα) in biological matter, wherein the method comprises administering at least one compound with a chemical formula selected from the following groups:

(n) Formula (I);

(j) Formula (IV);

(p) carbon monoxide;

(q) chalcogenide compound;

(r) H₂S or other sulfur containing compound;

(s) protective metabolic agent;

(t) oxygen antagonist;

or a salt, ester, or precursor thereof, wherein the compound inhibits hydroxylation of HIFα.

Another general embodiment of the present invention contemplates a method for stabilizing the alpha-subunit of hypoxia inducible factor (HIFα) in biological matter, the method comprising administering at least one compound with a chemical formula selected from the following groups:

(n) Formula (I);

(j) Formula (IV);

(p) carbon monoxide;

(q) chalcogenide compound;

(r) H₂S or other sulfur containing compound;

(s) protective metabolic agent;

(t) oxygen antagonist;

or a salt, ester, or precursor thereof, wherein the compound inhibits 2-oxoglutarate dioxygenase enzyme activity. In certain embodiments, the 2-oxoglutarate dioxygenase enzyme is selected from the group consisting of EGLN1, EGLN2, EGLN3, procollagen prolyl 4-hydroxylase, procollagen prolyl 3-hydroxylase, procollagen lysyl hydroxylase, PHD4, FIH-1, and any subunit or fragment thereof.

Another general embodiment of the present invention contemplates a method for stabilizing the alpha-subunit of hypoxia inducible factor (HIFα) in biological matter, the method comprising administering at least one compound with a chemical formula selected from the following groups:

(n) Formula (I);

(j) Formula (IV);

(p) carbon monoxide;

(q) chalcogenide compound;

(r) H₂S or other sulfur containing compound;

(s) protective metabolic agent;

(t) oxygen antagonist;

or a salt, ester, or precursor thereof, wherein the compound inhibits HIF prolyl hydroxylase enzyme activity. In certain embodiments, the HIF prolyl hydroxylase enzyme is selected from the group consisting of EGLN1, EGLN2, EGLN3, and any subunit or fragment thereof.

Another general embodiment of the present invention contemplates a method for treating, preventing, or pretreating a HIF-associated condition in biological matter, the method comprising administration of at least one compound with a chemical formula selected from the following groups:

(n) Formula (I);

(j) Formula (IV);

(p) carbon monoxide;

(q) chalcogenide compound;

(r) H₂S or other sulfur containing compound;

(s) protective metabolic agent;

(t) oxygen antagonist;

or a salt, ester, or precursor thereof; wherein HIFα is stabilized.

Another general embodiment of the present invention contemplates a method for treating, preventing, or pretreating a HIF-associated condition in biological matter, the method comprising administration of at least one compound with a chemical formula selected from the following groups:

(n) Formula (I);

(j) Formula (IV);

(p) carbon monoxide;

(q) chalcogenide compound;

(r) H₂S or other sulfur containing compound;

(s) protective metabolic agent;

(t) oxygen antagonist;

or a salt, ester, or precursor thereof; wherein 2-oxoglutarate dioxygenase enzyme activity is inhibited.

Another general embodiment of the present invention contemplates a method for treating, preventing, or pretreating a HIF-associated condition in biological matter, the method comprising administration of at least one compound with a chemical formula selected from the following groups:

(n) Formula (I);

(j) Formula (IV);

(p) carbon monoxide;

(q) chalcogenide compound;

(r) H₂S or other sulfur containing compound;

(s) protective metabolic agent;

(t) oxygen antagonist;

or a salt, ester, or precursor thereof; wherein HIF prolyl hydroxylase enzyme activity is inhibited.

In certain embodiments, methods for treating, preventing, or pretreating a HIF-associated condition in biological matter wherein the HIF-associated condition is associated with hypoxia or ischemia are contemplated. In particular embodiments, the method further comprises the stabilization of HIFα, the inhibition of 2-oxoglutarate dioxygenase enzyme activity, and/or the inhibition of HIF prolyl hydroxylase enzyme activity. In particular embodiments, the HIF-associated condition is associated with a pulmonary disorder, a cardiac disorder, a neurological disorder or with an ischemic event. The ischemic event may be an acute ischemic event. The acute ischemic event may be associated with surgery, organ transplantation, infarction, trauma, or injury. The ischemic event may be a chronic ischemic event. The chronic ischemic event may be associated with a condition selected from the group consisting of hypertension, diabetes, occlusive arterial disease, chronic venous insufficiency, Raynaud's disease, cirrhosis, congestive heart failure and systemic sclerosis.

Another general embodiment of the present invention contemplates a method of treating, preventing or pretreating a condition mediated at least in part by hypoxia inducible factor (HIF) and/or erythropoietin (EPO), said method comprising administering to a subject an effective amount of at least one compound with a chemical formula selected from the following groups:

(n) Formula (I);

(j) Formula (IV);

(p) carbon monoxide;

(q) chalcogenide compound;

(r) H₂S or other sulfur containing compound;

(s) protective metabolic agent;

(t) oxygen antagonist;

or a salt, ester, or precursor thereof; wherein HIFα is stabilized. The condition may be selected from the group consisting of anemic disorders; neurological disorders and/or injuries; including cases of stroke, trauma, epilepsy, neurodegenerative disease, myocardial infarction, liver ischemia, renal ischemia, and stroke; peripheral vascular disorders, ulcers, burns, and chronic wounds; pulmonary embolism; and ischemic-reperfusion injury.

Certain embodiments of the present invention further comprise providing to the biological matter at least one compound with a chemical formula selected from the following groups:

(a) compound of Formula (Ia);

(b) compound of Formula (Ib);

(c) compound of Formula (Ic);

(d) compound of Formula (Id);

(e) compound of Formula (II);

(f) compound of Formula (III);

(g) compound of Formula (V);

(h) compound of Formula (VI);

(i) compound of Formula (VII);

(j) compound of Formula (VIII);

(k) compound of Formula (IX);

(l) compound of Formula (X);

(m) compound of Formula (XI);

(u) ACE inhibitor (ACEI);

(v) angiotensin-II receptor blocker (ARB);

(w) diuretic;

(x) digoxin;

(y) statin;

(z) carnitine;

or a salt, ester, or precursor thereof. In certain embodiments, any one or more of the administered compounds inhibits 2-oxoglutarate dioxygenase enzyme activity and/or HIF prolyl hydroxylase activity.

Certain embodiments contemplate stabilization of HIFα by specifically inhibiting hydroxylation of at least one amino acid residue in HIFα using one or more of the Effective Compounds disclosed herein. In certain embodiments, one or more of the Effective Compounds are selected from the group consisting of:

(n) Formula (I);

(o) Formula (IV);

(p) carbon monoxide;

(q) chalcogenide compound;

(r) H₂S or other sulfur containing compound;

(s) protective metabolic agent;

(t) oxygen antagonist;

and a salt, ester, or precursor thereof. The amino acid residue may be selected from the group consisting of proline and asparagine.

Another general embodiment of the present invention contemplates a method for increasing expression of angiogenic factors in biological matter, the method comprising administration of at least one compound with a chemical formula selected from the following groups:

(n) Formula (I);

(o) Formula (IV);

(p) carbon monoxide;

(q) chalcogenide compound;

(r) H₂S or other sulfur containing compound;

(s) protective metabolic agent;

(t) oxygen antagonist;

or a salt, ester, or precursor thereof; wherein HIFα is stabilized.

Another general embodiment of the present invention contemplates a method for increasing expression of glycolytic factors in biological matter, the method comprising administration of at least one compound with a chemical formula selected from the following groups:

(n) Formula (I);

(o) Formula (IV);

(p) carbon monoxide;

(q) chalcogenide compound;

(r) H₂S or other sulfur containing compound;

(s) protective metabolic agent;

(t) oxygen antagonist;

or a salt, ester, or precursor thereof; wherein HIFα is stabilized.

Another general embodiment of the present invention contemplates a method for increasing expression of factors associated with oxidative stress in a subject, the method comprising administration of at least one compound with a chemical formula selected from the following groups:

(n) Formula (I);

(o) Formula (IV);

(p) carbon monoxide;

(q) chalcogenide compound;

(r) H₂S or other sulfur containing compound;

(s) protective metabolic agent;

(t) oxygen antagonist;

or a salt, ester, or precursor thereof; wherein HIFα is stabilized.

Another general embodiment of the present invention contemplates a method of treating a subject having a disorder associated with ischemic reperfusion injury, the method comprising administration of at least one compound with a chemical formula selected from the following groups:

(n) Formula (I);

(o) Formula (IV);

(p) carbon monoxide;

(q) chalcogenide compound;

(r) H₂S or other sulfur containing compound;

(s) protective metabolic agent;

(t) oxygen antagonist;

or a salt, ester, or precursor thereof; wherein HIFα is stabilized.

In certain embodiments, HIFα is selected from the group consisting of HIF-1α, HIF-2α, HIF-3α, and any fragment thereof.

Certain methods of the present invention further comprise providing to the organism at least one compound with a chemical formula selected from the following groups: heterocyclic carboxamides; phenanthrolines; hydroxamates; or a salt, ester, or precursor thereof. The heterocyclic carboxamides may be selected from the group consisting of pyridine carboxamides, quinoline carboxamides, isoquinoline carboxamines, cinnoline carboxamides and beta-carboline carboxamides.

In certain embodiments, the biological matter or organism utilized in one of the methods of the present invention is a cell, tissue or organ. The cell, tissue, or organ may be derived from a system selected from the group consisting of the renal, cardiac, hepatic, pulmonary, hematopoietic, gastrointestinal, neuronal and musculoskeletal systems. The biological matter may be an organ, tissue, or cell from the heart, lung, kidney, liver, bone marrow, pancreas, skin, bone, vein, artery, cornea, blood, small intestine, large intestine, brain, spinal cord, smooth muscle, skeletal muscle, ovary, testis, uterus, or umbilical cord. The biological matter may comprise the following cell types: platelet, myelocyte, erythrocyte, lymphocyte, adipocyte, fibroblast, epithelial cell, endothelial cell, smooth muscle cell, skeletal muscle cell, endocrine cell, glial cell, neuron, secretory cell, barrier function cell, contractile cell, absorptive cell, mucosal cell, limbus cell (from cornea), stem cell, unfertilized or fertilized oocyte, or sperm. The biological matter or organism may be a fly, fish, frog, or embryo thereof. The biological matter or organism may be a mammal, such as a human. In certain methods of the present invention, the biological matter or organism is provided or contacted with an effective amount of the compound. The effective amount may be a sublethal dose of the compound. The effective amount may be a near-lethal dose of the compound.

Certain methods of the present invention take place in vivo.

Other compositions and methods of the invention involve a HIF-1 antibody. In certain embodiments the invention relates to an antibody that specifically recognizes an epitope on HIF-1. In certain embodiments, the antibody immunologically reacts with a HIF-1 polypeptide, which may be from a mammal, such as a human or from a non-mammal, such as a nematode. In certain embodiments of the invention, the antibody recognizes the N-terminal portion of a HIF-1 polypeptide, which means it may recognize amino acids at positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or any combination thereof in a HIF-1 polypeptide. In particular embodiments, the antibody recognizes an epitope within amino acids 103-464 of the nematode HIF-1 polypeptide.

### V. DEFINITIONS

The term “HIF-associated conditions,” “HIF-related conditions,” “HIF-associated disorders” and “HIF-related disorders” and the like refer to any of the ischemic, hypoxic, and other conditions as described herein, and further refer to stasis.

The term “ischemia” refers to a reduction in blood flow. Ischemia is associated with a reduction in nutrients, including oxygen, delivered to tissues. Ischemia may arise due to conditions such as atherosclerosis, formation of a thrombus in an artery or vein, or blockage of an artery or vein by an embolus, vascular closure due to other causes, e.g., vascular spasm, etc. Such conditions may reduce blood flow, producing a state of hypoperfusion to an organ or tissue, or block blood flow completely. Other conditions that can produce ischemia include tissue damage due to trauma or injury, such as, e.g., spinal cord injury; viral infection, which can lead to, e.g., congestive heart failure, etc. The terms “ischemic conditions” and “ischemic disorders” refer to any condition, disease, or disorder that is associated with ischemia and may refer to acute ischemic conditions including, but not limited to, myocardial infarction, ischemic stroke, pulmonary embolism, perinatal hypoxia, circulatory shock including, e.g., hemorrhagic, septic, cardiogenic, etc., mountain sickness, acute respiratory failure, etc., chronic ischemic conditions including atherosclerosis, chronic venous insufficiency, chronic heart failure, cardiac cirrhosis, diabetes, macular degeneration, sleep apnea, Raynaud's disease, systemic sclerosis, nonbacterial thrombotic endocarditis, occlusive artery disease, angina pectoris, TIAs, chronic alcoholic liver disease, etc. Ischemic conditions may also result from angioplasty as well as when individuals are placed under general anesthesia, and can also cause tissue damage in organs prepared for transplant.

The terms “hypoxia” and “hypoxic” refer to an environment with levels of oxygen below normal. For example, hypoxia may occur when the normal physiologic levels of oxygen are not supplied to a tissue or cell. “Normoxia” refers to normal physiologic levels of oxygen for the particular cell type, cell state or tissue in question. “Anoxia” is the absence of oxygen. Hypoxia may be induced in cells by culturing the cells in a reduced oxygen environment, or cells may be treated with compounds that mimic hypoxia. Determining oxygen levels that define hypoxia in cell culture is well within the skill in the art.

The terms “hypoxic conditions” and “hypoxic disorders” refer to any condition, disease, or disorder that is associated with hypoxia including, but not limited to, ischemic disorders (ischemic hypoxia) such as those listed above, wherein hypoxia results from reduced circulation; pulmonary disorders (hypoxic hypoxia) such as COPD (chronic obstructive pulmonary disease), severe pneumonia, pulmonary edema, pulmonary hypertension, hyaline membrane disease, and the like, wherein hypoxia results from reduced oxygenation of the blood in the lungs; anemic disorders (anemic hypoxia) such as gastric or duodenal ulcers, liver or renal disease, thrombocytopenia or blood coagulation disorders, cancer or other chronic illness, cancer chemotherapy and other therapeutic interventions that produce anemia, and the like, wherein hypoxia results from a decreased concentration of hemoglobin or red blood cells; and altitude sickness, etc.

The terms “disorders,” “diseases,” and “conditions” are used inclusively and refer to any condition deviating from normal. Such ischemic and hypoxic disorders include, but are not limited to, those disorders described above. In certain embodiments, stasis is an example of an HIF-associated condition.

The term “HIFα” refers to the alpha subunit of hypoxia inducible factor protein. HIFα may be any human or other mammalian protein, or fragment thereof, including, but not limited to, human HIF-1α (Genbank Accession No. Q16665), HIF-2α (Genbank Accession No. AAB41495), and HIF-3α (Genbank Accession No. AAD22668); murine HIF-1α (Genbank Accession No. Q61221), HIF-2α (Genbank Accession No. BAA20130 and AAB41496), and HIF-3α (Genbank Accession No. AAC72734); rat HIF-1α (Genbank Accession No. CAA70701), HIF-2α (Genbank Accession No. CAB96612), and HIF-3α (Genbank Accession No. CAB96611); and cow HIF-1α (Genbank Accession No. BAA78675). HIFα may also be any non-mammalian protein or fragment thereof, including Xenopus laevis HIF-1α (Genbank Accession No. CAB96628), Drosophila melanogaster HIF-1α (Genbank Accession No. JC4851), and chicken HIF-1α (Genbank Accession No. BAA34234). HIFα gene sequences may also be obtained by routine cloning techniques, for example, by using all or part of a HIFα gene sequence described above as a probe to recover and determine the sequence of a HIFα gene in another species.

A fragment of HIFα includes any fragment retaining at least one functional or structural characteristic of HIFα. Fragments of HIFα include the regions defined by human HIF-1α from amino acid 401 to 603 (Huang et al., supra), amino acid 531 to 575 (Jiang et al. (1997)), amino acid 556 to 575 (Tanimoto et al., supra), amino acid 557 to 571 (Srinivas et al. (1999)), and amino acid 556 to 575 (Ivan and Kaelin (2001).) Further, a fragment of HIFα includes any fragment containing at least one occurrence of the motif LXXLAP, e.g., as occurs in the HIF-1 native sequence at L₃₉₇TLLAP and L₅₅₉EMLAP. For example, a HIF peptide may comprise [methoxycoumarin]-DLDLEALAPYIPA-DDDFQL-amide (SEQ ID NO:5).

The terms “HIF prolyl hydroxylase” and “HIF PH” refer to any enzyme capable of hydroxylating a proline residue in the HIF protein. The proline residue hydroxylated by HIF PH may include the proline found within the motif LXXLAP, e.g., as occurs in the human HIF-1α native sequence at L₃₉₇TLLAP and L₅₅₉EMLAP. HIF PH includes members of the Egl-Nine (EGLN) gene family described by Taylor (2001), and characterized by Aravind and Koonin (2001), Epstein et al. (2001), and Bruick and McKnight (2001). Examples of HIF PH enzymes include human SM-20 (EGLN1) (GenBank Accession No. AAG33965; Dupuy et al. (2000)), EGLN2 isoform 1 (GenBank Accession No. CAC42510; Taylor, supra), EGLN2 isoform 2 (GenBank Accession No. NP_{—}060025), and EGLN3 (GenBank Accession No. CAC42511; Taylor, supra); mouse EGLN1 (GenBank Accession No. CAC42515), EGLN2 (GenBank Accession No. CAC42511), and EGLN3 (SM-20) (GenBank Accession No. CAC42517); and rat SM-20 (GenBank Accession No. AAA19321). Additionally, HIF PH may include Caenorhabditis elegans EGL-9 (GenBank Accession No. AAD56365) and Drosophila melanogaster CGI 114 gene product (GenBank Accession No. AAF52050). HIF PH also includes any fragment retaining at least one structural or function feature of the foregoing full-length proteins, including a fragment having hydroxylase activity. HIF PH also includes any fragment of the foregoing full-length proteins that retain at least one structural or functional characteristic.

The terms “amino acid sequence” or “polypeptide” as used herein, typically refers to HIFα and fragments thereof, or HIF PH and fragments thereof, and contemplate an oligopeptide, peptide, or protein sequence, or to a fragment of any of these, and to naturally occurring or synthetic molecules. “Fragments” can refer to any portion of a sequence that retains at least one structural or functional characteristic of the protein. Immunogenic fragments or antigenic fragments are fragments of polypeptides, (e.g., fragments of about five to fifteen amino acids in length, that retain at least one biological or immunological activity. Where “amino acid sequence” is used to refer to the polypeptide sequence of a naturally occurring protein molecule, “amino acid sequence” and like terms are not meant to limit the amino acid sequence to the complete native sequence associated with the recited protein molecule.

The term “related proteins” as used herein typically refers to, for example, proteins related to HIFα prolyl hydroxylase, and may encompass other 2-oxoglutarate dioxygenase enzymes, especially those family members that similarly require Fe²⁺, 2-oxoglutarate, and oxygen to maintain hydroxylase activity. Such enzymes include, but are not limited to, e.g., procollagen lysyl hydroxylase, procollagen prolyl 4-hydroxylase, and Factor Inhibiting HIF (FIH), an asparaginyl hydroxylase responsible for regulating transactivation of HIFα. (GenBank Accession No. AAL27308; Mahon et al. (2001); Lando et al. (2002a); and Lando et al. (2002b). See, also, Elkins et al. (2002).

“Treatment” and “treating” refer to administration or application of an agent, drug, or remedy to a subject or performance of a procedure or modality on a subject for the purpose of obtaining a therapeutic benefit of a disease or health-related condition.

A “disease” or “health-related condition” can be any pathological condition of a body part, an organ, a tissue, or a system resulting from any cause, such as infection, genetic defect, and/or environmental stress. The cause may or may not be known. Examples of such conditions include, but are not limited to, premalignant states, dysplasias, cancer, and other hyperproliferative diseases as well as ischemic and hypoxic conditions and EPO-associated conditions. The cancer, for example, may be a recurrent cancer or a cancer that is known or suspected to be resistant to conventional therapeutic regimens and standard therapies.

The term “therapeutic benefit” used throughout this application refers to anything that promotes or enhances the well-being of the subject with respect to the medical treatment of his condition, which includes, but is not limited to, treatment of pre-cancer, dysplasia, cancer, and other hyperproliferative diseases, ischemic and hypoxic conditions and EPO-related conditions. A list of nonexhaustive examples of therapeutic benefit includes extension of the subject's life by any period of time, decrease or delay in the neoplastic development of the disease, decrease in hyperproliferation, reduction in tumor growth, delay of metastases or reduction in number of metastases, reduction in cancer cell or tumor cell proliferation rate, decrease or delay in progression of neoplastic development from a premalignant condition, a decrease in pain to the subject that can be attributed to the subject's condition, an enhanced ability of biological matter to enter stasis in response to an injury or disease condition, e.g., by reducing the time or level of injury or disease required to achieve stasis, and preservation of biological matter.

“Prevention” and “preventing” are used according to their ordinary and plain meaning to mean “acting before” or such an act. In the context of a particular disease or health-related condition, those terms refer to administration or application of an agent, such as an Effective Compound of the present invention, drug, or remedy to a subject or performance of a procedure or modality on a subject for the purpose of blocking the onset of a disease or health-related condition. In certain embodiments of the present invention, the methods involving delivery of an Effective Compound to prevent a disease or health-related condition in a subject. An amount of a pharmaceutical composition that is suitable to prevent a disease or condition is an amount that is known or suspected of blocking the onset of the disease or health-related condition.

The term “anemia” as used herein refers to any abnormality in hemoglobin or erythrocytes that leads to reduced oxygen levels in the blood. As such, anemia can be considered a hypoxia-related condition. Anemia can be associated with abnormal production, processing, or performance of erythrocytes and/or hemoglobin. The term anemia refers to any reduction in the number of red blood cells and/or level of hemoglobin in blood relative to normal blood levels.

Anemia can arise due to conditions such as acute or chronic kidney disease, infections, inflammation, cancer, irradiation, toxins, diabetes, and surgery. Infections may be due to, e.g., virus, bacteria, and/or parasites, etc. Inflammation may be due to infection, autoimmune disorders, such as rheumatoid arthritis, etc. Anemia can also be associated with blood loss due to, e.g., stomach ulcer, duodenal ulcer, hemorrhoids, cancer of the stomach or large intestine, trauma, injury, surgical procedures, etc. Anemia is further associated with radiation therapy, chemotherapy, and kidney dialysis. Anemia is also associated with HIV-infected patients undergoing treatment with azidothymidine (zidovudine) or other reverse transcriptase inhibitors, and can develop in cancer patients undergoing chemotherapy, e.g., with cyclic cisplatin- or non-cisplatin-containing chemotherapeutics. Aplastic anemia and myelodysplastic syndromes are diseases associated with bone marrow failure that result in decreased production of erythrocytes. Further, anemia can result from defective or abnormal hemoglobin or erythrocytes, such as in disorders including microcytic anemia, hypochromic anemia, etc. Anemia can result from disorders in iron transport, processing, and utilization, see, e.g., sideroblastic anemia, etc.

The terms “anemic conditions” and “anemic disorders” refer to any condition, disease, or disorder associated with anemia. Such disorders include, but are not limited to, those disorders listed above. Anemic disorders further include, but are not limited to, aplastic anemia, autoimmune hemolytic anemia, bone marrow transplantation, Churg-Strauss syndrome, Diamond Blackfan anemia, Fanconi's anemia, Felty syndrome, graft versus host disease, hematopoietic stem cell transplantation, hemolytic uremic syndrome, myelodysplastic syndrome, nocturnal paroxysmal hemoglobinuria, osteomyelofibrosis, pancytopenia, pure red-cell aplasia, purpura Schoenlein-Henoch, sideroblastic anemia, refractory anemia with excess of blasts, rheumatoid arthritis, Shwachman syndrome, sickle cell disease, thalassemia major, thalassemia minor, thrombocytopenic purpura, etc.

The term “erythropoietin-associated conditions” is used inclusively and refers to any condition associated with below normal, abnormal, or inappropriate modulation of erythropoietin. Erythropoietin-associated conditions include any condition wherein an increase in EPO level would provide therapeutic benefit. Levels of erythropoietin associated with such conditions can be determined by any measure accepted and utilized by those of skill in the art. Erythropoietin-associated conditions include anemic conditions such as those described above.

Erythropoietin-associated conditions further include neurological disorders and/or injuries, including cases of stroke, trauma, epilepsy, neurodegenerative disease and the like, wherein erythropoietin may provide a neuroprotective effect. Neurodegenerative diseases contemplated by the invention include Alzheimer's disease, Parkinson's disease, Huntington's disease, and the like.

The term “erythropoietin” refers to any recombinant or naturally occurring erythropoietin including, e.g., human erythropoietin (GenBank Accession No. AAA52400; Lin et al. (1985)), EPOETIN human recombinant erythropoietin (Amgen, Inc., Thousand Oaks Calif.), ARANESP human recombinant erythropoietin (Amgen), PROCRIT human recombinant erythropoietin (Ortho Biotech Products, L.P., Raritan N.J.), etc.

The term “agonist” refers to a molecule that increases or prolongs the duration of the effect of a particular molecule, e.g., an enzyme or protein, or a particular environment, e.g., hypoxia. Agonists may include proteins, nucleic acids, carbohydrates, or any other molecules that modulate the effects of the target molecule.

The term “antagonist” refers to a molecule which decreases the extent or duration of the effect of the biological or immunological activity of a particular molecule. Antagonists may include proteins, nucleic acids, carbohydrates, antibodies, or any other molecules that decrease the effect of the target molecule.

The term “excipient” as used herein means an inert or inactive substance used in the production of pharmaceutical products or other tablets, including without limitation any substance used as a binder, disintegrant, coating, compression/encapsulation aid, cream or lotion, lubricant, parenteral, sweetener or flavoring, suspending/gelling agent, or wet granulation agent. Binders include, e.g., carbopol, povidone, xanthan gum, etc.; coatings include, e.g., cellulose acetate phthalate, ethylcellulose, gellan gum, maltodextrin, etc.; compression/encapsulation aids include, e.g., calcium carbonate, dextrose, fructose dc, honey dc, lactose (anhydrate or monohydrate; optionally in combination with aspartame, cellulose, or microcrystalline cellulose), starch dc, sucrose, etc.; disintegrants include, e.g., croscarmnellose sodium, gellan gum, sodium starch glycolate, etc.; creams and lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; parenterals include, e.g., mannitol, povidone, etc.; plasticizers include, e.g., dibutyl sebacate, polyvinylacetate phthalate, etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

The term “sample” is used herein in its broadest sense. Samples may be derived from any source, for example, from bodily fluids, secretions, tissues, cells, or cells in culture including, but not limited to, saliva, blood, urine, serum, plasma, vitreous, synovial fluid, cerebral spinal fluid, amniotic fluid, and organ tissue (e.g., biopsied tissue); from chromosomes, organelles, or other membranes isolated from a cell; from genomic DNA, cDNA, RNA, mRNA, etc.; and from cleared cells or tissues, or blots or imprints from such cells or tissues. Samples may be derived from any source, such as, for example, a human subject, or a non-human mammalian subject, etc. Also contemplated are samples derived from any animal model of disease. A sample can be in solution or can be, for example, fixed or bound to a substrate. A sample can refer to any material suitable for testing for the presence of HIFα or of fragments of HIFα or suitable for screening for molecules that bind to HIFα or to fragments thereof. Methods for obtaining such samples are within the level of skill in the art.

The term “subject” is used herein in its broadest sense. Subjects may include isolated cells, either prokaryotic or eukaryotic, or tissues grown in culture. Subjects may include animals, such as a mammalian species including rat, rabbit, bovine, ovine, porcine, murine, equine, and primate, particularly human.

It is contemplated that any agent or solution used with a biological sample that is living and that will be used as a living material will be pharmaceutically acceptable or pharmacologically acceptable. The phrase “pharmaceutically-acceptable” or “pharmacologically-acceptable” refers to molecular entities and compositions that do not produce an allergic or similar untoward reaction when administered to a human.

It is contemplated that any embodiment discussed in this specification can be implemented with respect to any method, compound or composition of the invention, and vice versa. Furthermore, compounds and compositions of the invention can be used to achieve methods of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value. For example, “about” can be within 10%, preferably within 5%, more preferably within 1%, and most preferably within 0.5%. In any embodiment discussed in the context of a numerical value used in conjunction with the term “about,” it is specifically contemplated that the term about can be omitted.

Following long-standing patent law, the words “a” and “an,” when used in conjunction with the word “comprising” in the claims or specification, denotes one or more, unless specifically noted.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1. Genetic studies of sulfide activity and tolerability in sulfide adapted C. elegans.

FIG. 2. Genetic studies of sulfide activity and tolerability in C. elegans.

### DESCRIPTION OF ILLUSTRATIVE EMBODIMENTS

### I. Methods of Using the Effective Compounds for Treatment or Inducement of Conditions Associated with HIFα Stabilization

As discussed herein, stabilization of HIFα is known to be associated with hypoxic and ischemic events. Studies of stasis have focused on cellular metabolism, but stasis also exhibits some characteristics similar to those seen in hypoxic and ischemic events. The present inventors have discovered that HIF regulation is a common feature of hypoxia, ischemia and stasis. Accordingly, the present invention generally relates to compounds and methods relating to the modulation of HIF and HIF-related conditions.

Stabilization of HIFα is typically modulated by the inhibition of proline hydroxylation and such HIFα stabilization is effective for treating or preventing the development or persistence of hypoxic conditions such as altitude sickness and anemia, and ischemic conditions such as deep vein thrombosis, angina pectoris, pulmonary embolism, stroke, myocardial infarction, etc. The stabilization of HIFα, such as via the inhibition of one or more 2-oxoglutarate dioxygenases (e.g., a HIF prolyl hydroxylase), is also associated with stasis, as described herein. Thus, compounds that modulate HIF, such as those of the present invention, may, in certain embodiments, treat or induce HIF-related conditions.

The following sections describe certain HIF-related conditions (e.g., hypoxia, ischemia and stasis) in more detail, and a discussion of methods of using the Effective Compounds for the treatment or inducement of conditions associated with HIFα stabilization is also provided.

A. Hypoxic and Ischemic Conditions

As discussed, hypoxia, the condition that induces the production of HIFα, is a state of reduced oxygen, which can occur when the lungs are compromised or blood flow is reduced. Ischemia, reduction in blood flow, can be caused by the obstruction of an artery or vein by a blood clot (thrombus) or by any foreign circulating matter (embolus), or by a vascular disorder such as atherosclerosis. Reduction in blood flow can have a sudden onset and short duration (acute ischemia), or can have a slow onset with long duration or frequent recurrence (chronic ischemia). Acute ischemia is often associated with regional, irreversible tissue necrosis (an infarct), whereas chronic ischemia is usually associated with transient hypoxic tissue injury. If the decrease in perfusion is prolonged or severe, however, chronic ischemia can also be associated with an infarct. Infarctions commonly occur in the spleen, kidney, lungs, brain, and heart, producing disorders such as intestinal infarction, pulmonary infarction, ischemic stroke, and myocardial infarction.

Pathologic changes in ischemic disorders depend on the duration and severity of ischemia, and on the length of patient survival. Necrosis can be seen within the infarct in the first 24 hours, and an acute inflammatory response develops in the viable tissue adjacent to the infarct with leukocytes migrating into the area of dead tissue. Over succeeding days, there is a gradual breakdown and removal of cells within the infarct by phagocytosis, and replacement with a collagenous or glial scar.

Hypoperfusion or infarction in one organ often affects other organs. For example, ischemia of the lung, caused by, for example, a pulmonary embolism, not only affects the lung, but also puts the heart and other organs, such as the brain, under hypoxic stress. Myocardial infarction, which often involves coronary artery blockage due to thrombosis, arterial wall vasospasms, or viral infection of the heart, can lead to congestive heart failure and systemic hypotension. Secondary complications such as global ischemic encephalopathy can develop if the cardiac arrest is prolonged with continued hypoperfusion. Cerebral ischemia, most commonly caused by vascular occlusion due to atherosclerosis, can range in severity from transient ischemic attacks (TIAs) to cerebral infarction or stroke. While the symptoms of TIAs are temporary and reversible, TIAs tend to recur and are often followed by a stroke.

Occlusive arterial disease includes coronary artery disease, which can lead to myocardial infarction, and peripheral arterial disease, which can affect the abdominal aorta, its major branches, and arteries of the legs. Peripheral arterial disease includes Buerger's disease, Raynaud's disease, and acrocyanosis. Although peripheral arterial disease is commonly caused by atherosclerosis, other major causes include, e.g., diabetes, etc. Complications associated with peripheral arterial disease include severe leg cramps, angina, abnormal heart rhythms, heart failure, heart attack, stroke, and kidney failure.

Ischemic and hypoxic disorders are a major cause of morbidity and mortality. Cardiovascular diseases cause at least 15 million deaths every year and are responsible for 30% of deaths worldwide. Among the various cardiovascular diseases, ischemic heart disease and cerebrovascular diseases cause approximately 17% of deaths. Annually, 1.3 million cases of nonfatal acute myocardial infarction are reported, making the prevalence approximately 600 per 100,000 people. Further, an estimated five million Americans suffer from venous thrombosis every year, and approximately 600,000 of these cases result in pulmonary embolism. About one-third of the pulmonary embolisms end in death, making pulmonary embolism the third most common cause of death in the United States.

HIF induction results as an early response to tissue hypoxia, and thus is a critical regulator of cellular and systemic responses to low oxygen levels. Levels of HIFα protein are elevated in most cells in response to hypoxia and HIFα is induced in vivo when animals are subjected to anemia or hypoxia. HIFα levels rise within a few hours after the onset of hypoxia and return to baseline under continued hypoxic conditions. HIF has been implicated in numerous cellular and developmental processes including cell proliferation, angiogenesis, and cell cycle arrest. HIFα has also been associated with myocardial acute ischemia and early infarction, pulmonary hypertension, and inflammation. Although HIFα has been associated with tumor growth and metastasis, there is little indication that HIF is directly involved in tumorigenesis. Hypoxic preconditioning, in which a target organ is subjected to brief periods of hypoxia, has been shown to protect both myocardium and brain against hypoxic-ischemic injury. HIFα stabilization is closely associated with ischemia and is induced by preconditioning. (Wang and Semenza, (1993); Stroka, et al. (2001); Semenza et al. (1997); Carmeliet et al. (1998); Zhong et al. (1999); Lee et al. (2000); Sharp et al. (2000); Semenza et al. (2000); Thornton et al. (2000); Deindl and Schaper (1998); Bergeron et al. (2000).)

HIF is a basic helix-loop-helix (bHLH) PAS (Per/Arnt/Sim) transcriptional activator that mediates changes in gene expression in response to changes in cellular oxygen concentration. HIF is a heterodimer containing an oxygen-regulated alpha subunit (HIFα) and a constitutively expressed beta subunit (HIFβ), also known as aryl hydrocarbon receptor nuclear transporter (ARNT). In oxygenated (normoxic) cells, HIFα subunits are rapidly degraded by a mechanism that involves ubiquitination by the von Hippel-Lindau tumor suppressor (pVHL or VHL) E3 ligase complex. This mechanism involves hydroxylation of specific proline residues of the α-subunit of HIF by oxygen-dependent enzymes belonging to the Egl-9/PH superfamily of 2-oxoglutarate-dependent dioxygenases, such as Egl-9. Shen et al., 2005. This hydroxylation step increases the subunit's affinity for VHL and subsequent degradation. Under hypoxic conditions, HIFα is not degraded, and an active HIFα/β complex accumulates in the nucleus and activates the expression of several genes including glycolytic enzymes, glucose transporter (GLUT)-1, erythropoietin (EPO), and vascular endothelial growth factor (VEGF). (Jiang, et al. (1996); Iliopoulus, et al. (1996); Maxwell, et al. (1999); Sutter, et al. (2000); Cockman et al., (2000); and Tanimoto et al., (2000).)

Several investigators have studied the mechanism of interaction between HIFα and pVHL. An oxygen-dependent degradation domain (ODD) within HIF-1α from residue 401 to 603 was originally identified as sufficient to confer oxygen-dependent instability to chimeric protein constructs. A domain containing a portion of the ODD, from residue 526 to 652, was found to be required for pVHL-dependent degradation. Further, mutation of P₅₆₄YI to aspartic acids or mutation of K₅₃₂ to arginine within a region conserved among HIFα homologs (residue 556 to 574 in HIF-1α) rendered the full-length HIFα protein stable under normoxic conditions and resistant to pVHL-mediated degradation. (Huang et al. (1998); and Tanimoto et al. (2000).)

Three groups have recently reported that pVHL recognizes the ODD via a conserved proline residue that is hydroxylated exclusively under normoxic conditions (Ivan et al. (2001); Jaakkola et al. (2001); Yu et al. (2001).) Examination of cellular extracts prepared under normoxic conditions revealed the presence of a prolyl-4-hydroxylase activity capable of modifying a proline-containing peptide derived from the ODD (Ivan et al. (2001); Jaakkola et al. (2001); Yu et al. (2001).) This activity was greatly diminished in extracts prepared under hypoxic conditions or in the presence of “hypoxia mimics” such as CoCl₂ or the iron chelator deferoxamine mesylate (Ivan et al. (2001); Jaakkola et al. (2001); Yu et al. (2001).) As is the case for known prolyl-4-hydroxylases, this activity was enhanced by supplementation with Fe²⁺, ascorbate and 2-oxoglutarate (Jaakkola et al. (2001); Yu et al. (2001).)

Additionally, it has been shown that HIF-1α and a HIF-1α peptide corresponding to residues 556 to 575 [HIF(556-575)] preincubated with rabbit reticulocyte lysate (RRL) bind specifically to pVHL, and that such binding leads to the ubiquitination and degradation of HIF-1α. It has also been shown that mutation of the highly conserved colinear sequence M₅₆₁LAPYIPM within HIF(556-575) to eight consecutive alanines stabilized HIF(556-575) under normoxic conditions. (Srinivas et al., supra.) An alanine scan of the region showed that mutation of P₅₆₄ to alanine in the context of full-length HIF-1α or a glutathione S-transferase (GST)-HIFα oxygen degradation domain (ODD) fusion protein (Gal4-ODD) abrogated pVHL-binding activity. The modification of P₅₆₄ was identified as a hydroxylation by electrospray ion trap tandem mass spectrometry (MS/MS), and by thin layer chromatography of Gal4-HIF(555-575) that was in vitro translated using RRL in the presence of [³H]proline. The functional significance of the proline hydroxylation was demonstrated by showing that P₅₆₄-hydroxylated HIFα bound pVHL, while HIF-1α mutant containing a single point mutation of P₅₆₄ to alanine was stable in COS7 cells and was insensitive to the hypoxia mimetic desferrioxamine. See Ivan and Kaelin, supra; Jaakkola et al. (2001).

As HIFα is modified by proline hydroxylation, a reaction requiring oxygen and Fe²⁺, the present invention contemplates in one aspect that the enzyme responsible for HIFα hydroxylation is a member of the 2-oxoglutarate dioxygenase family. Such enzymes include, but are not limited to, procollagen lysyl hydroxylase, procollagen prolyl 3-hydroxylase, procollagen prolyl 4-hydroxylase α(I) and α(II), thymine 7-hydroxylase, aspartyl (asparaginyl) β-hydroxylase, .epsilon.-N-trimethyllysine hydroxylase, and .gamma.-butyrobetaine hydroxylase, etc. These enzymes require oxygen, Fe²⁺, 2-oxoglutarate, and ascorbic acid for their hydroxylase activity. (See, e.g., Majamaa et al. (1985); Myllyharju and Kivirikko (1997); Thornburg et al. (1993); and Jia et al. (1994).)

Several small molecule inhibitors of prolyl 4-hydroxylase have been identified. (See, e.g., Majamaa et al., 1985; Kivirikko and Myllyharju (1998); Bickel et al. (1998); Friedman et al. (2000); and Franklin et al. (2001); all incorporated by reference herein in their entirety.) The present invention contemplates the use of these compounds in the methods provided herein.

In certain embodiments, an Effective Compounds that can be used in the methods of the invention may inhibit the target 2-oxoglutarate dioxygenase enzyme family member competitively with respect to 2-oxoglutarate and noncompetitively with respect to iron. (Majamaa et al., 1984; and Majamaa et al., 1985)

Based on the common mechanism of action of the 2-oxoglutarate dioxygenase family members, such as dependence on Fe²⁺ and 2-oxoglutarate for activity, in certain aspects the invention is directed to use of compounds to inhibit HIFα hydroxylation and thus stabilize HIFα in an oxygen-independent manner. In specific embodiments, these compounds are used to produce a specific benefit in the prevention and treatment of ischemic and hypoxic conditions. In other embodiments, the compounds induce stasis.

Previous studies have shown that certain compounds used in the methods of the present invention are effective inhibitors of procollagen prolyl 4-hydroxylase. While it is recognized that recovery from an initial infarct or wound requires connective tissue deposition within the necrotic region, the present invention demonstrates no adverse affects of treatment with respect to scar formation. Thus, based on the benefits provided by certain compounds of the invention on treatment and prevention of hypoxic tissue damage and fibrosis, the present invention contemplates a “dual-therapy” approach to treatment or prevention of conditions involving ischemia or hypoxia, including ischemia or hypoxia associated with subsequent reactive fibrosis, e.g., myocardial infarction and resultant congestive heart failure. The method may use one compound that inhibits more than one 2-oxoglutarate dioxygenase enzyme, e.g., HIF prolyl hydroxylase and procollagen prolyl 4-hydroxylase, with either the same specificity or with different specificities. Alternatively, the method may use a combination of compounds wherein each compound specifically inhibits only one 2-oxoglutarate dioxygenase enzyme, e.g., one compound specifically inhibits HIF prolyl hydroxylase and a second compound specifically inhibits procollagen prolyl 4-hydroxylase.

In one aspect, a compound of the invention inhibits one or more 2-oxoglutarate dioxygenase enzymes. In one embodiment, the compound inhibits at least two 2-oxoglutarate dioxygenase family members, e.g., HIF prolyl hydroxylase and HIF asparagine-hydroxylase (FIH-1), with either the same specificity or with differential specificity. In another embodiment, the compound is specific for one 2-oxoglutarate dioxygenase, e.g., HIF prolyl hydroxylase, and shows little to no specificity for other family members.

The compounds can be administered in combination with various other therapeutic approaches. In one embodiment, the compound is administered with another 2-oxoglutarate dioxygenase inhibitor, wherein the two compounds have differential specificity for individual 2-oxoglutarate dioxygenase family members. The two compounds may be administered at the same time as a ratio of one relative to the other. Determination of a ratio appropriate to a given course of treatment or a particular subject is within the level of skill in the art. Alternatively, the two compounds may be administered consecutively during a treatment time course, e.g., following myocardial infarction. In a particular embodiment, one compound specifically inhibits HIF prolyl hydroxylase enzyme activity, and a second compound specifically inhibits procollagen prolyl 4-hydroxylase enzyme activity. In another specific embodiment, one compound specifically inhibits HIF prolyl hydroxylase enzyme activity, and a second compound specifically inhibits HIF asparaginyl-hydroxylase enzyme activity. In another embodiment, the compound is administered with another therapeutic agent having a different mode of action, e.g., an ACE inhibitor (ACEI), angiotensin-II receptor blocker (ARB), statin, diuretic, digoxin, carnitine, etc.

Hypoxia is a common natural stress and several well conserved responses exist that facilitate cellular adaptation to hypoxic environments. To compensate for the decrease in the capacity for aerobic energy production in hypoxia, the cell must either increase anaerobic energy production or decrease energy demand (Hochachka et al., 1996). Examples of both of these responses are common in metazoans and the particular response used depends, in general, on the amount of oxygen available to the cell.

In mild hypoxia, oxidative phosphorylation is still partially active, so some aerobic energy production is possible. The cellular response to this situation, which is mediated in part by the hypoxia-inducible transcription factor, HIF-1, is to supplement the reduced aerobic energy production by upregulating genes involved in anaerobic energy production, such as glycolytic enzymes and glucose transporters (Semenza, 2001; Guillemin et al., 1997). This response also promotes the upregulation of antioxidants such as catylase and superoxide dismutase, which guard against free radical-induced damage. As a result, the cell is able to maintain near normoxic levels of activity in mild hypoxia.

In an extreme form of hypoxia, referred to as “anoxia”-defined here as <0.001 kPa O₂-oxidative phosphorylation ceases and thus the capacity to generate energy is drastically reduced. In order to survive in this environment, the cell must decrease energy demand by reducing cellular activity (Hochachka et al., 2001). For example, in turtle hepatocytes deprived of oxygen, a directed effort by the cell to limit activities such as protein synthesis, ion channel activity, and anabolic pathways results in a 94% reduction in demand for ATP (Hochachka et al., 1996). In zebrafish (Danio rerio) embryos, exposure to anoxia leads to a complete arrest of the heartbeat, movement, cell cycle progression, and developmental progression (Padilla et al., 2001). Similarly, C. elegans respond to anoxia by entering into suspended animation, in which all observable movement, including cell division and developmental progression, ceases (Padilla et al., 2002; Van Voorhies et al., 2000). C. elegans can remain suspended for 24 hours or more and, upon return to normoxia, will recover with high viability. This response allows C. elegans to survive the hypoxic stress by reducing the rate of energetically expensive processes and preventing the occurrence of damaging, irrevocable events such as aneuploidy (Padilla et al., 2002; Nystul et al., 2003).

One recently discovered response is the hypoxia-induced generation of carbon monoxide by heme oxygenase-1 (Dulak et al., 2003). Endogenously produced carbon monoxide can activate signaling cascades that mitigate hypoxic damage through anti-apoptotic (Brouard et al., 2003) and anti-inflammatory (Otterbein et al., 2000) activity, and similar cytoprotective effects can be achieved in transplant models by perfusion with exogenous carbon monoxide (Otterbein et al., 2003; Amersi et al., 2002). At higher concentrations, carbon monoxide competes with oxygen for binding to iron-containing proteins, such as mitochondrial cytochromes and hemoglobin (Gorman et al., 2003), though the cytoprotective effect that this activity may have in hypoxia has not been investigated.

Despite the existence of these sophisticated defense mechanisms against hypoxic damage, hypoxia is still often a damaging stress. For example, mammals have both heme oxygenase-1 and HIF-1, and some evidence suggests that suspended animation is possible in mammals as well (Bellamy et al., 1996; Alam et al., 2002). Yet, hypoxic damage due to trauma such as heart attack, stroke or blood loss is a major cause of death. The understanding of the limitations of the two fundamental strategies for surviving hypoxic stress, remaining animated or suspending animation, is hampered by the fact that it has been based on studies in a variety of systems under a variety of conditions.

“Hypoxia” occurs when the normal physiologic levels of oxygen are not supplied to a cell or tissue. “Normoxia” refers to normal physiologic levels of oxygen for the particular cell type, cell state or tissue in question. “Anoxia” is the absence of oxygen. “Hypoxic conditions” are those leading to cellular hypoxia. These conditions depend on cell type, and on the specific architecture or position of a cell within a tissue or organ, as well as the metabolic status of the cell. For purposes of the present invention, hypoxic conditions include conditions in which oxygen concentration is at or less than normal atmospheric conditions, that is less that 20.8, 20, 19, 18, 17, 16, 15, 14, 13, 12, 11, 10, 9, 8, 7, 6, 5, 4, 3, 2, 1, 0.5, 0%; alternatively, these numbers could represent the percent of atmosphere at 1 atmosphere of pressure (101.3 kPa). An oxygen concentration of zero percent defines anoxic conditions. Thus, hypoxic conditions include anoxic conditions, although in some embodiments, hypoxic conditions of not less than 0.5% are implemented. As used herein, “normoxic conditions” constitute oxygen concentrations of around 20.8% or higher.

Standard methods of achieving hypoxia or anoxia are well established and include using environmental chambers that rely on chemical catalysts to remove oxygen from the chamber. Such chambers are available commercially from, for example, BD Diagnostic Systems (Sparks, Md.) as GASPAK Disposable Hydrogen+Carbon Dioxide Envelopes or BIO-BAG Environmental Chambers. Alternatively, oxygen may be depleted by exchanging the air in a chamber with a non-oxygen gas, such as nitrogen. Oxygen concentration may be determined, for example using a FYRITE Oxygen Analyzer (Bacharach, Pittsburgh Pa.).

It is contemplated that methods of the invention can use a combination of exposure to oxygen antagonist or other Effective Compound and alteration of oxygen concentrations compared to room air. Moreover, the oxygen concentration of the environment containing biological matter can be about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or any range derivable therein. Moreover, it is contemplated that a change in concentration can be any of the above percentages or ranges, in terms of a decrease or increase compared to room air or to a controlled environment.

The present invention provides, in certain embodiments, methods of stabilizing HIFα and/or inhibiting HIF prolyl hydroxylase, compounds that can be used in the methods, and to the use of the methods to prevent or treat disorders associated with HIF including, but not limited to, hypoxic, ischemic and/or EPO-associated disorders and uses of the methods to induce stasis. In certain embodiments of the present invention, the Effective Compounds comprise HIFα stabilizers and/or the HIF prolyl hydroxylase inhibitors. In other embodiments, a subset of the Effective Compounds comprising one or more Effective Compound is contemplated. The present invention further relates to the discovery that stabilization of the alpha subunit of hypoxia inducible factor (HIFα) is an effective therapeutic approach with unexpected benefits when applied to treatment or prevention of conditions associated with hypoxia and/or ischemia, e.g., myocardial infarction, stroke, occlusive arterial disease, angina pectoris, cardiac cirrhosis, atherosclerosis, shock, etc.

The present invention contemplates methods of stabilizing HIFα to augment angiogenesis, the response to acute hypoxia, and adaptation to chronic hypoxia. As tissue ischemia is a major cause of morbidity and mortality, the identification of methods that stabilize HIFα is beneficial in the treatment of hypoxic conditions. Further, the methods can be used to produce the beneficial effects of, e.g., a preconditioning hypoxic response, by stabilizing HIFα in a normoxic environment prior to an ischemic or hypoxic event. The methods can also be used to induce HIFα-specific effects, as described below, including therapeutic angiogenesis to restore blood flow to damaged tissues; neuroprotection to prevent, e.g., apoptotic loss of neurons associated with neurodegenerative diseases; and protection against oxidative damage produced by reactive oxygen species resulting from, e.g., reperfusion following an ischemic or hypoxic event.

When the methods of the invention are used to treat a disorder associated with ischemia and/or hypoxia, the disorder may be an acute ischemic disorder such as pulmonary, intestinal, cerebral, and/or myocardial infarction, or a chronic ischemic condition such as occlusive arterial disease, liver cirrhosis, congestive heart failure, etc. Further, the methods of the invention can be used to treat ischemia due to a transient or acute trauma, insult, or injury such as, e.g., a spinal cord injury, or to treat a patient diagnosed with, e.g., a pulmonary disorder such as pulmonary embolism and the like.

Methods for increasing expression of various HIF-related factors are specifically contemplated herein. In one aspect, the present invention provides a method for increasing expression of angiogenic factors in a subject, the method comprising stabilizing HIFα. In another aspect, the present invention provides a method of increasing expression of glycolytic factors in a subject, the method comprising stabilizing HIFα. In a further aspect, the invention provides a method of increasing expression of factors associated with oxidative stress in a subject, the method comprising stabilizing HIFα. A method of treating a subject having a disorder associated with ischemic reperfusion injury, the method comprising stabilizing HIFα, is also contemplated.

Stabilization of HIFα leads to HIF-dependent gene expression in vitro and in vivo, including genes encoding angiogenic factors such as VEGF, Flt-1, EG-VEGF, PAI-1, adrenomedullin, and Cyr61. Thus, the ability to stabilize HIFα has potential benefits in the induction of angiogenesis and prevention of tissue damage due to ischemia and hypoxia. For example, transgenic mice expressing constitutively active HIF-1α in the epidermis show enhanced expression of each VEGF isoform and a significant increase in dermal capillaries. Unlike overexpression of one VEGF isoform alone, the hypervascularity induced by HIFα shows no edema, inflammation, or vascular leakage. (See, Elson et al. (2001); Detmar et al. (1998); Larcher et al. (1998); and Thurston et al. (1999).) Therefore, in certain aspects, methods of the invention can be used to induce therapeutic angiogenesis, which involves the development of collateral blood vessels to revascularize ischemic tissues.

Additionally, the methods of the invention produce a dose-dependent decrease in oxygen consumption in cells without any affect on cell viability. Stable HIF complexes activate expression of proteins involved in glucose uptake and utilization, such as glucose transporter (GluT)-1 and GluT-3; aldolase-A, enolase-1, hexokinase-1 and -2, and phosphofructokinase-L and -C. The reduction in oxygen consumption associated with HIFα stabilization is potentially due to a shift in cellular metabolism from aerobic to anaerobic energy production. The present methods can thus be applied to generate energy under low oxygen conditions, beneficial in ischemic and hypoxic conditions such as, for example, peripheral arterial disease, DVT, angina pectoris, pulmonary embolism, stroke, and myocardial infarction. Methods of increasing glucose uptake and utilization by cells of the body, generally applicable to the treatment of other conditions, e.g., diabetes, are also provided.

Hypoxic preconditioning has been shown to effectively protect against subsequent acute ischemic insult. As the primary effect of hypoxia is stabilization of HIFα and subsequent activation of HIF-regulated genes, the methods of the invention will mimic hypoxic preconditioning in a normoxic environment. For example, the methods may be used prior to surgery, wherein ischemic-reperfusion injury may be expected to produce deleterious results in the patient. Such preventive therapy, when applied prior to an ischemic event, can be provided at any time point prior to the event, in a single or repeated dose format.

The methods of the invention also coordinately upregulate genes involved in oxidative stress and vascular tone. Such genes include, e.g., inducible nitric oxide synthase (iNOS), and heme oxygenase 1. Production of iNOS has also been associated with the beneficial effects of hypoxic preconditioning in several animal models. (See, e.g., Serracino-Inglott et al. (2002); Kuntscher et al. (2002).) Significantly, blocking iNOS activity attenuates but does not abrogate the beneficial effects of preconditioning, whereas nonspecifically blocking protein production completely abrogates the benefits of preconditioning. (Wang et al. (2002).) This suggests that iNOS is an important component of the physiological response to preconditioning, but is not the only factor. As the methods of the invention coordinately regulate various factors, including iNOS, involved in hypoxic response, the methods of the invention will more accurately replicate the beneficial effects of hypoxic preconditioning.

As discussed, the present invention provides, in certain embodiments, methods of inhibiting HIFα hydroxylation, thereby stabilizing HIFα and activating HIF-regulated gene expression. The methods can be applied to the prevention, pretreatment, or treatment of conditions associated with HIF including ischemic and hypoxic conditions. Such conditions include, for example, myocardial infarction, liver ischemia, renal ischemia, and stroke; peripheral vascular disorders, ulcers, burns, and chronic wounds; pulmonary embolism; and ischemic-reperfusion injury, including, for example, ischemic-reperfusion injury associated with surgery and organ transplantation. In one embodiment, the present invention provides methods of stabilizing HIFα before, during, or immediately after ischemia or hypoxia, particularly in association with myocardial infarction, stroke, or renal ischemic-reperfusion injury.

In one aspect, the invention provides methods for treating various ischemic and hypoxic conditions, in particular, using the compounds described herein. In one embodiment, the methods of the invention produce therapeutic benefit when Effective Compounds are administered following ischemia or hypoxia. In some embodiments, the Effective Compounds are selected from the group consisting of Formula I and Formula IV. In certain aspects, the methods of the invention produce a dramatic decrease in morbidity and mortality following myocardial infarction, and a significant improvement in heart architecture and performance. Further, the methods of the invention improve liver function when administered following hepatic toxic-ischemic injury. Hypoxia is a significant component of liver disease, especially in chronic liver disease associated with hepatotoxic compounds such as ethanol. Additionally, expression of genes known to be induced by HIFα, e.g., nitric oxide synthase and glucose transporter-1, is increased in alcoholic liver disease. (See, e.g., Areel et al. (1997); Strubelt (1984); Sato (1983); Nanji et al. (1995); and Morio et al. (2001).)

Therefore, the present invention provides methods of treating conditions associated with ischemia or hypoxia, the method comprising administering a therapeutically effective amount of a compound or a pharmaceutically acceptable salt thereof, alone or in combination with a pharmaceutically acceptable excipient, to a subject. In one embodiment, the compound is administered immediately following a condition producing acute ischemia, e.g., myocardial infarction, pulmonary embolism, intestinal infarction, ischemic stroke, and renal ischemic-reperfusion injury. In another embodiment, the compound is administered to a patient diagnosed with a condition associated with the development of chronic ischemia, e.g., cardiac cirrhosis, macular degeneration, pulmonary embolism, acute respiratory failure, neonatal respiratory distress syndrome, and congestive heart failure. In yet another embodiment, the compound is administered immediately after a trauma or injury.

HIFα levels are increased by a number of factors that mimic hypoxia, including iron chelators such as desferrioxamine (DFO) and divalent metal salts such as CoCl₂ HIFα levels are increased by angiotensin II, thrombin, and platelet-derived growth factor under normoxic conditions using a mechanism involving reactive oxygen species. Reports have also suggested HIFα is regulated by phosphorylation through pathways involving nitric oxide-activated phosphatidylinositol 3′-kinase (PI3K), hepatocyte growth factor, or mitogen-activated protein kinase. Glycogen-synthase kinase, which is a downstream target of PI3K, directly phosphorylates the HIFα ODD domain. (Richard et al. (2000); Sandau et al. (2000); Tacchini et al. (2001); and Sodhi et al. (2001).)

1. EPO-Associated Conditions

Erythropoietin (EPO), a naturally occurring hormone that is produced in response to HIFα, stimulates the production of red blood cells (erythrocytes), which carry oxygen throughout the body. EPO is normally secreted by the kidneys, and endogenous EPO is increased under conditions of reduced oxygen (hypoxia). All types of anemia are characterized by the blood's reduced capacity to carry oxygen, and thus are associated with similar signs and symptoms, including pallor of the skin and mucous membranes, weakness, dizziness, easy fatigability, and drowsiness, leading to a decrease in quality of life. Subjects with severe cases of anemia show difficulty in breathing and heart abnormalities. Anemia is typically associated with a condition in which the blood is deficient in red blood cells or in hemoglobin.

Common causes of anemia include deficiencies of iron, vitamin B₁₂, and folic acid. Anemia can also develop in association with chronic diseases, e.g., in inflammatory disorders, including disorders with consequent inflammatory suppression of marrow, etc. Anemia may be caused by loss of blood, for example, due to accidents, surgery, or gastrointestinal bleeding caused by medications such as aspirin and ibuprofen. Excessive blood loss can also be seen in women with heavy menstrual periods, and in people with stomach ulcers, duodenal ulcers, hemorrhoids, or cancer of the stomach or large intestine, etc.

Various conditions can cause the destruction of erythrocytes (hemolysis), thus leading to anemia. For example, allergic-type reactions to bacterial toxins and various chemical agents such as sulfonamides and benzene can cause hemolysis. Hemolytic anemia is often caused by chemical poisoning, parasites, infection, or sickle-cell anemia. In addition, there are unusual situations in which the body produces antibodies against its own erythrocytes, resulting in hemolysis. Any disease or injury to the bone marrow can cause anemia, since that tissue is the site of erythropoiesis, i.e. erythrocyte synthesis. Irradiation, disease, or various chemical agents can also cause bone marrow destruction, producing aplastic anemia. Cancer patients undergoing chemotherapy often have aplastic anemia. Anemia is also associated with renal dysfunction, the severity of the anemia correlating highly with the extent of the dysfunction. Most patients with renal failure undergoing dialysis suffer from chronic anemia.

In addition to being produced in the kidney, erythropoietin is produced by astrocytes and neurons in the central nervous system (CNS), and EPO and EPO receptors are expressed at capillaries of the brain-periphery interface. Furthermore, systemically administered EPO crosses the blood-brain barrier and reduces neuronal cell loss in response to cerebral and spinal chord ischemia, mechanical trauma, epilepsy, excitotoxins, and neuroinflammation. (Sakanaka (1998); Celik et al. (2002); Brines et al. (2000); Calapai et al. (2000); and Siren et al. (2001).)

In the late 1980s, Amgen introduced a genetically engineered EPO for the treatment of anemia in chronic renal failure patients. EPO is also administered to cancer patients undergoing radiation and/or chemotherapy, decreasing the need for blood transfusions. EPO is used to treat anemia associated with HIV infection or azidothymidine (AZT) therapy. Although the market for EPO therapy is increasing, future sales are adversely affected by the high cost of the product. In addition, recombinant EPO therapy requires intravenous administration of EPO one to three times per week for up to twelve weeks, a treatment regimen that limits self-administration and is inconvenient for the patient. Further, human serum EPO shows size heterogeneity due to extensive and varied glycosylation not reproduced in any recombinant human EPO.

In one aspect, then, the invention includes methods that provide neuroprotective benefits, e.g., by stabilizing HIFα. For example, both VEGF and erythropoietin (EPO) have been shown to be neuroprotective. See, e.g., Jin et al. (2000); Bocker-Meffert et al. (2002); Buemi et al. (2002); and Siren et al. (2001). EPO is a naturally occurring hormone that stimulates the production of red blood cells. See, e.g., U.S. published patent application No. 2003/0153503, incorporated herein by reference in its entirety. EPO also facilitates recovery from spinal cord injuries and provides neuroprotective benefits when induced prior to an ischemic event. (See, e.g., Gorio et al. (2002); and Dawson (2002).)

As the methods of the invention increase expression of neuroprotective factors such as VEGF and EPO, the methods provide neuroprotective benefit that can be applied to treatment, pretreatment, or prevention of conditions including, e.g., diabetic neuropathy, stroke, neurodegenerative disease, trauma, injury, e.g., concussions, spinal cord injuries, etc., or prior to surgical procedures, e.g., wherein cerebral ischemic reperfusion injury may result.

The invention further provides methods for increasing oxygen-carrying capacity by inducing erythropoiesis, and facilitating iron transport and utilization. Specifically, certain methods of the invention increase expression of EPO via administration of one or more Effective Compounds as described herein. Methods for increasing expression of enzymes and proteins involved in iron uptake, transport, and processing are specifically contemplated. Such enzymes and proteins include, but are not limited to, transferrin and transferrin receptor, which together facilitate iron transport to and uptake by, e.g., erythroid tissue; and ceruloplasmin, a ferroxidase required to oxidize ferrous iron to ferric iron. As transferrin can only bind and transport ferric iron, ceruloplasmin is important for supply of iron to tissues. The ability of the methods of the invention to increase both endogenous erythropoietin and transport and utilization of iron provides specific advantage in oxygen delivery in both normoxic and hypoxic environments.

The present invention provides, in certain embodiments, methods of increasing endogenous erythropoietin (EPO). These methods can be applied in vivo, e.g., in blood plasma, or in vitro, e.g., in cell culture conditioned media. The invention further provides methods of increasing endogenous EPO levels to prevent, pretreat, or treat EPO-associated conditions, including, e.g., conditions associated with anemia and neurological disorders. Conditions associated with anemia include disorders such as acute or chronic kidney disease, diabetes, cancer, ulcers, infection with virus, e.g., HIV, bacteria, or parasites; inflammation, etc. Anemic conditions can further include those associated with procedures or treatments including, e.g., radiation therapy, chemotherapy, dialysis, and surgery. Disorders associated with anemia additionally include abnormal hemoglobin and/or erythrocytes, such as found in disorders such as microcytic anemia, hypochromic anemia, aplastic anemia, etc.

The present methods can be used, in certain embodiments, to increase endogenous EPO in a subject undergoing a specific treatment or procedure, prophylactically or concurrently, for example, an HIV-infected anemic patient being treated with azidothymidine (zidovudine) or other reverse transcriptase inhibitors, an anemic cancer patient receiving cyclic cisplatin- or non-cisplatin-containing chemotherapeutics, or an anemic or non-anemic patient scheduled to undergo surgery. Methods of increasing endogenous EPO can also be used to prevent, pretreat, or treat EPO-associated conditions associated with nerve damage or neural tissue degeneration including, but not limited to, stroke, trauma, epilepsy, spinal cord injury, and neurodegenerative disorders.

Additionally, the methods can be used to increase endogenous EPO levels in an anemic or non-anemic patient scheduled to undergo surgery to reduce the need for allogenic blood transfusions or to facilitate banking of blood prior to surgery. The small decreases in hematocrit that typically occur after presurgical autologous blood donation do not stimulate an increase in endogenous EPO or in compensatory erythropoiesis. However, preoperative stimulation of endogenous EPO would effectively increase erythrocyte mass and autologous donation volumes while maintaining higher hematocrit levels, and such methods are specifically contemplated herein. In some surgical populations, particularly those individuals who experience surgical blood losses in excess of 2 liters, the methods of the invention could be applied to reduce allogeneic blood exposure (Crosby, 2002).

The methods of the invention can also be used to enhance athletic performance, improve exercise capacity, and facilitate or enhance aerobic conditioning. Such methods can be used, e.g., by athletes to facilitate training and by soldiers to improve, e.g., stamina and endurance.

The methods of the invention have been shown, in certain embodiments, to increase endogenous erythropoietin levels in media from cultured cells treated in vitro and in blood plasma from animals treated in vivo. Although the kidney is the major source of erythropoietin in the body, other organs, including brain, liver, and bone marrow, can and do synthesize erythropoietin upon appropriate stimulation. Using the methods of the invention, endogenous erythropoietin expression can be increased in various organs of the body, including brain, kidney, and liver. Indeed, methods of the invention even increase endogenous erythropoietin levels in animals that have undergone bilateral nephrectomy.

The methods of the invention demonstrate, in certain embodiments, that erythropoietin levels can be increased even when kidney function is compromised. Although the invention is not to be limited by the mechanism by which erythropoietin is produced, the decrease in erythropoietin secretion typically seen during kidney failure may be due to hyperoxia in renal tissue due to increased flowthrough/reperfusion (Priyadarshi et al., 2002).

Further, the methods of the invention increase, in certain embodiments, the hematocrit and blood hemoglobin level in animals treated in vivo. The increases in plasma EPO, hematocrit, and blood hemoglobin in response to the compounds used in the methods of the invention are dose-sensitive; however, dosing regimes can be established which produce a constant, controlled level of response to the compounds of the invention. Further, treatment with compounds of the invention can correct anemia, for example, induced by a toxic compound such as the chemotherapeutic agent cisplatin, or due to blood loss, e.g., trauma, injury, parasites, or surgery.

In certain embodiments, the increase in hematocrit and blood hemoglobin in animals treated with an Effective Compound is preceded by an increase in the percentage of circulating immature red blood cells (reticulocytes) within the blood. As such, the invention contemplates the use of the compounds of the invention in methods to increase reticulocyte levels in the blood of animals for production of cell-free reticulocyte lysates as described by, e.g., Pelham and Jackson (1976). Circulating reticulocyte levels are increased in animals, e.g., rabbits, etc., by treatment with compounds of the invention, alone or in combination with another compound such as, e.g., acetylphenylhydrazine, etc. The blood is collected, and reticulocytes are pelleted by centrifugation and lysed with distilled water. Extracts can be further processed using any appropriate methodology known to those skilled in the art. See, e.g., Jackson and Hunt (1983).

B. Stasis

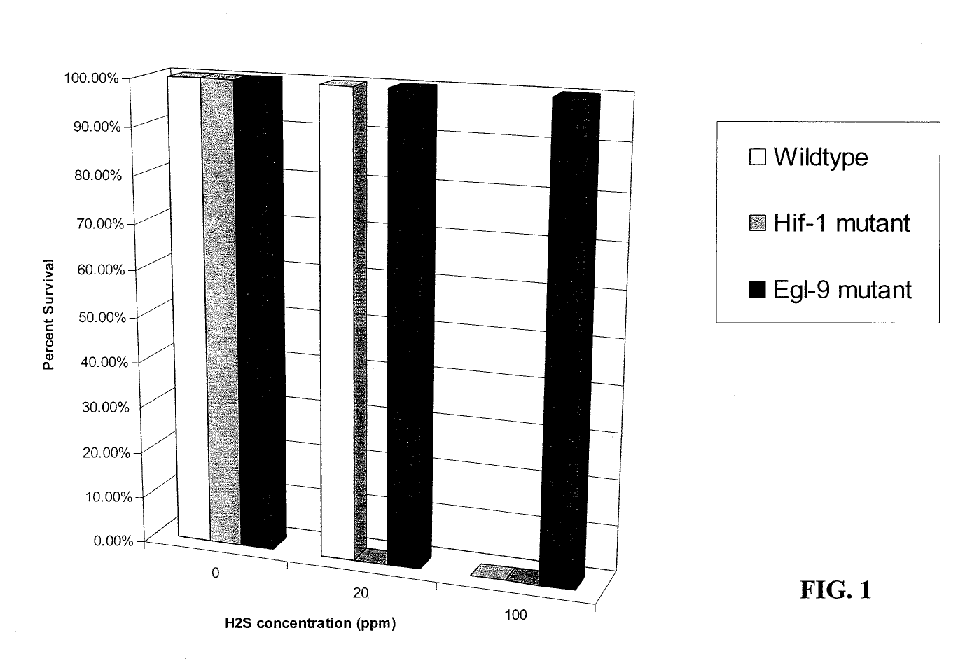

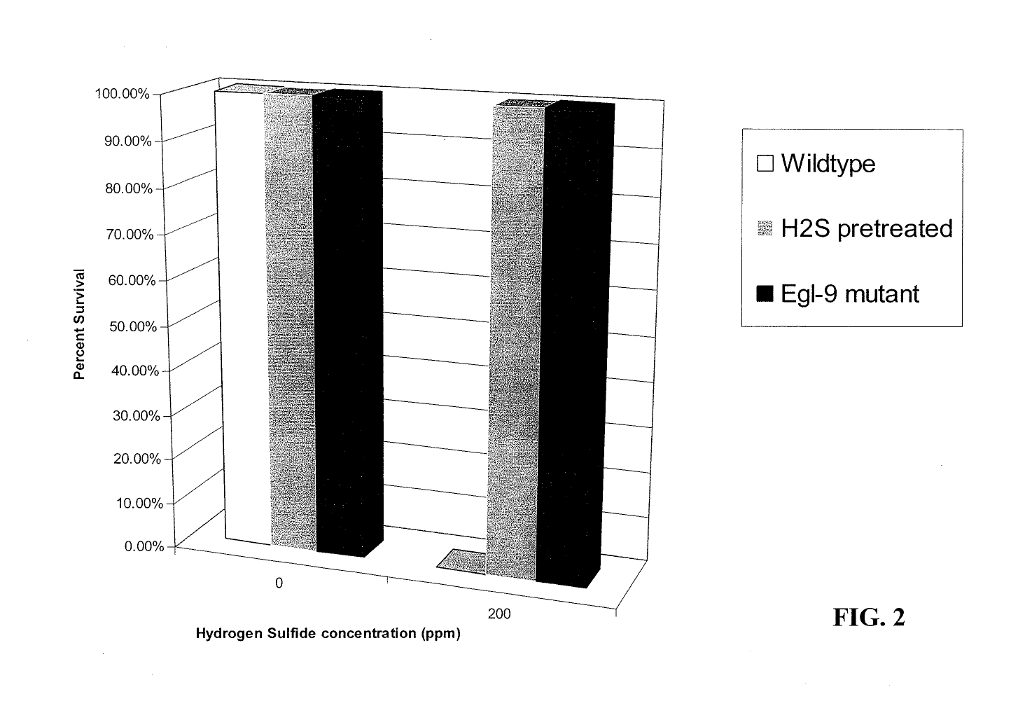

As discussed herein, compounds that induce stasis, such as H₂S, have been found to modulate HIF-1, such as via interaction with Egl-9, a HIF-prolyl hydroxylase. See FIG. 1 and FIG. 2. Some hypoxia- and ischemia-related conditions could be associated with stasis. Based on the discovered common feature of HIF regulation as among hypoxia- and ischemia-related conditions and stasis, the present invention accordingly contemplates compounds and methods that involve stasis-inducing compounds for the use of regulating hypoxia and ischemia-related conditions and vice versa. In certain embodiments, stasis occurs via the stabilization of HIFα, wherein one or more 2-oxoglutarate dioxygenase enzymes are inhibited. In some embodiments, one or more HIF prolyl hydroxylases are inhibited.

Stasis is a Latin term meaning “standstill.” In the context of stasis in living tissues, the most common forms of stasis relate to the preservation of tissues for transplant or reattachment. Typically, such tissues are immersed in a physiologic fluid, such as saline, and placed in the cold to reduce biochemical processes leading to cellular damage. This stasis is incomplete and cannot be relied upon for extended periods. In fact, the success of organ transplant and limb reattachment is inversely related to the time the organ or limb is out of contact with the intact organism.

A more extreme version of stasis involves placing an entire organism into what is known colloquially as “suspended animation.” Though still considered largely within the realm of science fiction, some notoriety has been achieved when wealthy individuals have sought to be cryopreserved after death, in the hope that future medical breakthroughs will permit their revival and cure of their fatal ailments. Allegedly, more than one hundred people have been cryopreserved since the first attempt in 1967, and more than one thousand people have made legal and financial arrangements for cryonics with one of several organizations, for example, Alcor Life Extension Foundation. Such methods involve the administration of anti-ischemic drugs, low temperature preservation, and methods to perfuse whole organisms with cryosuspension fluids. It has not yet been substantiated that this form of organismal stasis is reversible.

The utility of inducing stasis in biological matter as contemplated by the compositions, methods, or articles of manufacture described herein, is characterized by induction or onset of stasis followed by a period of time in which the stasis is maintained, followed then by reversion to a normal or near normal physiological state, or a state that one skilled in the art would recognize as a state that is better than the state of the biological matter had it never undergone stasis, in whole or in part. Stasis can also be defined as what it is not. Organismal stasis is not any of the following states: sleep, comatose, death, anesthetized, or grand mal seizure.

There are numerous reports of individuals who have survived apparent cessation of pulse and respiration after exposure to hypothermic conditions, usually in cold-water immersion. Though not fully understood by scientists, the ability to survive such situations likely derives from what is called the “mammalian diving reflex.” This reflex is believed to stimulate the vagal nervous system, which controls the lungs, heart, larynx and esophagus, in order to protect vital organs. Presumably, cold-water stimulation of nerve receptors on the skin causes shunting of blood to the brain and to the heart, and away from the skin, the gastro-intestinal tract and extremities. At the same time, a protective reflex bradycardia, or slowing of the heart beat, conserves the dwindling oxygen supplies within the body. Unfortunately, the expression of this reflex is not the same in all people, and is believed to be a factor in only 10-20% percent of cold-water immersion cases.

Compositions and methods that do not rely fully or at all on hypothermia and/or oxygen may be useful in the context of organ preservation, as well as for tissue or cell preservation. Cells and tissue are currently preserved using hypothermia, frequently at temperatures substantially below freezing, such as in liquid nitrogen. However, dependence on temperature can be problematic, as apparatuses and agents for producing such low temperatures may not be readily available when needed or they may require replacement.

1. Induction of Stasis

The inventors have discovered that stasis can be induced using compounds that interact with HIF and/or a HIF prolyl hydroxylase, such as Egl-9. See, e.g., FIG. 1 and FIG. 2. Thus, the present invention contemplates use of the Effective Compounds for the induction of reversible stasis via stabilization of HIFα, such as through the inhibition of one or more 2-oxoglutarate dioxygenase enzymes, such as HIF prolyl hydroxylase. In certain embodiments, a subset of the Effective Compounds are inducers of reversible stasis. In some embodiments, compounds of Formulas I, Ia-Id, II, III, IIIa, IV, V, VI, VII, VIII, IX and/or X are contemplated.

In “stasis” or “suspended animation,” a cell, tissue or organ, or organism (collectively referred to as “biological material”) is living, but cellular functions necessary for cell division, developmental progression, and/or metabolic state are slowed or even stopped. This state is desirable in a number of contexts. Stasis can be used as a method of preservation by itself, or it may be induced as part of a cryopreservation regimen. Biological materials may be preserved for research use, for transportation, for transplantation, for therapeutic treatment (such as ex vivo therapy), and to prevent the onset of trauma, for example. Stasis with respect to entire organisms has similar uses. For instance, transportation of organisms could be facilitated if they had entered stasis. This might reduce physical and physiological damage to the organism by reducing or eliminating stress or physical injury. These embodiments are discussed in further detail below. Stasis may be beneficial by decreasing the need of the biological material for oxygen and, therefore, bloodflow. It may extend the period of time that biological material can be isolated from a life-sustaining environment and exposed to a death-inducing environment.

While recovery has been reported from accidental hypothermia for a relatively prolonged period of time (Gilbert et al., 2000), there has been recent interest in intentionally inducing suspended animation in organisms. (The discussion of any reference is not to be construed as an admission that the reference constitutes prior art. In fact, some references discussed herein would not be prior art with respect to the priority applications.) Controlled hyperthermia has been explored, as well as the administration of a cold flush of a solution into the aorta (Tisherman, 2004), induction of cardiac arrest (Behringer et al., 2003), or nitric oxide-induced suspended animation (Teodoro et al., 2003).

An organism in stasis is distinguishable from an organism under general anesthesia. For example, an organism in mild stasis (between about 2- and about 5-fold decrease in cellular respiration) that is exposed to room air will begin to shiver, while an organism under anesthesia will not. Also, an organism in mild stasis is anticipated to respond to a toe squeeze, while an organism under anesthesia usually does not. Consequently, stasis is not the same thing as being under anesthesia as it is commonly practiced.

CO₂ production is a direct marker of cellular respiration related to metabolism of an organism. This may be distinguished from “CO₂ evolution,” which refers to the amount of CO₂ blown out of the lungs. Certain Effective Compounds, e.g., hydrogen sulfide, can inhibit carbonic anhydrase activity in the lungs, this inhibiting conversion of carbonate to CO₂ and its liberation from the pulmonary blood, thereby exhibiting an associated reduction in CO₂ evolution, without a corresponding decrease in cellular CO₂ production.

The present invention is based on the observation that certain types of compounds effectively induce reversible stasis in biological matter. Other patent applications discuss induction of stasis, including the following: U.S. patent application Ser. Nos. 10/971,576, 10/972,063; and 10/971,575, all of which are hereby incorporated by reference in their entireties.

a. Thermoregulation

Stasis in a warm-blooded animal will affect thermoregulation. Thermoregulation is a characteristic of so-called “warm-blooded” animals, which permits the organism to maintain a relatively constant core body temperature even when exposed to significantly altered (cold or hot) environmental temperatures. The ability to control thermoregulation by induction of stasis is one aspect of the invention, and permits uses similar to those discussed above.

Thermal regulation may be facilitated by placing of organisms, limbs or isolated organs or tissues into chambers/devices, the temperature of which can be controlled. For example, warm rooms or chamber-like devices similar to hyperbaric chambers may encompass an entire organism and be connected to thermo-regulatory apparti. Smaller devices such as blankets, sleeves, cuffs or gloves (e.g., CORE CONTROL cooling system by AVAcore Technologies, Palo Alto, Calif., U.S. Pat. No. 6,602,277, incorporated herein by reference in its entirety) are also contemplated. Such chambers/devices may be used both to increase or reduce ambient temperatures.

b. Biological Matter

Biological matter contemplated for use with all aspects of the present invention include material derived from invertebrates and vertebrates, including mammals; biological materials includes organisms. In addition to humans, the invention can be employed with respect to mammals of veterinary or agricultural importance including those from the following classes: canine, feline, equine, bovine, ovine, murine, porcine, caprine, rodent, lagomorph, lupine, and ursine. The invention also extends to fish, birds, reptiles, amphibians, invertebrates, fungi, plants, protists and prokaryotes. Such species are described in U.S. patent application Ser. No. 11/408,734, herein incorporated by reference in its entirety.

Moreover, the type of biological matter varies. It can be cells, tissues and organs, as well as organisms for which different compositions, methods, and apparatuses have relevance. The nonprovisional U.S. patent application Ser. Nos. 10/971,576, 10/972,063 and 10/971,575 are hereby incorporated by reference in their entireties.

(i) Different Types of Biological Matter

Methods and apparatuses of the invention can be applied to organisms. Stasis of the organism can be induced or stasis within cells, tissues, and/or organs of the organism can be induced. Biological matter in which stasis can be induced that are contemplated for use with methods and apparatuses of the invention are limited only insofar as the comprise cells utilizing oxygen to produce energy.

Stasis can be induced in cells, tissues, or organs involving the heart, lung, kidney, liver, bone marrow, pancreas, skin, bone, vein, artery, cornea, blood, small intestine, large intestine, brain, spinal cord, smooth muscle, skeletal muscle, ovary, testis, uterus, and umbilical cord.

Moreover, stasis can be induced in cells of the following type: platelet, myelocyte, erythrocyte, lymphocyte, adipocyte, fibroblast, epithelial cell, endothelial cell, smooth muscle cell, skeletal muscle cell, endocrine cell, glial cell, neuron, secretory cell, barrier function cell, contractile cell, absorptive cell, mucosal cell, limbus cell (from cornea), stem cell (totipotent, pluripotent or multipotent), unfertilized or fertilized oocyte, or sperm.

Moreover, stasis can be induced in plants or parts of plants, including fruit, flowers, leaves, stems, seeds, cuttings. Plants can be agricultural, medicinal, or decorative. Induction of stasis in plants may enhance the shelf life or pathogen resistance of the whole or part of the plant.

Methods and apparatuses of the invention can be used to induce stasis in in vivo biological matter. This can serve to protect and/or preserve the biological matter or the organism itself or to prevent damage or injury (or further damage or injury) to them or the organism overall.

c. Trauma

In certain embodiments, the present invention may find use in the treatment of patients who are undergoing, or who are susceptible to trauma. Trauma may be caused by external insults, such as burns, wounds, amputations, gunshot wounds, or surgical trauma, internal insults, such as stroke or heart attack that result in the acute reduction in circulation, or reductions in circulation due to non-invasive stress, such as exposure to cold or radiation. Trauma can also result from blood loss, resulting in one or more hypoxic conditions, such as EPO-related conditions (e.g., anemia), as described herein. On a cellular level, trauma often results in exposure of cells, tissues and/or organs to hypoxia, thereby resulting in induction of programmed cell death, or “apoptosis.” Systemically, trauma leads to the induction of a series of biochemical processes, such as clotting, inflammation, hypotension, and may give rise to shock, which if it persists may lead to organ dysfunction, irreversible cell damage and death. Biological processes are designed to defend the body against traumatic insult; however they may lead to a sequence of events that proves harmful and, in some instances, fatal.

Therefore, the present invention contemplates the placement of tissues, organs, limbs and even whole organisms into stasis as a way of protecting them from the detrimental effects of trauma. For example, one or more Effective Compounds may be administered to induce stasis via modulation of HIF (e.g., stabilization of HIFα) and/or one or more 2-oxoglutarate dioxygenase enzymes, such as HIF prolyl hydroxylase (e.g., HIF prolyl hydroxylase inhibition); such modulation could offer physiological protection from further detrimental effects of trauma.

In a specific scenario, where medical attention is not readily available, induction of stasis in vivo or ex vivo, alternatively in conjunction with reduction in the temperature of the tissue, organ or organism, can “buy time” for the subject, either by bringing medical attention to the subject, or by transporting the subject to the medical attention. The present invention also contemplates methods for inducing tissue regeneration and wound healing by prevention/delay of biological processes that may result in delayed wound healing and tissue regeneration. In this context, in scenarios in which there is a substantial wound to the limb or organism, the induction of stasis induction of stasis in vivo or ex vivo, alternatively in conjunction with reduction in the temperature of the tissue, organ or organism, can aid in the wound healing and tissue regeneration process by managing the biological processes that inhibit healing and regeneration.

In addition to wound healing and hemorrhagic shock discussed below, methods of the invention can be implemented to prevent or treat trauma such as cardiac arrest or stroke. The invention has particular importance with respect to the risk of trauma from emergency surgical procedures, such as thoractomy, laparotomy, and splenic transection.

(i) Wound Healing

In many instances, wounds and tissue damage are intractable or take excessive periods of time to heal. Examples are chronic open wounds (diabetic foot ulcers and stage 3 & 4 pressure ulcers), acute and traumatic wounds, flaps and grafts, and subacute wounds (i.e., dehisced incisions). This may also apply to other tissue damage, for example burns and lung damage from smoke/hot air inhalation.

Previous experiments show hibernation to be protective against injury (e.g., pin's in brains), therefore it may have healing effects. Consequently, this technology may be useful in the control of wound healing processes, by bringing the tissue into a more metabolically controlled environment. More particularly, the length of time that cells or tissue are kept in stasis can vary depending on the injury. In some embodiments of the invention, biological matter is exposed to an Effective Compound for about, at least about, or at most about 30 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, 1, 2, 3, 4, 5, 6, 7, 8, 9, 10, 11, 12 months or more.

(ii) Hematologic Shock (Hemorrhagic Shock)

Shock is a life-threatening condition that progresses rapidly when interventions are delayed. Shock is a state in which adequate perfusion to sustain the physiologic needs of organ tissues is not present. This is a condition of profound haemodynamic and metabolic disturbance characterized by failure of the circulatory system to maintain adequate perfusion of vital organs. It may result from inadequate blood volume (hypovolaemic shock), which relates to ischemia, inadequate cardiac function (cardiogenic shock) or inadequate vasomotor tone, also referred to as distributive shock (neurogenic shock, septic shock, anaphylactic shock). This often results in rapid mortality of the patient. Many conditions, including sepsis, blood loss, impaired autoregulation, and loss of autonomic tone, may produce shock or shocklike states. The present invention is anticipated to prevent detrimental effects of all the above states of shock, and sustain the life of the biological matter undergoing such shock.

In hemorrhagic shock, blood loss exceeds the body's ability to compensate and provide adequate tissue perfusion and oxygenation. This is frequently due to trauma, but may also be caused by spontaneous hemorrhage (e.g., gastrointestinal bleeding, childbirth), surgery, and other causes. Most frequently, clinical hemorrhagic shock is caused by an acute bleeding episode with a discrete precipitating event. Less commonly, hemorrhagic shock may be seen in chronic conditions with subacute blood loss. Such blood loss can be considered an ischemic condition, and it is therefore contemplated that compounds of the present invention can be used to treat or manage such blood loss, wherein one or more Effective Compounds modulates HIF and/or one or more 2-oxoglutarate dioxygenases (e.g., HIF prolyl hydroxylase). For example, by stabilizing HIFα using an Effective Compound, such as by inhibiting a HIF prolyl hydroxylase, stasis could be induced, thereby offering metabolic protection for a tissue, organ or subject.

Physiologic compensation mechanisms for hemorrhage include initial peripheral and mesenteric vasoconstriction to shunt blood to the central circulation. This is then augmented by a progressive tachycardia. Invasive monitoring may reveal an increased cardiac index, increased oxygen delivery (i.e., DO₂), and increased oxygen consumption (i.e., VO₂) by tissues. Lactate levels, the acid-base status, and other markers also may provide useful indicators of physiologic status. Age, medications, and comorbid factors all may affect a patient's response to hemorrhagic shock.

Failure of compensatory mechanisms in hemorrhagic shock can lead to death. Without intervention, a classic trimodal distribution of deaths is seen in severe hemorrhagic shock. An initial peak of mortality occurs within minutes of hemorrhage due to immediate exsanguination. Another peak occurs after 1 to several hours due to progressive decompensation. A third peak occurs days to weeks later due to sepsis and organ failure.

In the United States, accidental injury is the leading cause of morbidity and mortality in persons between the ages of 1 and 44 years. In 2001, 157,078 resident deaths occurred as the result of injuries. Of these, 64.6 percent were classified as unintentional, 19.5 percent were suicides, 12.9 percent were homicides, 2.7 percent were of undetermined intent, and 0.3 percent involved legal intervention or operations of war. The leading causes of injury death were motor vehicle traffic, firearm, and falls. A large proportion of these fatalities result from massive blood loss due to the trauma, leading to hemorrhagic shock.

In the majority of trauma injury cases, patients who come to a hospital's emergency department are treated by emergency physicians and discharged without requiring surgery or care by a trauma service. However, patients with serious injuries require stabilization within the “Golden Hour” after the injury occurred, to improve the chances of survival and to minimize disability.

As most shock cases are due to injury caused by an accident, pre-hospital care is critical to the survival of the patient. This involves rapid assessment, stabilization, and expeditious transport to an appropriate center for evaluation and definitive care. In all patients with shock syndrome, the maintenance of a patent airway, adequate breathing and adequate circulation are the primary focus of emergency treatment. Assessment is essential, as changes in client condition indicate progression of the shock syndrome. Early intervention is vital to minimize damage to tissues and organs and minimize permanent disability and early identification of the primary clinical cause is critical. Treatments are directed toward correcting the cause of the shock syndrome and slowing progression. Intravenous access and fluid resuscitation (typically IV saline) are standard, however, there is some debate over this. Rapid reversal of hypovolemia may increase hemorrhage, dislodge partially formed clots, and dilute clotting factors.

Once at the emergency department, the focus is on optimizing perfusion and oxygenation of vital organs. Diagnosis and management of the underlying hemorrhage must be performed rapidly and concurrently with management of shock. There are two major stages of shock: early compensation stage and progressive stage. It is contemplated that embodiments of the invention may be applied to patients in either or both stages.

When hypovolemic shock results from massive hemorrhage, the replacement fluid of choice is whole blood or packed red blood cells. Crystalloid solutions will temporarily improve circulating volume, but the patient also needs replacement of red blood cells to carry oxygen to the tissues. Management of shock focuses on fluid management, acid-base balance, and improving myocardial contraction. Treating the underlying cause of shock should also be treated in order to diminish the progression of the shock syndrome. Whole body hibernation was induced in mice, and there was an immediate drop in overall metabolic state (as measured by CO₂ evolution). This was reversible, and the mice seem to function normally, even after repeated exposures.

Accordingly, the invention concerns inducing a whole body hibernetic state using an Effective Compound to preserve the patient's vital organs and life. This will allow for transport to a controlled environment (e.g., surgery), where the initial cause of the shock can be addressed, and then the patient brought back to normal function in a controlled manner. For this indication, the first hour after injury, referred to as the “golden hour,” is crucial to a successful outcome. Stabilizing the patient in this time period is the major goal, and transport to a critical care facility (e.g., emergency room, surgery, etc.) where the injury can be properly addressed. Thus, it would be ideal to maintain the patient in stasis to allow for this and to address immediate concerns such as source of shock, replenish blood loss, and reestablish homeostasis. While this will vary significantly, in most cases, the amount of time stasis will be maintained is between about 6 and about 72 hours after injury. In some embodiments of the invention, biological matter is exposed to an Effective Compound for about, at least about, or at most about 30 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days or more, and any range or combination therein.

U.S. Provisional Patent Application Ser. No. 60/793,520 describes the treatment of shock and is hereby incorporated by reference in its entirety.

d. Hypothermia

In yet another embodiment, the present inventor proposes use of the present invention to treat people with extreme hypothermia, such uses involving modulation of HIF as described herein. The methods and compositions of the present invention are useful for inducing hypothermia in a mammal in need of hypothermia. Hypothermia can be mild, moderate or profound. Mild hypothermia comprises achievement of a core body temperature of approximately between 0.1 and 5 degrees Celsius below the normal core body temperature of the mammal. The normal core body temperature of a mammal is usually between 35 and 38 degrees Celsius. Moderate hypothermia comprises achievement of a core body temperature of approximately between 5 and 15 degrees Celsius below the normal core body temperature of the mammal. Profound hypothermia comprises achievement of a core body temperature of approximately between 15 and 37 degrees Celsius below the normal core body temperature of the mammal.

Mild hypothermia is known in the art to be therapeutically useful and effective in both non-human mammals and in humans. The therapeutic benefit of mild hypothermia has been observed in human clinical trials in the context of out-of-hospital cardiac arrest. Exposure of humans to mild hypothermia in the context of cardiac arrest results in a survival advantage and an improved neurological outcome compared to standard of care with normothermia, or absence of mild hypothermia (Bernard et al., 2002; The Hypothermia After Cardiac Arrest Study Group et al., 2002).

Methods and compositions of the present invention may have advantages over other methods known in the art, including, but not limited to, packing the subject in ice, or surrounding the subject with a “cooling tent” that circulates cool air or liquid, for inducing mild, moderate, or profound hypothermia in mammals or humans. In these cases, the subject resists the reduction of core body temperature below normothermia and tries to generate heat by shivering. Shivering, and the body heat engendered therein, can have a negative impact on the achievement of mild hypothermia by, for example, slowing the rate of decrease in the core body temperature that is achieved using the standard methods of hypothermia induction. Consequently, humans subjected to therapeutic levels of hypothermia are also treated with a drug that inhibits shivering (by blocking neurotransmission at the neuromuscular junctions) (Bernard et al., 2002).

In some embodiments, methods and compositions of the present invention are combined with invasive methods or medical devices known in the art to induce therapeutic hypothermia in mammals or humans. Such invasive methods and devices include, but are not limited to, flexible probes or catheters that can be inserted into the vasculature of the subject in need of hypothermia, wherein the temperature of the catheter is adjusted to below the normal body temperature of the subject, resulting in the cooling of blood which is in contact with the catheter. The cooled blood subsequently engenders a decrease in the core body temperature of the mammal. By incorporating feedback from a thermocouple monitoring the core body temperature of the mammal, the temperature of the catheter can be modulated so as to maintain a pre-specified core body temperature. Such medical devices for achieving and maintaining mild or moderate hypothermia, referred to in the art as endovascular temperature therapy, are known in the art and are described for example on the World Wide Web at innercool.com and radiantmedical.com.

The method provides that patients with extreme hypothermia are administered or exposed to a compound of the present invention and then gradually restored to normal temperature while withdrawing, in a controlled fashion, the compound. In this way, the compound buffers the biological systems within the subject so that they may be initiated gradually without shock (or harm) to the subject.

In one embodiment, a subject suffering from hypothermia with be given an oral or intravenous dose of a compound of the present invention. Intravenous provision may be preferred because of the potential non-responsiveness of the subject and the ability to provide a controlled dosage over a period of time. Alternatively, if available, the compound of the present invention may be provide in a gaseous state, for example, using a mask for inhalation or even a sealed chamber that can house the entire subject.

Ideally, the patient will be stabilized in terms of heart rate, respiration and temperature prior to effecting any change. Once stable, the ambient environmental temperature will be increased, again gradually. This may be accomplished simply by removing the subject from the hypothermic conditions. A more regulated increase in temperature may be effected by adding successive layers of clothing or blankets, by use of a thermal wrap with gradual increase in heat, or if possible, by placing the subject in chamber whose temperature may be gradually increased.

It is preferred that the vital signs of the subject are monitored over the course of the temperature increase. Also, in conjunction with increasing the temperature, the compound of the present invention is removed from the subject's environment. Both heat and treatment using a compound of the present invention are ceased at the appropriate endpoint, judged by the medical personnel monitoring the situation, but in any event at the time the subject's temperature and other vital signs return to a normal range. Continued monitoring following cessation of treatment is recommended for a period of at least 24 hrs.

e. Hyperthermia

Under certain conditions, which can result from genetic, infectious, drug, or environmental causes, patients can loose homeostatic temperature regulation resulting in severe uncontrollable fever (hyperthermia). This can result in mortality or long-term morbidity, especially brain damage, if it is not controlled properly.

The technology of the present invention could be used to control whole body temperature in certain states of hyperthermia. This would likely involve administration of a compound of the present invention through inhalation or perfused into the blood supply to induce a hibernation state via modulation of HIF. It would be useful to have the patient to be in stasis for between about 6 and about 24 hours, during which time the source of the fever can be addressed. In some embodiments of the invention, a patient is exposed to a compound of the present invention for about, at least about, or at most about 30 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days or more, and any range or combination therein.

This can be combined with some whole-body temperature regulation (ice bath/blanket/cooling system).

f. Cardioplegia and Coronary Heart Disease

In certain embodiments, the present invention may find use as solutions for the treatment of coronary heart disease (CHD) including a use for cardioplegia for cardiac bypass surgery (CABG). As described herein, CHD may be considered an ischemia-related condition, and thus it is contemplated that one or more Effective Compounds may be administered to treat CHD, wherein the Effective Compound modulates HIF (e.g., stabilization of HIFα) and/or one or more 2-oxoglutarate dioxygenase enzymes, such as a HIF prolyl hydroxylase (e.g., inhibition of HIF prolyl hydroxylase).

CHD results from atherosclerosis, a narrowing and hardening of the arteries that supply oxygen rich blood to the heart muscle. The arteries harden and become narrow due to the buildup of plaque on the inner walls or linings of the arteries. Blood flow to the heart is reduced as plaque narrows the coronary arteries. This decreases the oxygen supply to the heart muscle. This may manifest in 1) angina, which is chest pain or discomfort that happens when the heart is not getting enough blood; 2) heart attack, which can occur when a blood clot suddenly cuts off most or all blood supply to part of the heart and cells in the heart muscle that do not receive enough oxygen-carrying blood begin to die, potentially causing permanent damage to the heart muscle; 3) heart failure, which is when the heart is unable to pump blood effectively to the rest of the body; arrhythmias, which are changes in the normal rhythm of the heartbeats.

Since 1990, more people have died from CHD than any other cause. 3.8 million men and 3.4 million women die each year from CHD. In 2002, over 500,000 people in the United States alone died as a direct result of heart disease. Despite improvements in survival rates, 1 in 4 men, and 1 in 3 women in the U.S. still die within a year of a recognized first heart attack.

Medical treatment of CHD includes medications to reduce the risk of heart attack, heart failure and stroke, together with important lifestyle changes to prevent the further build-up of fatty deposits in the coronary arteries. Nonetheless, some type of surgical intervention is also frequently indicated.

About one-third of CHD patients will undergo coronary angioplasty and stenting. During balloon angioplasty, a balloon-tipped catheter is employed to push plaque back against the arterial wall to allow for improved blood flow in the artery. Coronary stenting often accompanies the angioplasty procedure. Stents are small wire-mesh metal tubes that provide scaffolding to support the damaged arterial wall, reducing the chance that the vessel will close again (restenosis) after angioplasty. In the United States, nearly one million balloon angioplasty procedures are performed each year. Not all patients are able to be treated by this technique; such patients must undergo heart surgery. Michaels et al., 2002.

About 10% of CHD patients will undergo coronary artery bypass graft (CABG) surgery. Patients with severe narrowing or blockage of the left main coronary artery or those with disease involving two or three coronary arteries are generally considered candidates for bypass surgery. In CABG, the surgeon uses a portion of a healthy vessel (either an artery or a vein) from another part of the body to create a detour (or bypass) around the blocked portion of the coronary artery. Patients typically receive from 1 to 5 bypasses in a given operation. During the procedure, generally the heart is placed in a state of paralysis, known as cardioplegia (CP), during which a heart-lung machine artificially maintains circulation. Patients are under general anesthesia during the operation, which usually lasts between 3 to 6 hours.

Approximately 13% of all patients will be re-admitted to the hospital within 30 days due to reasons related to the CABG. Hannan et al., 2003; Mehlhom et al., 2001. One of the main reasons for re-admission is heart failure, presumably due to ischemic damage during the surgery. Thus, much work is being done to improve the protection of the myocardium during the period when the heart is not being perfused normally.

Recent advances in cardiac surgery have centered upon optimization of cardioplegic parameters in the hope of preventing postoperative ventricular dysfunction and improving overall outcome. Cohen et al., 1999.

Cardioplegic solutions are perfused through the vessels and chambers of the heart and cause its intrinsic beating to cease, while maintaining the viability of the organ.

Cardioplegia (paralysis of the heart) is desirable during open-heart surgery and during the procurement, transportation, and storage of donor hearts for use in heart transplantation procedures.

Early cardioplegic techniques employed cold crystalloid solutions to initiate and maintain intraoperative cardiac arrest. However, it has become clear that blood cardioplegia facilitated aerobic myocardial metabolism during the cross-clamp period and reduced anaerobic lactate production. Furthermore, blood cardioplegia improves oxygen carrying capacity, enhanced myocardial oxygen consumption and preserved myocardial high-energy phosphate stores. Several different cardioplegic solutions are available and different techniques for using cardioplegia solutions are known in the art. For example, cardioplegic solutions often have varying amounts of potassium, magnesium, and several other minor components. Sometimes drugs are added to the cardioplegic solution to aid in muscle relaxation and protection from ischemia. Current approaches also include blood-only formulations with appropriate electrolyte supplementation, such as glutamate-aspartate. Specific examples of frequently used solutions are the St Thomas Hospital solution, University of Wisconsin Solution, Stanford Solution, and the Bretschneider Solution. Examples of other emerging solutions involve adenosine, insulin or L-arginine containing solutions mentioned earlier. Varying the temperature at which the cardioplegic solution is used may also have beneficial effects.

A combination of continuous retrograde along with intermittent antegrade cardioplegia reduces myocardial lactate production, preserved ATP stores, and improved metabolic recovery after cross-clamp release. Tepid (29° C.) cardioplegia reduces lactate and acid production during cardioplegic arrest, and improves post-operative ventricular function. Cardioplegic flows of at least 200 mL/min are required to washout detrimental metabolic end-products and improve ventricular function. It is abundantly clear now that future directions in cardioplegic management will involve the use of cardioplegic additives to further improve protective effects. For example, attempts have been made to harness the beneficial effects of ischemic pre-conditioning using adenosine. Similarly, insulin cardioplegia has been employed in order to enhance ventricular performance by stimulating early postoperative aerobic metabolism. Finally, L-arginine, a nitric oxide donor has been demonstrated to be beneficial in experimental studies and may represent a further option for the enhancement of intraoperative myocardial protection. Future benefit of cardioplegic supplementation is likely to be observed in high-risk with poor ventricular function, for which current protective techniques are inadequate. There is a steady increase in the incidence of high-risk patients presenting, and these cases, and consequent complications, place a disproportionate burden on the health care system. Thus, improvements in this area hold great promise for the advancement of care in this field.

Despite the protective effects provided by the current methods for inducing cardioplegia, there is still some degree of ischemic-reperfusion injury to the myocardium. Ischemic-reperfusion injury during cardiac bypass surgery results in poor outcomes (both morbidity and mortality), especially due to an already weakened state of the heart. Myocardial ischemia results in anaerobic myocardial metabolism. The end products of anaerobic metabolism rapidly lead to acidosis, mitochondrial dysfunction, and myocyte necrosis. High-energy phosphate depletion occurs almost immediately, with a 50 percent loss of ATP stores within 10 minutes. Reduced contractility occurs within 1 to 2 minutes, with development of ischemic contracture and irreversible injury after 30 to 40 minutes of normothermic (37° C.) ischemia.

Reperfusion injury is a well-known phenomenon following restoration of coronary circulation. Reperfusion injury is characterized by abnormal myocardial oxidative metabolism. In addition to structural changes created during ischemia, reperfusion may produce cytotoxic oxygen free radicals. These oxygen free radicals play a significant role in the pathogenesis of reperfusion injury by oxidizing sarcolemmal phospholipids and thus disrupting membrane integrity. Oxidized free fatty acids are released into the coronary venous blood and are a marker of myocardial membrane phospholipid peroxidation. Protamine induces complement activation, which activates neutrophils. Activated neutrophils and other leukocytes are an additional source of oxygen free radicals and other cytotoxic substances.

The present invention provides methods and compositions for inducing cardioplegia that will provide greater protection to the heart during bypass surgery. The induction of cardioplegia may involve, in certain embodiments, stabilization of HIFα via inhibiting one or more 2-oxoglutarate dioxygenase enzymes, such as a HIF prolyl hydroxylase. In certain embodiments, the present invention provides a cardioplegic solution comprising a compound of the present invention dissolved in solution or bubbled as a gas in the solution. In some embodiments, the invention further comprises at least a first device, such as a catheter or cannula, for introducing an appropriate dose of the cardioplegic solution to the heart. In certain aspects, the invention further comprises at least a second device, such as a catheter or cannula, for removing the cardioplegic solution from the heart.

Bypass surgery typically last for 3-6 hours, however, complications and multiple vessel CABG can extend the duration to 12 hours or longer. It is contemplated that the heart would be kept in stasis during the surgery. Thus, in some embodiments of the invention, the heart is exposed to a compound of the present invention for about, at least about, or at most about 30 seconds, 1, 2, 3, 4, 5, 10, 15, 20, 25, 30, 35, 40, 45, 50, 55 minutes, 1, 2, 3, 4, 5, 6, 7, 8, 9, 10, 11, 12, 13, 14, 15, 16, 17, 18 hours or more, and any range or combination therein.

g. Reducing Damage from Cancer Therapy

Cancer is a leading cause of mortality in industrialized countries around the world. The most conventional approach to the treatment of cancer is by administering a cytotoxic agent to the cancer patient (or treatment ex vivo of a tissue) such that the agent has a more lethal effect on the cancer cells than normal cells. The higher the dose or the more lethal the agent, the more effective it will be; however, by the same token, such agents are all that more toxic (and sometimes lethal) to normal cells. Hence, chemo- and radiotherapy are often characterized by severe side effects, some of which are life threatening, e.g., sores in the mouth, difficulty swallowing, dry mouth, nausea, diarrhea, vomiting, fatigue, bleeding, hair loss and infection, skin irritation and loss of energy (Curran, 1998; Brizel, 1998).

Recent studies suggest that transient and reversible lowering of the core body temperature, or “hypothermia,” may lead to improvements in the fight against cancer. Hypothermia of 28° C. was recently found to reduce radiation, doxorubicin- and cisplatin-induced toxicity in mice. The cancer fighting activity of these drugs/treatments was not compromised when administered to cooled animals; rather, it was enhanced, particularly for cisplatin (Lundgren-Eriksson et al., 2001). Based on this and other published work, the inventor proposes a further reduction in core temperature will provide benefit to cancer patients. Thus, the present invention contemplates the use of a compound of the present invention to induce stasis in normal tissues of a cancer patient, thereby reducing the potential impact of chemo- or radiotherapy on those tissues. It also permits the use of higher doses of chemo- and radiotherapy, thereby increasing the anti-cancer effects of these treatments.

Treatment of virtually any hyperproliferative disorder, including benign and malignant neoplasias, non-neoplastic hyperproliferative conditions, pre-neoplastic conditions, and precancerous lesions, is contemplated. Such disorders include restenosis, cancer, multi-drug resistant cancer, primary psoriasis and metastatic tumors, angiogenesis, rheumatoid arthritis, inflammatory bowel disease, psoriasis, eczema, and secondary cataracts, as well as oral hairy leukoplasia, bronchial dysplasia, carcinomas in situ, and intraepithelial hyperplasia. In particular, the present invention is directed at the treatment of human cancers including cancers of the prostate, lung, brain, skin, liver, breast, lymphoid system, stomach, testicles, ovaries, pancreas, bone, bone marrow, gastrointestine, head and neck, cervix, esophagus, eye, gall bladder, kidney, adrenal glands, heart, colon and blood. Cancers involving epithelial and endothelial cells are also contemplated for treatment.

Generally, chemo- and radiotherapy are designed to reduce tumor size, reduce tumor cell growth, induce apoptosis in tumor cells, reduce tumor vasculature, reduce or prevent metastasis, reduce tumor growth rate, accelerate tumor cell death, and kill tumor cells. The goals of the present invention are no different. Thus, it is contemplated that one will combine compositions comprising a compound of the present invention with secondary anti-cancer agents (secondary agents) effective in the treatment of hyperproliferative disease. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

Secondary anti-cancer agents include biological agents (biotherapy), chemotherapy agents, and radiotherapy agents. More generally, these other compositions are provided in a combined amount effective to kill or inhibit proliferation of the cancer or tumor cells, while at the same time reducing or minimizing the impact of the secondary agents on normal cells. This process may involve contacting or exposing the cells with a compound of the present invention and the secondary agent(s) at the same time. This may be achieved by contacting the cell with a single composition or pharmacological formulation that includes both agents, or by contacting or exposing the cell with two distinct compositions or formulations, at the same time, wherein one composition includes a compound of the present invention and the other includes the second agent(s).

Alternatively, the therapy comprising use of a compound of the present invention may precede or follow the secondary agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. In certain embodiments, it is envisioned that biological matter will be kept in stasis for between about 2 and about 4 hours while the cancer treatment is being administered. In some embodiments of the invention, biological matter is exposed to a compound of the present invention for about, at least about, or at most about 30 seconds, 1, 2, 3, 4, 5, 10, 15, 20, 25, 30, 35, 40, 45, 50, 55 minutes, 1, 2, 3, 4, 5, 6 hours or more, and any range or combination therein.

Various combinations may be employed; the Effective Compound is “A” and the secondary anti-cancer agent, such as radio- or chemotherapy, is “B”:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

Administration of a compound of the present invention to a patient will follow general protocols for the administration of chemotherapeutics, taking into account the toxicity, if any, of the compound. It is expected that the treatment cycles would be repeated as necessary. It also is contemplated that various standard therapies, as well as surgical intervention, may be applied in combination with the above-described anti-cancer therapy. It is further contemplated that any combination treatment contemplated for use with an Effective Compound and a non-Effective Compound (such as chemotherapy), may be applied with respect to multiple Effective Compounds.

(i) Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein transferase inhibitors, transplatinum, 5-fluorouracil, vincristine, vinblastine and methotrexate, Temazolomide (an aqueous form of DTIC), or any analog or derivative variant of the foregoing. The combination of chemotherapy with biological therapy is known as biochemotherapy.

(ii) Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a composition of the invention or a chemotherapeutic or radiotherapeutic agent is delivered to a target cell or are placed in direct juxtaposition with the target cell. In combination therapy, to achieve cell killing or stasis, both agents may be delivered to a cell in a combined amount effective to kill the cell or prevent it from dividing.

(iii) Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy could also be used as part of a combined therapy. The general approach for combined therapy is discussed below. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor such as mda-7 has been shown to enhance anti-tumor effects (Ju et al., 2000)

As discussed earlier, examples of immunotherapies currently under investigation or in use are immune adjuvants (e.g., Mycobacterium bovis, Plasmodium falciparum, dinitrochlorobenzene and aromatic compounds) (U.S. Pat. No. 5,801,005; U.S. Pat. No. 5,739,169; Hui and Hashimoto, 1998; Christodoulides et al., 1998), cytokine therapy (e.g., interferons α, β and γ; IL-1, GM-CSF and TNF) (Bukowski et al., 1998; Davidson et al., 1998; Hellstrand et al., 1998) gene therapy (e.g., TNF, IL-1, IL-2, p53) (Qin et al., 1998; Austin-Ward and Villaseca, 1998; U.S. Pat. No. 5,830,880 and U.S. Pat. No. 5,846,945) and monoclonal antibodies (e.g., anti-ganglioside GM2, anti-HER-2, anti-p185) (Pietras et al., 1998; Hanibuchi et al., 1998). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). Combination therapy of cancer with herceptin and chemotherapy has been shown to be more effective than the individual therapies. Thus, it is contemplated that one or more anti-cancer therapies may be employed with the anti-tumor therapies described herein.

h. Neurodegeneration

The present invention may be used to treat neurodegenerative diseases. Neurodegenerative diseases are characterized by degeneration of neuronal tissue, and are often accompanied by loss of memory, loss of motor function, and dementia. With dementing diseases, intellectual and higher integrative cognitive faculties become more and more impaired over time. It is estimated that approximately 15% of people 65 years or older are mildly to moderately demented. Neurodegenerative diseases include Parkinson's disease; primary neurodegenerative disease; Huntington's Chorea; stroke and other hypoxic or ischemic processes; neurotrauma; metabolically induced neurological damage; sequelae from cerebral seizures; hemorrhagic shock; secondary neurodegenerative disease (metabolic or toxic); Alzheimer's disease, other memory disorders; or vascular dementia, multi-infarct dementia, Lewy body dementia, or neurodegenerative dementia.

Evidence shows that the health of an organism, and especially the nervous system, is dependent upon cycling between oxidative and reductive states, which are intimately linked to circadian rhythms. That is, oxidative stress placed upon the body during consciousness is cycled to a reductive environment during sleep. This is thought to be a large part of why sleep is so important to health. Certain neurodegenerative disease states, such as Huntington's disease and Alzheimer's disease, as well as the normal processes of aging have been linked to a discord in this cycling pattern. There is also some evidence that brain H₂S levels are reduced in these conditions (Eto et al., 2002).

The present invention can be used to regulate and control the cycling between the oxidative and reduced states, for example, to prevent or reverse the effects of neurodegenerative diseases and processes. Controlling circadian rhythms can have other applications, for example, to adjust these cycling patterns after traveling from one time zone to another, so as to adjust to the new time zone. Furthermore, reduced metabolic activity overall has been shown to correlate with health in aged animals and humans. Therefore, the present invention would also be useful to suppress overall metabolic function to increase longevity and health in old age. It is contemplated that this type of treatment would likely be administered at night, during sleep for period of approximately 6 to 10 hours each day. This could require daily treatment for extended periods of time from months to years.

i. Aging

Furthermore, in certain states of stasis, including but not limited to states where the biological matter is in a state of suspended animation, aging itself may be thoroughly or completely inhibited for the period of time when the biological matter is in that state. Thus the present invention may inhibit aging of biological material, with respect to extending the amount of time the biological material would normally survive and/or with respect to progression from one developmental stage of life to another.

j. Blood Disease

A number of blood diseases and conditions may be addressed using compositions and methods of the invention. These diseases include, but are not limited to, thalassemia and sickle cell anemia.

(i) Thalassemia

Normal hemoglobin contains two alpha and two beta globin polypeptide (protein) chains, each bound to an iron containing heme ring. Thalassemia is a group of conditions in which there is an imbalance of alpha and beta chains leading to the unpaired chains precipitating on the normally fragile red blood cell membrane, leading to cell destruction. This leads to severe anemia that the marrow tries to compensate for by trying to make more red cells. Unfortunately due to toxicity from unpaired chains this process is very inefficient leading to massive expansion of the marrow space and spread of blood making to other parts of the body. This and the anemia lead to major toxicities. Several models exist as to why unpaired globin chains are so damaging but many entail that increased free radicals generated by the iron attached to the unpaired globin chains are central to the early destruction of the red cells. Thus any intervention that might decrease the oxidative damage from these free radicals could increase red cell lifespan, improve the anemia, lead to decreased need for making red cells, and less damage from marrow expansion and spread.

It is estimated that over 30,000 children are born with severe thalassemia each year, of which it is estimated that most living in developed countries live into their twenties, while in third world countries (where the majority of patients live) most die as young children. Based on the current results in other model systems presented here, it expected that exposing animals with thalassemia to sulfides will increase their red cells' ability to withstand oxidative damage, leading to prolonged red cell survival.

(ii) Sickle Cell Disease

Normal hemoglobin (HbA) contains two alpha and two beta globin polypeptide (protein) chains, each bound to an iron containing heme ring. In sickle cell disease (SCD; also called sickle cell anemia) is a group of conditions in which a mutant beta chain leads to an altered hemoglobin (HbS). Upon deoxygention HbS can polymerize (crystallize) and precipitate damaging the normally fragile red blood cell membrane, leading to cell destruction and anemia low red blood cells (RBC). In addition cells with polymerized HbS change shape (sickle) and become sticky and activate mechanisms leading to coagulation and blockage of blood flow. This can lead to hypoxic damage of the surrounding tissue resulting in pain, organ dysfunction and eventually premature death. Decreased stores of sulfur containing antioxidants are noted in patients. In addition oxidative damage and increased reactive oxygen species (ROS) have been implicated in crystallization, RBC membrane damage and tissue damage related to inadequate blood flow. Sulfides have been implicated in “re-charging” antioxidant stores, and potentially minimizing oxidative damage. There are reasons to think sulfides could prevent problems at several stages of sickle cell pathology. Furthermore, given the ability of oxygen antagonists to protect from hypoxia in other systems, suggests that it should also protect animals and humans subjected to the adverse conditions posed by this disease state.

Over 120,000 children are born with SCD each year. Patients in developed countries now live into their 40's and 50's however with tremendous problems with pain and organ damage including stroke, lung, heart and skin problems. In third world countries (where the majority of patients live) most die as young children. The hypothesis is that exposing animals and eventually humans with SCD to sulfides of the present invention will result in health improvements.

2. Testing for Stasis

Various compounds of the present invention that are useful for inducing stasis may be initially evaluated using a variety of different tests. See, e.g., U.S. patent application Ser. No. 11/408,734, herein incorporated by reference in its entirety.

C. Other HIF-Modulated Conditions

Other conditions or events associated with HIF activity that may be modulated by the Effective Compounds via stabilization of HIFα and/or inhibition of one or more 2-oxoglutarate dioxygenase enzymes, such as HIF prolyl hydroxylase, include, but are not limited to: hypertension; diabetes; chronic venous insufficiency; Raynaud's disease; cirrhosis (including, e.g., cardiac cirrhosis); systemic sclerosis; chronic skin ulcers; formation of thrombus; vascular closure; viral infection; ischemic stroke; prenatal hypoxia; circulatory shock; altitude or mountain sickness; acute respiratory failure; nonbacterial thrombus endocarditis; chronic heart failure; macular degeneration; angina pectoris; TIAs (transient ischemic attack); chronic alcoholic liver disease; COPD (chronic obstructive pulmonary disease); severe pneumonia; pulmonary edema; pulmonary hypertension; ulcers (including, e.g., gastric and duodenal ulcers); liver disease; renal disease; blood coagulation disorders; chronic illness; concussions; the upregulation of genes involved in oxidative stress and vascular tone; liver ischemia; renal ischemia; peripheral vascular disorders; neonatal respirator disease syndrome; and increases in vascularization. Any of these conditions, as well as anemia described below, may be associated with hypoxia, ischemia and/or stasis.

A further condition comprises anemia caused by or associated with acute kidney disease, chronic kidney disease, infections, inflammation (wherein the inflammation may be due to, for example, infection, autoimmune disorders, such as rheumatoid arthritis), cancer, irradiation, toxins, diabetes, surgery, virus (such as, e.g., HIV), bacteria and parasites, blood loss due to, e.g., stomach ulcer, duodenal ulcer, hemorrhoids, stomach or intestinal cancer, trauma, injury, surgical procedures, radiation therapy, chemotherapy, kidney dialysis, HIV-infected patients undergoing treatment with azidothymidine (zidovudine) or other reverse transcriptase inhibitors, and can develop in patients undergoing chemotherapy, e.g., with cyclic cisplatin- or non-cisplatin-containing chemotherapeutics, aplastic anemia, defective or abnormal hemoglobin or erythrocytes, such as in disorders including microcytic anemia and hypochromic anemia, disorders in iron transport, processing and utilization, such as sideroblastic anemia, as well as any other anemic conditions and anemic disorders as defined herein.

In another embodiment, an Effective Compounds may also be used in methods for treating a patient at risk of developing an ischemic or hypoxic condition, e.g., individuals at high risk for atherosclerosis, etc. Risk factors for atherosclerosis include, e.g., hyperlipidemia, cigarette smoking, hypertension, diabetes mellitus, hyperinsulinemia, and abdominal obesity. Therefore, the present invention provides methods of preventing ischemic tissue injury, the method comprising administering a therapeutically effective amount of a compound or a pharmaceutically acceptable salt thereof, alone or in combination with a pharmaceutically acceptable excipient, to a patient in need. In one embodiment, the compound can be administered based on predisposing conditions, e.g., hypertension, diabetes, occlusive arterial disease, chronic venous insufficiency, Raynaud's disease, chronic skin ulcers, cirrhosis, congestive heart failure, and systemic sclerosis.

### II. Effective Compounds

In general, compounds of the present invention modulate HIF and can thereby affect hypoxia, ischemia, and/or stasis as well as any other condition associated with HIFα stabilization as described herein, such as EPO-related conditions and hemorrhagic shock. Collectively, these compounds are called the Effective Compounds. Subsets of the Effective Compounds for various applications are specifically contemplated. In certain embodiments, the Effective Compounds or a subset thereof stabilize HIFα via interaction with a 2-oxoglutarate dioxygenase, such as HIF prolyl hydroxylase (e.g., Egl-9). In certain embodiments, the Effective Compounds or a subset thereof modulate EPO. Based on the inventor's observation that HIF regulation is a common feature among hypoxia, ischemia and stasis, any Effective Compound previously discovered to stabilize HIFα can be co-administered with any compound previously discovered to induce stasis. In other embodiments, one or more subsets of the Effective Compounds induce stasis. In yet other embodiments, one or more subsets of the Effective Compounds modulates hemorrhagic shock.

In certain embodiments, the Effective Compounds may be represented by any of Formulas I, Ia-Id, II, III, IIIa, IV, V, VI, VII, VIII, IX, X, XI, carbon monoxide, chalcogenide compounds, H₂S and other sulfur containing compounds, protective metabolic agents or oxygen antagonists as described herein. As mentioned, any subset of any of the Effective Compounds described herein is envisioned. Further, an Effective Compound may be described by one or more categories, e.g., a chalcogenide compound. A particular Effective Compound may fall into more than one descriptive category, e.g., an Effective Compound may be both a chalcogenide and a compound of Formula XI. In certain embodiments of the present invention, an Effective Compound has a chemical structure as set forth as Formula I, Ia-Id, II, III, IIIa, IV, V, VI, VII, VIII, IX, X or XI described herein, or is a precursor of Formula I, Ia-Id, II, III, IIIa, IV, V, VI, VII, VIII, IX, X or XI. For example, one embodiment contemplates the subset comprising those structures represented by Formulas I and IV. Another embodiment contemplates the subset comprising those structures represented by Formulas Ia-Id, II, III, IIIa, V, VI, VII, VIII, IX, X and XI. In another embodiment, the subset may comprise chalcogenides as described herein. In another embodiment, the subset may comprise oxygen antagonists as described herein.

In certain embodiments, the following definitions apply to terms used to describe certain Effective Compounds discussed herein. In some embodiments, these definitions apply to Formulas I and IV:

“Alkyl,” where used, either alone or within other terms such as “arylalkyl”, “aminoalkyl”, “thioalkyl” “cyanoalkyl” and “hydroxyalkyl”, may refer to linear or branched radicals having one to about twenty carbon atoms. The term “lower alkyl” may refer to C₁-C₆ alkyl radicals. As used herein the term alkyl typically includes those radicals that are substituted with groups such as hydroxy, halo (such as F, Cl, Br, I), haloalkyl, alkoxy, haloalkoxy, alkylthio, cyano, isocyano, carboxy (—COOH), alkoxycarbonyl, (—COOR), acyl, acyloxy, amino, alkylamino, urea (—NHCONHR), thiol, alkylthio, sulfoxy, sulfonyl, arylsulfonyl, alkylsulfonyl, sulfonamido, arylsulfonamido, heteroaryl, heterocyclyl, heterocycloalkyl, amidyl, alkylimino carbonyl, amidino, guanidino, hydrazino, hydrazide, sodium sulfonyl (—SO₃Na), sodium sulfonylalkyl (—RSO₃Na) or any combination thereof. Examples of such radicals may include, but are not limited to, methyl, ethyl, n-propyl, isopropyl, n-butyl, isobutyl, sec-butyl, tert-butyl, pentyl, iso-amyl, hexyl and the like.

“Hydroxyalkyl” may refer to an alkyl radical, as defined herein, substituted with one or more hydroxyl radicals. Examples of hydroxyalkyl radicals may include, but are not limited to, hydroxymethyl, 2-hydroxyethyl, 2-hydroxypropyl, 3-hydroxypropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl, and 2-(hydroxymethyl)-3-hydroxypropyl, and the like.

“Arylalkyl” may refer to the radical R′R— wherein an alkyl radical, “R” is substituted with an aryl radical “R′.” Examples of arylalkyl radicals may include, but are not limited to, benzyl, phenylethyl, 3-phenylpropyl, and the like.

“Aminoalkyl” may refer to the radical H₂NR′—, wherein an alkyl radical is substituted with an amino radical. Examples of such radicals include may aminomethyl, aminoethyl, and the like. “Alkylaminoalkyl” may refer to an alkyl radical substituted with an alkylamino radical.

“Alkylsulfonamido” may refer to a sulfonamido group (—S(O)₂—NRR′) appended to an alkyl group, as defined herein.

“Thioalkyl” may refer to an alkyl radical substituted with one or more thiol radicals. “Alkylthioalkyl” may refer to wherein an alkyl radical is substituted with one or more alkylthio radicals. Examples may include, but are not limited to, methylthiomethyl, ethylthioisopropyl, and the like. “Arylthioalkyl” may refer to wherein an alkyl radical, as herein defined, is substituted with one or more arylthio radicals.

“Carboxyalkyl” may refer to the radicals —RCO₂H, wherein an alkyl radical is substituted with a carboxyl radical. Examples may include, but are not limited to, carboxymethyl, carboxyethyl, carboxypropyl, and the like.

“Alkylene” may refer to bridging alkyl radicals.

The term “alkenyl” may refer to an unsaturated, acyclic hydrocarbon radical in so much as it contains at least one double bond. Such alkenyl radicals typically contain from about 2 to about 20 carbon atoms. The term “lower alkenyl” may refer to C₁-C₆ alkenyl radicals. As used herein, the term alkenyl radicals typically includes those radicals substituted as for alkyl radicals. Examples of suitable alkenyl radicals may include propenyl, 2-chloropropenyl, buten-1-yl, isobutenyl, pent-1-en-1-yl, 2-2-methyl-1-buten-1-yl, 3-methyl-1-buten-1-yl, hex-2-en-1-yl, 3-hydroxyhex-1-en-1-yl, hept-1-en-1-yl, and oct-1-en-1-yl, and the like.

The term “alkynyl” may refer to an unsaturated, acyclic hydrocarbon radical in so much as it contains one or more triple bonds, such radicals typically containing about 2 to about 20 carbon atoms. The term “lower alkynyl” may refer to C₁-C₆ alkynyl radicals. As used herein, the term alkynyl radicals typically includes those radicals substituted as for alkyl radicals. Examples of suitable alkynyl radicals may include ethynyl, propynyl, hydroxypropynyl, but-1-yn-1-yl, but-1-yn-2-yl, pent-1-yn-1-yl, pent-1-yn-2-yl, 4-methoxypent-1-yn-2-yl, 3-methylbut-1-yn-1-yl, hex-1-yn-1-yl, hex-1-yn-2-yl, hex-1-yn-3-yl, 3,3-dimethyl-1-butyn-1-yl radicals and the like.

“Alkoxy” may refer to the radical R′O—, wherein R′ is an alkyl radical as defined herein. Examples may include, but are not limited to, methoxy, ethoxy, propoxy, butoxy, isopropoxy, tert-butoxy alkyls, and the like. “Alkoxyalkyl” may refer to alkyl radicals substituted by one or more alkoxy radicals. Examples may include, but are not limited to, methoxymethyl, ethoxyethyl, methoxyethyl, isopropoxyethyl, and the like.

“Alkoxycarbonyl” may refer to the radical R—O—C(O)—, wherein R is an alkyl radical as defined herein. Examples of alkoxycarbonyl radicals may include, but are not limited to, methoxycarbonyl, ethoxycarbonyl, sec-butoxycarbonyl, isopropoxycarbonyl, and the like. Alkoxythiocarbonyl may refer to R—O—C(S)—.

“Aryl” may refer to the monovalent aromatic carbocyclic radical consisting of one individual ring, or one or more fused rings in which at least one ring is aromatic in nature, which can optionally be substituted with one or more, such as one or two, substituents such as hydroxy, halo (such as F, Cl, Br, I), haloalkyl, alkoxy, haloalkoxy, alkylthio, cyano, carboxy (—COOH), alkoxycarbonyl, (—COOR), acyl, acyloxy, amino, alkylamino, urea (—NHCONHR), thiol, alkylthio, sulfoxy, sulfonyl, arylsulfonyl, alkylsulfonyl, sulfonamido, arylsulfonamido, heteroaryl, heterocyclyl, heterocycloalkyl, amidyl, alkylimino carbonyl, amidino, guanidino, hydrazino, hydrazide, sodium sulfonyl (—SO₃Na), sodium sulfonylalkyl (—RSO₃Na), unless otherwise indicated. Alternatively, two adjacent atoms of the aryl ring may be substituted with a methylenedioxy or ethylenedioxy group. Examples of aryl radicals may include, but are not limited to, phenyl, naphthyl, biphenyl, indanyl, anthraquinolyl, tert-butyl-phenyl, 1,3-benzodioxolyl, and the like.

“Arylsulfonamido” may refer to a sulfonamido group, as defined herein, appended to an aryl group, as defined herein.

“Thioaryl” may refer to an aryl group substituted with one or more thiol radicals.

“Alkylamino” may refer to amino groups that are substituted with one or two alkyl radicals. Examples may include monosubstituted N-alkylamino radicals and N,N-dialkylamino radicals. Other examples may include N-methylamino, N-ethylamino, N,N-dimeythylamino N,N-diethylamino, N-methyl, N-ethyl-amino, and the like.

“Aminocarbonyl” may refer to the radical H₂NCO—. “Aminocarbonyalkyl” may refer to the substitution of an alkyl radical, as herein defined, by one or more aminocarbonyl radicals.

“Amidyl” may refer to RCO—NH—, wherein R is a H or alkyl, aryl, or heteroaryl, as defined herein.

“Imino carbonyl” may refer to a carbon radical having two of the four covalent bond sites shared with an imino group. Examples of such imino carbonyl radicals may include, for example, C═NH, C═NCH₃, C═NOH, and C═NOCH₃. The term “alkyliminocarbonyl” may refer to an imino radical substituted with an alkyl group. The term “amidino” may refer to a substituted or unsubstituted amino group bonded to one of two available bonds of an iminocarbonyl radical. Examples of such amidino radicals include, for example, NH₂—C═NH, NH₂—C═NCH₃, NH—C═NOCH₃ and NH(CH₃)—C═NOH. The term “guanidino” may refer to an amidino group bonded to an amino group as defined above where said amino group can be bonded to a third group. Examples of such guanidino radicals include, for example, NH₂—C(NH)—NH—, NH₂—C(NCH₃)—NH—, NH₂—C(NOCH₃)—NH—, and CH₃NH—C(NOH)—NH—. The term “hydrazino” may refer to —NH—NRR′, where R and R′ are independently hydrogen, alkyl and the like. “Hydrazide” may refer to —C(═O)—NH—NRR′.

The term “heterocyclyl” may refer to saturated and partially saturated heteroatom-containing ring-shaped radicals having from 4 through 15 ring members, herein referred to as “C₄-C₁₅ heterocyclyl” selected from carbon, nitrogen, sulfur and oxygen, wherein at least one ring atom is a heteroatom. Heterocyclyl radicals may contain one, two or three rings wherein such rings may be attached in a pendant manner or may be fused. Examples of saturated heterocyclic radicals may include saturated 3 to 6-membered heteromonocyclic group containing 1 to 4 nitrogen atoms [e.g. pyrrolidinyl, imidazolidinyl, piperidino, piperazinyl, etc]; saturated 3 to 6-membered heteromonocyclic groups containing 1 to 2 oxygen atoms and 1 to 3 nitrogen atoms [e.g. morpholinyl, etc.]; saturated 3 to 6-membered heteromonocyclic groups containing 1 to 2 sulfur atoms and 1 to 3 nitrogen atoms [e.g., thiazolidinyl, etc.]. Examples of partially saturated heterocyclyl radicals may include dihydrothiophene, dihydropyran, dihydrofuran and dihydrothiazole. Non-limiting examples of heterocyclic radicals may include 2-pyrrolinyl, 3-pyrrolinyl, pyrrolindinyl, 1,3-dioxolanyl, 2H-pyranyl, 4H-pyranyl, piperidinyl, 1,4-dioxanyl, morpholinyl, 1,4-dithianyl, thiomorpholinyl, and the like. Such heterocyclyl groups may be optionally substituted with groups such as substituents such as hydroxy, halo (such as F, Cl, Br, I), haloalkyl, alkoxy, haloalkoxy, alkylthio, cyano, carboxy (—COOH), alkoxycarbonyl, (—COOR), acyl, acyloxy, amino, alkylamino, urea (—NHCONHR), thiol, alkylthio, sulfoxy, sulfonyl, arylsulfonyl, alkylsulfonyl, sulfonamido, arylsulfonamido, heteroaryl, heterocyclyl, heterocycloalkyl, amidyl, alkylimino carbonyl, amidino, guanidino, hydrazino, hydrazide, sodium sulfonyl (—SO₃Na), sodium sulfonylalkyl (—RSO₃Na), and the like.

“Heteroaryl” may refer to monovalent aromatic cyclic radicals having one or more rings, such as one to three rings, of four to eight atoms per ring, incorporating one or more heteroatoms, such as one or two, within the ring (chosen from nitrogen, oxygen, or sulfur), which can optionally be substituted with one or more, such as one or two substituents selected from substituents such as hydroxy, halo (such as F, Cl, Br, I), haloalkyl, alkoxy, haloalkoxy, alkylthio, cyano, carboxy (—COOH), alkoxycarbonyl, (—COOR), acyl, acyloxy, amino, alkylamino, urea (—NHCONHR), thiol, alkylthio, sulfoxy, sulfonyl, arylsulfonyl, alkylsulfonyl, sulfonamido, arylsulfonamido, heteroaryl, heterocyclyl, heterocycloalkyl, amidyl, alkylimino carbonyl, amidino, guanidino, hydrazino, hydrazide, sodium sulfonyl (—SO₃Na), sodium sulfonylalkyl (—RSO₃Na), unless otherwise indicated. Examples of heteroaryl radicals may include, but are not limited to, imidazolyl, oxazolyl, thiazolyl, pyrazinyl, thienyl, furanyl, pyridinyl, quinolinyl, isoquinolinyl, benzofuryl, benzothiophenyl, benzothiopyranyl, benzimidazolyl, benzoxazolyl, benzothiazolyl, benzopyranyl, indazolyl, indolyl, isoindolyl, quinolinyl, isoquinolinyl, naphthyridinyl, benzenesulfonyl-thiophenyl, and the like.

“Heteroaryloxy” may refer to heteroaryl radicals attached to an oxy radical. Examples of such radicals may include, but are not limited to, 2-thiophenyloxy, 2-pyrimidyloxy, 2-pyridyloxy, 3-pyridyloxy, 4-pyridyloxy, and the like.

“Heteroaryloxyalkyl” may refer to alkyl radicals substituted with one or more heteroaryloxy radicals. Examples of such radicals may include 2-pyridyloxymethyl, 3-pyridyloxyethyl, 4-pyridyloxymethyl, and the like.

“Cycloalkyl” may refer to monovalent saturated carbocyclic radicals consisting of one or more rings, typically one or two rings, of three to eight carbons per ring, which can typically be substituted with one or more substitutents, such as hydroxy, halo (such as F, Cl, Br, I), haloalkyl, alkoxy, haloalkoxy, alkylthio, cyano, carboxy (—COOH), alkoxycarbonyl, (—COOR), acyl, acyloxy, amino, alkylamino, urea (—NHCONHR), thiol, alkylthio, sulfoxy, sulfonyl, arylsulfonyl, alkylsulfonyl, sulfonamido, arylsulfonamido, heteroaryl, heterocyclyl, heterocycloalkyl, amidyl, alkylimino carbonyl, amidino, guanidino, hydrazino, hydrazide, sodium sulfonyl (—SO₃Na), sodium sulfonylalkyl (—RSO₃Na), and the like. Examples of cycloalkyl radicals may include, but are not limited to, cyclopropyl, cyclobutyl, 3-ethylcyclobutyl, cyclopentyl, cycloheptyl, and the like.

“Cycloalkenyl” may refer to radicals having three to ten carbon atoms and one or more carbon-carbon double bonds. Typical cycloalkenyl radicals have three to seven carbon atoms. Examples may include cyclobutenyl, cyclopentenyl, cyclohexenyl, cycloheptenyl, and the like “Cycloalkenylalkyl” may refer to radicals wherein an alkyl radical, as defined herein, is substituted by one or more cycloalkenyl radicals.

“Cycloalkoxy” may refer to cycloalkyl radicals attached to an oxy radical. Examples may include, but are not limited to, cyclohexoxy, cyclopentoxy, and the like.

“Cycloalkoxyalkyl” may refer to alkyl radicals substituted one or more cycloalkoxy radicals. Examples may include cyclohexoxyethyl, cyclopentoxymethyl, and the like.

Sulfinyl” may refer to —S(O)—.

“Sulfonyl” may refer to —S(O)₂—, wherein “alkylsulfonyl” may refer to a sulfonyl radical substituted with an alkyl radical, RSO₂—, and “arylsulfonyl” may refer to aryl radicals attached to a sulfonyl radical. “Sulfonamido” may refer to —S(O)₂—NRR′.

“Sulfonic acid” may refer to —S(O)₂OH. “Sulfonic ester” may refer to —S(O)₂OR, wherein R is a group such as an alkyl as in sulfonic alkyl ester.

“Thio” may refer to —S—. “Alkylthio” may refer to RS— wherein a thiol radical is substituted with an alkyl radical R. Examples may include methylthio, ethylthio, butylthio, and the like. “Arylthio” may refer to R′S—, wherein a thio radical is substituted with an aryl radical, as herein defined. Examples may include, but are not limited to, phenylthio, phenylthiomethyl, and the like. “Alkylthiosulfonic acid” may refer to the radical HO₃SR′S—, wherein an alkylthioradical is substituted with a sulfonic acid radical.

“Thiosulfenyl” may refer to —S—SH.

“Acyl” may refer to a carbonyl or thiocarbonyl group bonded to a radical selected from, for example, hydrido, alkyl, alkenyl, alkynyl, haloalkyl, alkoxy, alkoxyalkyl, haloalkoxy, aryl, heterocyclyl, heteroaryl, alkylsulfinylalkyl, alkylsulfonylalkyl, aralkyl, cycloalkyl, cycloalkylalkyl, cycloalkenyl, alkylthio, arylthio, amino, alkylamino, dialkylamino, aralkoxy, arylthio, and alkylthioalkyl. Examples of “acyl” may include formyl, acetyl, benzoyl, trifluoroacetyl, phthaloyl, malonyl, nicotinyl, and the like.

The term “acylthiol” and “acyldisulfide” may refer to the radicals RCOS— and RCOSS— respectively.

The term “thiocarbonyl” may refer to the compounds and moieties which contain a carbon connected with a double bond to a sulfur atom —C(═S)—. “Alkylthiocarbonyl” may refer to wherein a thiocarbonyl group is substituted with an alkyl radical, R, as defined herein, to form the monovalent radical RC(═S)—. “Aminothiocarbonyl” may refer to a thiocarbonyl group substituted with an amino group, NH₂C(═S)—.

“Carbonyloxy” may refer to —OCOR.

“Alkoxycarbonyl” may refer to —COOR.

“Carboxyl” may refer to —COOH.

In certain embodiments, the following definitions apply to terms used to describe certain Effective Compounds discussed herein. In some embodiments, these definitions apply to Formulas Ia-Id, II, III, IIIa, V, VI, VII, VIII, IX and X:

“Alkyl” may refer to monovalent alkyl groups having from 1 to 10 carbon atoms, preferably from 1 to 5 carbon atoms and more preferably 1 to 3 carbon atoms. This term may be exemplified by groups such as methyl, ethyl, n-propyl, iso-propyl, n-butyl, t-butyl, n-pentyl and the like.

“Substituted alkyl” may refer to an alkyl group, of from 1 to 10 carbon atoms, preferably, 1 to 5 carbon atoms, having from 1 to 5 substituents, preferably 1 to 3 substituents, independently selected from the group consisting of alkoxy, substituted alkoxy, acyl, acylamino, acyloxy, amino, substituted amino, aminoacyl, aminocarbonylamino, aminothiocarbonylamino, aminocarbonyloxy, aryl, substituted aryl, aryloxy, substituted aryloxy, aryloxyaryl, substituted aryloxyaryl, cyano, halogen, hydroxyl, nitro, oxo, thioxo, carboxyl, carboxyl esters, cycloalkyl, substituted cycloalkyl, thiol, alkylthio, substituted alkylthio, arylthio, substituted arylthio, cycloalkylthio, substituted cycloalkylthio, heteroarylthio, substituted heteroarylthio, heterocyclicthio, substituted heterocyclicthio, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)₂-alkyl, —OS(O)₂-substituted alkyl, —OS(O)₂-aryl, —OS(O)₂-substituted aryl, OS(O)₂-heteroaryl, —OS(O)₂-substituted heteroaryl, —OS(O)₂-heterocyclic, —OS(O)₂-substituted heterocyclic, —OSO₂—NR⁴⁰R⁴⁰ where each R⁴⁰ is hydrogen or alkyl, —NR⁴¹S(O)₂-alkyl, —NR⁴⁰S(O)₂-substituted alkyl, —NR⁴⁰S(O)₂-aryl, —NR⁴⁰S(O)₂-substituted aryl, —NR⁴⁰S(O)₂-heteroaryl, —NR⁴⁰S(O)₂-substituted heteroaryl, —NR⁴⁰S(O)₂-heterocyclic, —NR⁴⁰S(O)₂-substituted heterocyclic, —NR⁴⁰S(O)₂—NR⁴⁰-alkyl, —NR⁴⁰S(O)₂—NR⁴⁰-substituted alkyl, —NR⁴⁰S(O)₂—NR⁴¹-aryl, —NR⁴⁰S(O)₂—NR⁴⁰-substituted aryl, —NR⁴⁰S(O)₂—NR⁴⁰-heteroaryl, —NR⁴⁰S(O)₂—NR⁴⁰-substituted heteroaryl, —NR⁴⁰S(O)₂—NR⁴⁰-heterocyclic, and —NR⁴⁰S(O)₂—NR⁴⁰-substituted heterocyclic where each R⁴⁰ is hydrogen or alkyl.

“Alkoxy” may refer to the group “alkyl-O—” which may include, by way of example, methoxy, ethoxy, n-propoxy, iso-propoxy, n-butoxy, t-butoxy, sec-butoxy, n-pentoxy and the like.

“Substituted alkoxy” may refer to the group “substituted alkyl-O—”.

“Acyl” may refer to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, aryl-C(O)—, substituted aryl-C(O)—, heteroaryl-C(O)—, substituted heteroaryl-C(O), heterocyclic-C(O)—, and substituted heterocyclic-C(O)— provided that a nitrogen atom of the heterocyclic or substituted heterocyclic is not bound to the —C(O)— group wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

The term “aminoacyl” or as a prefix “carbamoyl” or “carboxamide” or “substituted carbamoyl” or “substituted carboxamide” may refer to the group —C(O)NR⁴²R⁴² where each R⁴² is independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where each R⁴² is joined to form together with the nitrogen atom a heterocyclic or substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Acyloxy” may refer to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Alkenyl” may refer to alkenyl groups preferably having from 2 to 6 carbon atoms and more preferably 2 to 4 carbon atoms and having at least 1 and preferably from 1 to 2 sites of alkenyl unsaturation.

“Substituted alkenyl” may refer to alkenyl groups having from 1 to 3 substituents, and preferably 1 to 2 substituents, selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxyl, carboxyl esters, cycloalkyl, substituted cycloalkyl, heteroaryl, substituted heteroaryl, heterocyclic, and substituted heterocyclic.

“Alkynyl” may refer to alkynyl group preferably having from 2 to 6 carbon atoms and more preferably 2 to 3 carbon atoms and having at least 1 and preferably from 1-2 sites of alkynyl unsaturation.

“Substituted alkynyl” may refer to alkynyl groups having from 1 to 3 substituents, and preferably 1 to 2 substituents, selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxyl, carboxyl esters, cycloalkyl, substituted cycloalkyl, heteroaryl, substituted heteroaryl, heterocyclic, and substituted heterocyclic.

“Amino” may refer to the group —NH₂.

“Substituted amino” may refer to the group —NR⁴¹R⁴¹, where each R⁴¹ group is independently selected from the group consisting of hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclic, —SO₂-substituted heterocyclic, provided that both R⁴¹ groups are not hydrogen; or the R⁴¹ groups can be joined together with the nitrogen atom to form a heterocyclic or substituted heterocyclic ring.

“Acylamino” may refer to the groups —NR⁴⁵C(O)alkyl, —NR⁴⁵C(O)substituted alkyl, —NR⁴⁵C(O)cycloalkyl, —NR⁴⁵C(O)substituted cycloalkyl, —NR⁴⁵C(O)alkenyl, —NR⁴⁵C(O)substituted alkenyl, —NR⁴⁵C(O)alkynyl, —NR⁴⁵C(O)substituted alkynyl, NR⁴⁵C(O)aryl, —NR⁴⁵C(O)substituted aryl, —NR⁴⁵C(O)heteroaryl, —NR⁴⁵C(O)substituted heteroaryl, —NR⁴⁵C(O)heterocyclic, and —NR⁴⁵C(O)substituted heterocyclic where R⁴⁵ is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are defined herein.

“Carbonyloxyamino” may refer to the groups —NR⁴⁶C(O)O-alkyl, —NR⁴⁶C(O)O-substituted alkyl, —NR⁴⁶C(O)O-alkenyl, —NR⁴⁶C(O)O-substituted alkenyl, —NR⁴⁶C(O)O-alkynyl, —NR⁴⁶C(O)O-substituted alkynyl, —NR⁴⁶C(O)O-cycloalkyl, NR⁴⁶C(O)O-substituted cycloalkyl, —NR⁴⁶C(O)O-aryl, —NR⁴⁶C(O)O-substituted aryl, —NR⁴⁶C(O)O-heteroaryl, —NR⁴⁶C(O)O-substituted heteroaryl, —NR⁴⁶C(O)O-heterocyclic, and —NR⁴⁶C(O)O-substituted heterocyclic where R⁴⁶ is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminocarbonyloxy” or as a prefix “carbamoyloxy” or “substituted carbamoyloxy” may refer to the groups —OC(O)NR⁴⁷R⁴⁷ where each R⁴⁷ is independently hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic or where each R⁴⁷ is joined to form, together with the nitrogen atom a heterocyclic or substituted heterocyclic and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminocarbonylamino” may refer to the group —NR⁴⁹C(O)NR⁴⁹— where R⁴⁹ is selected from the group consisting of hydrogen and alkyl.

“Aryl” or “Ar” may refer to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is the aryl group. Preferred aryls may include phenyl and naphthyl.

“Substituted aryl” may refer to aryl groups, as defined herein, which are substituted with from 1 to 4, preferably 1-3, substituents selected from the group consisting of hydroxy, acyl, acylamino, carbonylaminothio, acyloxy, alkyl, substituted alkyl, alkoxy, substituted alkoxy, alkenyl, substituted alkenyl, alkynyl, substituted alkynyl, amidino, amino, substituted amino, aminoacyl, aminocarbonyloxy, aminocarbonylamino, aminothiocarbonylamino, aryl, substituted aryl, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxyl esters cyano, thiol, alkylthio, substituted alkylthio, arylthio, substituted arylthio, heteroarylthio, substituted heteroarylthio, cycloalkylthio, substituted cycloalkylthio, heterocyclicthio, substituted heterocyclicthio, cycloalkyl, substituted cycloalkyl, guanidino, halo, nitro, heteroaryl, substituted heteroaryl, heterocyclic, substituted heterocyclic, oxycarbonylamino, oxythiocarbonylamino, —S(O)₂-alkyl, —S(O)₂-substituted alkyl, —S(O)₂-cycloalkyl, —S(O)₂-substituted cycloalkyl, —S(O)₂-alkenyl, —S(O)₂-substituted alkenyl, —S(O)₂-aryl, —S(O)₂-substituted aryl, —S(O)₂-heteroaryl, —S(O)₂-substituted heteroaryl, —S(O)₂-heterocyclic, —S(O)₂-substituted heterocyclic, —OS(O)₂-alkyl, —OS(O)₂-substituted alkyl, —OS(O)₂-aryl, —OS(O)₂-substituted aryl, —OS(O)₂-heteroaryl, —OS(O)₂-substituted heteroaryl, —OS(O)₂-heterocyclic, —OS(O)₂-substituted heterocyclic, —OSO₂—NR⁵¹R⁵¹ where each R⁵¹ is hydrogen or alkyl, —NR⁵¹S(O)₂-alkyl, —NR⁵S(O)₂-substituted alkyl, —NR⁵¹S(O)₂-aryl, —NR⁵¹S(O)₂-substituted aryl, —NR⁵¹S(O)₂-heteroaryl, —NR⁵¹S(O)₂-substituted heteroaryl, —NR⁵¹S(O)₂-heterocyclic, —NR⁵¹S(O)₂-substituted heterocyclic, —NR⁵¹S(O)₂—NR⁵¹-alkyl, —NR⁵¹S(O)₂—NR⁵¹-substituted alkyl, —NR⁵¹S(O)₂—NR⁵¹-aryl, —NR⁵¹S(O)₂—NR⁵¹-substituted aryl, —NR⁵¹S(O)₂—NR⁵¹-heteroaryl, —NR⁵¹S(O)₂—NR⁵¹-substituted heteroaryl, —NR⁵¹S(O)₂—NR⁵¹-heterocyclic, —NR⁵¹S(O)₂—NR⁵¹-substituted heterocyclic where each R⁵¹ is hydrogen or alkyl, wherein each of the terms is as defined herein.

“Aryloxy” may refer to the group aryl-O— that may include, by way of example, phenoxy, naphthoxy, and the like.

“Substituted aryloxy” may refer to substituted aryl-O— groups.

“Aryloxyaryl” may refer to the group -aryl-O-aryl.

“Substituted aryloxyaryl” may refer to aryloxyaryl groups substituted with from 1 to 3 substituents on either or both aryl rings as defined above for substituted aryl.

“Carboxyl” may refer to —COOH or salts thereof.

“Carboxyl esters” may refer to the groups —C(O)O-alkyl, —C(O)O-substituted alkyl, —C(O)O-aryl, and —C(O)O-substituted aryl wherein alkyl, substituted alkyl, aryl and substituted aryl are as defined herein.

“Cycloalkyl” may refer to cyclic alkyl groups of from 3 to 10 carbon atoms having single or multiple cyclic rings that may include, by way of example, adamantyl, cyclopropyl, cyclobutyl, cyclopentyl, cyclooctyl and the like.

“Substituted cycloalkyl” may refer to a cycloalkyl group, having from 1 to 5 substituents selected from the group consisting of oxo (═O), thioxo (═S), alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxyl, carboxyl esters, cycloalkyl, substituted cycloalkyl, heteroaryl, substituted heteroaryl, heterocyclic, and substituted heterocyclic.

“Cycloalkoxy” may refer to —O-cycloalkyl groups.

“Substituted cycloalkoxy” may refer to —O-substituted cycloalkyl groups.

“Halo” or “halogen” may refer to fluoro, chloro, bromo and iodo and preferably is fluoro or chloro.

“Heteroaryl” may refer to an aromatic group of from 1 to 15 carbon atoms, preferably from 1 to 10 carbon atoms, and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl). Preferred heteroaryls may include pyridinyl, pyrrolyl, indolyl, thiophenyl, and furyl.

“Substituted heteroaryl” may refer to heteroaryl groups that are substituted with from 1 to 3 substituents selected from the same group of substituents defined for substituted aryl.

“Heteroaryloxy” may refer to the group —O-heteroaryl and “substituted heteroaryloxy” may refer to the group —O-substituted heteroaryl.

“Heterocycle” or “heterocyclic” may refer to a saturated or unsaturated group having a single ring or multiple condensed rings, from 1 to 10 carbon atoms and from 1 to 4 heteroatoms selected from the group consisting of nitrogen, sulfur or oxygen within the ring wherein, in fused ring systems, one or more the rings can be aryl or heteroaryl provided that the point of attachment is at the heterocycle.

“Substituted heterocyclic” may refer to heterocycle groups that are substituted with from 1 to 3 of the same substituents as defined for substituted cycloalkyl.

Examples of heterocycles and heteroaryls may include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydro-isoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), piperidinyl, pyrrolidine, tetrahydrofuranyl, and the like.

“Heterocyclyloxy” may refer to the group —O-heterocyclic and “substituted heterocyclyloxy” may refer to the group —O-substituted heterocyclic.

“Thiol” or “mercapto” may refer to the group —SH.

“Alkylsulfanyl” and “alkylthio” may refer to the groups —S-alkyl where alkyl is as defined above.

“Substituted alkylthio” and “substituted alkylsulfanyl” may refer to the group —S-substituted alkyl is as defined above.

“Cycloalkylthio” or “cycloalkylsulfanyl” may refer to the groups —S-cycloalkyl where cycloalkyl is as defined above.

“Substituted cycloalkylthio” may refer to the group —S-substituted cycloalkyl where substituted cycloalkyl is as defined above.

“Arylthio” may refer to the group —S-aryl and “substituted arylthio” may refer to the group —S-substituted aryl where aryl and substituted aryl are as defined above.

“Heteroarylthio” may refer to the group —S-heteroaryl and “substituted heteroarylthio” may refer to the group —S-substituted heteroaryl where heteroaryl and substituted heteroaryl are as defined above.

“Heterocyclicthio” may refer to the group —S-heterocyclic and “substituted heterocyclicthio” may refer to the group —S-substituted heterocyclic where heterocyclic and substituted heterocyclic are as defined above.

The term “amino acid” may refer to any of the naturally occurring amino acids, as well as synthetic analogs (e.g., D-stereoisomers of the naturally occurring amino acids, such as D-threonine) and derivatives thereof. α-Amino acids comprise a carbon atom to which is bonded an amino group, a carboxyl group, a hydrogen atom, and a distinctive group referred to as a “side chain”. The side chains of naturally occurring amino acids are well known in the art and include, for example, hydrogen (e.g., as in glycine), alkyl (e.g., as in alanine, valine, leucine, isoleucine, proline), substituted alkyl (e.g., as in threonine, serine, methionine, cysteine, aspartic acid, asparagine, glutamic acid, glutamine, arginine, and lysine), arylalkyl (e.g., as in phenylalanine and tryptophan), substituted arylalkyl (e.g., as in tyrosine), and heteroarylalkyl (e.g., as in histidine). Unnatural amino acids are also known in the art, as set forth in, for example, Williams (1989); Evans et al. (1990); Pu et al. (1991); Williams et al. (1991); and all references cited therein. The present invention includes the side chains of unnatural amino acids as well.

A. Carbon Monoxide

In certain aspects of the present invention, carbon monoxide may behave as an HIFα stabilizer and/or a HIF prolyl hydroxylase inhibitor.

Carbon monoxide (CO) is a colorless, odorless, and tasteless gas that can be toxic to animals, including humans. According to the Center for Disease Control, more than 450 people unintentionally die from carbon monoxide each year.

It can be toxic to organisms whose blood carries oxygen to sustain its survival. It may be poisonous by entering the lungs through normal breathing and displacing oxygen from the bloodstream. Interruption of the normal supply of oxygen jeopardizes the functions of the heart, brain and other vital functions of the body. However, the use of carbon monoxide for medical applications is being explored (Ryter and Otterbein, 2004).

At amounts of 50 parts per million (ppm), carbon monoxide presents no symptoms to humans exposed to it. However, at 200 ppm, within two-three hours the carbon monoxide can cause a slight headache; at 400 ppm, within one to two hours it can cause a frontal headache that may become widespread within three hours; and, at 800 ppm it can cause dizziness, nausea, and/or convulsions within 45 minutes, and render the subject insensible within two hours. At levels of around 1000 ppm, an organism can expire after exposure for more than around 1-2 minutes.

B. Chalcogenide Compounds

In certain aspects of the present invention, chalcogenide compounds may behave as HIFα stabilizers and/or HIF prolyl hydroxylase inhibitors.

Compounds containing a chalcogen element—those in Group 6 of the periodic table, but excluding oxides—are commonly termed “chalcogenides” or “chalcogenide compounds (used interchangeably herein). These elements are sulfur (S), selenium (Se), tellurium (Te) and polonium (Po). Common chalcogenides contain one or more of S, Se and Te, in addition to other elements. Chalcogenides include elemental forms such as micronized and/or nanomilled particles of S and Se.

Chalcogenides can be toxic, and at some levels lethal, to mammals. In accordance with the present invention, it is anticipated that the levels of chalcogenide should not exceed lethal levels in the appropriate environment. Lethal levels of chalcogenides may be found, for example in Material Safety Data Sheets for each chalcogenide or from information sheets available from the Occupational Safety and Health Administration (OSHA) of the US Government.

It also may prove useful to provide additional stimuli to a biological matter before withdrawing the chalcogenide. In particular, it is envisioned that one may subject an animal to increased ambient temperature prior to removing the source of chalcogenide.

C. H₂S and Other Sulfur Containing Compounds

In certain aspects of the present invention, H₂S and other sulfur-containing compounds may behave as HIFα stabilizers and/or HIF prolyl hydroxylase inhibitors.

Hydrogen sulfide (H₂S) is a potentially toxic gas that is often associated with petrochemical and natural gas, sewage, paper pulp, leather tanning, and food processing. The primary effect, at the cellular level, appears to be inhibition of cytochrome oxidase other oxidative enzymes, resulting in cellular hypoxia. Exposure to extreme levels (500 ppm) results in sudden collapse and unconsciousness, a so-called “knockdown” effect, followed by recovery. Post-exposure effects may persist for years, and include loss of coordination, memory loss, motor dysfunction, personality changes, hallucination and insomnia.

Most contact with H₂S, however, occurs well below such acute toxicity levels. Nonetheless, there is general concern over longterm contact at sub-acute levels. Some reports exist indicating persistent impairments in balance and memory, as well as altered sensory motor functions may occur in humans following chronic low-level H₂S exposure. Kilburn and Warshaw (1995); Kilburn (1999). Others have reported that perinatal exposure of rats to low (20 or 50 ppm) H₂S for 7 hours per day from gestation through post-natal day 21 resulted in longer dendritic branches with reduced aborization of cerebellar Purkinje cells. Other neurologic defects associated with relatively low levels of H₂S include altered brain neurotransmitter concentrations and altered neurologic responses, such as increased hippocampal theta EEG activity.

Behavioral toxicity was studied in rats exposed to moderate levels of H₂S. The results showed that H₂S inhibits discriminated avoidance responses immediately after the end of the exposure (Higuchi and Fukamachi, 1997), and also interferes with the ability of rats to learn a baited radial arm maze task (Partlo et al., 2001). In another perinatal study using 80 ppm H₂S, no neuropathological effects or altered motor activity, passive avoidance, or acoustic startle response in exposed rat pups was seen. Dorman et al. (2000). Finally, Struve et al. (2001) exposed rats to H₂S by gas at various levels for 3 hours per day on five consecutive days. Significant reductions in motor activity, water maze performance and body temperature following exposure to 80 ppm or greater H₂S were observed. Taken together, these reports indicate that H₂S can have a variety of effects on the biochemistry of mammalian tissues, but there is no clear pattern of response in terms of behavior.

Once dissolved in plasma, H₂S will be involved in a series of chemical reactions. The chemical reactions are: (1) the dissociation of the molecular H₂S to form the bisulfide ion, (2) the dissociation of the bisulfide ion to the sulfide ion, and (3) the self ionization of water. The reactions are given below:

H₂S_{(aq)} [symbol] HS_{(aq)}⁻+H_{(aq)}⁺

HS_{(aq)}⁻ [symbol] S_{(aq)}⁻²+H_{(aq)}⁺

H₂O [symbol] H_{(aq)}⁺+OH_{(aq)}⁻

Using the equilibrium constants K₁=1.039 E⁻⁰⁷, K₂=6.43 E⁻¹⁶ and K_{w}=1.019 E⁻¹⁴, at pH 7.4 the calculated amount of the different species relative to the total S concentration are approximately 23% H₂S and 77% HS⁻, while the amount of S²⁻ tends to zero.

The inventor uses an extractive alkylation technique coupled with gas chromatography and mass specific detection to quantify hydrogen sulfide (adapted from Hyspler et al., 2002). This method involves firstly adding a 50 μL sample of blood, serum or tissue extract that has been diluted in nitrogen purged deoxygenated water to a concentration of 1 mg/mL, together with 150 μL of a reaction buffer consisting of 5 mM benzalkonium chloride (BZK) in a saturated borate buffer. Added to this is first, 100 μL of a 15 μM solution of 4-chloro-benzyl methyl sulfide (4CBMS) in ethyl acetate and then 100 μL of a 20 mM solution of pentafluorobenzylbromide (PFBBr) in toluene. This solution is then sealed and incubated at 55° C. with rotation or shaking for 2 hr. After this incubation period, 200 μL of a saturated solution of KH₂PO₄ is then added, and the organic phase is removed and analyzed by gas chromatography and mass specific detection according to the methods described in Hyspler et al., 2002. These measurements are then compared to a standard curve generated using the same method described above, beginning with known standard concentrations ranging from 1 μM to 1 mM of Na₂S prepared in nitrogen purged deoxygenated H₂O, in order to determine the concentration of endogenous hydrogen sulfide levels. In order to analyze bound and/or oxidized sulfide levels, the same method is applied, except that a denaturing/reducing reaction buffer is used, which consists of 5 mM BZK with 1% tetraethylammonium hydroxide (TEAH) and 1 mM tris(2-carboxyethyl)-phosphine hydrochloride (TCEP) in saturated borate buffer, instead of the reaction buffer described above.

Typical levels of hydrogen sulfide contemplated for use in accordance with the present invention include values of about 1 to about 150 ppm, about 10 to about 140 ppm, about 20 to about 130 ppm, and about 40 to about 120 ppm, or the equivalent oral, intravenous or transdermal dosage thereof. Other relevant ranges include about 10 to about 80 ppm, about 20 to about 80 ppm, about 10 to about 70 ppm, about 20 to about 70 ppm, about 20 to about 60 ppm, and about 30 to about 60 ppm, or the equivalent oral, intravenous or transdermal thereof. It also is contemplated that, for a given animal in a given time period, the chalcogenide atmosphere should be reduced to avoid a potentially lethal build up of chalcogenide in the subject. For example, an initial environmental concentration of 80 ppm may be reduced after 30 min to 60 ppm, followed by further reductions at 1 hr (40 ppm) and 2 hrs (20 ppm).

1. H₂S Precursors

The present invention also concerns the use of compounds and agents that can yield H₂S under certain conditions, such as upon exposure, or soon thereafter, to biological matter. It is contemplated that such precursors yield H₂S upon one or more enzymatic or chemical reactions.

D. Other Chalcogenides

In certain embodiments, the HIFα stabilizer and/or HIF prolyl hydroxylase inhibitor is dimethylsulfoxide (DMSO), dimethylsulfide (DMS), methylmercaptan or methanethiol (CH₃SH or MeSH), mercaptoethanol, thiocyanate, hydrogen cyanide, or CS₂. In particular embodiments, the HIFα stabilizer and/or HIF prolyl hydroxylase inhibitor is CS₂, MeSH, or DMS. Compounds on the order of the size of these molecules are particularly contemplated (that is, within about 50% of their molecular weights).

Additional HIFα stabilizers and/or HIF prolyl hydroxylase inhibitors that are envisioned include, but are not limited to, the following structures, many of which are readily available and known to those of skill in the art (identified by CAS number): 104376-79-6 (Ceftriaxone Sodium Salt); 105879-42-3; 1094-08-2 (Ethopropatine HCl); 1098-60-8 (Triflupromazine HCl); 111974-72-2; 113-59-7; 113-98-4 (Penicillin G K⁺); 115-55-9; 1179-69-7; 118292-40-3; 119478-56-7; 120138-50-3; 121123-17-9; 121249-14-7; 1229-35-2; 1240-15-9; 1257-78-9 (Prochlorperazine Edisylate Salt); 128345-62-0; 130-61-0 (Thioridazine HCl) 132-98-9 (Penicillin V K⁺); 13412-64-1 (Dicloxacillin Na⁺ Hydrate); 134678-17-4; 144604-00-2; 146-54-3; 146-54-5 (Fluphenazine 2HCl); 151767-02-1; 159989-65-8; 16960-16-0 (Adrenocorticotropic Hormone Fragment 1-24); 1982-37-2; 21462-39-5 (Clindamycin HCl); 22189-31-7; 22202-75-1; 23288-49-5 (Probucol); 23325-78-2; 24356-60-3 (Cephapirin); 24729-96-2 (Clindamycin); 25507-04-4; 26605-69-6; 27164-46-1 (Cefazolin Na⁺); 2746-81-8; 29560-58-8; 2975-34-0; 32672-69-8 (Mesoridazine Benzene Sulfonate); 32887-01-7; 33286-22-5 ((+)-cis-Diltiazem HCl); 33564-30-6 (Cefoxitin Na⁺); 346-18-9; 3485-14-1; 3511-16-8; 37091-65-9 (Azlocillin Na⁺); 37661-08-8; 3819-00-9; 38821-53-3 (Cephradine); 41372-02-5; 42540-40-9 (Cefamandole Nafate); 4330-99-8 (Trimeprazine hemi-(+)-tartrate Salt); 440-17-5 Trifluoperazine 2HCl; 4697-14-7 (Ticarcillin 2Na⁺); 4800-94-6 (Carbenicillin 2Na⁺); 50-52-2; 50-53-3; 5002-47-1; 51481-61-9 (Cimetidine); 52239-63-1 (6-propyl-2-thiouracil); 53-60-1 (Promazine HCl); 5321-32-4; 54965-21-8 (Albendazole); 5591-45-7 (Thiothixene); 56238-63-2 (Cefuroxime Na⁺); 56796-39-5 (Cefmetazole Na⁺); 5714-00-1; 58-33-3 (Promethazine HCl); 58-38-8; 58-39-9 (Perphenazine); 58-71-9 Cephalothin Na⁺); 59703-84-3 (Piperacillin Na⁺); 60-99-1 (Methotrimeprazine Maleate Salt); 60925-61-3; 61270-78-8; 6130-64-9 (Penicillin G Procaine Salt Hydrate); 61318-91-0 Sulconazole Nitrate Salt); 61336-70-7 Amoxicillin Trihydrate); 62893-20-3 Cefoperazone Na⁺); 64485-93-4 (Cefotaxime Na⁺); 64544-07-6; 64872-77-1; 64953-12-4 Moxalactam Na⁺); 66104-23-2 (Pergolide Mesylate Salt); 66309-69-1; 66357-59-3 (Ranitidine HCl); 66592-87-8 (Cefodroxil); 68401-82-1; 69-09-0 (Chlorpromazine HCl); 69-52-3 (Ampicillin Na⁺); 69-53-4 (Ampicillin); 69-57-8 Penicillin G Na⁺); 70059-30-2; 70356-03-5; 7081-40-5; 7081-44-9 (Cloxacillin Na⁺H₂O); 7177-50-6 Nafcillin Na⁺ H₂O); 7179-49-9; 7240-38-2 (Oxacillin Na H₂O); 7246-14-2; 74356-00-6; 74431-23-5; 74849-93-7; 75738-58-8; 76824-35-6 (Famotidine); 76963-41-2; 79350-37-1; 81129-83-1; 84-02-6 (Prochlorperazine Dimaleate Salt); 87-08-1 (Phenoxymethylpenicillinic Acid); 87239-81-4; 91-33-8 (Benzthiazide); 91832-40-5; 94841-17-5; 99294-94-7; 154-42-7 (6-Thioguanine); 36735-22-5; 536-33-4 (Ethionamide); 52-67-5 (D-Penicillamine); 304-55-2 (Meso-2,3-Dimercaptosuccinic Acid); 59-52-9 2,3-Dimercapto⁺ propanol 6112-76-1 (6-mercaptopurine); 616-91-1 (N-acetyl-L-cysteine); 62571-86-2 (Captopril); 52-01-7 (spironolactone); and, 80474-14-2 (fluticasone propionate).

E. Protective Metabolic Agents

In some embodiments, an HIFα stabilizer and/or HIF prolyl hydroxylase inhibitor may be a protective metabolic agent. Selectively targeting mitochondria is considered an embodiment of the invention in some aspects so as to enhance activity. Such selective mitochondrial targeting has been accomplished by conjugating agents to a lipophilic triphenylphosphonium cation, which readily cross lipid bilayers and accumulate approximately a 1000 fold within the mitochondrial matrix drive by the large potential (150 to −180 mv) across the mitochondrial inner membrane. Analogs of both vitamin E and ubiquinone have been prepared and used to successfully target mitochondria. (Smith et al., 1999; Kelso et al., 2001; Dhanasekaran et al., 2004). A thiol, thibutyltriphosphonium bromide (shown below), has been prepared and used to target mitochondria wherein it accumulated several hundred-fold (Burns et al., 1995; Burns & Murphey, 1997).

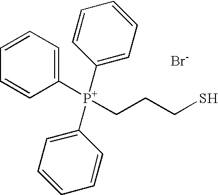

Such conjugates would appear to be suitable candidates for Effective Compounds. In addition to free thiol agents, thiosulfenyl substituted compounds, (H—S—S—R) may be useful. It is contemplated that in some embodiments a protective metabolic agent has the structure of Formula XI:

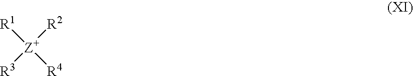

where Z is P or N; R¹, R² and R³ are aryl, heteroaryl, alkylaryl, cycloalkyl, or alkyl (suitably phenyl, benzyl, tolyl, pyridyl, cyclohexyl, C₃-C₁₀ alkyl, optionally halogenated);

and R⁴ is —R⁵SR⁶, wherein R⁵ is C₁-C₁₀ alkyl, R⁶ is H or SH, SO₃H, or PO₃H.

F. Oxygen Antagonists

In certain embodiments, an Effective Compound is an oxygen antagonist. The term “oxygen antagonist” refers to a substance that competes with oxygen insofar as it is used by a biological matter that requires oxygen for it to be alive (“oxygen-utilizing biological matter”). Oxygen is typically used or needed for various cellular processes that create the biological matter's primary source of readily utilizable energy. An oxygen antagonist effectively reduces or eliminates the amount of oxygen that is available to the oxygen-utilizing biological matter, and/or the amount of oxygen that can be used by the oxygen-utilizing biological matter. In one embodiment, an oxygen antagonist may achieve its oxygen antagonism directly. In another embodiment, an oxygen antagonist may achieve its oxygen antagonism indirectly.

A direct oxygen antagonist competes with molecular oxygen for the binding to a molecule (e.g., a protein) that has an oxygen binding site or oxygen binding capacity. Antagonism may be competitive, non-competitive, or uncompetitive as known in the art of pharmacology or biochemistry. Examples of direct oxygen antagonists include, but are not limited to, carbon monoxide (CO), which competes for oxygen binding to hemoglobin and to cytochrome c oxidase.

An indirect oxygen antagonist influences the availability or delivery of oxygen to cells that use oxygen for energy production (e.g., in cellular respiration) in the absence of directly competing for the binding of oxygen to an oxygen-binding molecule. Examples of indirect oxygen antagonists include, but are not limited to, (i) carbon dioxide, which, through a process known as the Bohr effect, reduces the capacity of hemoglobin (or other globins, like myoglobin) to bind to oxygen in the blood or hemolymph of oxygen-utilizing animals, thereby reducing the amount of oxygen that is delivered to oxygen-utilizing cells, tissues, and organs of the organism, thereby reducing the availability of oxygen to cells that use oxygen; (ii) inhibitors of carbonic anhydrase (Supuran et al., 2003, incorporated by reference in its entirety) which, by virtue of inhibiting the hydration of carbon dioxide in the lungs or other respiratory organs, increase the concentration of carbon dioxide, thereby reducing the capacity of hemoglobin (or other globins, like myoglobin) to bind to oxygen in the blood or hemolymph of oxygen-utilizing animals, thereby reducing the amount of oxygen that is delivered to oxygen-utilizing cells, tissues, and organs of the organism, thereby reducing the availability of oxygen to cells that use oxygen; and, (iii) molecules that bind to oxygen and sequester it from or rendering it unavailable to bind to oxygen-binding molecules, including, but not limited to oxygen chelators, antibodies, and the like.

In some embodiments, an oxygen antagonist is both a direct and an indirect oxygen antagonist. Examples include, but are not limited to, compounds, drugs, or agents that directly compete for oxygen binding to cytochrome c oxidase and are also capable of binding to and inhibiting the enzymatic activity of carbonic anhydrase. Thus, in some embodiments an oxygen antagonist inhibits or reduces the amount of cellular respiration occurring in the cells, for instance, by binding sites on cytochrome c oxidase that would otherwise bind to oxygen. Cytochrome c oxidase specifically binds oxygen and then converts it to water. In some embodiments, such binding to cytochrome c oxidase is preferably releasable and reversible binding (e.g., has an in vitro dissociation constant, K_{d}, of at least 10⁻², 10⁻³, or 10⁻⁴ M, and has an in vitro dissociation constant, K_{d}, not greater than 10⁻⁶, 10⁻⁷, 10⁻⁸, 10⁻⁹, 10⁻¹, or 10⁻¹¹ M). In some embodiments, an oxygen antagonist is evaluated by measuring ATP and/or carbon dioxide output.

G. Other Effective Compounds Moreover, in some methods of the invention, the HIFα stabilizers and/or one or more 2-oxoglutarate dioxygenase inhibitors (e.g., a HIF prolyl hydroxylase inhibitor) may be selected from compounds that have a chemical structure of (referred to as Formula I):

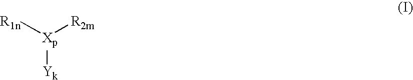

wherein X is N, O, Po, S, Se, or Te;

wherein Y is N or O;

wherein R₁ is H, C, lower alkyl, a lower alcohol, or CN;

wherein R₂ is H, C, lower alkyl, or a lower alcohol, or CN;

wherein n is 0 or 1;

wherein m is 0 or 1;

wherein k is 0, 1, 2, 3, or 4; and,

wherein p is 1 or 2.

The terms “lower alkyl” and “lower alcohol” as used to describe Formula I are used according to their ordinary meanings and the symbols are the ones used to refer to chemical elements. The term “lower,” in this context, is meant to refer to 1, 2, 3, 4, 5, or 6 carbon atoms, or any range derivable therein. In certain embodiments, k is 0. Moreover, in other embodiments, the R₁ and/or R₂ groups can be an amine or lower alkyl amine. In others, R₁ and/or R₂ could be a short chain alcohol or a short chain ketone. Additionally, R₁ and R₂ may be a linear of branched chain bridge and/or the compound may be a cyclic compound. In still further embodiments, X may also be a halogen. Moreover, R₁ and/or R₂ may be other small organic groups, including, C₂-C₅ esters, amides, aldehydes, ketones, carboxylic acids, ethers, nitrites, anhydrides, halides, acyl halides, sulfides, sulfones, sulfonic acids, sulfoxides, and/or thiols. Such substitutions are clearly contemplated with respect to R₁ and/or R₂. In certain other embodiments, R₁ and/or R₂ may be short chain versions of the small organic groups discussed above. “Short chain” means 1, 2, 3, 4, 5, 6, 7, 8, 9, 10, 11, or 12 carbon molecules, or any range derivable therein.

It is contemplated that the HIFα stabilizer and/or one or more 2-oxoglutarate dioxygenase inhibitors (e.g., a HIF prolyl hydroxylase inhibitor) can be a chalcogenide compound in some cases. In certain embodiments, the chalcogenide compound has an alkyl chain with an exposed chalcogenide. In others, the chalcogenide compound has a chalcogenide that becomes exposed once it is taken up by the biological matter. In this respect, the chalcogenide compound is similar to a prodrug as an HIFα stabilizer and/or HIF prolyl hydroxylase inhibitor. Therefore, one or more sulfur, selenium, oxygen, tellurium, polonium, or ununhexium atoms/molecules on the compound becomes available subsequent to exposure of the biological matter to the chalcogenide compound. In this context, “available” means that the sulfur, selenide, oxygen, tellurium, polonium, or ununhexium will retain a negative charge.

In certain embodiments, the chalcogenide is a salt, such as salts wherein the chalcogen is in a −2 oxidation state. Sulfide salts encompassed by embodiments of the invention include, but are not limited to, sodium sulfide (Na₂S), sodium hydrogen sulfide (NaHS), potassium sulfide (K₂S), potassium hydrogen sulfide (KHS), lithium sulfide (Li₂S), rubidium sulfide (Rb₂S), cesium sulfide (Cs₂S), ammonium sulfide ((NH₄)₂S), ammonium hydrogen sulfide (NH₄)HS, beryllium sulfide (BeS), magnesium sulfide (MgS), calcium sulfide (CaS), strontium sulfide (SrS), barium sulfide (BaS), and the like. In like fashion, embodiments of the present invention encompass, but are not limited to, corresponding selenide and telluride salts. It is specifically contemplated that the invention includes compositions containing a chalcogenide salt (chalcogenide compound that is a salt) with a pharmaceutically acceptable carrier or prepared as a pharmaceutically acceptable formulation. In still further embodiments, the HIFα stabilizer and/or HIF prolyl hydroxylase inhibitor is selected from the group consisting of H₂S, H₂Se, H₂Te, and H₂Po. In some cases, the HIFα stabilizer and/or HIF prolyl hydroxylase inhibitor of Formula (I) has an X that is an S. In others, X is Se, or X is Te, or X is Po, or X is O. Furthermore, k in the HIFα stabilizer and/or HIF prolyl hydroxylase inhibitor is 0 or 1 in some embodiments. In certain embodiments, the HIFα stabilizer and/or HIF prolyl hydroxylase inhibitor is dimethylsulfoxide (DMSO), dimethylsulfide (DMS), carbon monoxide, methylmercaptan or methanethiol (CH₃SH or MeSH), mercaptoethanol, thiocyanate, hydrogen cyanide, or CS₂. In particular embodiments, the HIFα stabilizer and/or HIF prolyl hydroxylase inhibitor is H₂S, H₂Se, CS₂, MeSH, or DMS. Compounds on the order of the size of these molecules are particularly contemplated (that is, within 50% of the average of their molecular weights).

In certain embodiments, a selenium-containing compound such as H₂Se is employed. The amount of H₂Se may be in the range of 1 to 1000 parts per billion in some embodiments of the invention. It is further contemplated that any embodiment of the present invention discussed in the context of a sulfur-containing compound may be implemented with a selenium-containing compound. This includes substituting one or more sulfur atoms in a sulfur-containing molecule with a corresponding selenium atom.

A further aspect of the invention encompasses compounds represented by Formula IV:

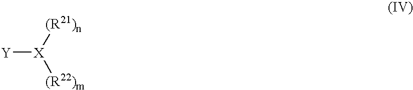

wherein:
X is N, O, P, Po, S, Se, Te, O—O, Po—Po, S—S, Se—Se, or Te—Te;
n and m are independently 0 or 1; and
wherein R²¹ and R²² are independently hydrogen, halo, cyano, phosphate, thio, alkyl, alkenyl, alkynyl, alkoxy, aminoalkyl, cyanoalkyl, hydroxyalkyl, haloalkyl, hydroxyhaloalkyl, alkylsulfonic acid, thiosulfonic acid, alkylthiosulfonic acid, thioalkyl, alkylthio, alkylthioalkyl, alkylaryl, carbonyl, alkylcarbonyl, haloalkylcarbonyl, alkylthiocarbonyl, aminocarbonyl, aminothiocarbonyl, alkylaminothiocarbonyl, haloalkylcarbonyl, alkoxycarbonyl, aminoalkylthio, hydroxyalkylthio, cycloalkyl, cycloalkenyl, aryl, aryloxy, heteroaryloxy, heterocyclyl, heterocyclyloxy, sulfonic acid, sulfonic alkyl ester, thiosulfate, or sulfonamido; and
Y is cyano, isocyano, amino, alkyl amino, aminocarbonyl, aminocarbonyl alkyl, alkylcarbonylamino, amidino, guanidino, hydrazino, hydrazide, hydroxyl, alkoxy, aryloxy, heteroaryloxy, cycloalkyloxy, carbonyloxy, alkylcarbonyloxy, haloalkylcarbonyloxy, arylcarbonyloxy, carbonylperoxy, alkylcarbonylperoxy, arylcarbonylperoxy, phosphate, alkylphosphate esters, sulfonic acid, sulfonic alkyl ester, thiosulfate, thiosulfenyl, sulfonamide, —R²³R²⁴, wherein R²³ is S, SS, Po, Po—Po, Se, Se—Se, Te, or Te—Te, and R²⁴ is defined as for R²¹ herein, or Y is

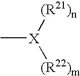

wherein X, R²¹ and R²², are as defined herein.

In certain embodiments, compounds used in the methods of the invention are selected from a compound of the formula (V)

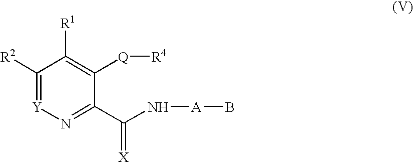

wherein:

A is 1,2-arylidene, 1,3-arylidene, 1,4-arylidene; or (C₁-C₄)-alkylene, optionally substituted by one or two halogen, cyano, nitro, trifluoromethyl, (C₁-C₆)-alkyl, (C₁-C₆)-hydroxyalkyl, (C₁-C₆)-alkoxy, —O—[CH₂]ₓ—C_{f}H_{(2f+1-g)}Hal_{g}, (C₁-C₆)-fluoroalkoxy, (C₁-C₈)-fluoroalkenyloxy, (C₁-C₈)-fluoroalkynyloxy, —OCF₂Cl, —O—CF₂—CHFCl; (C₁-C₆)-alkylmercapto, (C₁-C₆)-alkylsulfinyl, (C₁-C₆)-alkylsulfonyl, (C₁-C₆)-alkylcarbonyl, (C₁-C₆)-alkoxycarbonyl, carbamoyl, N-(C₁-C₄)-alkylcarbamoyl, N,N-di-(C₁-C₄)-alkylcarbamoyl, (C₁-C₆)-alkylcarbonyloxy, (C₃-C₈)-cycloalkyl, phenyl, benzyl, phenoxy, benzyloxy, anilino, N-methylanilino, phenylmercapto, phenylsulfonyl, phenylsulfinyl, sulfamoyl, N-(C₁-C₄)-alkylsulfamoyl, N,N-di-(C₁-C₄)-alkylsulfamoyl; or by a substituted (C₆-C₁₂)-aryloxy, (C₇-C₁₁)-aralkyloxy, (C₆-C₁₂)-aryl, (C₇-C₁₁)-aralkyl radical, which carries in the aryl moiety one to five identical or different substituents selected from halogen, cyano, nitro, trifluoromethyl, (C₁-C₆)-alkyl, (C₁-C₆)-alkoxy, —O—[CH₂]ₓ—C_{f}H_{(2f+1-g)}Hal_{g}, —OCF₂Cl, —O—CF₂—CHFCl, (C₁-C₆)-alkylmercapto, (C₁-C₆)-alkylsulfinyl, (C₁-C₆)-alkylsulfonyl, (C₁-C₆)-alkylcarbonyl, (C₁-C₆)-alkoxycarbonyl, carbamoyl, N-(C₁-C₄)-alkylcarbamoyl, N,N-di-(C₁-C₄)-alkylcarbamoyl, (C₁-C₆)-alkylcarbonyloxy, (C₃-C₈)-cycloalkyl, sulfamoyl, N-(C₁-C₄)-alkylsulfamoyl, N,N-di-(C₁-C₄)-alkylsulfamoyl; or wherein A is —CR⁵R⁶ and R⁵ and R⁶ are each independently selected from hydrogen, (C₁-C₆)-alkyl, (C₃-C₇)-cycloalkyl, aryl, or a substituent of the α-carbon atom of an α-amino acid, wherein the amino acid is a natural L-amino acid or its D-isomer;

B is —CO₂H, —NH₂, —NHSO₂CF₃, tetrazolyl, imidazolyl, 3-hydroxyisoxazolyl, —CONHCOR′″, —CONHSOR′″, CONHSO₂R′″, where R′″ is aryl, heteroaryl, (C₃-C₇)-cycloalkyl, or (C₁-C₄)-alkyl, optionally monosubstituted by (C₆-C₁₂)-aryl, heteroaryl, OH, SH, (C₁-C₄)-alkyl, (C₁-C₄)-alkoxy, (C₁-C₄)-thioalkyl, (C₁-C₄)-sulfinyl, (C₁-C₄)-sulfonyl, CF₃, Cl, Br, F, I, NO₂, —COOH, (C₂-C₅)-alkoxycarbonyl, NH₂, mono-(C₁-C₄-alkyl)-amino, di-(C₁-C₄-alkyl)-amino, or (C₁-C₄)-perfluoroalkyl; or wherein B is a CO₂-G carboxyl radical, where G is a radical of an alcohol G-OH in which G is selected from (C₁-C₂₀)-alkyl radical, (C₃-C₈) cycloalkyl radical, (C₂-C₂₀)-alkenyl radical, (C₃-C₈)-cycloalkenyl radical, retinyl radical, (C₂-C₂₀)-alkynyl radical, (C₄-C₂₀)-alkenynyl radical, where the alkenyl, cycloalkenyl, alkynyl, and alkenynyl radicals contain one or more multiple bonds; (C₆-C₁₆)-carbocyclic aryl radical, (C₇-C₁₆)-carbocyclic aralkyl radical, heteroaryl radical, or heteroaralkyl radical, wherein a heteroaryl radical or heteroaryl moiety of a heteroaralkyl radical contains 5 or 6 ring atoms; and wherein radicals defined for G are substituted by one or more hydroxyl, halogen, cyano, trifluoromethyl, nitro, carboxyl, (C₁-C₁₂)-alkyl, (C₃-C₈)-cycloalkyl, (C₅-C₈)-cycloalkenyl, (C₆-C₁₂)-aryl, (C₇-C₁₆)-aralkyl, (C₂-C₁₂)-alkenyl, (C₂-C₁₂)-alkynyl, (C₁-C₁₂)-alkoxy, (C₁-C₁₂)-alkoxy-(C₁-C₁₂)-alkyl, (C₁-C₁₂)-alkoxy-(C₁-C₁₂)-alkoxy, (C₆-C₁₂)-aryloxy, (C₇-C₁₆)-aralkyloxy, (C₁-C₈)-hydroxyalkyl, —O—[CH₂]ₓ—C_{f}H_{(2f+1-g)}—F_{g}, —OCF₂Cl, —OCF₂—CHFCl, (C₁-C₁₂)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₁₂)-alkenylcarbonyl, (C₂-C₁₂)-alkynylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₁₂)-alkenyloxycarbonyl, (C₂-C₁₂)-alkynyloxycarbonyl, acyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆) aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N-(C₁-C₁₂)-alkylcarbamoyl, N,N-di(C₁-C₁₂)-alkylcarbamoyl, N-(C₃-C₈)-cycloalkyl-carbamoyl, N-(C₆-C₁₆)-arylcarbamoyl, N-(C₇-C₁₆)-aralkylcarbamoyl, N-(C₁-C₁₀)-alkyl-N-(C₆-C₁₆)-arylcarbamoyl, N-(C₁-C₁₀)-alkyl-N-(C₇-C₁₆)-aralkylcarbamoyl, N-((C₁-C₁₀)-alkoxy-(C₁-C₁₀)-alkyl)-carbamoyl, N-((C₆-C₂)-aryloxy-(C₁-C₁₀)alkyl)-carbamoyl, N-((C₇-C₁₆)-aralkyloxy-(C₁-C₁₀)-alkyl)-carbamoyl, N-(C₁-C₁₀)-alkyl-N-((C₁-C₁₀)-alkoxy-(C₁-C₁₀)-alkyl)-carbamoyl, N-(C₁-C₁₀)-alkyl-N-((C₆-C₁₆)-aryloxy-(C₁-C₁₀)-alkyl)-carbamoyl, N-(C₁-C₁₀)-alkyl-N-((C₇-C₁₆)-aralkyloxy-(C₁-C₁₀)-alkyl)-carbamoyl, carbamoyloxy, N-(C₁-C₁₂)-alkylcarbamoyloxy, N,N-di-(C₁-C₁₂)-alkylcarbamoyloxy, N-(C₃-C₈)-cycloalkylcarbamoyloxy, N-(C₆-C₁₂)-arylcarbamoyloxy, N-(C₇-C₁₆)-aralkylcarbamoyloxy, N-(C₁-C₁₀)-alkyl-N-(C₆-C₁₂)-arylcarbamoyloxy, N(C₁-C₁₀)-alkyl-N-(C₇-C₁₆)-aralkylcarbamoyloxy, N-((C₁-C₁₀)-alkyl)-carbamoyloxy, N-((C₆-C₁₂)-aryloxy-(C₁-C₁₀)-alkyl)-carbamoyloxy, N-((C₇-C₁₆)-aralkyloxy-(C₁-C₁₀)-alkyl)-carbamoyloxy, N-(C₁-C₁₀)-alkyl-N-((C₁-C₁₀)-alkoxy-(C₁-C₁₀)-alkyl)-carbamoyloxy, N-(C₁-C₁₀)-alkyl-N-((C₆-C₁₂)-aryloxy-(C₁-C₁₀)-alkyl)-carbamoyloxy, N-(C₁-C₁₀)-alkyl-N-((C₇-C₁₆)-aralkyloxy-(C₁-C₁₀)-alkyl)-carbamoyloxyamino, (C₁-C₂)-alkylamino, di-(C₁-C₁₂)-alkylamino, (C₃-C₈)-cycloalkylamino, (C₂-C₁₂)-alkenylamino, (C₂-C₁₂)-alkynylamino, N-(C₆-C₁₂)-arylamino, N-(C-C₁₁)-aralkylamino, N-alkyl-aralkylamino, N-alkyl-arylamino, (C₁-C₁₂)-alkoxyamino, (C₁-C₁₂)-alkoxy-N-(C₁-C₁₀)-alkylamino, (C₁-C₁₂)-alkylcarbonylamino, (C₃-C₈)-cycloalkylcarbonylamino, (C₆-C₁₂) arylcarbonylamino, (C₇-C₁₆)-aralkylcarbonylamino, (C₁-C₁₂)-alkylcarbonyl-N-(C₁-C₁₀)-alkylamino, (C₃-C₈)-cycloalkylcarbonyl-N-(C₁-C₁₀)-alkylamino, (C₆-C₁₂)-arylcarbonyl-N-(C₁-C₁₀)alkylamino, (C₇-C₁₁)-aralkylcarbonyl-N-(C₁-C₁₀)-alkylamino, (C₁-C₁₂)-alkylcarbonylamino-(C₁-C₈)-alkyl, (C₃-C₈)-cycloalkylcarbonylamino-(C₁-C₈)alkyl, (C₆-C₁₂)-arylcarbonylamino-(C₁-C₈)-alkyl, (C₇-C₁₂)-aralkylcarbonylamino(C₁-C₈)-alkyl, amino-(C₁-C₁₀)-alkyl, N-(C₁-C₁₀) alkylamino-(C₁-C₁₀)-alkyl, N,N-di-(C₁-C₁₀)-alkylamino-(C₁-C₁₀)-alkyl, (C₃-C₈)cycloalkylamino-(C₁-C₁₀)-alkyl, (C₁-C₂)-alkylmercapto, (C₁-C₁₂)-alkylsulfinyl, (C₁-C₁₂)-alkylsulfonyl, (C₆-C₁₆)-arylmercapto, (C₆-C₁₆)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, sulfamoyl, N-(C₁-C₁₀)-alkylsulfamoyl, N,N-di(C₁-C₁₀)-alkylsulfamoyl, (C₃-C₈)-cycloalkylsulfamoyl, N-(C₆-C₁₂)-alkylsulfamoyl, N-(C₇-C₁₆)-aralkylsulfamoyl, N-(C₁-C₁₀)-alkyl-N-(C₆-C₁₂)-arylsulfamoyl, N-(C₁-C₁₀)-alkyl-N-(C₇-C₁₆)-aralkylsulfamoyl, (C₁-C₁₀)-alkylsulfonamido, N-((C₁-C₁₀)-alkyl)-(C₁-C₁₀)-alkylsulfonamido, (C₇-C₁₆)-aralkylsulfonamido, or N-((C₁-C₁₀)-alkyl-(C₇-C₁₆)-aralkylsulfonamido; wherein radicals which are aryl or contain an aryl moiety, may be substituted on the aryl by one to five identical or different hydroxyl, halogen, cyano, trifluoromethyl, nitro, carboxyl, (C₁-C₁₂)-alkyl, (C₃-C₈)-cycloalkyl, (C₆-C₁₂)-aryl, (C₇-C₁₆)-aralkyl, (C₁-C₁₂)-alkoxy, (C₁-C₁₂)-alkoxy-(C₁-C₁₂)alkyl, (C₁-C₁₂)-alkoxy-(C₁-C₁₂)alkoxy, (C₆-C₁₂)-aryloxy, (C₇-C₁₆)-aralkyloxy, (C₁-C₈)-hydroxyalkyl, (C₁-C₁₂)-alkylcarbonyl, (C₃-C₈)-cycloalkyl-carbonyl, (C₆-C₁₂)-arylcarbonyl, (C₇-C₁₆) aralkylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₁₂)-alkenyloxycarbonyl, (C₂-C₁₂)-alkynyloxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N-(C₁-C₁₂)-alkylcarbamoyl, N,N-di-(C₁-C₁₂)-alkylcarbamoyl, N-(C₃-C₈)-cycloalkylcarbamoyl, N-(C₆-C₁₂)-arylcarbamoyl, N-(C₇-C₁₆)-aralkylcarbamoyl, N-(C₁-C₁₀)-alkyl-N-(C₆-C₁₂)-arylcarbamoyl, N-(C₁-C₁₀)-alkyl-N-(C₇-C₁₆)-aralkylcarbamoyl, N-((C₁-C₁₀)-alkoxy-(C₁-C₁₀)-alkyl)-carbamoyl, N-((C₆-C₁₂)-aryloxy-(C₁-C₁₀)-alkyl)-carbamoyl, N-((C₇-C₁₆)-aralkyloxy-(C₁-C₁₀)-alkyl)-carbamoyl, N-(C₁-C₁₀)-alkyl-N-((C₁-C₁₀)-alkoxy-(C₁-C₁₀)-alkyl)-carbamoyl, N-(C₁-C₁₀)-alkyl-N-((C₆-C₁₂)-aryloxy-(C₁-C₁₀)-alkyl)-carbamoyl, N-(C₁-C₁₀)-alkyl-N-((C₇-C₁₆)-aralkyloxy-(C₁-C₁₀)-alkyl)-carbamoyl, carbamoyloxy, N-(C₁-C₁₂)-alkylcarbamoyloxy, N,N-di-(C₁-C₁₂)-alkylcarbamoyloxy, N-(C₃-C₈)-cycloalkylcarbamoyloxy, N-(C₆-C₁₂)-arylcarbamoyloxy, N-(C₇-C₁₆)-aralkylcarbamoyloxy, N-(C₁-C₁₀)-alkyl-N-(C₆-C₁₂)-arylcarbamoyloxy, N(C₁-C₁₀)-alkyl-N-(C₇-C₁₆)-aralkylcarbamoyloxy, N-((C₁-C₁₀)-alkyl)-carbamoyloxy, N-((C₆-C₁₂)-aryloxy-(C₁-C₁₀)-alkyl)-carbamoyloxy, N-((C₇-C₁₆)-aralkyloxy-(C₁-C₁₀)-alkyl)-carbamoyloxy, N-(C₁-C₁₀)-alkyl-N-((C₁-C₁₀)-alkoxy-(C₁-C₁₀)-alkyl)-carbamoyloxy, N-(C₁-C₁₀)-alkyl-N-((C₆-C₂)-aryloxy-(C₁-C₁₀)-alkyl)-carbamoyloxy, N-(C₁-C₁₀)-alkyl-N-((C₇-C₁₆)-aralkyloxy-(C₁-C₁₀)-alkyl)-carbamoyloxyamino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N-(C₆-C₁₂)-arylamino, N-(C₇-C₁₁)-aralkylamino, N-alkylaralkylamino, N-alkyl-arylamino, (C₁-C₁₂)-alkoxyamino, (C₁-C₁₂)-alkoxy-N-(C₁-C₁₀)-alkylamino, (C₁-C₁₂)-alkylcarbonylamino, (C₃-C₈)-cycloalkylcarbonylamino, (C₆-C₁₂)-arylcarbonylamino, (C₇-C₁₆)-alkylcarbonylamino, (C₁-C₂)-alkylcarbonyl-N-(C₁-C₁₀)-alkylamino, (C₃-C₈)-cycloalkylcarbonyl-N-(C₁-C₁₀)-alkylamino, (C₆-C₁₂)-arylcarbonyl-N-(C₁-C₁₀)-alkylamino, (C₇-C₁₁)-aralkylcarbonyl-N-(C₁-C₁₀)-alkylamino, (C₁-C₁₂)-alkylcarbonylamino-(C₁-C₈)-alkyl, (C₃-C₈)-cycloalkylcarbonylamino-(C₁-C₈)-alkyl, (C₆-C₁₂)-arylcarbonylamino-(C₁-C₈)-alkyl, (C₇-C₁₆)-aralkylcarbonylamino-(C₁-C₈)-alkyl, amino-(C₁-C₁₀)-alkyl, N-(C₁-C₁₀)-alkylamino-(C₁-C₁₀)alkyl, N,N-di-(C₁-C₁₀)-alkylamino-(C₁-C₁₀)-alkyl, (C₃-C₈)-cycloalkylamino-(C₁-C₁₀)-alkyl, (C₁-C₁₂)-alkylmercapto, (C₁-C₁₂)-alkylsulfinyl, (C₁-C₁₂)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, or (C₇-C₁₆)-aralkylsulfonyl;

X is O or S;

Q is O, S, NR′, or a bond;

where, if Q is a bond, R⁴ is halogen, nitrile, or trifluoromethyl; or where, if Q is O, S, or NR′, R⁴ is hydrogen, (C₁-C₁₀)-alkyl radical, (C₂-C₁₀)-alkenyl radical, (C₂-C₁₀)-alkynyl radical, wherein alkenyl or alkynyl radical contains one or two C—C multiple bonds; unsubstituted fluoroalkyl radical of the formula —[CH₂]ₓ—C_{f}H_{(2f+1-g)}—F_{g}, (C₁-C₈)-alkoxy-(C₁-C₆)-alkyl radical, (C₁-C₆)-alkoxy-(C₁-C₄)-alkoxy-(C₁-C₄)-alkyl radical, aryl radical, heteroaryl radical, (C₇-C₁,)-aralkyl radical, or a radical of the Formula Z:

[CH₂]ᵥ—[O]_{w}—[CH₂]ₜ-E (Z)

where

E is a heteroaryl radical, a (C₃-C₈)-cycloalkyl radical, or a phenyl radical of the formula F

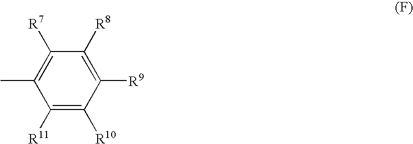

v is 0-6,

w is 0 or 1,

t is 0-3, and

R⁷, R⁸, R⁹, R¹⁰, and R¹¹ are identical or different and are hydrogen, halogen, cyano, nitro, trifluoromethyl, (C₁-C₆)-alkyl, (C₃-C₈)-cycloalkyl, (C₁-C₆)-alkoxy, —O—[CH₂]ₓ—C_{f}H_{(2f+1-g)}—F_{g}, —OCF₂—Cl, —O—CF₂—CHFCl, (C₁-C₆)-alkylmercapto, (C₁-C₆)-hydroxyalkyl, (C₁-C₆)-alkoxy-(C₁-C₆)-alkoxy, (C₁-C₆)-alkoxy-(C₁-C₆)-alkyl, (C₁-C₆)-alkylsulfinyl, (C₁-C₆)-alkylsulfonyl, (C₁-C₆)-alkylcarbonyl, (C₁-C₈)-alkoxycarbonyl, carbamoyl, N-(C₁-C₈)-alkylcarbamoyl, N,N-di-(C₁-C₈)-alkylcarbamoyl, or (C₇-C₁₁)-aralkylcarbamoyl, optionally substituted by fluorine, chlorine, bromine, trifluoromethyl, (C₁-C₆)-alkoxy, N-(C₃-C₈)-cycloalkylcarbamoyl, N-(C₃-C₈)-cycloalkyl-(C₁-C₄)-alkylcarbamoyl, (C₁-C₆)-alkylcarbonyloxy, phenyl, benzyl, phenoxy, benzyloxy, NR^{Y}R^{Z} wherein R^{Y} and R^{Z} are independently selected from hydrogen, (C₁-C₁₂)-alkyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₃-C₁₀)-cycloalkyl, (C₃-C₁₂)-alkenyl, (C₃-C₁₂)-alkynyl, (C₆-C₁₂)-aryl, (C₇-C₁₁)-aralkyl, (C₁-C₁₂)-alkoxy, (C₇-C₁₂)aralkoxy, (C₁-C₁₂)-alkylcarbonyl, (C₃-C₈)-cycloalkylcarbonyl, (C₆-C₁₂) arylcarbonyl, (C₇-C₁₆)-aralkylcarbonyl; or further wherein R^{Y} and R^{Z} together are —[CH₂]ₕ, in which a CH₂ group can be replaced by O, S, N-(C₁-C₄)-alkylcarbonylimino, or N-(C₁-C₄)-alkoxycarbonylimino; phenylmercapto, phenylsulfonyl, phenylsulfinyl, sulfamoyl, N-(C₁-C₈)-alkylsulfamoyl, or N,N-di-(C₁-C₈)-alkylsulfamoyl; or alternatively R⁷ and R⁸, R⁸ and R⁹, R⁹ and R¹⁰, or R¹⁰ and R¹¹, together are a chain selected from —[CH₂]ₙ— or —CH.dbd.CH—CH.dbd.CH—, where a CH₂ group of the chain is optionally replaced by O, S, SO, SO₂, or NR^{Y}; and n is 3, 4, or 5; and if E is a heteroaryl radical, said radical can carry 1-3 substituents selected from those defined for R⁷—R″, or if E is a cycloalkyl radical, the radical can carry one substituent selected from those defined for R⁷-R¹¹;

or where, if Q is NR′, R⁴ is alternatively R″, where R′ and R″ are identical or different and are hydrogen, (C₆-C₁₂)-aryl, (C₇-C₁₁)-aralkyl, (C₁-C₈)-alkyl, (C₁-C₈)-alkoxy-(C₁-C₈)-alkyl, (C₇-C₁₂)-aralkoxy-(C₁-C₈)-allyl, (C₆-C₁₂)-aryloxy-(C₁-C₈)-alkyl, (C₁-C₁₀)-alkylcarbonyl, optionally substituted (C₇-C₁₆)-aralkylcarbonyl, or optionally substituted C₆-C₁₂)-arylcarbonyl; or R′ and R″ together are —[CH₂]ₕ, in which a CH₂ group can be replaced by O, S, N-acylimino, or N-(C₁-C₁₀)-alkoxycarbonylimino, and h is 3 to 7.

Y is N or CR³;

R¹, R² and R³ are identical or different and are hydrogen, hydroxyl, halogen, cyano, trifluoromethyl, nitro, carboxyl, (C₁-C₂₀)-alkyl, (C₃-C₈)-cycloalkyl, (C₃-C₈)cycloalkyl-(C₁-C₁₂)-alkyl, (C₃-C₈)-cycloalkoxy, (C₃-C₈)-cycloalkyl-(C₁-C₁₂)-alkoxy, (C₃-C₈)-cycloalkyloxy-(C₁-C₁₂)-alkyl, (C₃-C₈)-cycloalkyloxy-(C₁-C₁₂)-alkoxy, (C₃-C₈)-cycloalkyl-(C₁-C₈)-alkyl-(C₁-C₆)-alkoxy, (C₃-C₈)-cycloalkyl-(C₁-C₈)-alkoxy-(C₁-C₆)-alkyl, (C₃-C₈)-cycloalkyloxy-(C₁-C₈)-alkoxy-(C₁-C₆)-alkyl, (C₃-C₈)-cycloalkoxy-(C₁-C₈)-alkoxy-(C₁-C₈)-alkoxy, (C₆-C₁₂)-aryl, (C₇-C₁₆)-aralkyl, (C₇-C₁₆)-aralkenyl, (C₇-C₁₆)-aralkynyl, (C₂-C₂₀)-alkenyl, (C₂-C₂₀)-alkynyl, (C₁-C₂₀)-alkoxy, (C₂-C₂₀)-alkenyloxy, (C₂-C₂₀)-alkynyloxy, retinyloxy, (C₁-C₂₀)-alkoxy-(C₁-C₁₂)-alkyl, (C₁-C₁₂)-alkoxy-(C₁-C₁₂)-alkoxy, (C₁-C₁₂)-alkoxy-(C₁-C₈)-alkoxy-(C₁-C₈)-alkyl, (C₆-C₁₂)-aryloxy, (C₇-C₁₆)-aralkyloxy, (C₆-C₁₂)-aryloxy-(C₁-C₆)-alkoxy, (C₇-C₁₆)-aralkoxy-(C₁-C₆)-alkoxy, (C₁-C₁₆)-hydroxyalkyl, (C₆-C₁₆)-aryloxy-(C₁-C₈)-alkyl, (C₇-C₁₆)-aralkoxy-(C₁-C₈)-alkyl, (C₆-C₁₂)-aryloxy-(C₁-C₈)-alkoxy-(C₁-C₆)-alkyl, (C₇-C₁₂)-aralkyloxy-(C₁-C₈)-alkoxy-(C₁-C₆)-alkyl, (C₂-C₂₀)-alkenyloxy-(C₁-C₆)-alkyl, (C₂-C₂₀)-alkynyloxy-(C₁-C₆)-alkyl, retinyloxy-(C₁-C₆)-alkyl, —O—[CH₂]ₓC_{f}H_{(2f+1-g)}F_{g}, —OCF₂Cl, —OCF₂—CHFCl, (C₁-C₂₀)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₂₀)-alkenylcarbonyl, (C₂-C₂₀)-alkynylcarbonyl, (C₁-C₂₀)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₂₀)-alkenyloxycarbonyl, retinyloxycarbonyl, (C₂-C₂₀)-alkynyloxycarbonyl, (C₆-C₁₂)-aryloxy-(C₁-C₆)-alkoxycarbonyl, (C₇-C₁₆)-aralkoxy-(C₁-C₆)-alkoxycarbonyl, (C₃-C₈)-cycloalkyl-(C₁-C₆)-alkoxycarbonyl, (C₃-C₈)-cycloalkoxy-(C₁-C₆)-alkoxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N-(C₁-C₁₂)-alkylcarbamoyl, N,N-di-(C₁-C₁₂)-alkylcarbamoyl, N-(C₃-C₈)-cycloalkylcarbamoyl, N,N-dicyclo-(C₃-C₈)-alkylcarbamoyl, N-(C₁-C₁₀)-alkyl-N-(C₃-C₈)-cycloalkylcarbamoyl, N-((C₃-C₈)-cycloalkyl-(C₁-C₆)-alkyl)-carbamoyl, N-(C₁-C₆)-alkyl-N-((C₃-C₈)-cycloalkyl-(C₁-C₆)-alkyl)-carbamoyl, N-(+)-dehydroabietylcarbamoyl, N-(C₁-C₆)-alkyl-N-(+)-dehydroabietylcarbamoyl, N-(C₆-C₁₂)-arylcarbamoyl, N-(C₇-C₁₆)-aralkylcarbamoyl, N-(C₁-C₁₀)-alkyl-N-(C₆-C₁₆)-arylcarbamoyl, N-(C₁-C₁₀)-alkyl-N-(C₇-C₁₆)-aralkylcarbamoyl, N-((C₁-C₁₈)-alkoxy-(C₁-C₁₀)-alkyl)-carbamoyl, N-((C₆-C₁₆)-aryloxy-(C₁-C₁₀)-alkyl)-carbamoyl, N-((C₇-C₁₆)-aralkyloxy-(C₁-C₁₀)-alkyl)-carbamoyl, N-(C₁-C₁₀)-alkyl-N-((C₁-C₁₀)-alkoxy-(C₁-C₁₀)-alkyl)-carbamoyl, N-(C₁-C₁₀)-alkyl-N-((C₆-C₁₂)-aryloxy-(C₁-C₁₀)-alkyl)-carbamoyl, N-(C₁-C₁₀)-alkyl-N-((C₇-C₁₆)-aralkyloxy-(C₁-C₁₀)-alkyl)-carbamoyl; CON(CH₂)ₕ, in which a CH₂ group can be replaced by O, S, N-(C₁-C₈)-alkylimino, N-(C₃-C₈)-cycloalkylimino, N-(C₃-C₈)-cycloalkyl-(C₁-C₄)-alkylimino, N-(C₆-C₁₂)-arylimino, N-(C₇-C₁₆)-aralkylimino, N-(C₁-C₄)-alkoxy-(C₁-C₆)-alkylimino, and h is from 3 to 7; a carbamoyl radical of the formula R

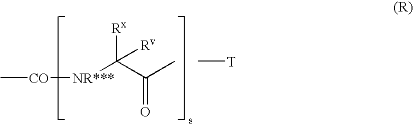

in which

R^{x} and R^{v} are each independently selected from hydrogen, (C₁-C₆)-alkyl, (C₃-C₇)-cycloalkyl, aryl, or the substituent of an α-carbon of an α-amino acid, to which the L- and D-amino acids belong,

s is 1-5,

T is OH, or NR*R**, and R*, R** and R*** are identical or different and are selected from hydrogen, (C₆-C₁₂)-aryl, (C₇-C₁₁)-aralkyl, (C₁-C₈)-alkyl, (C₃-C₈)-cycloalkyl, (+)-dehydroabietyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₁-C₁₀)-alkanoyl, optionally substituted (C₇-C₁₆)-aralkanoyl, optionally substituted (C₆-C₁₂)-aroyl; or R* and R** together are —[CH₂]ₕ, in which a CH₂ group can be replaced by O, S, SO, SO₂, N-acylamino, N-(C₁-C₁₀)-alkoxycarbonylimino, N-(C₁-C₈)-alkylimino, N-(C₃-C₈)-cycloalkylimino, N-(C₃-C₈)-cycloalkyl-(C₁-C₄)-alkylimino, N-(C₆-C₁₂)-arylimino, N-(C₇-C₁₆)-aralkylimino, N-(C₁-C₄)-alkoxy-(C₁-C₆)-alkylimino, and h is from 3 to 7;

carbamoyloxy, N-(C₁-C₁₂)-alkylcarbamoyloxy, N,N-di-(C₁-C₁₂)-alkylcarbamoyloxy, N-(C₃-C₈)-cycloalkylcarbamoyloxy, N-(C₆-C₁₂)-arylcarbamoyloxy, N-(C₇-C₁₆)-aralkylcarbamoyloxy, N-(C₁-C₁₀)-alkyl-N-(C₆-C₁₂)-arylcarbamoyloxy, N-(C₁-C₁₀)-alkyl-N-(C₇-C₁₆)-aralkylcarbamoyloxy, N-((C₁-C₁₀)-alkyl)-carbamoyloxy, N-((C₆-C₁₂)-aryloxy-(C₁-C₁₀)-alkyl)-carbamoyloxy, N-((C₇-C₁₆)-aralkyloxy-(C₁-C₁₀)-alkyl)-carbamoyloxy, N-(C₁-C₁₀)-alkyl-N-((C₁-C₁₀)-alkoxy-(C₁-C₁₀)-alkyl)-carbamoyloxy, N-(C₁-C₁₀)-alkyl-N-((C₆-C₁₂)-aryloxy-(C₁-C₁₀)-alkyl)-carbamoyloxy, N-(C₁-C₁₀)-alkyl-N-((C₇-C₁₆)-aralkyloxy-(C₁-C₁₀)-alkyl)-carbamoyloxyamino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N-(C₆-C₁₂)-arylamino, N-(C₇-C₁₁)-aralkylamino, N-alkyl-aralkylamino, N-alkyl-arylamino, (C₁-C₂)-alkoxyamino, (C₁-C₁₂)-alkoxy-N-(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N-(C₁-C₁₀)-alkylamino, (C₃-C₈)-cycloalkanoyl-N-(C₁-C₁₀)-alkylamino, (C₆-C₁₂)-aroyl-N-(C₁-C₁₀)-alkylamino, (C₇-C₁₁)-aralkanoyl-N-(C₁-C₁₀)-alkylamino, (C₁-C₁₂)-alkanoylamino-(C₁-C₈)-alkyl, (C₃-C₈)-cycloalkanoylamino-(C₁-C₈)-alkyl, (C₆-C₁₂)-aroylamino-(C₁-C₈)-alkyl, (C₇-C₁₆)-aralkanoylamino-(C₁-C₈)-alkyl, amino-(C₁-C₁₀)-alkyl, N-(C₁-C₁₀)-alkylamino-(C₁-C₁₀)-alkyl, N,N-di(C₁-C₁₀)-alkylamino-(C₁-C₁₀)-alkyl, (C₃-C₈)-cycloalkylamino(C₁-C₁₀)-alkyl, (C₁-C₂₀)-alkylmercapto, (C₁-C₂₀)-alkylsulfinyl, (C₁-C₂₀)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, (C₁-C₁₂)-alkylmercapto-(C₁-C₆)-alkyl, (C₁-C₁₂)-alkylsulfinyl-(C₁-C₆)-alkyl, (C₁-C₁₂)-alkylsulfonyl-(C₁-C₆)-alkyl, (C₆-C₁₂)-arylmercapto-(C₁-C₆)-alkyl, (C₆-C₁₂)-arylsulfinyl-(C₁-C₆)-alkyl, (C₆-C₁₂)-arylsulfonyl-(C₁-C₆)-alkyl, (C₇-C₁₆)-aralkylmercapto-(C₁-C₆)-alkyl, (C₇-C₁₆)-aralkylsulfinyl-(C₁-C₆)-alkyl, (C₇-C₁₆)-aralkylsulfonyl-(C₁-C₆)-alkyl, sulfamoyl, N-(C₁-C₁₀)-alkylsulfamoyl, N,N-di-(C₁-C₁₀)-alkylsulfamoyl, (C₃-C₈)-cycloalkylsulfamoyl, N-(C₆-C₁₂)-arylsulfamoyl, N-(C₇-C₁₆)-aralkylsulfamoyl, N-(C₁-C₁₀)-alkyl-N-(C₆-C₁₂)-arylsulfamoyl, N-(C₁-C₁₀)-alkyl-N-(C₇-C₁₆)-aralkylsulfamoyl, (C₁-C₁₀)-alkylsulfonamido, N-((C₁-C₁₀)-alkyl)-(C₁-C₁₀)-alkylsulfonamido, (C₇-C₁₆)-aralkylsulfonamido, and N-((C₁-C₁₀)-alkyl-(C₇-C₁₆)-aralkylsulfonamido; where an aryl radical may be substituted by 1 to 5 substituents selected from hydroxyl, halogen, cyano, trifluoromethyl, nitro, carboxyl, (C₂-C₁₆)-alkyl, (C₃-C₈)-cycloalkyl, (C₃-C₈)-cycloalkyl-(C₁-C₁₂)-alkyl, (C₃-C₈)-cycloalkoxy, (C₃-C₈)-cycloalkyl-(C₁-C₁₂)-alkoxy, (C₃-C₈)-cycloalkyloxy-(C₁-C₁₂)-alkyl, (C₃-C₈)-cycloalkyloxy-(C₁-C₁₂)-alkoxy, (C₃-C₈)-cycloalkyl-(C₁-C₈)-alkyl-(C₁-C₆)-alkoxy, (C₃-C₈)-cycloalkyl(C₁-C₈)-alkoxy-(C₁-C₆)-alkyl, (C₃-C₈)-cycloalkyloxy-(C₁-C₈)-alkoxy-(C₁-C₆)-alkyl, (C₃-C₈)-cycloalkoxy-(C₁-C₈)-alkoxy-(C₁-C₈)-alkoxy, (C₆-C₁₂)-aryl, (C₇-C₁₆)-aralkyl, (C₂-C₁₆)-alkenyl, (C₂-C₁₂)-alkynyl, (C₁-C₁₆)-alkoxy, (C₁-C₁₆)-alkenyloxy, (C₁-C₁₂)-alkoxy-(C₁-C₁₂)-alkyl, (C₁-C₁₂)-alkoxy-(C₁-C₁₂)-alkoxy, (C₁-C₁₂)-alkoxy(C₁-C₈)-alkoxy-(C₁-C₈)-alkyl, (C₆-C₁₂)-aryloxy, (C₇-C₆)-aralkyloxy, (C₆-C₁₂)-aryloxy-(C₁-C₆)-alkoxy, (C₇-C₁₆)-aralkoxy-(C₁-C₆)-alkoxy, (C₁-C₈)-hydroxyalkyl, (C₆-C₁₆)-aryloxy-(C₁-C₈)-alkyl, (C₇-C₁₆)-aralkoxy-(C₁-C₈)-alkyl, (C₆-C₁₂)-aryloxy-(C₁-C₈)-alkoxy-(C₁-C₆)-alkyl, (C₇-C₁₂)-aralkyloxy-(C₁-C₈)-alkoxy-(C₁-C₆)-alkyl, —O—[CH₂]ₓC_{f}H_{(2f+1-g)}F_{g}, —OCF₂Cl, —OCF₂—CHFCl, (C₁-C₁₂)-alkylcarbonyl, (C₃-C₈)-cycloalkylcarbonyl, (C₆-C₁₂)-arylcarbonyl, (C₇-C₁₆)-aralkylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₁₂)-alkenyloxycarbonyl, (C₂-C₁₂)-alkynyloxycarbonyl, (C₆-C₁₂)-aryloxy-(C₁-C₆)-alkoxycarbonyl, (C₇-C₁₆)-aralkoxy-(C₁-C₆)-alkoxycarbonyl, (C₃-C₈)-cycloalkyl-(C₁-C₆)-alkoxycarbonyl, (C₃-C₈)-cycloalkoxy-(C₁-C₆)-alkoxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₁-C₁₂)-alkenyloxycarbonyloxy, (C₂-C₁₂)-alkynyloxycarbonyloxy, carbamoyl, N-(C₁-C₁₂)-alkylcarbamoyl, N,N-di(C₁-C₂)-alkylcarbamoyl, N-(C₃-C₈)-cycloalkylcarbamoyl, N,N-dicyclo-(C₃-C₈)-alkylcarbamoyl, N-(C₁-C₁₀)-alkyl-N-(C₃-C₈)-cycloalkylcarbamoyl, N-((C₃-C₈)-cycloalkyl-(C₁-C₆)-alkyl)carbamoyl, N-(C₁-C₆)-alkyl-N-((C₃-C₈)-cycloalkyl-(C₁-C₆)-alkyl)carbamoyl, N-(+)-dehydroabietylcarbamoyl, N-(C₁-C₆)-alkyl-N-(+)-dehydroabietylcarbamoyl, N-(C₆-C₁₂)-arylcarbamoyl, N-(C₇-C₁₆)-aralkylcarbamoyl, N-(C₁-C₁₀)-alkyl-N-(C₆-C₁₆)-arylcarbamoyl, N-(C₁-C₁₀)-alkyl-N-(C₇-C₁₆)-aralkylcarbamoyl, N-((C₁-C₁₆)-alkoxy-(C₁-C₁₀)-alkyl)carbamoyl, N-((C₆-C₁₆)-aryloxy-(C₁-C₁₀)-alkyl)carbamoyl, N-((C₇-C₁₆)-aralkyloxy-(C₁-C₁₀)-alkyl)carbamoyl, N-(C₁-C₁₀)-alkyl-N-((C₁-C₁₀)-alkoxy-(C₁-C₁₀)-alkyl)carbamoyl, N-(C₁-C₁₀)-alkyl-N-((C₆-C₁₂)-aryloxy-(C₁-C₁₀)-alkyl)carbamoyl, N-(C₁-C₁₀)-alkyl-N-((C₇-C₁₆)-aralkyloxy-(C₁-C₁₀)-alkyl)-carbamoyl, CON(CH₂)ₕ, in which a CH₂ group can be replaced by, O, S, N-(C₁-C₈)-alkylimino, N-(C₃-C₈)-cycloalkylimino, N-(C₃-C₈)-cycloalkyl-(C₁-C₄)-alkylimino, N-(C₆-C₁₂)-arylimino, N-(C₇-C₁₆)-aralkylimino, N-(C₁-C₄)-alkoxy-(C₁-C₆)-alkylimino, and h is from 3 to 7; carbamoyloxy, N-(C₁-C₂)-alkylcarbamoyloxy, N,N-di-(C₁-C₁₂)-alkylcarbamoyloxy, N-(C₃-C₈)-cycloalkylcarbamoyloxy, N-(C₆-C₁₆)-arylcarbamoyloxy, N-(C₇-C₁₆)-aralkylcarbamoyloxy, N-(C₁-C₁₀)-alkyl-N-(C₆-C₁₂)-arylcarbamoyloxy, N-(C₁-C₁₀)-alkyl-N-(C₇-C₁₆)-aralkylcarbamoyloxy, N-((C₁-C₁₀)-alkyl)carbamoyloxy, N-((C₆-C₁₂)-aryloxy-(C₁-C₁₀)-alkyl)carbamoyloxy, N-((C₇-C₁₆)-aralkyloxy-(C₁-C₁₀)-alkyl)carbamoyloxy, N-(C₁-C₁₀)-alkyl-N-((C₁-C₁₀)-alkoxy-(C₁-C₁₀)-alkyl)carbamoyloxy, N-(C₁-C₁₀)-alkyl-N-((C₆-C₁₂)-aryloxy-(C₁-C₁₀)-alkyl)carbamoyloxy, N-(C₁-C₁₀)-alkyl-N-((C₇-C₁₆)-aralkyloxy-(C₁-C₁₀)-alkyl)carbamoyloxy, amino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N-(C₆-C₁₂)-arylamino, N-(C₇-C₁₁)-aralkylamino, N-alkyl-aralkylamino, N-alkyl-arylamino, (C₁-C₁₂)-alkoxyamino, (C₁-C₁₂)-alkoxy-N-(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N-(C₁-C₁₀)-alkylamino, (C₃-C₈)-cycloalkanoyl-N-(C₁-C₁₀)-alkylamino, (C₆-C₁₂)-aroyl-N-(C₁-C₁₀)-alkylamino, (C₇-C₁₁)-aralkanoyl-N-(C₁-C₁₀)-alkylamino, (C₁-C₁₂)-alkanoylamino-(C₁-C₈)-alkyl, (C₃-C₈)-cycloalkanoylamino-(C₁-C₈)-alkyl, (C₆-C₁₂)-aroylamino-(C₁-C₈)-alkyl, (C₇-C₁₆)-aralkanoylamino-(C₁-C₈)-alkyl, amino-(C₁-C₁₀)-alkyl, N-(C₁-C₁₀)-alkylamino-(C₁-C₁₀)-alkyl, N,N-di-(C₁-C₁₀)-alkylamino-(C₁-C₁₀)-alkyl, (C₃-C₈)-cycloalkylamino-(C₁-C₁₀)-alkyl, (C₁-C₁₂)-alkylmercapto, (C₁-C₁₂)-alkylsulfinyl, (C₁-C₁₂)-alkylsulfonyl, (C₆-C₁₆)-arylmercapto, (C₆-C₁₆)-arylsulfinyl, (C₆-C₁₆)-arylsulfonyl, (C₇-C₁₆)-aralkylmercapto, (C₇-C₁₆)-aralkylsulfinyl, or (C₇-C₁₆)-aralkylsulfonyl;

or wherein R¹ and R², or R² and R³ form a chain [CH₂]ₒ, which is saturated or unsaturated by a C.dbd.C double bond, in which 1 or 2 CH₂ groups are optionally replaced by O, S, SO, SO₂, or NR′, and R′ is hydrogen, (C₆-C₁₂)-aryl, (C₁-C₈)-alkyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₁-C₁₀)-alkanoyl, optionally substituted (C₇-C₁₆)-aralkanoyl, or optionally substituted (C₆-C₁₂)-aroyl; and o is 3, 4 or 5;

or wherein the radicals R¹ and R², or R² and R³, together with the pyridine or pyridazine carrying them, form a 5,6,7,8-tetrahydroisoquinoline ring, a 5,6,7,8-tetrahydroquinoline ring, or a 5,6,7,8-tetrahydrocinnoline ring;

or wherein R¹ and R², or R² and R³ form a carbocyclic or heterocyclic 5- or 6-membered aromatic ring;

or where R¹ and R², or R² and R³, together with the pyridine or pyridazine carrying them, form an optionally substituted heterocyclic ring system selected from thienopyridines, furanopyridines, pyridopyridines, pyrimidinopyridines, imidazopyridines, thiazolopyridines, oxazolopyridines, quinoline, isoquinoline, and cinnoline; where quinoline, isoquinoline or cinnoline preferably satisfy the formulae Ia, Ib and Ic:

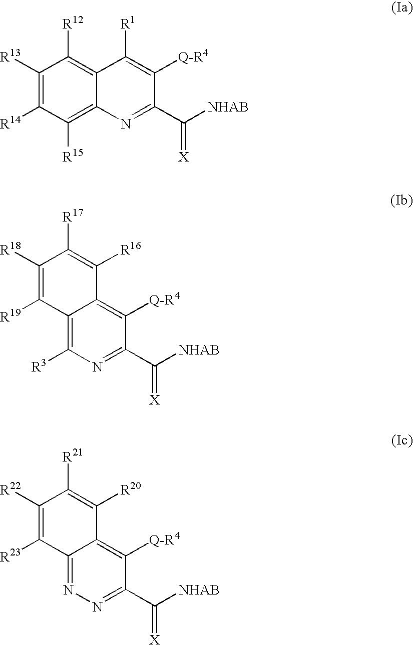

and the substituents R¹² to R²³ in each case independently of each other have the meaning of R¹, R² and R³;

or wherein the radicals R¹ and R², together with the pyridine carrying them, form a compound of Formula Id:

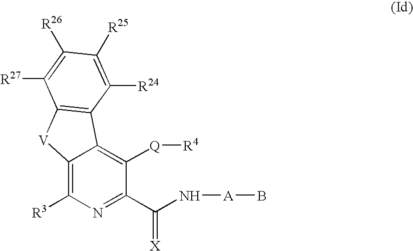

where V is S, O, or NR^{k}, and R^{k} is selected from hydrogen, (C₁-C₆)-alkyl, aryl, or benzyl; where an aryl radical may be optionally substituted by 1 to 5 substituents as defined above; and

R²4, R²5, R²6, and R²7 in each case independently of each other have the meaning of R¹, R² and R³;

f is 1 to 8;

g is 0 or 1 to (2f+1);

x is 0 to 3; and

h is 3 to 7;

including the physiologically active salts and prodrugs derived therefrom.

Exemplary compounds according to Formula (V) are described in European Patent Nos. EP0650960 and EP0650961. All compounds listed in EP0650960 and EP0650961, in particular, those listed in the compound claims and the final products of the working examples, are hereby incorporated into the present application by reference herein. Exemplary compounds of Formula (V) include, but are not limited to, [(3-Hydroxy-pyridine-2-carbonyl)-amino]-acetic acid (Compound G) and [(3-methoxy-pyridine-2-carbonyl)-amino]-acetic acid (Compound P).

Additionally, exemplary compounds according to Formula (V) are described in U.S. Pat. No. 5,658,933. All compounds listed in U.S. Pat. No. 5,658,933, in particular, those listed in the compound claims and the final products of the working examples, are hereby incorporated into the present application by reference herein in their entireties. Exemplary compounds of Formula (V) include, but are not limited to, 3-methoxypyridine-2-carboxylic acid N-(((hexadecyloxy)-carbonyl)-methyl)-amide hydrochloride, 3-methoxypyridine-2-carboxylic acid N-(((1-octyloxy)-carbonyl)-methyl)-amide, 3-methoxypyridine-2-carboxylic acid N-(((hexyloxy)-carbonyl)-methyl)-amide, 3-methoxypyridine-2-carboxylic acid N-(((butyloxy)-carbonyl)-methyl)-amide, 3-methoxypyridine-2-carboxylic acid N-(((2-nonyloxy)-carbonyl)-methyl)-amide racemate, 3-methoxypyridine-2-carboxylic acid N-(((heptyloxy)-carbonyl)-methyl)-amide, 3-benzyloxypyridine-2-carboxylic acid N-(((octyloxy)-carbonyl)-methyl)-amide, 3-benzyloxypyridine-2-carboxylic acid N-(((butyloxy)-carbonyl)-methyl)-amide, 5-(((3-(1-butyloxy)-propyl)-amino)-carbonyl)-3-methoxypyridine-2-carboxylic acid N-((benzyloxycarbonyl)-methyl)-amide, 5-(((3-(1-butyloxy)-propyl)-amino)-carbonyl)-3-methoxypyridine-2-carboxylic acid N-(((1-butyloxy)-carbonyl)-methyl)-amide, and 5-(((3-lauryloxy)-propyl)ammo)-carbonyl)-3-methoxypyridine-2-carboxylic acid N-(((benzyloxy)-carbonyl)-methyl)-amide.

Additional compounds according to Formula (V) are substituted heterocyclic carboxyamides described in U.S. Pat. No. 5,620,995; 3-hydroxypyridine-2-carboxamidoesters described in U.S. Pat. No. 6,020,350; sulfonamidocarbonylpyridine-2-carboxamides described in U.S. Pat. No. 5,607,954; and sulfonamidocarbonyl-pyridine-2-carboxamides and sulfonamidocarbonyl-pyridine-2-carboxamide esters described in U.S. Pat. Nos. 5,610,172 and 5,620,996. All compounds listed in these patents, in particular, those compounds listed in the compound claims and the final products of the working examples, are hereby incorporated into the present application by reference herein.

Exemplary compounds according to Formula (Ia) are described in U.S. Pat. Nos. 5,719,164 and 5,726,305. All compounds listed in the foregoing patents, in particular, those listed in the compound claims and the final products of the working examples, are hereby incorporated into the present application by reference herein. Exemplary compounds of Formula (Ia) include, but are not limited to, N-((3-hydroxy-6-isopropoxy-quinoline-2-carbonyl)-amino)-acetic acid (Compound H), N-((6-(1-butyloxy)-3-hydroxyquinolin-2-yl)-carbonyl)-glycine, [(3-hydroxy-6-trifluoromethoxy-quinoline-2-carbonyl)-amino]-acetic acid (Compound I), N-((6-chloro-3-hydroxyquinolin-2-yl)-carbonyl)-glycine, N-((7-chloro-3-hydroxyquinolin-2-yl)-carbonyl)-glycine, and [(6-chloro-3-hydroxy-quinoline-2-carbonyl)-amino]-acetic acid (Compound O).

Exemplary compounds according to Formula (Ib) are described in U.S. Pat. No. 6,093,730. All compounds listed in U.S. Pat. No. 6,093,730, in particular, those listed in the compound claims and the final products of the working examples, are hereby incorporated into the present application by reference herein. Exemplary compounds of Formula (Ib) include, but are not limited to, N-((1-chloro-4-hydroxy-7-(2-propyloxy) isoquinolin-3-yl)-carbonyl)-glycine, N-((1-chloro-4-hydroxy-6-(2-propyloxy) isoquinolin-3-yl)-carbonyl)-glycine, N-((1-chloro-4-hydroxy-isoquinoline-3-carbonyl)-amino)-acetic acid (Compound B), N-((1-chloro-4-hydroxy-7-methoxyisoquinolin-3-yl)-carbonyl)-glycine, N-((1-chloro-4-hydroxy-6-methoxy-isoquinolin-3-yl)-carbonyl)-glycine, N-((7-butyloxy)-1-chloro-4-hydroxyisoquinolin-3-yl)-carbonyl)-glycine, N-((6-benzyloxy-1-chloro-4-hydroxy-isoquinoline-3-carbonyl)-amino)-acetic acid (Compound J), ((7-benzyloxy-1-chloro-4-hydroxy-isoquinoline-3-carbonyl)-amino)-acetic acid methyl ester (Compound K), N-((7-benzyloxy-1-chloro-4-hydroxy-isoquinoline-3-carbonyl)-amino)-acetic acid (Compound L), N-((8-chloro-4-hydroxyisoquinolin-3-yl)-carbonyl)-glycine, N-((7-butoxy-4-hydroxy-isoquinoline-3-carbonyl)-amino)-acetic acid (Compound M).

Additionally, compounds related to Formula (V) that can also be used in the methods of the invention include, but are not limited to, 6-cyclohexyl-1-hydroxy-4-methyl-1H-pyridin-2-one (Compound N), 7-(4-methyl-piperazin-1-ylmethyl)-5-phenylsulfanylmethyl-quinolin-8-ol (Compound D), 4-nitro-quinolin-8-ol (Compound E), and 5-butoxymethyl-quinolin-8-ol (Compound F). Further, the invention provides additional exemplary compounds wherein, e.g., position A and B together may be, e.g., hexanoic acid, cyanomethyl, 2-aminoethyl, benzoic acid, 1H-benzoimidazol-2-ylmethyl, etc.

In other embodiments, compounds used in the methods of the invention are selected from a compound of the formula (II)

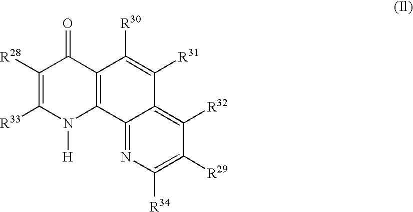

where

R²⁸ is hydrogen, nitro, amino, cyano, halogen, (C₁-C₄)-alkyl, carboxy or a metabolically labile ester derivative thereof; (C₁-C₄)-alkylamino, di-(C₁-C₄)-alkylamino, (C₁-C₆)-alkoxycarbonyl, (C₂-C₄)-alkanoyl, hydroxy-(C₁-C₄)-alkyl, carbamoyl, N-(C₁-C₄)-alkylcarbamoyl, (C₁-C₄)-alkylthio, (C₁-C₄)-alkylsulfinyl, (C₁-C₄)-alkylsulfonyl, phenylthio, phenylsulfinyl, phenylsulfonyl, said phenyl or phenyl groups being optionally substituted with 1 to 4 identical or different halogen, (C₁-C₄)-alkyoxy, (C₁-C₄)-alkyl, cyano, hydroxy, trifluoromethyl, fluoro-(C₁-C₄)-alkylthio, fluoro-(C₁-C₄)-alkylsulfinyl, fluoro-(C₁-C₄)-alkylsulfonyl, (C₁-C₄)-alkoxy-(C₂-C₄)-alkoxycarbonyl, N,N-di-[(C₁-C₄)-alkyl]carbamoyl-(C₁-C₄)-alkoxycarbonyl, (C₁-C₄)-alkylamino-(C₂-C₄)-alkoxycarbonyl, di-(C₁-C₄)-alkylamino-(C₂-C₄)-alkoxycarbonyl, (C₁-C₄)-alkoxy-(C₂-C₄)-alkoxy-(C₂-C₄)-alkoxycarbonyl, (C₂-C₄)-alkanoyloxy-C₁-C₄)-alkyl, or N-[amino-(C₂-C₈)-alkyl]-carbamoyl;

R²⁹ is hydrogen, hydroxy, amino, cyano, halogen, (C₁-C₄)-alkyl, carboxy or metabolically labile ester derivative thereof, (C₁-C₄)-alkylamino, di-(C₁-C₄)-alkylamino, (C₁-C₆)-alkoxycarbonyl, (C₂-C₄)-alkanoyl, (C₁-C₄)-alkoxy, carboxy-(C₁-C₄)-alkoxy, (C₁-C₄)-alkoxycarbonyl-(C₁-C₄)-alkoxy, carbamoyl, N-(C₁-C₈)-alkylcarbamoyl, N,N-di-(C₁-C₈)-alkylcarbamoyl, N-[amino-(C₂-C₈)-alkyl)-carbamoyl, N-[(C₁-C₄)-alkylamino-(C₁-C₈)-alkyl]-carbamoyl, N-[di-(C₁-C₄)-alkylamino-(C₁-C₈)-alkyl)]-carbamoyl, N-cyclohexylcarbamoyl, N-[cyclopentyl]-carbamoyl, N-(C₁-C₄)-alkylcyclohexylcarbamoyl, N-(C₁-C₄)-alkylcyclopentylcarbamoyl, N-phenylcarbamoyl, N-(C₁-C₄)-alkyl-N-phenylcarbamoyl, N,N-diphenylcarbamoyl, N-[phenyl-(C₁-C₄)-alkyl]-carbamoyl, N-(C₁-C₄)-alkyl-N-[phenyl-(C₁-C₄)-alkyl]-carbamoyl, or N,N-di-[phenyl-(C₁-C₄)-alkyl]-carbamoyl, said phenyl or phenyl groups being optionally substituted with 1 to 4 identical different halogen, (C₁-C₄)-alkyoxy, (C₁-C₄)-alkyl, cyano, hydroxy, trifluoromethyl, N-[(C₂-C₄)-alkanoyl]-carbamoyl, N-[(C₁-C₄)-alkoxycarbonyl]-carbamoyl, N-[fluoro-(C₂-C₆)-alkyl]-carbamoyl, N,N-[fluoro-(C₂-C₆)-alkyl]-N-(C₁-C₄)-alkylcarbamoyl, N,N-[di-fluoro-(C₂-C₆)-alkyl]carbamoyl, pyrrolidin-1-ylcarbonyl, piperidinocarbonyl, piperazin-1-ylcarbonyl, morpholinocarbonyl, wherein the heterocyclic group, is optionally substituted with 1 to 4, (C₁-C₄)-alkyl, benzyl, 1,2,3,4-tetrahydro-isoquinolin-2-ylcarbonyl, N,N-[di-(C₁-C₄)-alkyl]-thiocarbamoyl, N-(C₂-C₄)-alkanoylamino, or N-[(C₁-C₄)-alkoxycarbonyl]-amino;

R³⁰ is hydrogen, (C₁-C₄)-alkyl, (C₂-C₄)-alkoxy, halo, nitro, hydroxy, fluoro-(14C)alkyl, or pyridinyl;

R³¹ is hydrogen, (C₁-C₄)-alkyl, (C₂-C₄)-alkoxy, halo, nitro, hydroxy, fluoro-(C₁-C₄)-alkyl, pyridinyl, or methoxy;

R³² is hydrogen, hydroxy, amino, (C₁-C₄)-alkylamino, di-(C₁-C₄)-alkylamino, halo, (C₁-C₄)-alkoxy-(C₂-C₄)-alkoxy, fluoro-(C₁-C₆)-alkoxy, pyrrolidin-1-yl, piperidino, piperazin-1-yl, or morpholino, wherein the heterocyclic group is optionally substituted with 1 to 4 identical or different (C₁-C₄)-alkyl or benzyl; and

R³³ and R³⁴ are individually selected from hydrogen, (C₁-C₄)-alkyl, and (C₁-C₄)-alkoxy; including pharmaceutically-acceptable salts and pro-drugs derived therefrom.

Exemplary compounds of Formula (II) are described in U.S. Pat. Nos. 5,916,898 and 6,200,974, and International Publication No. WO 99/21860. All compounds listed in the foregoing patents and publication, in particular, those listed in the compound claims and the final products of the working examples, are hereby incorporated into the present application by reference herein. Exemplary compounds of Formula (II) include 4-oxo-1,4-dihydro-[1,10]phenanthroline-3-carboxylic acid (Compound A) (see, e.g., Seki et al. (1974)), 3-carboxy-5-hydroxy-4-oxo-3,4-dihydro-1,10-phenanthroline, 3-carboxy-5-methoxy-4-oxo-3,4-dihydro-1,10-phenanthroline, 5-methoxy-4-oxo-1,4-dihydro-[1,10]phenanthroline-3-carboxylic acid ethyl ester, 5-methoxy-4-oxo-1,4-dihydro-[1,10]phenanthroline-3-carboxylic acid (Compound Q), and 3-carboxy-8-hydroxy-4-oxo-3,4-dihydro-1,10-phenanthroline.

In other embodiments, compounds used in the methods of the invention are selected from a compound of the formula (III)

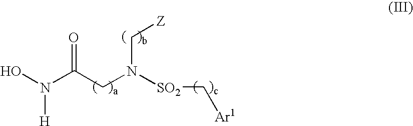

or pharmaceutically acceptable salts thereof, wherein:

a is an integer from 1 to 4;

b is an integer from 0 to 4;

c is an integer from 0 to 4;

Z is selected from the group consisting of (C₃-C₁₀) cycloalkyl, (C₃-C₁₀) cycloalkyl independently substituted with one or more Y¹, 3-10 membered heterocycloalkyl and 3-10 membered heterocycloalkyl independently substituted with one or more Y¹; (C₅-C₂₀) aryl, (C₅-C₂₀) aryl independently substituted with one or more Y¹, 5-20 membered heteroaryl and 5-20 membered heteroaryl independently substituted with one or more Y¹;

Ar¹ is selected from the group consisting of (C₅-C₂₀) aryl, (C₅-C₂₀) aryl independently substituted with one or more Y¹, 5-20 membered heteroaryl and 5-20 membered heteroaryl independently substituted with one or more Y¹;

each Y¹ is independently selected from the group consisting of a lipophilic functional group, (C₅-C₂0) aryl, (C₆-C₂6) alkaryl, 5-20 membered heteroaryl and 6-26 membered alk-heteroaryl;

each Y² is independently selected from the group consisting of —R′, —OR′, —OR″, —SR′, —SR″, —NR′R′, —NO₂, —CN, -halogen, -trihalomethyl, trihalomethoxy, —C(O)R′, —C(O)OR′, —C(O)NR′R′, —C(O)NR′OR′, —C(NR′R′).dbd.NOR′, —NR′—C(O)R′, —SO₂R′, —SO₂R″, —NR′—SO₂—R′, —NR′—C(O)—NR′R′, tetrazol-5-yl, —NR′—C(O)—OR′, —C(NR′R′).dbd.NR′, —S(O)—R′, —S(O)—R″, and —NR′—C(S)—NR′R′; and

each R′ is independently selected from the group consisting of —H, (C₁-C₈) alkyl, (C₂-C₈) alkenyl, and (C₂-C₈) alkynyl; and

each R″ is independently selected from the group consisting of (C₅-C₂₀) aryl and (C₅-C₂₀) aryl independently substituted with one or more —OR′, —SR′, —NR′R′, —NO₂, —CN, halogen or trihalomethyl groups,

or wherein c is 0 and Ar¹ is an N′ substituted urea-aryl, the compound has the structural formula (IIIa):

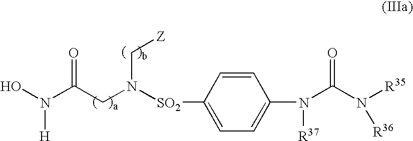

or pharmaceutically acceptable salts thereof, wherein:

a, b, and Z are as defined above; and

R³⁵ and R³⁶ are each independently selected from the group consisting of hydrogen, (C₁-C₈) alkyl, (C₂-C₈) alkenyl, (C₂-C₈) alkynyl, (C₃-C₁₀) cycloalkyl, (C₅-C₂0) aryl, (C₅-C₂0) substituted aryl, (C₆-C₂6) alkaryl, (C₆-C₂6) substituted alkaryl, 5-20 membered heteroaryl, 5-20 membered substituted heteroaryl, 6-26 membered alk-heteroaryl, and 6-26 membered substituted alk-heteroaryl; and

R³⁷ is independently selected from the group consisting of hydrogen, (C₁-C₈) alkyl, (C₂-C₈) alkenyl, and (C₂-C₈) alkynyl.

Exemplary compounds of Formula (III) are described in International Publication No. WO 00/50390. All compounds listed in WO 00/50390, in particular, those listed in the compound claims and the final products of the working examples, are hereby incorporated into the present application by reference herein. Exemplary compounds of Formula (III) include 3-{[4-(3,3-dibenzyl-ureido)-benzenesulfonyl]-[2-(4-methoxy-phenyl)-ethyl]-amino}-N-hydroxy-propionamide (Compound C), 3-{{4-[3-(4-chloro-phenyl)-ureido]-benzenesulfonyl}-[2-(4-methoxy-phenyl)-ethyl]-amino}-N-hydroxy-propionamide, and 3-{{4-[3-(1,2-diphenyl-ethyl)-ureido]-benzenesulfonyl}-[2-(4-methoxy-phenyl)-ethyl]-amino}-N-hydroxy-propionamide.

In one of its compound aspects, there is provided compounds represented by formula VI:

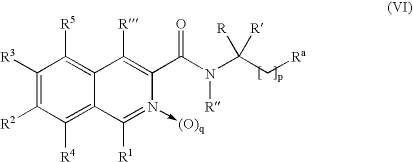

wherein:

q is zero or one;

p is zero or one;

R^{a} is —COOH or —WR⁸; provided that when R^{a} is —COOH then p is zero and when R^{a} is —WR⁸ then p is one;

W is selected from the group consisting of oxygen, —S(O)ₙ— and —NR⁹— where n is zero, one or two, R⁹ is selected from the group consisting of hydrogen, alkyl, substituted alkyl, acyl, aryl, substituted aryl, heteroaryl, substituted heteroaryl, heterocyclic and substituted heterocyclic and R⁸ is selected from the group consisting of hydrogen, alkyl, substituted alkyl, aryl, substituted aryl, heteroaryl, substituted heteroaryl, heterocyclic and substituted heterocyclic, or when W is —NR⁹— then R⁸ and R⁹, together with the nitrogen atom to which they are bound, can be joined to form a heterocyclic or a substituted heterocyclic group, provided that when W is —S(O)ₙ— and n is one or two, then R⁸ is not hydrogen;

R¹ is selected from the group consisting of hydrogen, alkyl, substituted alkyl, alkoxy, substituted alkoxy, amino, substituted amino, aminoacyl, aryl, substituted aryl, halo, heteroaryl, substituted heteroaryl, heterocyclic, substituted heterocyclic, and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl or, when X is —NR⁷—, then R⁷ and R⁸, together with the nitrogen atom to which they are bound, can be joined to form a heterocyclic or substituted heterocyclic group;

R² and R³ are independently selected from the group consisting of hydrogen, alkyl, substituted alkyl, aryl, substituted aryl, heteroaryl, substituted heteroaryl, halo, hydroxy, cyano, —S(O)ₙ—(R⁶)—R⁶ where n is 0, 1, or 2, —NR⁶C(O)NR⁶R⁶, —XR⁶ where X is oxygen, —S(O)ₙ— or —NR⁷— where n is zero, one or two, each R⁶ is independently selected from the group consisting of hydrogen, alkyl, substituted alkyl, aryl, substituted aryl, cycloalkyl, substituted cycloalkyl, heteroaryl, substituted heteroaryl, heterocyclic and substituted heterocyclic provided that when X is —SO— or —SO₂—, then R₆ is not hydrogen, and R⁷ is selected from the group consisting of hydrogen, alkyl, aryl, or R², R³ together with the carbon atom pendent thereto, form an aryl substituted aryl, heteroaryl, or substituted heteroaryl;

R⁴ and R⁵ are independently selected from the group consisting of hydrogen, halo, alkyl, substituted alkyl alkoxy, substituted alkoxy, aryl, substituted aryl, heteroaryl, substituted heteroaryl and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl or, when X is —NR⁷—, then R⁷ and R⁸, together with the nitrogen atom to which they are bound, can be joined to form a heterocyclic or substituted heterocyclic group;

R is selected from the group consisting of hydrogen, deuterium and methyl;

R′ is selected from the group consisting of hydrogen, deuterium, alkyl and substituted alkyl; alternatively, R and R′ and the carbon pendent thereto can be joined to form cycloalkyl, substituted cycloalkyl, heterocyclic or substituted heterocyclic group;

R″ is selected from the group consisting of hydrogen and alkyl or R″ together with R′ and the nitrogen pendent thereto can be joined to form a heterocyclic or substituted heterocyclic group;

R′″ is selected from the group consisting of hydroxy, alkoxy, substituted alkoxy, acyloxy, cycloalkoxy, substituted cycloalkoxy, aryloxy, substituted aryloxy, heteroaryloxy, substituted heteroaryloxy, aryl, —S(O)ₙ—R¹⁰ wherein R¹⁰ is selected from the group consisting of alkyl, substituted alkyl, cycloalkyl, substituted cycloalkyl, aryl, substituted aryl, heteroaryl and substituted heteroaryl and n is zero, one or two;

and pharmaceutically acceptable salts, esters and prodrugs thereof;

with the proviso that when R, R′ and R″ are hydrogen and q is zero, and R^{a} is either —COOH (p is zero) or —WR⁸ (p is one) and W is oxygen and R⁸ is hydrogen then at least one of the following occurs:

1) R¹ is fluoro, bromo, iodo, alkyl, substituted alkyl, alkoxy, aminoacyl, substituted alkoxy, aryl, substituted aryl, heteroaryl, substituted heteroaryl, heterocyclic, substituted heterocyclic, and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl; or

2) R² is substituted alkyl, aryl, substituted aryl, heteroaryl, substituted heteroaryl, fluoro, bromo, iodo, cyano, —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl provided that:

a) when R² is substituted alkyl such a substituent does not include trifluoromethyl;

b) —XR⁶ is not alkoxy; and

c) when —XR⁶ is substituted alkoxy such a substituent does not include benzyl or benzyl substituted by a substituent selected from the group consisting of (C₁-C₅)alkyl and (C₁-C₅)alkoxy or does not include a fluoroalkoxy substituent of the formula:

—O—[CH₂]ₓ—C_{f}H_{(2f+1-g)}F_{g}

where x is zero or one; .function. is an integer of from 1 to 5; and g is an integer of from 1 to (2.function.+1); or

3) R³ is substituted alkyl, aryl, substituted aryl, heteroaryl, substituted heteroaryl, bromo, iodo, —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl provided that:

a) when R³ is substituted alkyl such a substituent does not include trifluoromethyl;

b) —XR⁶ is not alkoxy; and

c) when —XR⁶ is substituted alkoxy such a substituent does not include benzyl or benzyl substituted by a substituent selected from the group consisting of (C₁-C₅)alkyl and (C₁-C₅)alkoxy or does not include a fluoroalkoxy substituent of the formula:

—O—[CH₂]ₓ—C_{f}H_{(2.function.+1-g)}F_{g}

where x is zero or one; .function. is an integer of from 1 to 5; and g is an integer of from 1 to (2.function.+1); or

4) R⁴ is iodo, substituted alkyl, aryl, substituted aryl, heteroaryl, substituted heteroaryl, —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl provided that:

a) when R⁴ is substituted alkyl such a substituent does not include trifluoromethyl;

b) —XR⁶ is not alkoxy; and

c) when —XR⁶ is substituted alkoxy such a substituent does not include a fluoroalkoxy substituent of the formula:

—O—[CH₂]ₓ—C_{f}H_{(2.function.+1-g)}F_{g}

where x is zero or one; .function. is an integer of from 1 to 5; and g is an integer of from 1 to (2.function.+1); or

5) R⁵ is iodo, substituted alkyl, aryl, substituted aryl, heteroaryl, substituted heteroaryl, —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl provided that:

a) when R⁵ is substituted alkyl such a substituent does not include trifluoromethyl;

b) —XR⁶ is not alkoxy; and

c) when —XR⁶ is substituted alkoxy such a substituent does not include a fluoroalkoxy substituent of the formula:

—O—[CH₂]ₓ—C_{f}H_{(2.function.+1-g)}F_{g}

where x is zero or one; .function. is an integer of from 1 to 5; and g is an integer of from 1 to (2.function.+1);

and with the further following proviso:

that when R¹, R³, R⁴, and R⁵ are hydrogen, then R² is not bromo.

In an alternative embodiment, the compounds of formula VI are represented by formula VII:

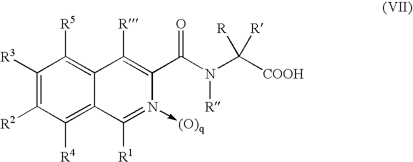

wherein R¹, R², R³, R⁴, R⁵, R, R′, R″, R′″ and q are as defined above; and

pharmaceutically acceptable salts, esters, prodrugs thereof.

In an another alternative embodiment, the compounds of formula VI are represented by the formula VIII:

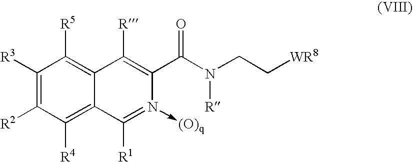

wherein R¹, R², R³, R⁴, R⁵, R″, R′″, WR⁸ and q are as defined above; and

pharmaceutically acceptable salts, esters, prodrugs thereof.

In an another alternative embodiment, the invention is directed to compounds represented by the formula IX:

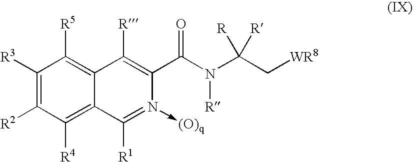

wherein R¹, R², R³, R⁴, R⁵, R, R′, R″, R′″, WR⁸ and q are as defined above; and

pharmaceutically acceptable salts, esters, prodrugs thereof.

In yet another alternative embodiment, the invention is directed to compounds represented by the formula X:

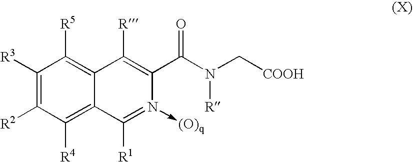

wherein R¹, R², R³, R⁴, R⁵, R, R′, R″, R′″ and q are as defined above; and

pharmaceutically acceptable salts, esters, prodrugs thereof.

In compounds of formulae VI, VII, VIII, IX, and X, R¹ may be selected from the group consisting of hydrogen, alkyl, substituted alkyl, halo, alkoxy, aryloxy, substituted aryloxy, substituted aryl, alkylthio, aminoacyl, aryl, substituted amino, heteroaryl, heteroaryloxy, —S(O)ₙ-aryl, —S(O)ₙ-substituted aryl, —S(O)ₙ-heteroaryl-, and —S(O)ₙ-substituted heteroaryl, where n is zero, one or two.

For example, R¹ may be selected from the group consisting of: (3-methoxyphenyl)sulfanyl; (4-chlorophenyl)sulfanyl; (4-methylphenyl)sulfanyl; 2-fluorophenoxy; 2-methoxyphenoxy; (2-methoxyphenyl)sulfanyl 3-fluorophenoxy; 3-methoxyphenoxy; 4-(methylcarbonylamino)phenoxy; 4-(methylsulfonamido)phenoxy; 4-fluorophenoxy; 4-methoxyphenoxy; 4-methoxyphenylsulfanyl; 4-methylphenyl; bromo; chloro; dimethylaminomethyl; ethoxy; ethylsulfanyl; hydrogen; isopropyl; methoxy; methoxymethyl; methyl; N,N-dimethylaminocarbonyl; naphth-2-yloxy; naphthylsulfanyl; phenoxy; phenyl; phenylamino; phenylsulfinyl; phenylsulfanyl; pyridin-2-yloxy; pyridin-2-yl; and pyridin-2-ylsulfanyl.

In compounds of formulae VI, VII, VIII, IX, and X, R² may be selected from the group consisting of substituted amino, aryloxy, substituted aryloxy, alkoxy, substituted alkoxy, halo, hydrogen, alkyl, substituted alkyl, aryl, —S(O)ₙ-aryl, —S(O)ₙ-substituted aryl, —S(O)ₙ-cycloalkyl, where n is zero, one or two, aminocarbonylamino, heteroaryloxy, and cycloalkyloxy.

For example, R² may be selected from the group consisting of: (4-methoxy)phenylsulfonylamino; 2,6-dimethylphenoxy; 3,4-difluorophenoxy; 3,5-difluorophenoxy; 3-chloro-4-fluorophenoxy; 3-methoxy-4-fluorophenoxy; 3-methoxy-5-fluorophenoxy; 4-(methylsulfonamido)phenoxy; 4-(phenylsulfonamido)phenoxy; 4-CF₃—O-phenoxy; 4-CF₃-phenoxy; 4-chlorophenoxy; 4-fluorophenoxy; 4-(4-fluorophenoxy)phenoxy; 4-methoxyphenoxy; 4-nitrophenoxy; benzyloxy; bromo; butoxy; CF₃; chloro; cyclohexyloxy; cyclohexylsulfanyl; cyclohexylsulfonyl; fluoro; hydrogen; iodo; isopropoxy; methyl; phenoxy; phenyl; phenylsulfanyl; phenylsulfinyl; phenylsulfonyl; phenylurea; pyridin-1-ylsulfanyl; pyridin-3-yloxy; and pyridin-4-ylsulfanyl.

In compounds of formulae VI, VII, VIII, IX, and X, R³ may be selected from the group consisting of: substituted aryloxy, substituted alkoxy, alkoxy, substituted alkyl, alkyl, amino, cycloalkyloxy, hydrogen, halo, aryl, —S(O)ₙ-aryl, —S(O)ₙ-substituted aryl, —S(O)ₙ-heteroaryl, and —S(O)ₙ-substituted heteroaryl, where n is zero, one or two, aminocarbonylamino, and heteroaryloxy.

For example, R³ may be selected from the group consisting of: amino; (4-methyl)phenylsulfonylaminophenoxy; 3,4-difluorophenoxy; 3,5-difluorophenoxy; 3-fluoro-5-methoxy-phenoxy; 3-chloro-4-fluorophenoxy; 4-CF₃—O-phenoxy; 4-CF₃-phenoxy; 4-chlorophenoxy; 4-fluorophenoxy; 4-(4-fluorophenoxy)phenoxy; 4-methoxyphenoxy; benzyloxy; bromo; butoxy; CF₃; chloro; cyclohexyloxy; hydrogen; iodo; isopropoxy; phenoxy; phenyl; phenylsulfanyl; phenylsulfonyl; phenylsulfinyl; phenylurea; pyridin-1-ylsulfanyl; pyridin-3-yloxy; and pyridin-4-ylsulfanyl.

Alternatively, R² and R³, combined with the carbon atoms pendent thereto, are joined to form an aryl group, such as phenyl.

In compounds of formulae VI, VII, VIII, IX, and X, R⁴ may be selected from the group consisting of: substituted arylthio, halo, hydrogen, substituted alkyl and aryl.

For example, R⁴ may be selected from the group consisting of: 4-chlorophenyl sulfanyl; chloro; hydrogen; methoxymethyl; and phenyl.

In compounds of formulae VI, VII, VIII, IX, and X, R⁵ may be hydrogen or aryl, such as phenyl.

In compounds of formulae VI, VII, and IX, R may, in some embodiments, be selected from the group consisting of hydrogen, deuterium, aryl and alkyl. For example, R may be selected from the group consisting of phenyl, hydrogen, deuterium and methyl.

In compounds of formulae VI, VII, and IX, R′ may be selected from the group consisting of hydrogen, deuterium, alkyl, substituted alkyl, and substituted amino. For example, R′ may be selected from the group consisting of: 4-aminobutyl; 4-hydroxybenzyl; benzyl; carboxylmethyl; deuterium; hydroxymethyl; imidazol-4-ylmethyl; isopropyl; methyl; and propyl.

Alternatively, R, R′ and the carbon atom pendent thereto join to form a cycloalkyl, such as cyclopropyl.

In compounds of formulae VI, VII, and IX, R″ may be hydrogen, alkyl or substituted alkyl. For example, R″ may be hydrogen, methyl or carboxylmethyl (—CH₂C(O)OH). Alternatively, R′, R″ and the carbon atom and nitrogen atom respectively pendent thereto join to form a heterocyclic group, such as pyrrolidinyl.

In compounds of formulae VI, VII, VIII, IX, and X, R″ may be selected from the group consisting of hydrogen, hydroxy, alkoxy, substituted alkoxy, cycloalkoxy, substituted cycloalkoxy, thiol, acyloxy and aryl. For example, R′″ is selected from the group consisting of: hydroxy; benzyloxy; ethoxy; thiol; methoxy; methylcarbonyloxy; and phenyl.

In compounds of formulae VI, VIII, and IX, WR⁸ may be selected from the group consisting of amino, substituted amino, aminoacyl, hydroxy, and alkoxy. For example, WR⁸ may be selected from the group consisting of: amino; dimethylamino; hydroxy; methoxy; and methylcarbonylamino.

Representative compounds for this application are presented in Tables A-D, wherein said table letter corresponds to formula letter (i.e., representative compounds of Formula VI are in Table A).

TABLE A

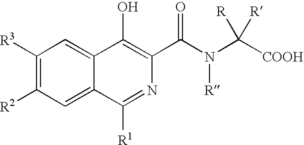

| No. | R¹ | R² | R³ | R | R′ | R″ |
|---|---|---|---|---|---|---|
| 1 | Cl | H | benzyloxy | H | methyl | H |
| 2 | Cl | H | H | H | hydroxymethyl | H |
| 3 | Cl | H | H | H | hydroxymethyl | H |
| 4 | Cl | H | isopropoxy | H | hydroxymethyl | H |
| 5 | Cl | H | isopropoxy | H | hydroxymethyl | H |
| 6 | Cl | isopropoxy | H | H | hydroxymethyl | H |
| 7 | Cl | isopropoxy | H | H | hydroxymethyl | H |
| 8 | Cl | H | H | methyl | methyl | H |
| 9 | Cl | H | isopropoxy | methyl | methyl | H |
| 10 | Cl | H | H | H | imidazol-4- | H |
| | | | | | ylmethyl | |
| 11 | Cl | H | H | H | imidazol-4- | H |
| | | | | | ylmethyl | |
| 12 | Cl | H | H | H | isopropyl | H |
| 13 | Cl | H | H | H | isopropyl | H |
| 14 | Cl | H | isopropoxy | H | isopropyl | H |
| 15 | Cl | H | isopropoxy | H | isopropyl | H |
| 16 | Cl | isopropoxy | H | H | isopropyl | H |
| 17 | Cl | isopropoxy | H | H | isopropyl | H |
| 18 | Cl | H | benzyloxy | H | isopropyl | H |
| 19 | Cl | H | H | H | benzyl | H |
| 20 | Cl | H | H | H | benzyl | H |
| 21 | Cl | H | isopropoxy | H | benzyl | H |
| 22 | Cl | H | isopropoxy | H | benzyl | H |
| 23 | Cl | isopropoxy | H | H | benzyl | H |
| 24 | Cl | isopropoxy | H | H | benzyl | H |
| 25 | Cl | H | H | H | 4-hydroxybenzyl | H |
| 26 | Cl | H | H | H | 4-hydroxybenzyl | H |
| 27 | Cl | H | isopropoxy | H | 4-hydroxybenzyl | H |
| 28 | Cl | H | isopropoxy | H | 4-hydroxybenzyl | H |
| 29 | Cl | isopropoxy | H | H | 4-hydroxybenzyl | H |
| 30 | Cl | isopropoxy | H | H | 4-hydroxybenzyl | H |
| 31 | Cl | H | isopropoxy | H | propyl | H |
| 32 | Cl | H | isopropoxy | H | propyl | H |
| 33 | Cl | H | H | H | R′ and R″ and the | — |
| | | | | | carbon and | |
| | | | | | nitrogen atom | |
| | | | | | respectively | |
| | | | | | pendent to which | |
| | | | | | R″ is attached join | |
| | | | | | to form a | |
| | | | | | pyrrolidinyl | |
| 34 | Cl | H | H | H | R′ and R″ and the | — |
| | | | | | carbon and | |
| | | | | | nitrogen atom | |
| | | | | | respectively | |
| | | | | | pendent to which | |
| | | | | | R″ is attached join | |
| | | | | | to form a | |
| | | | | | pyrrolidinyl | |
| 35 | Cl | H | isopropoxy | H | R′ and R″ and the | — |
| | | | | | carbon and | |
| | | | | | nitrogen atom | |
| | | | | | respectively | |
| | | | | | pendent to which | |
| | | | | | R″ is attached join | |
| | | | | | to form a | |
| | | | | | pyrrolidinyl | |
| 36 | Cl | H | isopropoxy | H | R′ and R″ and the | — |
| | | | | | carbon and | |
| | | | | | nitrogen atom | |
| | | | | | respectively | |
| | | | | | pendent to which | |
| | | | | | R″ is attached join | |
| | | | | | to form a | |
| | | | | | pyrrolidinyl | |
| 37 | Cl | H | H | H | 4-aminobutyl | H |
| 38 | Cl | H | H | H | 4-aminobutyl | H |
| 39 | Cl | H | isopropoxy | H | 4-aminobutyl | H |
| 40 | Cl | H | isopropoxy | H | 4-aminobutyl | H |
| 41 | Cl | isopropoxy | H | H | 4-aminobutyl | H |
| 42 | Cl | isopropoxy | H | H | 4-aminobutyl | H |
| 43 | Cl | H | H | H | carboxymethyl | H |
| 44 | Cl | H | H | H | carboxymethyl | H |
| 45 | Cl | H | isopropoxy | H | carboxylmethyl | H |
| 46 | Cl | H | isopropoxy | H | carboxylmethyl | H |
| 47 | Cl | isopropoxy | H | H | carboxylmethyl | H |
| 48 | Cl | H | H | — | R, R′ together with | H |
| | | | | | the carbon by which | |
| | | | | | they are attached | |
| | | | | | join to form | |
| | | | | | cyclopropyl | |
| 49 | Cl | H | isopropoxy | — | R, R′ together with | H |
| | | | | | the carbon to which | |
| | | | | | they are attached | |
| | | | | | join to form | |
| | | | | | cyclopropyl | |
| 50 | Cl | H | H | O | O | H |
| 51 | Cl | H | benzyloxy | H | methyl | H |
| 52 | Cl | benzyloxy | H | H | methyl | H |
| 53 | Cl | benzyloxy | H | H | methyl | H |
| 54 | Cl | H | H | H | methyl | H |
| 55 | Cl | H | H | H | methyl | H |
| 56 | Cl | H | isopropoxy | H | methyl | H |
| 57 | Cl | H | isopropoxy | H | methyl | H |
| 58 | Cl | isopropoxy | H | H | methyl | H |
| 59 | Cl | isopropoxy | H | H | methyl | H |
| 60 | H | 4-chlorophenoxy | H | H | methyl | H |
| 61 | H | H | 4-chlorophenoxy | H | methyl | H |
| 62 | H | 3,4-difluorophenoxy | H | H | methyl | H |
| 63 | H | phenylsulfanyl | H | H | methyl | H |
| 64 | H | phenylsulfanyl | H | H | methyl | H |
| 65 | H | phenoxy | H | H | methyl | H |
| 66 | H | 4-methoxyphenoxy | H | H | methyl | H |
| 67 | H | phenylsulfanyl | H | H | methyl | H |
| 68 | methoxymethyl | phenoxy | H | H | methyl | H |
| 69 | methoxymethyl | phenoxy | H | H | methyl | H |
| 70 | H | phenoxy | H | H | methyl | H |
| 71 | 4-chlorophenyl | H | H | H | methyl | H |
| | sulfanyl | | | | | |
| 72 | 4-chlorophenyl | H | H | H | methyl | H |
| | sulfanyl | | | | | |
| 73 | H | 3-methoxy-4- | H | H | methyl | H |
| | | fluorophenoxy | | | | |
| 74 | H | cyclohexyloxy | H | H | methyl | H |
| 75 | methyl | 4-fluorophenoxy | H | H | methyl | H |
| 76 | H | 4-fluorophenoxy | H | H | methyl | H |
| 77 | methyl | phenoxy | H | H | methyl | H |
| 78 | methyl | phenylsulfanyl | H | H | methyl | H |
| 79 | H | 4-trifluoromethyl- | H | H | methyl | H |
| | | phenoxy | | | | |

TABLE B

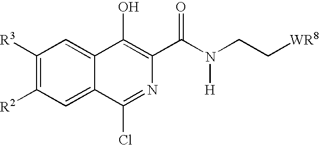

| No. | R² | R³ | WR⁸ |
|---|---|---|---|
| 1 | H | H | methoxy |
| 2 | isopropoxy | H | amino |
| 3 | H | isopropoxy | methoxy |
| 4 | H | H | amino |
| 5 | H | H | hydroxy |
| 6 | H | isopropoxy | hydroxy |
| 7 | H | H | dimethylamino |
| 8 | H | H | methylcarbonylamino |
| 9 | H | isopropoxy | amino |
| 10 | H | isopropoxy | dimethylamino |
| 11 | isopropoxy | H | methoxy |
| 12 | isopropoxy | H | dimethylamino |
| 13 | isopropoxy | H | hydroxy |

TABLE C

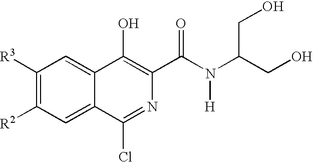

| No. | R² | R³ |
|---|---|---|
| 1 | isopropoxy | H |
| 2 | H | isopropoxy |
| 3 | H | H |

TABLE D

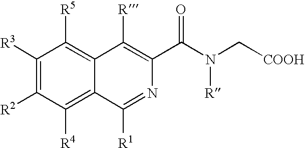

| No. | R¹ | R² | R³ | R⁴ | R⁵ | R″ | R′′′ |
|---|---|---|---|---|---|---|---|
| 1 | Br | 2,6- | H | H | H | H | OH |
| | | di(CH₃)phenyloxy | | | | | |
| 2 | Br | butoxy | H | H | H | H | OH |
| 3 | Br | phenoxy | H | H | H | H | OH |
| 4 | Cl | Br | H | H | H | H | OH |
| 5 | Br | Cl | H | H | H | H | OH |
| 6 | Cl | I | H | H | H | H | OH |
| 7 | Cl | H | I | H | H | H | OH |
| 8 | Cl | phenoxy | H | H | H | H | OH |
| 9 | Cl | phenylsulfanyl | H | H | H | H | OH |
| 10 | Br | —CF₃ | H | H | H | H | OH |
| 11 | Br | H | phenoxy | H | H | H | OH |
| 12 | Cl | H | H | phenyl | H | H | OH |
| 13 | Cl | 2,6- | H | H | H | H | OH |
| | | di(CH₃)phenyloxy | | | | | |
| 14 | Br | H | CF₃ | H | H | H | OH |
| 15 | Br | Br | H | H | H | H | OH |
| 16 | Br | phenylsulfanyl | H | H | H | H | OH |
| 17 | Cl | H | phenylsulfanyl | H | H | H | OH |
| 18 | 4-methoxy | H | H | H | H | H | OH |
| | phenyl- | | | | | | |
| | sulfanyl | | | | | | |
| 19 | Br | H | H | phenyl | H | H | OH |
| 20 | Cl | phenyl | H | H | H | H | OH |
| 21 | Br | H | H | H | H | H | OH |
| 22 | Br | methyl | H | H | H | H | OH |
| 23 | Br | H | butoxy | H | H | H | OH |
| 24 | Br | H | Cl | H | H | H | OH |
| 25 | Cl | H | phenoxy | H | H | H | OH |
| 26 | Br | H | phenoxy | H | H | H | OH |
| 27 | H | I | H | H | H | H | OH |
| 28 | Br | phenyl | H | H | H | H | OH |
| 29 | Br | H | phenyl | H | H | H | OH |
| 30 | ethyl sulfanyl | H | H | H | H | H | OH |
| 31 | phenoxy | H | H | H | H | H | OH |
| 32 | H | H | phenyl | H | H | H | OH |
| 33 | Br | H | H | H | phenyl | H | OH |
| 34 | Br | F | H | H | H | H | OH |
| 35 | H | 2,6-di(CH₃) | H | H | H | H | OH |
| | | phenyloxy | | | | | |
| 36 | Cl | H | phenyl | H | H | H | OH |
| 37 | H | phenoxy | H | H | H | H | OH |
| 38 | H | phenylsulfanyl | H | H | H | H | OH |
| 39 | H | phenyl | H | H | H | H | OH |
| 40 | H | H | phenoxy | H | H | H | OH |
| 41 | H | H | phenylsulfanyl | H | H | H | OH |
| 42 | H | H | H | phenyl | H | H | OH |
| 43 | Cl | H | H | H | phenyl | H | OH |
| 44 | H | H | H | H | phenyl | H | OH |
| 45 | Cl | F | H | H | H | H | OH |
| 46 | H | F | H | H | H | H | OH |
| 47 | H | H | Br | H | H | H | OH |
| 48 | H | R²/R³ = phenyl | — | H | H | H | OH |
| 49 | Br | H | benzyloxy | H | H | methyl | OH |
| 50 | Cl | H | H | H | H | methyl | OH |
| 51 | Cl | H | isopropoxy | H | H | methyl | OH |
| 52 | Cl | isopropoxy | H | H | H | methyl | OH |
| 53 | Cl | H | H | H | H | CH₂COOH | OH |
| 54 | Cl | H | isopropoxy | H | H | CH₂COOH | OH |
| 55 | naphth-2- | H | H | H | H | H | OH |
| | yloxy | | | | | | |
| 56 | pyridin-3- | H | H | H | H | H | OH |
| | yloxy | | | | | | |
| 57 | 4-methoxy | H | H | H | H | H | OH |
| | phenoxy | | | | | | |
| 58 | 3-methoxy | H | H | H | H | H | OH |
| | phenoxy | | | | | | |
| 59 | 3- | H | H | H | H | H | OH |
| | fluorophenoxy | | | | | | |
| 60 | 4- | H | H | H | H | H | OH |
| | fluorophenoxy | | | | | | |
| 61 | 2- | H | H | H | H | H | OH |
| | fluorophenoxy | | | | | | |
| 62 | 2-methoxy | H | H | H | H | H | OH |
| | phenoxy | | | | | | |
| 63 | 4-(methyl | H | H | H | H | H | OH |
| | carbonyl | | | | | | |
| | amino) | | | | | | |
| | phenoxy | | | | | | |
| 64 | 4-(methyl | H | H | H | H | H | OH |
| | sulfonamido) | | | | | | |
| | phenoxy | | | | | | |
| 65 | phenylamino | H | H | H | H | H | OH |
| 66 | H | H | pyridin-3-yloxy | H | H | H | OH |
| 67 | H | pyridin-3-yloxy | H | H | H | H | OH |
| 68 | Cl | H | H | H | H | H | methoxy |
| 69 | Cl | H | H | H | H | H | ethoxy |
| 70 | methoxy | H | H | H | H | H | OH |
| 71 | ethoxy | H | H | H | H | H | OH |
| 72 | phenyl | H | H | H | H | H | methyl- |
| | | | | | | | carbonyloxy |
| 73 | phenyl | H | H | H | H | H | OH |
| 74 | ethoxy | H | H | H | H | H | phenyl |
| 75 | Cl | H | H | H | H | H | phenyl |
| 76 | H | H | H | H | H | H | phenyl |
| 77 | methyl | H | H | H | H | H | OH |
| 78 | methoxy | H | H | H | H | H | OH |
| | methyl | | | | | | |
| 79 | N,N-dimethyl | H | H | H | H | H | OH |
| | amino | | | | | | |
| | carbonyl | | | | | | |
| 80 | methyl | H | phenoxy | H | H | H | OH |
| 81 | methyl | phenoxy | H | H | H | H | OH |
| 82 | methyl | phenoxy | H | H | H | H | benzyloxy |
| 83 | methyl | phenoxy | H | H | H | H | ethoxy |
| 84 | N,N-dimethyl | phenoxy | H | H | H | H | OH |
| | amino | | | | | | |
| | carbonyl | | | | | | |
| 85 | methoxy | phenoxy | H | H | H | H | OH |
| | methyl | | | | | | |
| 86 | 4-methyl | H | H | H | H | H | OH |
| | phenyl | | | | | | |
| 87 | methyl | 4-fluoro phenoxy | H | H | H | H | OH |
| 88 | Cl | 4-methoxy | H | H | H | H | OH |
| | | phenoxy | | | | | |
| 89 | H | 4-methoxy | H | H | H | H | OH |
| | | phenoxy | | | | | |
| 90 | Cl | H | 4-methoxy-phenoxy | H | H | H | OH |
| 91 | H | H | 4-methoxy-phenoxy | H | H | H | OH |
| 92 | Cl | 4-CF₃-phenoxy | H | H | H | H | OH |
| 93 | H | 4-CF₃-phenoxy | H | H | H | H | OH |
| 94 | Cl | H | 4-CF₃-phenoxy | H | H | H | OH |
| 95 | H | H | 4-CF₃-phenoxy | H | H | H | OH |
| 96 | Cl | 4-fluorophenoxy | H | H | H | H | OH |
| 97 | H | 4-fluorophenoxy | H | H | H | H | OH |
| 98 | Cl | H | 4-fluoro-phenoxy | H | H | H | OH |
| 99 | H | H | 4-fluoro-phenoxy | H | H | H | OH |
| 100 | H | pyridin-4-yl | H | H | H | H | OH |
| | | sulfanyl | | | | | |
| 101 | H | H | pyridin-4-yl sulfanyl | H | H | H | OH |
| 102 | H | phenylsulfinyl | H | H | H | H | OH |
| 103 | H | phenylsulfonyl | H | H | H | H | OH |
| 104 | H | H | phenyl sulfinyl | H | H | H | OH |
| 105 | H | H | phenyl-sulfonyl | H | H | H | OH |
| 106 | H | H | amino | H | H | H | OH |
| 107 | H | (4-methoxy) | H | H | H | H | OH |
| | | phenylsulfanyl | | | | | |
| | | amino | | | | | |
| 108 | H | phenylurea | H | H | H | H | OH |
| 109 | H | H | phenylurea | H | H | H | OH |
| 110 | phenyl | H | H | H | H | H | OH |
| | sulfanyl | | | | | | |
| 111 | (4-chloro | H | H | H | H | H | OH |
| | phenyl) | | | | | | |
| | sulfanyl | | | | | | |
| 112 | (4-methyl | H | H | H | H | H | OH |
| | phenyl) | | | | | | |
| | sulfanyl | | | | | | |
| 113 | pyridin-2- | H | H | H | H | H | OH |
| | ylsulfanyl | | | | | | |
| 114 | (3-methoxy | H | H | H | H | H | OH |
| | phenyl) | | | | | | |
| | sulfanyl | | | | | | |
| 115 | 2-methoxy | H | H | H | H | H | OH |
| | phenyl | | | | | | |
| | sulfanyl | | | | | | |
| 116 | naphthyl | H | H | H | H | H | OH |
| | sulfonyl | | | | | | |
| 117 | phenyl | H | H | H | H | H | OH |
| | sulfinyl | | | | | | |
| 118 | phenyl | H | H | H | H | H | OH |
| | sulfonyl | | | | | | |
| 119 | H | pyridin-2-yl | H | H | H | H | OH |
| | | sulfonyl | | | | | |
| 120 | H | H | pyridin-2-yl | H | H | H | OH |
| | | | sulfanyl | | | | |
| 121 | Cl | phenoxy | phenoxy | H | H | H | OH |
| 122 | H | phenoxy | phenoxy | H | H | H | OH |
| 123 | H | H | (4-methyl)phenyl | H | H | H | OH |
| | | | SO₂—NH-phenoxy | | | | |
| 124 | H | 4-nitrophenyl | H | H | H | H | OH |
| 125 | H | phenoxy | H | H | H | H | thiol |
| 126 | H | CF₃ | H | H | H | H | thiol |
| 127 | H | 4-(phenylsulfon | H | H | H | H | OH |
| | | amido)phenoxy | | | | | |
| 128 | H | 4-(methylsulfon | H | H | H | H | OH |
| | | amido) phenoxy | | | | | |
| 129 | H | 4-chlorophenoxy | H | H | H | H | OH |
| 130 | H | H | 4-chloro-phenoxy | H | H | H | OH |
| 131 | H | H | 3-fluoro-5-methoxy- | H | H | H | OH |
| | | | phenoxy | | | | |
| 132 | H | 3-methoxy-5- | H | H | H | H | OH |
| | | fluorophenoxy | | | | | |
| 133 | H | 3,4- | H | H | H | H | OH |
| | | difluorophenoxy | | | | | |
| 134 | H | H | 3,4-difluoro- | H | H | H | OH |
| | | | phenoxy | | | | |
| 135 | H | 4-CF₃—O-phenoxy | H | H | H | H | OH |
| 136 | H | H | 4-CF₃—O-phenoxy | H | H | H | OH |
| 137 | H | 3,5- | H | H | H | H | OH |
| | | difluorophenoxy | | | | | |
| 138 | H | H | 3,5-difluorophenoxy | H | H | H | OH |
| 139 | H | 4-(4- | H | H | H | H | OH |
| | | fluorophenoxy) | | | | | |
| | | phenoxy | | | | | |
| 140 | H | H | 4-(4- | H | H | H | OH |
| | | | fluorophenoxy) | | | | |
| | | | phenoxy | | | | |
| 141 | H | 3-chloro-4- | H | H | H | H | OH |
| | | fluorophenoxy | | | | | |
| 142 | H | H | 3-chloro-4- | H | H | H | OH |
| | | | fluorophenoxy | | | | |
| 143 | methyl | 4-chlorophenoxy | H | H | H | H | OH |
| 144 | methyl | H | 4-chlorophenoxy | H | H | H | OH |
| 145 | methyl | 3,5- | H | H | H | H | OH |
| | | difluorophenoxy | | | | | |
| 146 | methyl | 4-methoxy | H | H | H | H | OH |
| | | phenoxy | | | | | |
| 147 | methyl | H | 4-methoxyphenoxy | H | H | H | OH |
| 148 | H | H | cyclohexyloxy | H | H | H | OH |
| 149 | H | cyclohexyloxy | H | H | H | H | OH |
| 150 | methyl | cyclohexyloxy | H | H | H | H | OH |
| 151 | H | cyclohexyl | H | H | H | H | OH |
| | | sulfanyl | | | | | |
| 152 | H | cyclohexyl | H | H | H | H | OH |
| | | sulfonyl | | | | | |
| 153 | isopropyl | H | H | H | H | H | OH |
| 154 | pyridin-2-yl | H | H | H | H | H | OH |
| 155 | ethyl | phenoxy | H | H | H | H | OH |
| 156 | dimethyl | phenylsulfanyl | H | H | H | H | OH |
| | amino methyl | | | | | | |
| 157 | methyl | phenylsulfanyl | H | H | H | H | OH |
| 158 | methyl | 4-trifluoromethyl | H | H | H | H | OH |
| | | phenoxy | | | | | |

Effective Compounds included within the scope of this invention include, for example, those set forth below:
{[4-Hydroxy-1-(naphthalen-2-yloxy)-isoquinoline-3-carbonyl]-amino}-acetic acid;
{[4-Hydroxy-1-(pyridin-3-yloxy)-isoquinoline-3-carbonyl]-amino}-acetic acid;
{[4-Hydroxy-1-(4-methoxy-phenoxy)-isoquinoline-3-carbonyl]-amino}-acetic acid;
{[4-Hydroxy-1-(3-methoxy-phenoxy)-isoquinoline-3-carbonyl]-amino}-acetic acid;
{[1-(3-Fluoro-phenoxy)-4-hydroxy-isoquinoline-3-carbonyl]-amino}-acetic acid;
{[1-(4-Fluoro-phenoxy)-4-hydroxy-isoquinoline-3-carbonyl]-amino}-acetic acid;
{[1-(2-Fluoro-phenoxy)-4-hydroxy-isoquinoline-3-carbonyl]-amino}-acetic acid;
{[4-Hydroxy-1-(2-methoxy-phenoxy)-isoquinoline-3-carbonyl]-amino}-acetic acid;
{[1-(4-Acetylamino-phenoxy)-4-hydroxy-isoquinoline-3-carbonyl]-amino}-acetic acid;
{[4-Hydroxy-1-(4-methanesulfonylamino-phenoxy)-isoquinoline-3-carbonyl]-amino}-acetic acid;
[(4-Hydroxy-1-phenylamino-isoquinoline-3-carbonyl)-amino]-acetic acid;
{[4-Hydroxy-6-(pyridin-3-yloxy)-isoquinoline-3-carbonyl]-amino}-acetic acid;
{[4-Hydroxy-7-(pyridin-3-yloxy)-isoquinoline-3-carbonyl]-amino}-acetic acid;
[(1-Chloro-4-methoxy-isoquinoline-3-carbonyl)-amino]-acetic acid;
[(1-Chloro-4-ethoxy-isoquinoline-3-carbonyl)-amino]-acetic acid;
[(4-Hydroxy-1-methoxy-isoquinoline-3-carbonyl)-amino]-acetic acid;
[(1-Ethoxy-4-hydroxy-isoquinoline-3-carbonyl)-amino]-acetic acid;
[(4-Acetoxy-1-phenyl-isoquinoline-3-carbonyl)-amino]-acetic acid;
[(4-Hydroxy-1-phenyl-isoquinoline-3-carbonyl)-amino]-acetic acid;
[(1-Ethoxy-4-phenyl-isoquinoline-3-carbonyl)-amino]-acetic acid;
[(1-Chloro-4-phenyl-isoquinoline-3-carbonyl)-amino]-acetic acid;
[(4-Phenyl-isoquinoline-3-carbonyl)-amino]-acetic acid;
[(4-Hydroxy-1-methyl-isoquinoline-3-carbonyl)-amino]-acetic acid;
[(4-Hydroxy-1-methoxymethyl-isoquinoline-3-carbonyl)-amino]-acetic acid;
[(1-Dimethylcarbamoyl-4-hydroxy-isoquinoline-3-carbonyl)-amino]-acetic acid;
[(4-Hydroxy-1-methyl-6-phenoxy-isoquinoline-3-carbonyl)-amino]-acetic acid;
[(4-Hydroxy-1-methyl-7-phenoxy-isoquinoline-3-carbonyl)-amino]-acetic acid;
[(4-Benzyloxy-1-methyl-7-phenoxy-isoquinoline-3-carbonyl)-amino]-acetic acid;
[(4-Ethoxy-1-methyl-7-phenoxy-isoquinoline-3-carbonyl)-amino]-acetic acid;
[(1-Dimethylcarbamoyl-4-hydroxy-7-phenoxy-isoquinoline-3-carbonyl)-amino]-acetic acid;
[(4-Hydroxy-1-methoxymethyl-7-phenoxy-isoquinoline-3-carbonyl)-amino]-acetic acid;
[(4-Hydroxy-1-p-tolyl-isoquinoline-3-carbonyl)-amino]-acetic acid;
{[7-(4-Fluoro-phenoxy)-4-hydroxy-1-methyl-isoquinoline-3-carbonyl]-amino}-acetic acid;
{[1-Chloro-4-hydroxy-7-(4-methoxy-phenoxy)-isoquinoline-3-carbonyl]-amino}-acetic acid;
{[4-Hydroxy-7-(4-methoxy-phenoxy)-isoquinoline-3-carbonyl]-amino}-acetic acid;
{[1-Chloro-4-hydroxy-6-(4-methoxy-phenoxy)-isoquinoline-3-carbonyl]-amino}-acetic acid;
{[4-Hydroxy-6-(4-methoxy-phenoxy)-isoquinoline-3-carbonyl]-amino}-acetic acid;
{[1-Chloro-4-hydroxy-7-(4-trifluoromethyl-phenoxy)-isoquinoline-3-carbonyl]-amino}-acetic acid;
{[4-Hydroxy-7-(4-trifluoromethyl-phenoxy)-isoquinoline-3-carbonyl]-amino}-acetic acid;
{[1-Chloro-4-hydroxy-6-(4-trifluoromethyl-phenoxy)-isoquinoline-3-carbonyl]-amino}-acetic acid;
{[4-Hydroxy-6-(4-trifluoromethyl-phenoxy)-isoquinoline-3-carbonyl]-amino}-acetic acid;
{[1-Chloro-7-(4-fluoro-phenoxy)-4-hydroxy-isoquinoline-3-carbonyl]-amino}-acetic acid;
7-(4-Fluoro-phenoxy)-4-hydroxy-isoquinoline-3-carbonyl]-amino}-acetic acid;
{[1-Chloro-6-(4-fluoro-phenoxy)-4-hydroxy-isoquinoline-3-carbonyl]-amino}-acetic acid;
{[6-(4-Fluoro-phenoxy)-4-hydroxy-isoquinoline-3-carbonyl]-amino}-acetic acid;
{[4-Hydroxy-7-(pyridin-4-ylsulfanyl)-isoquinoline-3-carbonyl]-amino}-acetic acid;
{[4-Hydroxy-6-(pyridin-4-ylsulfanyl)-isoquinoline-3-carbonyl]-amino}-acetic acid;
[(7-Benzenesulfinyl-4-hydroxy-isoquinoline-3-carbonyl)-amino]-acetic acid;
[(7-Benzenesulfonyl-4-hydroxy-isoquinoline-3-carbonyl)-amino]-acetic acid;
[(6-Benzenesulfinyl-4-hydroxy-isoquinoline-3-carbonyl)-amino]-acetic acid;
[(6-Benzenesulfonyl-4-hydroxy-isoquinoline-3-carbonyl)-amino]-acetic acid;
[(6-Amino-4-hydroxy-isoquinoline-3-carbonyl)-amino]-acetic acid;
{[4-Hydroxy-7-(4-methoxy-benzenesulfonylamino)-isoquinoline-3-carbonyl]-amino}-acetic acid;
{[4-Hydroxy-7-(3-phenyl-ureido)-isoquinoline-3-carbonyl]-amino}-acetic acid;
{[4-Hydroxy-6-(3-phenyl-ureido)-isoquinoline-3-carbonyl]-amino}-acetic acid;
[(4-Hydroxy-1-phenylsulfanyl-isoquinoline-3-carbonyl)-amino]-acetic acid;
{[1-(4-Chloro-phenylsulfanyl)-4-hydroxy-isoquinoline-3-carbonyl]-amino}-acetic acid;
[(4-Hydroxy-1-p-tolylsulfanyl-isoquinoline-3-carbonyl)-amino]-acetic acid;
{[4-Hydroxy-1-(pyridin-2-ylsulfanyl)-isoquinoline-3-carbonyl]-amino}-acetic acid;
{[4-Hydroxy-1-(3-methoxy-phenylsulfanyl)-isoquinoline-3-carbonyl]-amino}-acetic acid;
{[4-Hydroxy-1-(2-methoxy-phenylsulfanyl)-isoquinoline-3-carbonyl]-amino}-acetic acid;
{[4-Hydroxy-1-(naphthalen-2-ylsulfanyl)-isoquinoline-3-carbonyl]-amino}-acetic acid;
[(1-Benzenesulfinyl-4-hydroxy-isoquinoline-3-carbonyl)-amino]-acetic acid;
[(1-Benzenesulfonyl-4-hydroxy-isoquinoline-3-carbonyl)-amino]-acetic acid;
{[4-Hydroxy-7-(pyridin-2-ylsulfanyl)-isoquinoline-3-carbonyl]-amino}-acetic acid;
{[4-Hydroxy-6-(pyridin-2-ylsulfanyl)-isoquinoline-3-carbonyl]-amino}-acetic acid;
[(1-Chloro-4-hydroxy-6,7-diphenoxy-isoquinoline-3-carbonyl)-amino]-acetic acid;
[(4-Hydroxy-6,7-diphenoxy-isoquinoline-3-carbonyl)-amino]-acetic acid;
({4-Hydroxy-7-[4-(toluene-4-sulfonylamino)-phenoxy]-isoquinoline-3-carbonyl}-amino)-acetic acid;
{[4-Hydroxy-7-(4-nitro-phenoxy)-isoquinoline-3-carbonyl]-amino}-acetic acid;
[(4-Mercapto-7-phenoxy-isoquinoline-3-carbonyl)-amino]-acetic acid;
[(4-Mercapto-7-trifluoromethyl-isoquinoline-3-carbonyl)-amino]-acetic acid;
{[7-(4-Benzenesulfonylamino-phenoxy)-4-hydroxy-isoquinoline-3-carbonyl]-amino}-acetic acid;
{[4-Hydroxy-7-(4-methanesulfonylamino-phenoxy)-isoquinoline-3-carbonyl]-amino}-acetic acid;
{[7-(4-Chloro-phenoxy)-4-hydroxy-isoquinoline-3-carbonyl]-amino}-acetic acid;
{[6-(4-Chloro-phenoxy)-4-hydroxy-isoquinoline-3-carbonyl]-amino}-acetic acid;
{[6-(3-Fluoro-5-methoxy-phenoxy)-4-hydroxy-isoquinoline-3-carbonyl]-amino}-acetic acid;
{[7-(3-Fluoro-5-methoxy-phenoxy)-4-hydroxy-isoquinoline-3-carbonyl]-amino}-acetic acid;
{[7-(3,4-Difluoro-phenoxy)-4-hydroxy-isoquinoline-3-carbonyl]-amino}-acetic acid;
{[6-(3,4-Difluoro-phenoxy)-4-hydroxy-isoquinoline-3-carbonyl]-amino}-acetic acid;
{[4-Hydroxy-7-(4-trifluoromethoxy-phenoxy)-isoquinoline-3-carbonyl]-amino}-acetic acid;
{[4-Hydroxy-6-(4-trifluoromethoxy-phenoxy)-isoquinoline-3-carbonyl]-amino}-acetic acid;
2-(S)-{[7-(4-Chloro-phenoxy)-4-hydroxy-isoquinoline-3-carbonyl]-amino}-propionic acid;
2-(S)-{[6-(4-Chloro-phenoxy)-4-hydroxy-isoquinoline-3-carbonyl]-amino}-propionic acid;
2-{[7-(3,4-Difluoro-phenoxy)-4-hydroxy-isoquinoline-3-carbonyl]-amino}-propionic acid;
2-(S)-[(4-Hydroxy-7-phenylsulfanyl-isoquinoline-3-carbonyl)-amino]amino propionic acid;
2-(R)-[(4-Hydroxy-7-phenylsulfanyl-isoquinoline-3-carbonyl)-amino]-propionic acid;
2-(R)-[(4-Hydroxy-7-phenoxy-isoquinoline-3-carbonyl)-amino]-propionic acid;
2-(S)-{[4-Hydroxy-7-(4-methoxy-phenoxy)-isoquinoline-3-carbonyl]-amino}-propionic acid;
2-(S)-[(7-Benzenesulfonyl-4-hydroxy-isoquinoline-3-carbonyl)-amino]-propionic acid;
(R)-2-[(4-Hydroxy-1-methoxymethyl-7-phenoxy-isoquinoline-3-carbonyl)-amino]-propionic acid;
(S)-2-[(4-Hydroxy-1-methoxymethyl-7-phenoxy-isoquinoline-3-carbonyl)-amino]-propionic acid;
(S)-2-[(4-Mercapto-7-phenoxy-isoquinoline-3-carbonyl)-amino]-propionic acid;
(S)-2-{[1-(4-Chloro-phenylsulfanyl)-4-hydroxy-isoquinoline-3-carbonyl]-amino}-propionic acid;
(R)-2-{[1-(4-Chloro-phenylsulfanyl)-4-hydroxy-isoquinoline-3-carbonyl]-amino}-propionic acid;
[(4-Hydroxy-7-phenylsulfanyl-isoquinoline-3-carbonyl)-amino]-acetic acid;
[(4-Hydroxy-6-phenylsulfanyl-isoquinoline-3-carbonyl)-amino]-acetic acid;
[(1-Chloro-4-hydroxy-7-phenylsulfanyl-isoquinoline-3-carbonyl)-amino]-acetic acid;
[(1-Chloro-4-hydroxy-6-phenylsulfanyl-isoquinoline-3-carbonyl)-amino]-acetic acid;
[(1-Bromo-4-hydroxy-7-phenylsulfanyl-isoquinoline-3-carbonyl)-amino]-acetic acid;
[(1-Bromo-4-hydroxy-6-phenylsulfanyl-isoquinoline-3-carbonyl)-amino]-acetic acid;
[(4-Hydroxy-7-phenoxy-isoquinoline-3-carbonyl)-amino]-acetic acid;
[(4-Hydroxy-6-phenoxy-isoquinoline-3-carbonyl)-amino]-acetic acid;
[(1-Chloro-4-hydroxy-7-phenoxy-isoquinoline-3-carbonyl)-amino]-acetic acid;
[(1-Chloro-4-hydroxy-6-phenoxy-isoquinoline-3-carbonyl)-amino]-acetic acid;
[(1-Bromo-4-hydroxy-7-phenoxy-isoquinoline-3-carbonyl)-amino]-acetic acid;
[(1-Bromo-4-hydroxy-6-phenoxy-isoquinoline-3-carbonyl)-amino]-acetic acid;
{[7-(2,6-Dimethyl-phenoxy)-4-hydroxy-isoquinoline-3-carbonyl]-amino}-acetic acid;
{[1-Chloro-7-(2,6-dimethyl-phenoxy)-4-hydroxy-isoquinoline-3-carbonyl]-amino}-acetic acid;
{[1-Bromo-7-(2,6-dimethyl-phenoxy)-4-hydroxy-isoquinoline-3-carbonyl]-amino}-acetic acid;
[(1-Bromo-7-chloro-4-hydroxy-isoquinoline-3-carbonyl)-amino]-acetic acid;
[(1-Bromo-6-chloro-4-hydroxy-isoquinoline-3-carbonyl)-amino]-acetic acid;
[(1-Bromo-4-hydroxy-7-trifluoromethyl-isoquinoline-3-carbonyl)-amino]-acetic acid;
[(1-Bromo-4-hydroxy-6-trifluoromethyl-isoquinoline-3-carbonyl)-amino]-acetic acid;
[(4-Hydroxy-1-phenoxy-isoquinoline-3-carbonyl)-amino]-acetic acid;
[(1,7-dibromo-4-hydroxy-isoquinoline-3-carbonyl)-amino]-acetic acid;
[(7-Bromo-1-chloro-4-hydroxy-isoquinoline-3-carbonyl)-amino]-acetic acid;
[(6-Bromo-4-hydroxy-isoquinoline-3-carbonyl)-amino]-acetic acid;
[(1-Bromo-7-fluoro-4-hydroxy-isoquinoline-3-carbonyl)-amino]-acetic acid;
[(7-Fluoro-4-hydroxy-isoquinoline-3-carbonyl)-amino]-acetic acid;
[(1-Chloro-7-fluoro-4-hydroxy-isoquinoline-3-carbonyl)-amino]-acetic acid;
[(1-Chloro-4-hydroxy-benzo[g]isoquinoline-3-carbonyl)-amino]-acetic acid;
[(1-Bromo-4-hydroxy-isoquinoline-3-carbonyl)-amino]-acetic acid;
[(4-Hydroxy-6-phenyl-isoquinoline-3-carbonyl)-amino]-acetic acid;
[(4-Hydroxy-7-phenyl-isoquinoline-3-carbonyl)-amino]-acetic acid;
[(1-Chloro-4-hydroxy-6-phenyl-isoquinoline-3-carbonyl)-amino]-acetic acid;
[(1-Chloro-4-hydroxy-7-phenyl-isoquinoline-3-carbonyl)-amino]-acetic acid;
[(1-Bromo-4-hydroxy-6-phenyl-isoquinoline-3-carbonyl)-amino]-acetic acid;
[(1-Bromo-4-hydroxy-7-phenyl-isoquinoline-3-carbonyl)-amino]-acetic acid;
[(4-Hydroxy-5-phenyl-isoquinoline-3-carbonyl)-amino]-acetic acid;
[(4-Hydroxy-8-phenyl-isoquinoline-3-carbonyl)-amino]-acetic acid;
[(1-Chloro-4-hydroxy-5-phenyl-isoquinoline-3-carbonyl)-amino]-acetic acid;
[(1-Chloro-4-hydroxy-8-phenyl-isoquinoline-3-carbonyl)-amino]-acetic acid;
[(1-Bromo-4-hydroxy-5-phenyl-isoquinoline-3-carbonyl)-amino]-acetic acid;
[(1-Bromo-4-hydroxy-8-phenyl-isoquinoline-3-carbonyl)-amino]-acetic acid;
[(1-Ethylsulfanyl-4-hydroxy-isoquinoline-3-carbonyl)-amino]-acetic acid;
{[4-Hydroxy-1-(4-methoxy-phenylsulfanyl)-isoquinoline-3-carbonyl]-amino}-acetic acid;
[(1-Chloro-4-hydroxy-7-iodo-isoquinoline-3-carbonyl)-amino]-acetic acid;
[(1-Chloro-4-hydroxy-6-iodo-isoquinoline-3-carbonyl)-amino]-acetic acid;
[(4-Hydroxy-7-iodo-isoquinoline-3-carbonyl)-amino]-acetic acid;
[(1-Bromo-4-hydroxy-7-methyl-isoquinoline-3-carbonyl)-amino]-acetic acid;
[(1-Bromo-7-butoxy-4-hydroxy-isoquinoline-3-carbonyl)-amino]-acetic acid;
[(1-Bromo-6-butoxy-4-hydroxy-isoquinoline-3-carbonyl)-amino]-acetic acid;
[(6-Benzyloxy-1-chloro-4-hydroxy-isoquinoline-3-carbonyl)-methyl-amino]-acetic acid;
[(1-Chloro-4-hydroxy-isoquinoline-3-carbonyl)-methyl-amino]-acetic acid;
[(1-Chloro-4-hydroxy-6-isopropoxy-isoquinoline-3-carbonyl)-methyl-amino]-acetic acid;
[(1-Chloro-4-hydroxy-7-isopropoxy-isoquinoline-3-carbonyl)-methyl-amino]-acetic acid;
[Carboxymethyl-(1-chloro-4-hydroxy-isoquinoline-3-carbonyl)-amino]-acetic acid;
[Carboxymethyl-(1-chloro-4-hydroxy-6-isopropoxy-isoquinoline-3-carbonyl)-amino]-acetic acid;
1-Chloro-4-hydroxy-isoquinoline-3-carboxylic acid (2-amino-ethyl)-amide (trifluoroacetic acid salt);
1-Chloro-4-hydroxy-isoquinoline-3-carboxylic acid (2-methoxy-ethyl)-amide;
1-Chloro-4-hydroxy-isoquinoline-3-carboxylic acid (2-hydroxy-ethyl)-amide;
1-Chloro-4-hydroxy-isoquinoline-3-carboxylic acid (2-dimethylamino-ethyl)-amide;
1-Chloro-4-hydroxy-isoquinoline-3-carboxylic acid (2-acetylamino-ethyl)-amide;
1-Chloro-4-hydroxy-6-isopropoxy-isoquinoline-3-carboxylic acid (2-hydroxy-ethyl)-amide;
1-Chloro-4-hydroxy-6-isopropoxy-isoquinoline-3-carboxylic acid (2-methoxy-ethyl)-amide;
1-Chloro-4-hydroxy-6-isopropoxy-isoquinoline-3-carboxylic acid (2-amino-ethyl)-amide (trifluoro-acetic acid salt);
1-Chloro-4-hydroxy-6-isopropoxy-isoquinoline-3-carboxylic acid (2-dimethylamino-ethyl)-amide;
1-Chloro-4-hydroxy-7-isopropoxy-isoquinoline-3-carboxylic acid (2-amino-ethyl)-amide (trifluoro-acetic acid salt);
1-Chloro-4-hydroxy-7-isopropoxy-isoquinoline-3-carboxylic acid (2-methoxy-ethyl)-amide;
1-Chloro-4-hydroxy-7-isopropoxy-isoquinoline-3-carboxylic acid (2-dimethylamino-ethyl)-amide;
1-Chloro-4-hydroxy-7-isopropoxy-isoquinoline-3-carboxylic acid (2-hydroxy-ethyl)-amide;
(S)-2-[(6-Benzyloxy-1-chloro-4-hydroxy-isoquinoline-3-carbonyl)-amino]-propionic acid;
(R)-2-[(1-Chloro-4-hydroxy-isoquinoline-3-carbonyl)-amino]-3-hydroxy-propionic acid;
(S)-2-[(1-Chloro-4-hydroxy-isoquinoline-3-carbonyl)-amino]-3-hydroxy-propionic acid;
(R)-2-[(1-Chloro-4-hydroxy-6-isopropoxy-isoquinoline-3-carbonyl)-amino]-3-hydroxy-propionic acid;
(S)-2-[(1-Chloro-4-hydroxy-6-isopropoxy-isoquinoline-3-carbonyl)-amino]-3-hydroxy-propionic acid;
(R)-2-[(1-Chloro-4-hydroxy-7-isopropoxy-isoquinoline-3-carbonyl)-amino]-3-hydroxy-propionic acid;
(S)-2-[(1-Chloro-4-hydroxy-7-isopropoxy-isoquinoline-3-carbonyl)-amino]-3-hydroxy-propionic acid;
2-[(1-Chloro-4-hydroxy-isoquinoline-3-carbonyl)-amino]-2-methyl-propionic acid;
2-[(1-Chloro-4-hydroxy-6-isopropoxy-isoquinoline-3-carbonyl)-amino]-2-methyl-propionic acid;
(R)-2-[(1-Chloro-4-hydroxy-isoquinoline-3-carbonyl)-amino]-3-(1H-imidazol-4-yl)-propionic acid (trifluoroacetic acid salt);
(S)-2-[(1-Chloro-4-hydroxy-isoquinoline-3-carbonyl)-amino]-3-(1H-imidazol-4-yl)-propionic acid (trifluoroacetic acid salt);
(R)-2-[(1-Chloro-4-hydroxy-isoquinoline-3-carbonyl)-amino]-3-methyl-butyric acid;
(S)-2-[(1-Chloro-4-hydroxy-isoquinoline-3-carbonyl)-amino]-3-methyl-butyric acid;
(R)-2-[(1-Chloro-4-hydroxy-6-isopropoxy-isoquinoline-3-carbonyl)-amino]-3-methyl-butyric acid;
(S)-2-[(1-Chloro-4-hydroxy-6-isopropoxy-isoquinoline-3-carbonyl)-amino]-3-methyl-butyric acid;
(R)-2-[(1-Chloro-4-hydroxy-7-isopropoxy-isoquinoline-3-carbonyl)-amino]-3-methyl-butyric acid;
(S)-2-[(1-Chloro-4-hydroxy-7-isopropoxy-isoquinoline-3-carbonyl)-amino]-3-methyl-butyric acid;
(S)-2-[(6-Benzyloxy-1-chloro-4-hydroxy-isoquinoline-3-carbonyl)-amino]-3-methyl-butyric acid;
(R)-2-[(1-Chloro-4-hydroxy-isoquinoline-3-carbonyl)-amino]-3-phenyl-propionic acid;
(S)-2-[(1-Chloro-4-hydroxy-isoquinoline-3-carbonyl)-amino]-3-phenyl-propionic acid;
(R)-2-[(1-Chloro-4-hydroxy-6-isopropoxy-isoquinoline-3-carbonyl)-amino]-3-phenyl-propionic acid;
(S)-2-[(1-Chloro-4-hydroxy-6-isopropoxy-isoquinoline-3-carbonyl)-amino]-3-phenyl-propionic acid;
(R)-2-[(1-Chloro-4-hydroxy-7-isopropoxy-isoquinoline-3-carbonyl)-amino]-3-phenyl-propionic acid;
(S)-2-[(1-Chloro-4-hydroxy-7-isopropoxy-isoquinoline-3-carbonyl)-amino]-3-phenyl-propionic acid;
(R)-2-[(1-Chloro-4-hydroxy-isoquinoline-3-carbonyl)-amino]-3-(4-hydroxy-phenyl)-propionic acid;
(S)-2-[(1-Chloro-4-hydroxy-isoquinoline-3-carbonyl)-amino]-3-(4-hydroxy-phenyl)-propionic acid;
(R)-2-[(1-Chloro-4-hydroxy-6-isopropoxy-isoquinoline-3-carbonyl)-amino]-3-(4-hydroxy-phenyl)-propionic acid;
(S)-2-[(1-Chloro-4-hydroxy-6-isopropoxy-isoquinoline-3-carbonyl)-amino]-3-(4-hydroxy-phenyl)-propionic acid;
(R)-2-[(1-Chloro-4-hydroxy-7-isopropoxy-isoquinoline-3-carbonyl)-amino]-3-(4-hydroxy-phenyl)-propionic acid;
(S)-2-[(1-Chloro-4-hydroxy-7-isopropoxy-isoquinoline-3-carbonyl)-amino]-3-(4-hydroxy-phenyl)-propionic acid;
(R)-2-[(1-Chloro-4-hydroxy-6-isopropoxy-isoquinoline-3-carbonyl)-amino]-pentanoic acid;
(S)-2-[(1-Chloro-4-hydroxy-6-isopropoxy-isoquinoline-3-carbonyl)-amino]-pentanoic acid;
(R)-1-(1-Chloro-4-hydroxy-isoquinoline-3-carbonyl)-pyrrolidine-2-carboxylic acid;
(S)-1-(1-Chloro-4-hydroxy-isoquinoline-3-carbonyl)-pyrrolidine-2-carboxylic acid;
(R)-1-(1-Chloro-4-hydroxy-6-isopropoxy-isoquinoline-3-carbonyl)-pyrrolidine-2-carboxylic acid;
(S)-1-(1-Chloro-4-hydroxy-6-isopropoxy-isoquinoline-3-carbonyl)-pyrrolidine-2-carboxylic acid;
(R)-6-Amino-2-[(1-chloro-4-hydroxy-isoquinoline-3-carbonyl)-amino]-hexanoic acid (trifluoroacetic acid salt);
(S)-6-Amino-2-[(1-chloro-4-hydroxy-isoquinoline-3-carbonyl)-amino]-hexanoic acid (trifluoroacetic acid salt);
(R)-6-Amino-2-[(1-chloro-4-hydroxy-6-isopropoxy-isoquinoline-3-carbonyl)-amino]-hexanoic acid; trifluoroacetic acid salt;
(S)-6-Amino-2-[(1-chloro-4-hydroxy-6-isopropoxy-isoquinoline-3-carbonyl)-amino]-hexanoic acid (trifluoroacetic acid salt);
(R)-6-Amino-2-[(1-chloro-4-hydroxy-7-isopropoxy-isoquinoline-3-carbonyl)-amino]-hexanoic acid; trifluoroacetic acid salt;
(S)-6-Amino-2-[(1-chloro-4-hydroxy-7-isopropoxy-isoquinoline-3-carbonyl)-amino]-hexanoic acid (trifluoroacetic acid salt);
(R)-2-[(1-Chloro-4-hydroxy-isoquinoline-3-carbonyl)-amino]-succinic acid;
(S)-2-[(1-Chloro-4-hydroxy-isoquinoline-3-carbonyl)-amino]-succinic acid;
(R)-2-[(1-Chloro-4-hydroxy-6-isopropoxy-isoquinoline-3-carbonyl)-amino]-succinic acid;
(S)-2-[(1-Chloro-4-hydroxy-6-isopropoxy-isoquinoline-3-carbonyl)-amino]-succinic acid;
(R)-2-[(1-Chloro-4-hydroxy-7-isopropoxy-isoquinoline-3-carbonyl)-amino]-succinic acid;
1-[(1-Chloro-4-hydroxy-isoquinoline-3-carbonyl)-amino]-cyclopropane-carboxylic acid;
1-[(1-Chloro-4-hydroxy-6-isopropoxy-isoquinoline-3-carbonyl)-amino]-cyclopropanecarboxylic acid;
Dideutero-[(1-chloro-4-hydroxy-isoquinoline-3-carbonyl)-amino]-acetic acid;
(R)-2-[(6-Benzyloxy-1-chloro-4-hydroxy-isoquinoline-3-carbonyl)-amino]-propionic acid;
(S)-2-[(7-Benzyloxy-1-chloro-4-hydroxy-isoquinoline-3-carbonyl)-amino]-propionic acid;
(R)-2-[(7-Benzyloxy-1-chloro-4-hydroxy-isoquinoline-3-carbonyl)-amino]-propionic acid;
(S)-2-[(1-Chloro-4-hydroxy-isoquinoline-3-carbonyl)-amino]-propionic acid;
(R)-2-[(1-Chloro-4-hydroxy-isoquinoline-3-carbonyl)-amino]-propionic acid;
(S)-2-[(6-Isopropoxy-1-chloro-4-hydroxy-isoquinoline-3-carbonyl)-amino]-propionic acid;
(R)-2-[6-Isopropoxy-1-chloro-4-hydroxy-isoquinoline-3-carbonyl)-amino]-propionic acid;
(S)-2-[(7-Isopropoxy-1-chloro-4-hydroxy-isoquinoline-3-carbonyl)-amino-propionic acid;
(R)-2-[(7-Isopropoxy-1-chloro-4-hydroxy-isoquinoline-3-carbonyl)-amino]propionic acid;
1-Chloro-4-hydroxy-6-isopropoxy-isoquinoline-3-carboxylic acid (2-hydroxy-1-hydroxymethyl-ethyl)-amide;
1-Chloro-4-hydroxy-7-isopropoxy-isoquinoline-3-carboxylic acid (2-hydroxy-1-hydroxymethyl-ethyl)-amide;
1-Chloro-4-hydroxy-isoquinoline-3-carboxylic acid (2-hydroxy-1-hydroxymethyl-ethyl)-amide;
{[7-(3,5-Difluoro-phenoxy)-4-hydroxy-isoquinoline-3-carbonyl]-amino}-acetic acid;
{[6-(3,5-Difluoro-phenoxy)-4-hydroxy-isoquinoline-3-carbonyl]-amino}-acetic acid;
({7-[4-(4-Fluoro-phenoxy)-phenoxy]-4-hydroxy-isoquinoline-3-carbonyl}-amino)-acetic acid;
({6-[4-(4-Fluoro-phenoxy)-phenoxy]-4-hydroxy-isoquinoline-3-carbonyl}-amino)-acetic acid;
{[7-(3-Chloro-4-fluoro-phenoxy)-4-hydroxy-isoquinoline-3-carbonyl]amino}-acetic acid;
{[6-(3-Chloro-4-fluoro-phenoxy)-4-hydroxy-isoquinoline-3-carbonyl]amino}-acetic acid;
(S)-2-{[7-(3-Fluoro-5-methoxy-phenoxy)-4-hydroxy-isoquinoline-3-carbonyl]-amino}-propionic acid;
2-(S)-[(7-Cyclohexyloxy-4-hydroxy-isoquinoline-3-carbonyl)-amino]-propionic acid;
2-(S)-{[7-(4-Fluoro-phenoxy)-4-hydroxy-1-methyl-isoquinoline-3-carbonyl]-amino}-propionic acid;
2-(S)-{[7-(4-Fluoro-phenoxy)-4-hydroxy-isoquinoline-3-carbonyl]-amino}-propionic acid;
2-(S)-[(4-Hydroxy-1-methyl-7-phenoxy-isoquinoline-3-carbonyl)-amino-]-propionic acid;
2-(S)-[(4-Hydroxy-1-methyl-7-phenylsulfanyl-isoquinoline-3-carbonyl-)-amino]-propionic acid;
2-(S)-{[4-Hydroxy-7-(4-trifluoromethyl-phenoxy)-isoquinoline-3-carbonyl]-amino}-propionic acid;
{[7-(4-Chloro-phenoxy)-4-hydroxy-1-methyl-isoquinoline-3-carbonyl]amino}-acetic acid;
{[6-(4-Chloro-phenoxy)-4-hydroxy-1-methyl-isoquinoline-3-carbonyl]amino}-acetic acid;
{[7-(3,5-Difluoro-phenoxy)-4-hydroxy-1-methyl-isoquinoline-3-carbonyl]-amino}-acetic acid;
{[4-Hydroxy-7-(4-methoxy-phenoxy)-1-methyl-isoquinoline-3-carbonyl]-amino}-acetic acid;
{[4-Hydroxy-6-(4-methoxy-phenoxy)-1-methyl-isoquinoline-3-carbonyl]-amino}-acetic acid;
[(6-Cyclohexyloxy-4-hydroxy-isoquinoline-3-carbonyl)-amino]-acetic acid;
[(7-Cyclohexyloxy-4-hydroxy-isoquinoline-3-carbonyl)-amino]-acetic acid;
[(7-Cyclohexyloxy-4-hydroxy-1-methyl-isoquinoline-3-carbonyl)-amino-]-acetic acid;
[(7-Cyclohexylsulfanyl-4-hydroxy-isoquinoline-3-carbonyl)-amino]-acetic acid;
[(7-Cyclohexanesulfonyl-4-hydroxy-isoquinoline-3-carbonyl)-amino]-acetic acid;
[(4-Hydroxy-1-isobutyl-isoquinoline-3-carbonyl)-amino]-acetic acid;
[(4-Hydroxy-1-pyridin-2-yl-isoquinoline-3-carbonyl)-amino]-acetic acid;
[(1-Ethyl-4-hydroxy-7-phenoxy-isoquinoline-3-carbonyl)-amino]-acetic acid;
[(1-Dimethylaminomethyl-4-hydroxy-7-phenylsulfanyl-isoquinoline-3-carbonyl)-amino]-acetic acid;
[(4-Hydroxy-1-methyl-7-phenylsulfanyl-isoquinoline-3-carbonyl)-amino]-acetic acid;
{[4-Hydroxy-1-methyl-7-(4-trifluoromethyl-phenoxy)-isoquinoline-3-c-arbonyl]-amino}-acetic acid; and pharmaceutically acceptable salts, esters and prodrugs thereof.

In still another embodiment of the invention, a pharmaceutical composition is provided comprising a pharmaceutically acceptable excipient or carrier and a therapeutically effective amount of any of the Effective Compounds or a mixture of such compounds.

Also provided are methods for treating, preventing or pretreating a condition mediated at least in part by HIF and/or EPO is provided. The method comprises administering to a mammalian patient a therapeutically effective amount of any of the Effective Compounds with the proviso that in certain embodiments, the compound is not selected from the group consisting of:
N-((1-chloro-4-hydroxy-7-(2-propyloxy)isoquinolin-3-yl)-carbonyl)glycine,
N-((1-chloro-4-hydroxy-6-(2-propyloxy)isoquinolin-3-yl)-carbonyl)glycine,
N-((1-chloro-4-hydroxy-isoquinoline-3-carbonyl)-amino)acetic acid,
N-((1-chloro-4-hydroxy-7-methoxyisoquinolin-3-yl)-carbonyl)-glycine,
N-((1-chloro-4-hydroxy-6-methoxyisoquinolin-3-yl)-carbonyl)-glycine,
N-((7-butyloxy-1-chloro-4-hydroxyisoquinolin-3-yl)-carbonyl)-glycine,
N-((6-benzyloxy-1-chloro-4-hydroxy-isoquinoline-3-carbonyl)-amino)acetic acid,
N-((7-benzyloxy-1-chloro-4-hydroxy-isoquinoline-3-carbonyl)-amino)acetic acid,
N-((8-chloro-4-hydroxyisoquinolin-3-yl)-carbonyl)-glycine,
N-((7-butoxy-4-hydroxy-isoquinoline-3-carbonyl)-amino)acetic acid, and
((7-benzyloxy-1-chloro-4-hydroxy-isoquinoline-3-carbonyl)amino)acetic acid methyl ester.

Moreover, it is contemplated that in some embodiments of the invention, biological matter (as defined herein) is provided with a precursor compound that becomes the active version of any of the Effective Compounds by exposure to biological matter, such as by chemical or enzymatic means. In addition, the compound may be provided to the biological matter as a salt of the compound in the form of a free radical, or a negatively charged, positively charged or multiply charged species. In certain embodiments, some compounds may be defined or categorized by more than one formula or category (e.g., chalcogenide) and in such cases, the use of phrases such as “Formula I or Formula IV” is not intended to connote the exclusion of such compounds.

In other embodiments, it is specifically contemplated that the HIFα stabilizer and/or 2-oxoglutarate dioxygenase inhibitor (e.g., a HIF prolyl hydroxylase inhibitor) is sodium sulfide, sodium thiomethoxide, cysteamine, sodium thiocyanate, cysteamine-S-phosphate sodium salt, or tetrahydrothiopyran-4-ol. In some embodiments, the HIFα stabilizer and/or HIF prolyl hydroxylase inhibitor is dimethylsulfoxide, thioacetic acid, selenourea, 2-(3-aminopropyl)-aminoethanethiol-dihydrogen-phosphate-ester, 2-mercapto-ethanol, thioglycolicether, sodium selenide, sodium methane sulfinate, thiourea, or dimethylsulfide.

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in Greene and Wuts (1991), and references cited therein.

Furthermore, the compounds of this invention will typically contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

H. Diagnostic Applications of the Effective Compounds

Sulfites are produced by all cells in the body during normal metabolism of sulfur containing amino acids. Sulfite oxidase removes, and thus regulates, the levels of sulfites. Differential activities of these enzymes would lead to different levels of sulfites evolved in tissue specific manner. In the example described above, for solid tumors in hypoxic conditions, sulfites may be produced at higher levels to provide local protective state to the tumor cells through the reduction of metabolic state as well as the inhibition of immune surveillance. Therefore, it would be beneficial to measure sulfite levels and incorporate this as part of diagnosis for several disease states such as solid tumors. Furthermore, since we propose using sulfites for various applications, it would be useful to follow this using some sort of imaging or other monitoring process.

It is possible to measure sulfite levels in serum to get a total sulfite level using current technology (e.g., HPLC). It is worth exploring the possibility of imaging sulfites. Alternatively, a proteomic approach may allow an understanding of how the regulation of the enzymes involved in sulfite metabolism may be altered in certain disease states, allowing for this approach to diagnostics.

I. Screening Applications of the Effective Compounds

In still further embodiments, the present invention provides methods for identifying Effective Compounds, such as a compound of Formula I, Ia-Id, II, III, IIIa, IV, V, VI, VII, VIII, IX, X, or XI, that act in a like fashion with respect to, for example, treating a hypoxic or ischemic condition or inducing stasis. In some cases, the Effective Compounds being sought works like a chalcogenide compound in reducing core body temperature or preserving viability in hypoxic or anoxic environments that would otherwise kill the biological matter if it were not for the presence of the Effective Compound. These assays may comprise random screening of large libraries of candidate substances; alternatively, the assays may be used to focus on particular classes of compounds selected with an eye towards attributes that are believed to make them more likely to act as Effective Compounds. In certain embodiments, the screening methods commences with providing a candidate Effective Compound, and then;
(a) admixing the candidate Effective Compound with a biological matter;
(b) measuring one or more cellular responses characteristic of Effective Candidate treatment; and
(c) comparing the one or more responses with the biological matter in the absence of the candidate Effective Compound.

Assays may be conducted with isolated cells, tissues/organs, or intact organisms.

It will, of course, be understood that all the screening methods of the present invention are useful in themselves notwithstanding the fact that effective candidates may not be found. The invention provides methods for screening for such candidates, not solely methods of finding them. However, it will also be understand that candidate Effective Compound may be identified as an effective Effective Compound according to one or more assays, meaning that the candidate Effective Compound appears to have some ability to act as an Effective Compound by modulating HIF (e.g., inducing stasis in a biological matter). Screening, in some embodiments, involves using an assay described in the Examples or elsewhere in the disclosure to identify a modulator. Moreover, in addition to or instead of the method described in this section, a candidate Effective Compound may be tested for activity either as an oxygen antagonist or as another compound having a property of an Effective Compound, such as protective metabolic agent or therapeutic substance. Some embodiments of screening methods are provided above.

An effective Effective Compound may be further characterized or assayed. Moreover, the effective Effective Compound may be used in an in vivo animal or animal model (as discussed below) or be used in further in vivo animals or animal models, which may involve the same species of animals or in different animal species.

Furthermore, it is contemplated that an Effective Compound identified according to embodiments of the invention may also be manufactured after screening. Also, biological matter may be exposed to or contacted with an effective Effective Compound according to methods of the invention, particularly with respect to therapeutic or preservation embodiments.

1. In Vivo Assays

In vivo assays involve the use of various animal models. Due to their size, ease of handling, and information on their physiology and genetic make-up, mice are typically a preferred embodiment. However, other animals are suitable as well, including rats, rabbits, hamsters, guinea pigs, gerbils, woodchucks, mice, cats, dogs, sheep, goats, pigs, cows, horses and monkeys (including chimps, gibbons and baboons). Fish are also contemplated for use with in vivo assays, as are nematodes. Assays for modulators may be conducted using an animal model derived from any of these species.

In such assays, one or more candidate substances are administered to an animal, and the ability of the candidate substance(s) to treat a hypoxic or an ischemic condition, for example, or to induce stasis, reduce core body temperature, or endow the biological material the ability to survive hypoxic or anoxic environmental conditions, as compared to an inert vehicle (negative control) and H₂S (positive control), identifies a modulator. Treatment of animals with test compounds will involve the administration of the compound, in an appropriate form, to the animal. Administration of the candidate compound (gas or liquid) will be by any route that could be utilized for clinical or non-clinical purposes, including but not limited to oral, nasal (inhalation or aerosol), buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

J. Methods of Pretreatment Using the Effective Compounds

In another aspect, the invention provides methods for treating a patient at risk of developing an ischemic or hypoxic condition, e.g., individuals at high risk for atherosclerosis, etc., using the compounds described herein. Risk factors for atherosclerosis include, e.g., hyperlipidemia, cigarette smoking, hypertension, diabetes mellitus, hyperinsulinemia, and abdominal obesity. Therefore, the present invention provides methods of preventing ischemic tissue injury, the method comprising administering a therapeutically effective amount of an Effective Compound, or a pharmaceutically acceptable salt thereof, alone or in combination with a pharmaceutically acceptable excipient, to a patient in need. In one embodiment, the Effective Compound can be administered based on predisposing conditions, e.g., hypertension, diabetes, occlusive arterial disease, chronic venous insufficiency, Raynaud's disease, chronic skin ulcers, cirrhosis, congestive heart failure, and systemic sclerosis.

When the methods of the invention are used to prevent tissue damage caused by HIF-associated disorders including, but not limited to, ischemic and hypoxic disorders, treatment may be predicated on predisposing conditions, e.g., hypertension, diabetes, occlusive arterial disease, chronic venous insufficiency, Raynaud's disease, systemic sclerosis, cirrhosis, congestive heart failure, etc. Similarly, the methods of the invention can be used as a pretreatment to decrease or prevent the tissue damage caused by HIF-associated disorders including, but not limited to, ischemic and hypoxic disorders. The need for pretreatment may be based on a patient's history of recurring episodes of an ischemic condition, e.g., myocardial infarction or transient ischemic attacks; based on symptoms of impending ischemia, e.g., angina pectoris; or based on physical parameters implicating possible or likely ischemia or hypoxia, such as is the case with, e.g., individuals placed under general anesthesia or temporarily working at high altitudes. The methods may also be used in the context of organ transplants to pretreat organ donors and to maintain organs removed from the body prior to implantation in a recipient.

In one specific embodiment, the methods are used to increase vascularization and/or granulation tissue formation in damaged tissue, wounds, and ulcers. For example, compounds of the invention have been shown to be effective in stimulating granulation tissue formation in wound healing. Granulation tissue contains newly formed, leaky blood vessels and a provisional stroma of plasma proteins, such as fibrinogen and plasma fibronectin. Release of growth factors from inflammatory cells, platelets, and activated endothelium, stimulates fibroblast and endothelial cell migration and proliferation within the granulation tissue. Ulceration can occur if vascularization or neuronal stimulation is impaired. The methods of the invention are effective at promoting granulation tissue formation. Thus, the invention provides methods for treating a patient having tissue damage due to, e.g., an infarct, having wounds induced by, e.g., trauma or injury, or having chronic wounds or ulcers produced as a consequence of a disorder, e.g., diabetes. The method comprises administering a therapeutically effective amount of an Effective Compound, or a pharmaceutically acceptable salt thereof, alone or in combination with a pharmaceutically acceptable excipient, to a patient in need.

In another aspect, the invention provides methods of using an Effective Compound to pretreat a subject to decrease or prevent the development of tissue damage associated with ischemia or hypoxia. The methods of the invention may produce therapeutic benefit when administered immediately before a condition involving ischemia or hypoxia. For example, application of the methods of the invention prior to induction of myocardial infarction may show improvement in heart architecture and performance. Further, the methods of the invention may produce therapeutic benefit when administered immediately before and during ischemic-reperfusion injury, significantly reducing diagnostic parameters associated with renal failure.

Therefore, the invention provides methods of pretreating a subject to decrease or prevent the tissue damage associated with ischemia or hypoxia, the method comprising administering a therapeutically effective amount of an Effective Compound, or a pharmaceutically acceptable salt thereof, alone or in combination with a pharmaceutically acceptable excipient, to a patient with a history of ischemic disorders, e.g., myocardial infarctions, or having symptoms of impending ischemia, e.g., angina pectoris. In another embodiment, an Effective Compound can be administered based on physical parameters implicating possible ischemia, e.g., individuals placed under general anesthesia or temporarily working at high altitudes. In yet another embodiment, an Effective Compounds may be used in organ transplants to pretreat organ donors and to maintain organs removed from the body prior to implantation in the recipient.

In particular embodiments, methods of the present invention are used to induce stasis or pre-stasis in biological matter, e.g., cells, tissues, organs, and/or organisms, after an injury (e.g., a traumatic injury) or after the onset or progression of a disease, in order to protect the biological matter from damage associated with the injury or disease prior to, during, or following treatment of the injury or disease. In other embodiments, methods of the present invention are used to induce or promote stasis or pre-stasis in biological matter prior to subjection to an injurious event (e.g., an elective surgery) or prior to the onset or progression of a disease, in order to protect the biological matter from damage associated with adverse conditions such as injury or disease. In such cases, such methods are generally referred to as “pre-treatment” with an Effective Compound. Pre-treatment may include methods wherein biological matter is provided with an Effective Compound both before and during, and before, during and after biological matter is subjected to adverse conditions (e.g., an injury or onset or the progression of a disease), and methods wherein biological matter is provided with an Effective Compound only before biological matter is subjected to adverse conditions.

### III. Modes of Administration and Pharmaceutical Compositions

A. Effective Amount

An effective amount of a pharmaceutical composition of an Effective Compound of the present invention, generally, is defined as that amount sufficient to detectably ameliorate, reduce, minimize or limit the extent of the condition of interest. More rigorous definitions may apply, including elimination, eradication or cure of disease.

B. Administration

The routes of administration of a compound of the present invention will vary, naturally, with the location and nature of the condition to be treated, and include, e.g., inhalation, intradermal, transdermal, parenteral, intravenous, intramuscular, intranasal, subcutaneous, percutaneous, intratracheal, intraperitoneal, intratumoral, perfusion, lavage, direct injection, and oral administration and formulation. As detailed below, Effective Compounds may be administered as medical gases by inhalation or intubation, as injectable liquids by intravascular, intravenous, intra-arterial, intracerobroventicular, intraperitoneal, subcutaneous administration, as topical liquids or gels, or in solid oral dosage forms.

Moreover, the amounts may vary depending on the type of biological matter (cell type, tissue type, organism genus and species, etc.) and/or its size (weight, surface area, etc.). It will generally be the case that the larger the organism, the larger the dose. Therefore, an effective amount for a mouse will generally be lower than an effective amount for a rat, which will generally be lower than an effective amount for a dog, which will generally be lower than an effective amount for a human. The effective concentration of a compound of the present invention to achieve stasis, for example, in a human depends on the dosage form and route of administration. For inhalation, in some embodiments effective concentrations are in the range of 50 ppm to 500 ppm, delivered continuously. For intravenous administration, in some embodiments effective concentrations are in the range of 0.5 to 50 milligrams per kilogram of body weight delivered continuously.

Similarly, the length of time of administration may vary depending on the type of biological matter (cell type, tissue type, organism genus and species, etc.) and/or its size (weight, surface area, etc.) and will depend in part upon dosage form and route of administration. In particular embodiments, a compound of the present invention may be provided for about or at least 30 seconds, 1 minute, 2 minutes, 3 minutes, 5 minutes, 10 minutes, 15 minutes, 30 minutes, 1 hour, 2 hours, 3 hours, four hours five hours, six hours, eight hours, twelve hours, twenty-four hours, or greater than twenty-four hours. A compound of the present invention may be administered in a single dos or multiple doses, with varying amounts of time between administered doses.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time. Alternatively, the amount specified may be the amount administered as the average daily, average weekly, or average monthly dose.

In the case of transplant, the present invention may be used pre- and or post-operatively to render host or graft materials quiescent. In a specific embodiment, a surgical site may be injected or perfused with a formulation comprising an Effective Compound. The perfusion may be continued post-surgery, for example, by leaving a catheter implanted at the site of the surgery.

An Effective Compound can be administered to the patient in a dose or doses of about or of at least about 0.5, 1, 5, 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mg or more, or any range derivable therein. Alternatively, the amount specified may be the amount administered as the average daily, average weekly, or average monthly dose, or it may be expressed in terms of mg/kg, where kg refers to the weight of the patient and the mg is specified above. In other embodiments, the amount specified is any number discussed above but expressed as mg/m² (with respect to, for example, patient surface area).

Concentrations of an Effective Compound can be in doses of about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 or more mg or mg/m² (with respect to, for example, patient surface area), or any range derivable therein. Alternatively, the amount specified may be the amount administered as the average daily, average weekly, or average monthly dose, or it may be expressed in terms of mg/kg, where kg refers to the weight of the patient and the mg is specified above.

C. Injectable Compositions and Formulations

In some embodiments, the preferred methods for the delivery of an Effective Compound of the present invention may comprise inhalation, intravenous injection, perfusion of a particular area, and/or oral administration. However, the pharmaceutical compositions disclosed herein may alternatively be administered parenterally, intradermally, intramuscularly, transdermally or even intraperitoneally as described in U.S. Pat. No. 5,543,158; U.S. Pat. No. 5,641,515 and U.S. Pat. No. 5,399,363 (each specifically incorporated herein by reference in its entirety).

Injection of an Effective Compound may be delivered by syringe or any other method used for injection of a solution, as long as the solution can pass through the particular gauge of needle required for injection. A novel needeless injection system has recently been described (U.S. Pat. No. 5,846,233, incorporated herein by reference in its entirety) having a nozzle defining an ampule chamber for holding the solution and an energy device for pushing the solution out of the nozzle to the site of delivery.

Solutions of the Effective Compounds may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In certain formulations, a water-based formulation is employed while in others, it may be lipid-based.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the Effective Compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are typically vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

Effective Compounds and their related compositions as described herein may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of about 0.5% to about 10%, preferably about 1% to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%.

In certain formulations, an Effective Compound is formulated as a dry power. It is a further object of the present invention to use, for the process, readily accessible cheap raw materials in the form of dairy by-products, in place of pure carbohydrates.

The Effective Compounds are administered in a manner compatible with the dosage formulation, and in such amount as will be therapeutically effective. The quantity to be administered depends on the subject and condition to be treated, including, e.g., the aggressiveness of the cancer; the size of any tumor(s); the area, organ, tissue, etc. to be subject to stasis; and/or the previous or other courses of treatment. Precise amounts of an Effective Compound required to be administered depend on the judgment of the practitioner. Suitable regimes for initial administration and subsequent administration are also variable, but are typified by an initial administration followed by other administrations. Such administration may be systemic, as a single dose, continuous over a period of time spanning 10, 20, 30, 40, 50, 60 minutes, and/or 1, 2, 3, 4, 5, 6, 7, 8, 9, 10, 11, 12, 13, 14, 15, 16, 17, 18, 19, 20, 21, 22, 23, 24 or more hours, and/or 1, 2, 3, 4, 5, 6, 7, days or more. Moreover, administration may be through a time release or sustained release mechanism, implemented by formulation and/or mode of administration.

The manner of application may be varied widely. Any of the conventional methods for administration are applicable. These are believed to include oral application on a solid physiologically acceptable base or in a physiologically acceptable dispersion, parenterally, by injection or the like. The dosage will depend on the route of administration and will vary according to the size of the host.

In many instances, it will be desirable to have multiple administrations of one or more Effective Compounds.

D. Intravenous Formulations

In one embodiment, Effective Compounds of the invention may be formulated for parenteral administration (e.g., intravenous, intra-arterial). In the cases where the Effective Compound is a gas at room temperature, a solution containing a known and desired concentration of the gas molecule dissolved in a liquid or a solution for parenteral administration is contemplated. Preparation of the Effective Compound solution may be achieved by, for example, contacting (e.g., bubbling or infusing) the gas with the solution to cause the gas molecules to dissolve in the solution. Those skilled in the art will recognize that the amount of gas that dissolves in the solution will depend on a number of variables including, but not limited to, the solubility of the gas in the liquid or solution, the chemical composition of the liquid or solution, its temperature, its pH, its ionic strength, as well as the concentration of the gas and the extent of contacting (e.g., rate of and duration of bubbling or infusing). The concentration of the Effective Compound in the liquid or solution for parenteral administration can be determined using methods known to those skilled in the art. The stability of the Effective Compound in the liquid or solution can be determined by measuring the concentration of the dissolved Effective Compound after varying intervals of time following preparation or manufacture of the Effective Compound solution, where a decrease in the concentration of the Effective Compound compared to the starting concentration is indicative of loss or chemical conversion of the Effective Compound.

In some embodiments, there is a solution containing an Effective Compound, such as a chalcogenide, is produced by dissolving a salt form of the Effective Compound into sterile water or saline (0.9% sodium chloride) to yield a pharmaceutically acceptable intravenous dosage form. The intravenous liquid dosage form may be buffered to a certain pH to enhance the solubility of the Effective Compound or to influence the ionization state of the Effective Compound. Any of a number of salt forms known to those skilled in the art may suffice, including, but not limited to, sodium, calcium, barium, lithium, or potassium. In another embodiment, sodium sulfide or sodium selenide is dissolved in sterile phosphate buffered saline and the pH is adjusted to 7.0 with hydrochloric acid to yield a solution of known concentration which can be administered to a subject intravenously or intrarterially.

It is contemplated that in some embodiments, a pharmaceutical composition of the invention is a saturated solution with respect to the Effective Compound. The solution can be any pharmaceutically acceptable formulation, many of which are well known, such as Ringer's solution. In certain embodiments, the concentration of the Effective Compound is about, at least about, or at most about 0.001, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0 M or more, or any range derivable therein (at standard temperature and pressure (STP).) With H₂S, for example, in some embodiments, the concentration can be from about 0.01 to about 0.5 M (at STP). It is specifically contemplated the above concentrations may be applied with respect to carbon monoxide and carbon dioxide in a solution separately or together.

Furthermore, when administration is intravenous, it is contemplated that the following parameters may be applied. A flow rate of about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 gtts/min or μgtts/min, or any range derivable therein. In some embodiments, the amount of the solution is specified by volume, depending on the concentration of the solution. An amount of time may be about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 minutes, 1, 2, 3, 4, 5, 6, 7, 8, 9, 10, 11, 12, 13, 14, 15, 16, 17, 18, 19, 20, 21, 22, 23, 24 hours, 1, 2, 3, 4, 5, 6, 7 days, 1, 2, 3, 4, 5 weeks, and/or 1, 2, 3, 4, 5, 6, 7, 8, 9, 10, 11, 12 months, or any range derivable therein.

Volumes of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mls or liters, or any range therein, may be administered overall or in a single session.

In some embodiments, the solution of the Effective Compound for parenteral administration is prepared in a liquid or solution in which the oxygen has been removed prior to contacting the liquid or solution with the Effective Compound. Certain Effective Compounds, in particular certain chalcogenide compounds as described herein (e.g., hydrogen sulfide, hydrogen selenide), are not stable in the presence of oxygen due to their ability to react chemically with oxygen, leading to their oxidation and chemical transformation. Oxygen can be removed from liquids or solutions using methods known in the art, including, but not limited to, application of negative pressure (vacuum degasing) to the liquid or solution, or contacting the solution or liquid with a reagent which causes oxygen to be bound or “chelated”, effectively removing it from solution.

In another embodiment, the solution of the Effective Compound for parenteral administration may be stored in a gas-tight container. This is particularly desirable when the oxygen has previously been removed from the solution to limit or prevent oxidation of the Effective Compound. Additionally, storage in a gas-tight container will inhibit the volatilization of the Effective Compound from the liquid or solution, allowing a constant concentration of the dissolved Effective Compound to be maintained. Gas-tight containers are known to those skilled in the art and include, but are not limited to, “i.v. bags” comprising a gas impermeable construction material, or a sealed glass vial. To prevent exposure to air in the gas-tight storage container, an inert gas, such as nitrogen or argon, may be introduced into the container prior to closure.

1. Bolus Administration

Bolus administration generally refers to a single dose comprising one or more Effective Compounds. The term “bolus” is intended to exclude dosage forms such as sustained release, pulsed release, and time release, and includes any dosage form which can be used to deliver a single dose. Typically, bolus administration takes place intravenously by direct infusion, injection or gravity drip but other means, such as oral dosage forms, are also envisioned. In some embodiments, a bolus administration may comprise a single dose of a concentrated form of one or more Effective Compounds given over a period of time. In some embodiments, a bolus administration may comprise a single large dose given over a short period of time. A bolus may be administered to a patient in need of treatment once daily, such as in the morning. The bolus dosages of the present invention may be administered in any conventional form known to those of skill in the art.

E. Topical Formulations and Uses Thereof

Methods and compositions of the present invention are useful for inducing stasis in superficial layers of the skin and oral mucosa, including, but not limited to, hair follicle cells, capillary endothelial cells, and epithelial cells of the mouth and tongue. Radiation therapy and chemotherapy for the treatment of cancer damage normal cells in the hair follicles and oral mucosa, leading to the unintended, but debilitating side effects of cancer therapy, hair loss and oral mucositis, respectively. Induction of stasis in hair follicle cells and/or the vascular cells that supply blood to the hair follicles may slow, limit or prevent damage to hair follicle cells and the resultant hair loss that accompanies radiation therapy and chemotherapy, or other alopecia, male-pattern baldness, female-pattern baldness, or other absence of the hair from skin areas where it normally is present. Induction of stasis in oral epithelial and mesenchymal cells may slow, limit or prevent damage to cells lining the mouth, esophagus and tongue and the resultant painful condition of oral mucositis.

In certain embodiments the Effective Compound is administered topically. This is achieved by formulating the Effective Compound in a cream, gel, paste, or mouthwash and applying such formulation directly to the areas that require exposure to the Effective Compound (e.g., scalp, mouth, tongue, throat).

The topical compositions of this invention can be formulated as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The preferred carriers are those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762, each of which is incorporated herein by reference in its entirety.

Creams are preferably formulated from a mixture of mineral oil, self-emulsifying beeswax and water in which mixture the active ingredient, dissolved in a small amount of an oil such as almond oil, is admixed. A typical example of such a cream is one which includes about 40 parts water, about 20 parts beeswax, about 40 parts mineral oil and about 1 part almond oil.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight.

Lotions may be conveniently prepared by dissolving the active ingredient, in a suitable high molecular weight alcohol such as propylene glycol or polyethylene glycol.

Possible pharmaceutical preparations that can be used rectally include, for example, suppositories, which consist of a combination of one or more of the Effective Compounds with a suppository base. Suitable suppository bases are, for example, natural or synthetic triglycerides, or paraffin hydrocarbons. In addition, it is also possible to use gelatin rectal capsules which consist of a combination of the Effective Compounds with a base. Possible base materials include, for example, liquid triglycerides, polyethylene glycols, or paraffin hydrocarbons.

F. Solid Dosage Forms

Pharmaceutical compositions include solid dosage forms in which the Effective Compound is trapped, or sequestered, in a porous carrier framework that is capable of achieving a crystalline, solid state. Such solid dosage forms with the capacity for gas storage are known in the art and can be produced in pharmaceutically acceptable forms (e.g., Yaghi et al. 2003). A particular advantage of such a pharmaceutical composition pertains to chalcogenide compounds (e.g., hydrogen sulfide, carbon monoxide, hydrogen selenide), which can be toxic to certain mammals at certain concentrations in their free form. In certain embodiments, the compound may be formulated for oral administration.

G. Perfusion Systems

A perfusion system for cells may be used to expose a tissue or organ to an Effective Compound in the form of a liquid or a semi-solid. Perfusion refers to continuous flow of a solution through or over a population of cells. It implies the retention of the cells within the culture unit as opposed to continuous-flow culture, which washes the cells out with the withdrawn media (e.g., chemostat). Perfusion allows for better control of the culture environment (pH, pO₂, nutrient levels, Effective Compound levels, etc.) and is a means of significantly increasing the utilization of the surface area within a culture for cell attachment.

The technique of perfusion was developed to mimic the cells milieu in vivo where cells are continuously supplied with blood, lymph, or other body fluids. Without perfusion of a physiological nutrient solution, cells in culture go through alternating phases of being fed and starved, thus limiting full expression of their growth and metabolic potential. In the context of the present invention, a perfusion system may also be used to perfuse cells with an Effective Compound to induce stasis.

Those of skill in the art are familiar with perfusion systems, and there are a number of perfusion systems available commercially. Any of these perfusion systems may be employed in the present invention. One example of a perfusion system is a perfused packed-bed reactor using a bed matrix of a non-woven fabric (CelliGen™, New Brunswick Scientific, Edison, N.J.; Wang et al., 1992; Wang et al., 1993; Wang et al., 1994). Briefly described, this reactor comprises an improved reactor for culturing of both anchorage- and non-anchorage-dependent cells. The reactor is designed as a packed bed with a means to provide internal recirculation. Preferably, a fiber matrix carrier is placed in a basket within the reactor vessel. A top and bottom portion of the basket has holes, allowing the medium to flow through the basket. A specially designed impeller provides recirculation of the medium through the space occupied by the fiber matrix for assuring a uniform supply of nutrient and the removal of wastes. This simultaneously assures that a negligible amount of the total cell mass is suspended in the medium. The combination of the basket and the recirculation also provides a bubble-free flow of oxygenated medium through the fiber matrix. The fiber matrix is a non-woven fabric having a “pore” diameter of from 10 μm to 100 μm, providing for a high internal volume with pore volumes corresponding to 1 to 20 times the volumes of individual cells.

The perfused packed-bed reactor offers several advantages. With a fiber matrix carrier, the cells are protected against mechanical stress from agitation and foaming. The free medium flow through the basket provides the cells with optimum regulated levels of oxygen, pH, and nutrients. Products can be continuously removed from the culture and the harvested products are free of cells and can be produced in low-protein medium, which facilitates subsequent purification steps. This technology is explained in detail in WO 94/17178 (Aug. 4, 1994, Freedman et al.), which is hereby incorporated by reference in its entirety.

The Cellcube™ (Corning-Costar) module provides a large styrenic surface area for the immobilization and growth of substrate attached cells. It is an integrally encapsulated sterile single-use device that has a series of parallel culture plates joined to create thin sealed laminar flow spaces between adjacent plates.

The Cellcube™ module has inlet and outlet ports that are diagonally opposite each other and help regulate the flow of media. During the first few days of growth the culture is generally satisfied by the media contained within the system after initial seeding. The amount of time between the initial seeding and the start of the media perfusion is dependent on the density of cells in the seeding inoculum and the cell growth rate. The measurement of nutrient concentration in the circulating media is a good indicator of the status of the culture. When establishing a procedure it may be necessary to monitor the nutrients composition at a variety of different perfusion rates to determine the most economical and productive operating parameters.

Other commercially available perfusion systems include, for example, CellPerf® (Laboratories MABIO International, Tourcoing, France) and the Stovall Flow Cell (Stovall Life Science, Inc., Greensboro, N.C.)

The timing and parameters of the production phase of cultures depends on the type and use of a particular cell line. Many cultures require a different media for production than is required for the growth phase of the culture. The transition from one phase to the other will likely require multiple washing steps in traditional cultures. However, one of the benefits of a perfusion system is the ability to provide a gentle transition between various operating phases. The perfusion system can also facilitate the transition from a growth phase to a static phase induced by an Effective Compound. Likewise, the perfusion system can facilitate the transition from a static phase to a growth phase by replacing the solution comprising an Effective Compound with, for example, a physiological nutrient media.

H. Catheters

In certain embodiments, a catheter is used to provide an Effective Compound to an organism. Of particular interest is the administration of such an agent to the heart or vasculature system. Frequently, a catheter is used for this purpose. Yaffe et al., 2004 discusses catheters particularly in the context of suspended animation, though the use of catheters were generally known prior to this publication.

I. Delivery of Gases

Gas delivery systems are known to those of skill in the art. Non-limiting examples of such systems are described in U.S. patent application Ser. No. 11/408,734, herein incorporated by reference in its entirety.

J. Other Apparatuses

Within certain embodiments of the invention, it may be desirable to supplement the methods of the present invention for the treatment of patients who will be or have been subjected to trauma with the ability to externally manipulate the core body temperature of the patient. In this regard, the core body temperature of a patient may be, in combination with the methods of the present invention, manipulated by invasive or non-invasive routes. Invasive methods for the manipulation of core body temperature include, for example, the use of a heart-lung pump to heat or cool the patient's blood thus raising or cooling the patient's core body temperature. Non-invasive routes to manipulate core body temperature include systems and apparatuses that transfer heat into or out of the patient's body.

K. Further Delivery Devices or Apparatuses

In some embodiments it is contemplated that methods or compositions will involve a specific delivery device or apparatus. Any method discussed herein can be implemented with any device for delivery or administration including, but not limited, to those discussed herein.

For topical administration of Effective Compounds of the invention may be formulated as solutions, gels, ointments, creams, suspensions, etc. as are well-known in the art. Systemic formulations may include those designed for administration by injection or infusion, e.g., subcutaneous, intravenous, intramuscular, intrathecal or intraperitoneal injection, as well as those designed for transdermal, transmucosal, oral or pulmonary administration.

For oral administration, the Effective Compounds of the invention to be formulated as tablets, pills, dragees, capsules, liquids, gels, syrups, slurries, suspensions and the like, for oral ingestion by a patient to be treated or oral liquid preparations such as, for example, suspensions, elixirs and solutions.

For buccal administration, the compositions may take the form of tablets, lozenges, etc. formulated in conventional manner. Other intramucosal delivery might be by suppository or intranasally.

For administration directly to the lung by inhalation the compound of invention may be conveniently delivered to the lung by a number of different devices. For example,

Metered-Dose Inhalers (MDIs): a Metered Dose Inhaler (“MDI”) which utilizes canisters that contain a suitable low boiling propellant, e.g., dichlorodifluoromethane, trichlorofluoromethane, dichlorotetrafluoroethane, carbon dioxide or other suitable gas may be used to deliver the compound of invention directly to the lung. MDI devices are available from a number of suppliers such as 3M Corporation (e.g., on the world wide web at 3m.com/us/healthcare/manufacturers/dds/pdf/idd_valve_canister_brochure.pdf), Nasacort from Aventis (e.g., world wide web at products.sanofi-aventis.us/Nasacort_HFA/nasacort_HFA.html-63k), Boehringer Ingelheim, (e.g., world wide web at.boehringer-ingelheim.com/corporate/home/download/r_and_d2003.pdf) Aerobid from Forest Laboratories, (e.g., world wide web at.frx.com/products/aerobid.aspx) Glaxo-Wellcome, (for example, on the world wide web at .gsk.com/research/newmedicines/newmedicines_pharma.html) and Schering Plough, (world wide web at .schering-plough.com/schering_plough/pc/allergy_respiratory.jsp).

Dry Powder Inhalers (DPIs): DPI devices typically use a mechanism such as a burst of gas to create a cloud of dry powder inside a container, which may then be inhaled by the patient. DPI devices are also well known in the art and may be purchased from a number of vendors which include, for example, Foradil aerolizer from Schering Corporation, (e.g., world wide web .spfiles.com/piforadil.pdf) Advair Diskus from Glaxo-Wellcome. (e.g., world wide web at us.gsk.com/products/assets/us_advair.pdf) A popular variation is the multiple dose DPI (“MDDPI”) system, which allows for the delivery of more than one therapeutic dose. MDDPI devices are available from companies such as Plumicort Turbuhaler from AstraZeneca, (e.g., world wide web at.twistclickinhale.com/) GlaxoWellcome, (e.g., world wide web at us.gsk.com/products/assets/us_advair.pdf) and Schering Plough, (e.g., world wide web at schering-plough.com/schering_plough/pc/allergy_respiratory.jsp). It is further contemplated that such devices, or any other devices discussed herein, may be altered for single use.

Electrohydrodynamic (EHD) aerosol delivery: EHD aerosol devices use electrical energy to aerosolize liquid drug solutions or suspensions (see e.g., Noakes et al., U.S. Pat. No. 4,765,539; Coffee, U.S. Pat. No. 4,962,885; Coffee, PCT Application, WO 94/12285; Coffee, PCT Application, WO 94/14543; Coffee, PCT Application, WO 95/26234, Coffee, PCT Application, WO 95/26235, Coffee, PCT Application, WO 95/32807, each of which is incorporated herein by reference in its entirety). EHD aerosol devices may more efficiently deliver drugs to the lung than existing pulmonary delivery technologies.

Nebulizers: Nebulizers create aerosols from liquid drug formulations by using, for example, ultrasonic energy to form fine particles that may be readily inhaled. Examples of nebulizers include devices supplied by Sheffield/Systemic Pulmonary Delivery Ltd. (See, Armer et al., U.S. Pat. No. 5,954,047; van der Linden et al., U.S. Pat. No. 5,950,619; van der Linden et al., U.S. Pat. No. 5,970,974), Intal nebulizer solution by Aventis, (e.g., world wide web at .fda.gov/medwatch/SAFETY/2004/feb_PI/Intal_Nebulizer_PI.pdf). Each of the patent listed are incorporated herein by reference in its entirety.

For administration of a gas directly to the lungs by inhalation various delivery methods currently available in the market for delivering oxygen may be used. For example, a resuscitator such as an ambu-bag may be employed (see U.S. Pat. Nos. 5,988,162 and 4,790,327, each of which is incorporated herein by reference in its entirety). An ambu-bag consists of a flexible squeeze bag attached to a face mask, which is used by the physician to introduce air/gas into the casualty's lungs.

A portable, handheld medicine delivery device capable producing atomized agents that are adapted to be inhaled through a nebulizer by a patient suffering from a respiratory condition. In addition, such delivery device provides a means wherein the dose of the inhaled agent can be remotely monitored and, if required altered, by a physician or doctor. See U.S. Pat. No. 7,013,894, incorporated herein by reference in its entirety. Delivery of the compound of invention may be accomplished by a method for the delivery of supplemental gas to a person combined with the monitoring of the ventilation of the person with both being accomplished without the use of a sealed face mask such as described in U.S. Pat. No. 6,938,619, incorporated herein by reference in its entirety. A pneumatic oxygen conserving device for efficiently dispensing oxygen or other gas used during respiratory therapy such that only the first part of the patient's breath contains the oxygen or other therapeutic gas. (See U.S. Pat. No. 6,484,721, incorporated herein by reference in its entirety). A gas delivery device is used which is triggered when the patient begins to inhale. A tail of gas flow is delivered to the patient after the initial inhalation timed period to prevent pulsing of gas delivery to the patient. In this manner gas is only delivered to the patient during the first portion of inhalation preventing gas from being delivered which will only fill the air passageways to the patient's lungs. By efficiently using the oxygen, cylinder bottles of oxygen used when a patient is mobile will last longer and be smaller and easier to transport. By pneumatically delivering the gas to the patient no batteries or electronics are used.

All the devices described here may have an exhaust system to bind or neutralize the compound of invention.

Transdermal administration of the compound of the invention can be achieved by medicated device or patch which is affixed to the skin of a patient. The patch allows a medicinal compound contained within the patch to be absorbed through the skin layers and into the patient's blood stream. Such patches are commercially available as Nicoderm CQ patch from Glaxo Smithkline, (world wide web at nicodermcq.com/NicodermCQ.aspx) and as Ortho Evra from Ortho-McNeil Pharmaceuticals, (world wide web at .ortho-mcneilpharmaceutical.com/healthinfo/womenshealth/products/orthoevra.html). Transdermal drug delivery reduces the pain associated with drug injections and intravenous drug administration, as well as the risk of infection associated with these techniques. Transdermal drug delivery also avoids gastrointestinal metabolism of administered drugs, reduces the elimination of drugs by the liver, and provides a sustained release of the administered drug. Transdermal drug delivery also enhances patient compliance with a drug regimen because of the relative ease of administration and the sustained release of the drug.

Other modifications of the patch include the Ultrasonic patch which is designed with materials to enable the transmission of ultrasound through the patch, effecting the delivery of medications stored within the patch, and to be used in conjunction with ultrasonic drug delivery processes (see U.S. Pat. No. 6,908,448, incorporated herein by reference in its entirety). Patch in a bottle (U.S. Pat. No. 6,958,154, incorporated herein by reference in its entirety) includes a fluid composition, e.g., an aerosol spray in some embodiments, that is applied onto a surface as a fluid, but subsequently dries to form a covering element, such as a patch, on a surface of a host. The covering element so formed has a tack free outer surface covering and an underlying tacky surface that helps adhere the patch to the substrate.

Another drug delivery system comprises one or more ball semiconductor aggregations and facilitating release of a drug stored in a reservoir. The first aggregate is used for sensing and memory, and a second aggregation for control aspects, such as for pumping and dispensing of the drug. The system may communicate with a remote control system, or operate independently on local power over a long period for delivery of the drug based upon a request of the patient, timed-release under control by the system, or delivery in accordance with measured markers. See U.S. Pat. No. 6,464,687, incorporated herein by reference in its entirety.

PUMPS and Infusion Devices: An infusion pump or perfusor infuses fluids, medication or nutrients into a patient's circulatory system. Infusion pumps can administer fluids in very reliable and inexpensive ways. For example, they can administer as little as 0.1 mL per hour injections (too small for a drip), injections every minute, injections with repeated boluses requested by the patient, up to maximum number per hour (e.g. in patient-controlled analgesia), or fluids whose volumes vary by the time of day. Various types of infusion devices have been described in the following patent applications before the United States Patent and Trademark Office. These include but are not limited to U.S. Pat. No. 7,029,455, U.S. Pat. No. 6,805,693, U.S. Pat. No. 6,800,096, U.S. Pat. No. 6,764,472, U.S. Pat. No. 6,742,992, U.S. Pat. No. 6,589,229, U.S. Pat. No. 6,626,329, U.S. Pat. No. 6,355,019, U.S. Pat. No. 6,328,712, U.S. Pat. No. 6,213,738, U.S. Pat. No. 6,213,723, U.S. Pat. No. 6,195,887, U.S. Pat. No. 6,123,524 and U.S. Pat. No. 7,022,107. Each of these patents are incorporated herein in its entirety. In addition, infusion pumps are also available from Baxter International Inc. (world wide web at .baxter.com/products/medication_management/infusion_pumps/), Alaris Medical Systems (world wide web at alarismed.com/products/infusion.shtml) and from B Braun Medical Inc. (world wide web at bbraunusa.com/index.cfm?uuid=001AA837D0B759A1E34666434FF604ED).

Oxygen/Gas bolus delivery device: Such a device for delivering gas to Chronic Obstructive Pulmonary Disease (COPD) patients is a available from Tyco Healthcare (world wide web at. tycohealth-ece.com/files/d0004/ty_zt7ph2.pdf). It can also be used to deliver the compound of invention. The above device is cost-effective, lightweight, inconspicuous and portable.

“Patch in a bottle” (U.S. Pat. No. 6,958,154, incorporated herein by reference in its entirety) includes a fluid composition, e.g., an aerosol spray in some embodiments, that is applied onto a surface as a fluid, but subsequently dries to form a covering element, such as a patch, on a surface of a host. The covering element so formed has a tack free outer surface covering and an underlying tacky surface that helps adhere the patch to the substrate.

Implantable Drug Delivery System: Another drug delivery system comprises one or more ball semiconductor aggregations and facilitating release of a drug stored in a reservoir. The first aggregate is used for sensing and memory, and a second aggregation for control aspects, such as for pumping and dispensing of the drug. The system may communicate with a remote control system, or operate independently on local power over a long period for delivery of the drug based upon a request of the patient, timed-release under control by the system, or delivery in accordance with measured markers. See U.S. Pat. No. 6,464,687, incorporated herein by reference in its entirety.

The contents of each of the cited patents and web addresses discussed in this section are hereby incorporated by reference.

### VI. Combination Therapies

The compounds and methods of the present invention may be used in the context of a number of therapeutic and diagnostic applications. In order to increase the effectiveness of a treatment with the compositions of the present invention, such as HIF prolyl hydroxylase inhibitors and stasis inducers, it may be desirable to combine these compositions with other agents effective in the treatment of those diseases and conditions (secondary therapy). For example, the treatment of stroke (antistroke treatment) typically involves an antiplatelet (aspirin, clopidogrel, dipyridamole, ticlopidine), an anticoagulant (heparin, warfarin), or a thrombolytic (tissue plasminogen activator).

Various combinations may be employed; for example, an Effective Compound, such as H₂S, is “A” and the secondary therapy is “B”:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

Administration of the Effective Compounds of the present invention to biological matter will follow general protocols for the administration of that particular secondary therapy, taking into account the toxicity, if any, of the Effective Compound treatment. It is expected that the treatment cycles would be repeated as necessary. It also is contemplated that various standard therapies, as well as surgical intervention, may be applied in combination with the described therapies.

### VII. EXAMPLES

The embodiments in the Example section are understood to be embodiments of the invention that are applicable to all aspects of the invention. The following examples are included to demonstrate some embodiments of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1

### Genetic Studies of Sulfide Activity and Tolerability in C. elegans (FIG. 1)

To define the mechanism by which hydrogen sulfide mediates its effects, the viability of nematodes (Caenorhabditis elegans) with either a wild type genome, a mutation in the gene Hif-1, or a mutation in the gene Egl-9 was tested in air and air with hydrogen sulfide (H₂S) gas at two concentrations. Nematodes of each strain in the fourth larval stage were moved onto fresh nematode growth plates. These plates were then placed in a chamber containing either air, air plus 20 parts per million (ppm) H₂S, or air plus 100 ppm H₂S. The nematodes were removed the next morning and the percent surviving nematodes was determined.

The results shown in FIG. 1 indicate that nematodes lacking the Hif-1 gene were unable to survive in 20 ppm H₂S, whereas wild type nematodes were able to survive exposure to 20 ppm H₂S. In addition, this figure shows that nematodes lacking the Egl-9 gene were resistant to H₂S and survived levels of 100 ppm H₂S, in contrast to wild type nematodes. These results demonstrate that the two genes tested, Hif-1 and Egl-9, mediate the response to H₂S.

### Example 2

### Genetic Studies of Sulfide Activity and Tolerability in Sulfide Adapted C. elegans (FIG. 2)

We tested the ability of nematodes (Caenorhabditis elegans) to survive exposure to H₂S following pre-treatment with H₂S gas. We compared the effects of pre-treatment with H₂S gas using either a wild type genome, or nematodes with a mutation in the gene Egl-9. Wild type nematodes were maintained in either air or air plus 60 ppm H₂S (“H₂S pretreated”). Wild type nematodes, H₂S pretreated nematodes, and Egl-9 mutant nematodes in the fourth larval stage were moved onto fresh nematode growth plates. These plates were then placed in a chamber containing either air or air plus 200 ppm H₂S. The nematodes were removed the next morning and the percent surviving nematodes was determined.

The results shown in FIG. 1 indicate that wild type nematodes raised in air are unable to survive in 200 ppm H₂S, whereas wild type nematodes raised in 60 ppm H₂S are able to survive exposure to 200 ppm H₂S. Nematodes with a mutation in the Egl-9 gene survived exposure 200 ppm H₂S. These results demonstrate that H₂S pretreatment of wild type nematodes is permissive for survival in H₂S.

### Example 3

### HIF-1 GFP Reporter Results

Hif-1 is a transcription factor that can promote transcription of target genes when it is active. In humans, this can result in increased hematocrit and vascular growth, which are beneficial in some circumstances. Using the nematode C. elegans as a model system, we have shown that hydrogen sulfide is capable of inducing HIF-1 activity. In the nematode, nhr-57 protein is known to be produced in a hif-1-dependent manner.

We have used a strain of C. elegans (ZG120) that has an integrated copy of nhr-57 fused to green fluorescent protein (GFP) and which fluoresces green when HIF-1 has been activated. This is confirmed by the fact that worms lacking hif-1 display no fluorescence. Also, worms with constitutively high HIF-1 activity (because they are lacking functional copies of egl-9 or vhl-1, and thus are incapable of degrading hif-1) are highly fluorescent. As assayed using GFP, vhl-1 deletion worms have elevated HIF-1 activity in the gut. In contrast, egl-9 mutant worms have increased HIF-1 activity throughout the body, especially in the head and tail tissue, surrounding the neurons. When wildtype worms bearing nhr-57::GFP are exposed to hypoxia, GFP reaches maximal intensity after five hours of exposure. The resultant GFP pattern is similar to the vhl-1 mutants, with increased GFP throughout the intestine. By contrast, exposure to hydrogen sulfide results in GFP throughout the worm, especially in the head and tail regions surrounding the neurons, with maximum GFP 90 to 150 minutes to following exposure to sulfide. HIF-1 is known to be activated by hypoxia, but this is the first demonstration of induction by sulfide. It is notable, then, that exposure to sulfide results in a more rapid and more robust induction than hypoxia. This supports the concept that sulfide can activate HIF-1 on a timescale that hypoxia cannot, and that the resultant activity can surpass that of hypoxia in both magnitude and tissue distribution.

### Example 4

### HIF Nuclear Localization by Immunostaining

HIF-1 must be transported from the cytoplasm to the nucleus as it is activated, because the nucleus is the site of transcription. To confirm that the GFP activity observed reflected an actual increase in HIF-1 activity a monoclonal antibody was made using amino acids 103-465 of C. elegans HIF-1 (the N-terminus) as an antigen.

Worms were immunostained for HIF-1 protein. As expected, hif-1 null worms have no immunostaining. Egl-9 and vhl-1 mutant worms have greatly increased nuclear HIF-1 localization in most cells throughout the body. Treatment of wildtype worms with sulfide also increases nuclear localization of HIF-1, especially in the head and the tail (including the neurons), with maximum nuclear localization after 30 minutes of sulfide exposure. Previously published data show that maximum hif-1 levels are present after 4 hours of hypoxia exposure, suggesting that hydrogen sulfide has a quicker effect on HIF-1 activation than hypoxia. These data show that there is a discrepancy between cells with nuclear HIF-1 and cells with HIF-1 activity. For instance, vhl-1 null worms have nuclear localized HIF-1 throughout the body, but only display HIF-1 dependent GFP in the gut. Hydrogen sulfide also appears to be capable of localizing HIF-1 in more than just the GFP positive cells. Specifically, sulfide appears to move HIF-1 to the nucleus in the neurons, but transcriptional activity is most evident surrounding the neurons. This suggests that sulfide is capable of affecting more aspects of HIF-1 activation than solely its nuclear localization. In this respect, it may prove to be beneficial in offering a degree of specificity to HIF-1 activation.

### REFERENCES

The following references, to the extent that they provide exemplary procedural or other details supplementary to those set forth herein, are specifically incorporated herein by reference.
U.S. Pat. No. 3,777,507
U.S. Pat. No. 3,881,990
U.S. Pat. No. 3,989,816
U.S. Pat. No. 3,995,444
U.S. Pat. No. 4,034,753
U.S. Pat. No. 4,186,565
U.S. Pat. No. 4,266,573
U.S. Pat. No. 4,292,817
U.S. Pat. No. 4,442,856
U.S. Pat. No. 4,444,762
U.S. Pat. No. 4,447,415
U.S. Pat. No. 4,473,637
U.S. Pat. No. 4,502,295
U.S. Pat. No. 4,559,258
U.S. Pat. No. 4,723,974
U.S. Pat. No. 4,745,759
U.S. Pat. No. 4,765,539
U.S. Pat. No. 4,790,327
U.S. Pat. No. 4,798,824
U.S. Pat. No. 4,828,976
U.S. Pat. No. 4,938,961
U.S. Pat. No. 4,951,482
U.S. Pat. No. 4,962,885
U.S. Pat. No. 5,066,578
U.S. Pat. No. 5,157,930
U.S. Pat. No. 5,217,860
U.S. Pat. No. 5,231,025
U.S. Pat. No. 5,285,657
U.S. Pat. No. 5,326,706
U.S. Pat. No. 5,370,989
U.S. Pat. No. 5,395,314
U.S. Pat. No. 5,399,363
U.S. Pat. No. 5,405,742
U.S. Pat. No. 5,434,045
U.S. Pat. No. 5,466,468
U.S. Pat. No. 5,470,738
U.S. Pat. No. 5,476,763
U.S. Pat. No. 5,543,158
U.S. Pat. No. 5,552,267
U.S. Pat. No. 5,568,910
U.S. Pat. No. 5,569,579
U.S. Pat. No. 5,580,781
U.S. Pat. No. 5,599,659
U.S. Pat. No. 5,607,954
U.S. Pat. No. 5,610,172
U.S. Pat. No. 5,620,995
U.S. Pat. No. 5,620,996
U.S. Pat. No. 5,636,643
U.S. Pat. No. 5,641,515
U.S. Pat. No. 5,645,081
U.S. Pat. No. 5,658,933
U.S. Pat. No. 5,693,462
U.S. Pat. No. 5,699,793
U.S. Pat. No. 5,719,164
U.S. Pat. No. 5,719,174
U.S. Pat. No. 5,726,305
U.S. Pat. No. 5,736,397
U.S. Pat. No. 5,739,169
U.S. Pat. No. 5,752,929
U.S. Pat. No. 5,801,005
U.S. Pat. No. 5,830,880
U.S. Pat. No. 5,846,233
U.S. Pat. No. 5,846,945
U.S. Pat. No. 5,912,019
U.S. Pat. No. 5,916,898
U.S. Pat. No. 5,950,619
U.S. Pat. No. 5,952,168
U.S. Pat. No. 5,954,047
U.S. Pat. No. 5,970,974
U.S. Pat. No. 5,988,162
U.S. Pat. No. 6,013,256
U.S. Pat. No. 6,020,350
U.S. Pat. No. 6,046,046
U.S. Pat. No. 6,054,261
U.S. Pat. No. 6,057,148
U.S. Pat. No. 6,093,730
U.S. Pat. No. 6,100,082
U.S. Pat. No. 6,123,524
U.S. Pat. No. 6,187,529
U.S. Pat. No. 6,195,887
U.S. Pat. No. 6,200,974
U.S. Pat. No. 6,213,723
U.S. Pat. No. 6,213,738
U.S. Pat. No. 6,328,712
U.S. Pat. No. 6,355,019
U.S. Pat. No. 6,365,338
U.S. Pat. No. 6,464,687
U.S. Pat. No. 6,484,721
U.S. Pat. No. 6,490,880
U.S. Pat. No. 6,492,103
U.S. Pat. No. 6,524,785
U.S. Pat. No. 6,552,083
U.S. Pat. No. 6,566,088
U.S. Pat. No. 6,589,229
U.S. Pat. No. 6,602,277
U.S. Pat. No. 6,626,329
U.S. Pat. No. 6,742,992
U.S. Pat. No. 6,764,472
U.S. Pat. No. 6,790,603
U.S. Pat. No. 6,800,096
U.S. Pat. No. 6,805,693
U.S. Pat. No. 6,908,448
U.S. Pat. No. 6,938,619
U.S. Pat. No. 6,958,154
U.S. Pat. No. 7,013,894
U.S. Pat. No. 7,022,107
U.S. Pat. No. 7,029,455
U.S. application Ser. No. 10/480,430
U.S. application Ser. No. 10/971,575
U.S. application Ser. No. 10/971,576
U.S. application Ser. No. 10/972,063
U.S. application Ser. No. 11/408,734
U.S. Pat. Ser. 60/673,037
U.S. Pat. Ser. 60/673,295
U.S. Pat. Ser. 60/713,073
U.S. Pat. Ser. 60/731,549
U.S. Pat. Ser. 60/762,462
U.S. Pat. Ser. 60/793,520
U.S. Publn. 2002/0177203
U.S. Publn. 2003/0153503
U.S. Publn. 2003/0176317
U.S. Publn. 2004/0254215
U.S. Publn. 2005/0227948
U.S. Publn. 2005/0053912
Alam, Antioxid Redox Signal, 4(4):559-62, 2002.
Amersi et al., Hepatology, 35(4):815-823, 2002.
Aravind and Koonin, Genome Biol., 2:RES.0007, 2001.
Areel et al., Hepatology, 25:920-926, 1997.
Austin-Ward and Villaseca, Revista Medica de Chile, 126(7):838-845, 1998.
Behringer et al., Crit. Care Med., 31(5):1523-1531, 2003.
Bellamy et al., Crit. Care Med., 24(2 Suppl):S24-47, 1996.
Bergeron et al., Ann. Neurol. 48:285-296, 2000.
Bernard et al., J. Thorac. Cardiovasc. Surg., 90:235-242, 1985.
Bernard et al., J. Thorac. Cardiovasc. Surg., 90:235-242, 1985.
Bernard et al., N. Engl. J. Med., 346(8):557-563, 2002.
Bickel et al., Hepatology, 28:404-411, 1998.
Bocker-Meffert et al., Invest. Opthalmol. Vis. Sci., 43:2021-2026, 2002.
Brines et al., Proc. Natl. Acad. Sci. USA, 97:10526-10531, 2000.
Brizel, Semin. Radiat. Oncol., 8(4):237-246, 1998.
Brouard et al., J. Biol. Chem., 277(20):17950-17961, 2002.
Bruick and McKnight, Science, 294:1337-1340, 2001.
Buemi et al., Clin. Sci. (Lond), 103:275-282, 2002.
Bukowski et al., Clinical Cancer Res., 4(10):2337-2347, 1998.
Burns & Murphey, Arch. Biochem. Biophys., 339:33-39, 1997.1997
Burns et al., Arch. Biochem. Bipophys, 10:60-68, 1995.
Calapai et al., Eur. J. Pharmacol., 401:349-356, 2000.
Carmeliet et al., Nature, 394:485-490, 1998.
Celik et al., Proc. Natl. Acad. Sci. USA, 99:2258-2263, 2002.
Christodoulides et al., Microbiology, 144(Pt 11):3027-3037, 1998.
Cockman et al., J. Biol. Chem., 275:25733-25741, 2000.
Cohen et al., Ann. Thorac. Surg., 67(5):1489-1491, 1999.
Crosby, Amer. J. Therap., 9:371-376, 2002.
Curran, Seminars Radiation Oncol., 8(4Suppl):2-4, 1998.
Davidson et al., J. Immunother., 21(5):389-398, 1998.
Davis et al., In: Basic Methods in Molecular Biology, 2^{nd} ed., Appleton and Lange (Eds.), Sec. 6-8, 1994.
Dawson, Lancet., 359:96-97, 2002.
Deindl and Schaper, Mol. Cell. Biochem., 186:43-51, 1998.
Detmar et al., J. Invest. Derm., 111:1-6, 1998.
Dhanasekaran et al., J. Biol. Chem., 279:37575-37587, 2004.
Dillman, Cancer Biother. Radiopharm., 14(1):5-10, 1999.
Dorman et al. Neurotoxicol. Teratol., 22(1):71-84, 2000.
Dulak et al., Antioxid. Redox Signal, 4(2):229-240, 2002.
Dupuy et al., Genomics, 69:348-54, 2000.
Elkins et al., J. Biol. Chem., C200644200, 2002.
Elson et al., Genes Dev., 15:2520-2532, 2001.
EP 0650960
EP 0650961
Epstein et al., Cell, 107:43-54, 2001.
Eto et al., Biochem. Biphys. Res. Commun., 293:1483-1488, 2002.
Evans et al., J. Amer. Chem. Soc., 112:4011-4030, 1990.
Franklin et al., Biochem. J., 353:333-338, 2001.
Freeman et al., Mol. Cells, 16:1-12, 2003.
Friedman et al., Proc. Natl. Acad. Sci. USA, 97:4736-4741, 2000.
Gilbert et al., LANCET, 355:375-376, 2000.
Gorio et al., Proc. Natl. Acad. Sci. USA, 99:9450-9455, 2002.
Gorman et al., Toxicology, 187(1):25-38, 2003.
Greene and Wuts, Protecting Groups in Organic Synthesis, 2^{nd} Ed., Wiley, New York, 1991.
Guillemin et al., Cell, 89(1):9-12, 1997.
Hanibuchi et al., Int. J. Cancer, 78(4):480-485, 1998.
Hannan et al., JAMA, 290(6):773-780, 2003.
Hellstrand et al., Acta Oncologica, 37(4):347-353, 1998.
Higuchi and Fukamachi, Folia Pharmacologica Japonica, 73(3):307-319, 1977.
Hochachka et al., Comp. Biochem. Physiol. B Biochem. Mol. Biol., 130(4):435-459, 2001.
Hochachka et al., Proc. Natl. Acad. Sci. USA, 93(18):9493-94938, 1996.
Huang et al., Proc. Natl. Acad. Sci. USA, 95:7987-7992, 1998.
Hui and Hashimoto, Infection Immun., 66(11):5329-5336, 1998.
Hyspler et al., J. Chromatography, 770:255-259, 2002.
Iliopoulus et al., Proc. Natl. Acad. Sci. USA, 93:10595-10599, 1996.
Ivan and Kaelin, Science, 292:464-468, 2001.
Ivan et al., Proc. Natl. Acad. Sci. USA, 99:13459-13464, 2002.
Jaakkola et al., Science, 292:468-472, 2001.
Jackson and Hunt, Methods Enzymol., 96:50-74, 1983.
Jia et al., Proc. Natl. Acad. Sci. USA, 91:7227-7231, 1994.
Jiang et al., J. Biol. Chem., 271:17771-17778, 1996.
Jiang et al., J. Biol. Chem., 272:19253-19260, 1997.
Jin et al., Proc. Natl. Acad. Sci. USA, 97:10242-10247, 2000.
Ju et al., Gene Ther., 7(19):1672-1679, 2000.
Kelso et al., J. Biol. Chem., 276:4588-4596, 2001.
Kilburn and Warshaw, Toxicology Indust. Health, 11(2):185-197, 1995.
Kilburn, Environ. Health, 54(3):150, 1999.
Kilburn, Environ. Res., 81(2):92-99, 1999.
Kivirikko and Myllyharju, Matrix Biol., 16:357-368, 1998.
Kuntscher et al., Br. J. Plast. Surg., 55:430-433, 2002.
Kuroda et al., Transplantation, 46(3):457-460, 1988.
Lando et al., Genes Dev., 16:1466-1471, 2002a.
Lando et al., Science, 295:858-861, 2002b.
Larcher et al., Oncogene, 17:303-311, 1998.
Ledingham et al., Circulation, 82(Part 2):351-358, 1990.
Ledingham et al., J. Thorac. Cardiobasc. Surg., 93:240-246, 1987.
Lee et al., N. Engl. J. Med., 343:148-149, 2000.
Lin et al., Proc. Natl. Acad. Sci. USA, 82:7580-7584, 1985.
Lundgren-Eriksson et al., Anticancer Res., 21(5):3269-3274, 2001.
Mahon et al., Genes Dev., 15:2675-2686, 2001.
Majamaa et al., Biochem. J, 229:127-133, 1985.
Majamaa et al., Eur. J. Biochem., 138:239-245, 1984.
Maxwell et al., Nature, 399:271-275, 1999.
Mehlhorn et al., Cardiovasc Surg., 9(5):482-486, 2001.
Menasche et al., Eur. J. Cardio. Thorax. Surg., 8:207-213, 1994.
Michaels et al., Circulation, 106(23):e187-190, 2002.
Morio et al., Toxicol. Appl. Pharmacol., 172:44-51, 2001.
Myllyharju and Kivirikko, EMBO J., 16:1173-1180, 1997.
Nanji et al., Am. J. Pathol., 146:329-334, 1995.
Nystul et al., Science, 302(5647): 1038-1041, 2003.
Otterbein et al., Am. J. Physiol. Lung Cell Mol. Physiol., 279(6):L1029-L1037, 2000.
Otterbein et al., Trends Immunol., 24(8):449-455, 2003.
Padilla et al., Molec. Biology of the Cell, 13:1473-1483, 2002.
Padilla et al., Proc. Natl. Acad. Sci. USA, 98(13):7331-7335., 2001.
Partlo et al., Neurotoxicology, 22(2):177-189, 2001.
PCT Appln. WO 00/50390
PCT Appln. WO 94/12285
PCT Appln. WO 94/14543
PCT Appln. WO 94/17178
PCT Appln. WO 95/26234
PCT Appln. WO 95/26235
PCT Appln. WO 95/32807
PCT Appln. WO 99/21860
Pelham and Jackson. Eur. J. Biochem. 67:247-256, 1976.
Pietras et al., Oncogene, 17(17):2235-2249, 1998.
Priyadarshi et al., Kidney Int., 61:542-546, 2002.
Pu et al., J. Amer. Chem. Soc., 56:1280-1283, 1991.
Punch et al., 2001
Qin et al., Proc. Natl. Acad. Sci. USA, 95(24):14411-14416, 1998.
Rager et al., NC Med. J., 65(1):18-25, 2004.
Remington's Pharmaceutical Sciences” 15th Edition, pages 1035-1038 and 1570-1580
Richard et al., J. Biol. Chem., 275:26765-26771, 2000.
Ryter and Otterbein, BioEssays, 26:270-280, 2004.
Sakanaka, Proc. Natl. Acad. Sci. USA, 95:4635-4640, 1998.
Sandau et al., Biochem. Biophys. Res. Commun. 278:263-267, 2000.
Sato, Pharmacol. Biochem. Behav., 18(1):443-447, 1983.
Seki et al., Chem. Abstracts, 81(21):424, 1974.
Semenza et al., Adv. Exp. Med. Biol., 475:123-130, 2000.
Semenza et al., Kidney Int., 51:553-555, 1997.
Semenza, Trends Mol. Med., 7(8):345-350, 2001.
Serracino-Inglott et al., BMC Gastroenterol., 2:22-27, 2002.
Sharp et al., J. Cereb. Blood Flow Metab., 20:1011-1032, 2000.
Shaw (1996)
Shen et al., J. Biol. Chem., 280:20580-20588, 2005.
Siddiq et al., J. Biol. Chem., 16:41732-43, 2005.
Siren et al., Proc. Natl. Acad. Sci. USA, 98:4044-4049, 2001.
Smith et al., Eur. J. Biochem., 263:709-716, 1999.
Sodhi et al., Biochem. Biophys. Res. Commun., 287:292-300, 2001.
Srinivas et al., Biochem. Biophys. Res. Commun., 260:557-561, 1999.
Stroka et al., FASEB. J., 15:2445-2453, 2001.
Strubelt, Fundam. Appl. Toxicol., 4:144-151, 1984.
Struve et al., Neurotoxicology, 22(3):375-385, 2001.
Sutter et al., Proc. Natl. Acad. Sci. USA, 97:4748-4753, 2000.
Tacchini et al., Carcinogenesis, 22:1363-1371, 2001.
Tanimoto et al., EMBO. J., 19:4298-4309, 2000.
Taylor, Gene, 275:125-132, 2001.
Teodoro and O'Farrell, EMBO J., 22(3):580-587, 2003.
The Hypothermia After Cardiac Arrest Study Group et al, 2002.
Thornburg et al., Biochem., 32(50):14023-14033, 1993.
Thornton et al., Biochem. J., 350:307-312, 2000.
Thurston et al., Science, 286:2511-2514, 1999.
Tisherman, Crit. Care Med., 32(2):S46-S50, 2004.
Van Voorhies et al., J. Exp. Biol., 203(Pt 16):2467-2478, 2000.
Wang and Semenza, Proc. Natl. Acad. Sci. USA, 90:4304-4308, 1993.
Wang et al., Am. J. Physiol., 263(4 Pt 1):G480-486, 1992.
Wang et al., Cardiovasc. Res., 56:33-42, 2002.
Wang et al., FEBS Letters, 353:138-142, 1994.
Wang et al., Science, 259(5098):1173-1175, 1993.
Welsh et al., Mol Cancer Therapeutics, 2:235-43, 2003.
Williams et al., J. Amer. Chem. Soc., 113:9276-9286, 1991.
Williams, In: Synthesis of Optically Active α-Amino Acids, Pergamon Press, 1989.
Yaffe et al., Crit. Care Med., 32(2):S51-55, 2004.
Yaghi et al., Nature, 423(6941):705-714, 2003.
Yu et al., Proc. Natl. Acad. Sci. USA, 98:9630, 2001.
Zhong et al., Cancer Res., 59:5830-5835, 1999.

1. A method comprising:
(i) identifying biological matter in need of a enhanced survivability, b) prevention or reduction of damage to the biological matter, c) preservation, d) reversibly inhibition of metabolism in the biological matter, e) induction of sleep, f) anesthesia, g) protection from an injury pr prevention of the onset or progression of a disease or death, h) prevention from bleeding to death, i) induction of stasis j) protection from cellular damage from a surgery, k) protection from suffering cellular damage from a disease or adverse medical condition, l) induction of apnea, m) reduction of oxygen demand, n) delaying the effects of a trauma, o) treatment or prevention of hemorrhagic shock, p) induction of hibernation, q) protection from radiation therapy or chemotherapy, r) treatment of a hyperproliferative disease, s) inhibition of rejection of an organ transplant, t) treatment of hypothermia, u) induction of cardioplegia in biological matter undergoing bypass surgery, v) prevention of hematologic shock, w) wound healing, x) prevention or treatment of neurodegeneration; and
(ii) providing to the biological matter at least one compound with a chemical formula selected from the following groups:
(a) compound of Formula (Ia);
(b) compound of Formula (Ib);
(c) compound of Formula (Ic);
(d) compound of Formula (Id);
(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
or a salt, ester, or precursor thereof,
wherein stasis or pre-stasis is induced or wherein the alpha-subunit of hypoxia inducible factor (HIFα) is stabilized.

2.-27. (canceled)

28. The method of claim 1, wherein preserving the biological material comprises perfusing the biological matter with a cryoprotectant and lowering the temperature of the biological matter.

29. The method of claim 1, wherein the biological matter is provided or contacted with the compound in an amount and for a time sufficient to cause the biological matter to enter stasis.

30. The method of claim 1, wherein the biological matter is provided or contacted with the compound in an amount and for a time sufficient to stabilize the alpha-subunit of hypoxia inducible factor (HIFα) in cells of the biological matter.

31. The method of claim 1, further comprising incubating the biological matter under hypoxic or anoxic conditions.

32. (canceled)

33. The method of claim 31, wherein the hypoxic or anoxic conditions would damage the biological matte in the absence of the compound.

34.-37. (canceled)

38. The method of claim 1, wherein the biological matter is provided with a combination of compounds.

39. The method of claim 38, further comprising providing the biological matter with at least one compound with a chemical formula selected from the following groups:
(n) Formula (I);
(o) Formula (IV);
(p) carbon monoxide;
(q) chalcogenide compound;
(r) H₂S or other sulfur containing compound;
(s) protective metabolic agent;
(t) oxygen antagonist;
or a salt, ester, or precursor thereof.

40.-50. (canceled)

51. The method of claim 1, wherein the biological matter is provided the compound by administration to the biological matter or organism intravenously, intradermally, intraarterially, intraperitoneally, intralesionally, intracranially, intraarticularly, intraprostaticaly, intrapleurally, intratracheally, intranasally, intrathecally, intravitreally, intravaginally, intrarectally, intratumorally, intramuscularly, intraperitoneally, intraocularly, subcutaneously, subconjunctival, intravesicularlly, mucosally, intrapericardially, intraumbilically, intraocularally, orally, topically, locally, by inhalation, by injection, by infusion, by continuous infusion, by localized perfusion, via a catheter, via a lavage, or through catheterization, immersion, absorption, or adsorption.

52.-55. (canceled)

56. The method of claim 1, wherein one or more compounds comprise a cationic structure capable of targeting the one or more compounds to mitochondria.

57.-59. (canceled)

60. The method of claim 1, wherein the biological matter includes platelets.

61.-62. (canceled)

63. The method of claim 1, wherein the biological matter is at risk for hemorrhagic shock.

64. (canceled)

65. The method of claim 1, wherein the biological matter is hemorrhaging.

66. The method of claim 65, wherein the biological matter goes into hemorrhagic shock.

67.-69. (canceled)

70. A method for stabilizing the alpha-subunit of hypoxia inducible factor (HIFα) in biological matter, the method comprising administering at least one compound with a chemical formula selected from the following groups:
(n) Formula (I);
(o) Formula (IV);
(p) carbon monoxide;
(q) chalcogenide compound;
(r) H₂S or other sulfur containing compound;
(s) protective metabolic agent;
(t) oxygen antagonist;
or a salt, ester, or precursor thereof,
wherein the compound inhibits 2-oxoglutarate dioxygenase enzyme activity, wherein the compound inhibits hydroxylation of HIFα, and/or wherein the compound inhibits HIF prolyl hydroxylase enzyme activity.

71. The method of claim 70, wherein the 2-oxoglutarate dioxygenase enzyme is selected from the group consisting of EGLN1, EGLN2, EGLN3, procollagen prolyl 4-hydroxylase, procollagen prolyl 3-hydroxylase, procollagen lysyl hydroxylase, PHD4, FIH-1, and any subunit or fragment thereof.

72. (canceled)

73. The method of claim 70, wherein the HIF prolyl hydroxylase enzyme is selected from the group consisting of EGLN1, EGLN2, EGLN3, and any subunit or fragment thereof.

74. A method for treating, preventing, or pretreating a HIF-associated condition in biological matter, the method comprising administration of at least one compound with a chemical formula selected from the following groups:
(n) Formula (I);
(o) Formula (IV);
(p) carbon monoxide;
(q) chalcogenide compound;
(r) H₂S or other sulfur containing compound;
(s) protective metabolic agent;
(t) oxygen antagonist;
or a salt, ester, or precursor thereof;
wherein HIFα is stabilized wherein 2-oxoglutarate dioxygenase enzyme activity is inhibited, and/or wherein HIF prolyl hydroxylase enzyme activity is inhibited.

75.-76. (canceled)

77. The method of claim 74, wherein the HIF-associated condition is associated with hypoxia or ischemia.

78. The method of claim 77, wherein the HIF-associated condition is associated with a pulmonary disorder, a cardiac disorder, a neurological disorder or with an ischemic event.

79.-82. (canceled)

83. A method of treating, preventing or pretreating a condition mediated at least in part by hypoxia inducible factor (HIF) and/or erythropoietin (EPO), said method comprising administering to a subject an effective amount of at least one compound with a chemical formula selected from the following groups:
(n) Formula (I);
(o) Formula (IV);
(p) carbon monoxide;
(q) chalcogenide compound;
(r) H₂S or other sulfur containing compound;
(s) protective metabolic agent;
(t) oxygen antagonist;
or a salt, ester, or precursor thereof;
wherein HIFα is stabilized.

84. The method of claim 83, wherein said condition is selected from the group consisting of anemic disorders; neurological disorders and/or injuries; including cases of stroke, trauma, epilepsy, neurodegenerative disease, myocardial infarction, liver ischemia, renal ischemia, and stroke; peripheral vascular disorders, ulcers, burns, and chronic wounds; pulmonary embolism; and ischemic-reperfusion injury.

85. The method of claim 83, further comprising providing to the biological matter at least one compound with a chemical formula selected from the following groups:
(a) compound of Formula (Ia);
(b) compound of Formula (Ib);
(c) compound of Formula (Ic);
(d) compound of Formula (Id);
(e) compound of Formula (II);
(f) compound of Formula (III);
(g) compound of Formula (V);
(h) compound of Formula (VI);
(i) compound of Formula (VII);
(j) compound of Formula (VIII);
(k) compound of Formula (IX);
(l) compound of Formula (X);
(m) compound of Formula (XI);
(u) ACE inhibitor (ACEI);
(v) angiotensin-II receptor blocker (ARB);
(w) diuretic;
(x) digoxin;
(y) statin;
(z) carnitine;
or a salt, ester, or precursor thereof.

86.-88. (canceled)

89. A method comprising administration of at least one compound with a chemical formula selected from the following groups:
(n) Formula (I);
(o) Formula (IV);
(p) carbon monoxide;
(q) chalcogenide compound;
(r) H₂S or other sulfur containing compound;
(s) protective metabolic agent;
(t) oxygen antagonist;
or a salt, ester, or precursor thereof;
wherein HIFα is stabilized and wherein expression of angiogenic factors is increased in the biological matter, expression of glycolytic factors is increased in the biological matter, expression of factors associated with oxidative stress is increased in the biological matter.

90.-91. (canceled)

92. A method of treating a subject having a disorder associated with ischemic reperfusion injury, the method comprising administration of at least one compound with a chemical formula selected from the following groups:
(n) Formula (I);
(o) Formula (IV);
(p) carbon monoxide;
(q) chalcogenide compound;
(r) H₂S or other sulfur containing compound;
(s) protective metabolic agent;
(t) oxygen antagonist;
or a salt, ester, or precursor thereof;
wherein HIFα is stabilized.

93. (canceled)

94. The method of claim 83, further comprising providing to the organism at least one compound with a chemical formula selected from the following groups:
(aa) heterocyclic carboxamides;
(bb) phenanthrolines;
(cc) hydroxamates;
or a salt, ester, or precursor thereof.

95. The method of claim 94, wherein the heterocyclic carboxamides are selected from the group consisting of pyridine carboxamides, quinoline carboxamides, isoquinoline carboxamines, cinnoline carboxamides and beta-carboline carboxamides.

96.-97. (canceled)

98. The method of claim 1, wherein the biological matter is an organ, tissue, or cell from the heart, lung, kidney, liver, bone marrow, pancreas, skin, bone, vein, artery, cornea, blood, small intestine, large intestine, brain, spinal cord, smooth muscle, skeletal muscle, ovary, testis, uterus, or umbilical cord.

99. The method of claim 1, wherein the biological matter comprises the following cell types: platelet, myelocyte, erythrocyte, lymphocyte, adipocyte, fibroblast, epithelial cell, endothelial cell, smooth muscle cell, skeletal muscle cell, endocrine cell, glial cell, neuron, secretory cell, barrier function cell, contractile cell, absorptive cell, mucosal cell, limbus cell (from cornea), stem cell, unfertilized or fertilized oocyte, or sperm.

100. (canceled)

101. The method of claim 1, wherein the biological matter or organism is a mammal.

102.-106. (canceled)
